SEQUENCE LISTING (4 sequences)

SEQ ID NO: 1 - RNA, 22 nt, Artificial Sequence
  miRNA
ugagguaguagguuguaugguu

SEQ ID NO: 2 - RNA, 22 nt, Artificial Sequence
  miRNA
ugagguaggagguuguauaguu

SEQ ID NO: 3 - RNA, 48 nt, Artificial Sequence
  miRNA
uuuuuuuuuuuuuuuuuuuuuuuugagacggagucucgcucugucgcc

SEQ ID NO: 4 - RNA, 15 nt, Artificial Sequence
  miRNA
guuguguuguguugu

(19) United States
(12) Patent Application Publication
(10) Pub. No.: US 2020/0316051 A1
(43) Pub. Date: Oct. 8, 2020

(54) TLR7/8 ANTAGONISTS AND USES THEREOF

(71) Applicant: Merck Patent GmbH, Darmstadt (DE)

(72) Inventors: Brian A. Sherer, Nashua, NH (US); Jaromir Vlach, Westford, MA (US)

(73) Assignee: Merck Patent GmbH, Darmstadt (DE)

(21) Appl. No.: 16/946,347

(22) Filed: Jun. 17, 2020

Related U.S. Application Data
(63) Continuation of application No. PCT/US2018/065112, filed on Dec. 12, 2018.
(60) Provisional application No. 62/607,406, filed on Dec. 19, 2017.

(51) Int. Cl. A61K 31/4545; A61K 31/4985; A61K 31/519; A61K 31/4709; A61K 31/5377; A61K 31/498; A61K 31/551; A61P 25/28

(52) U.S. Cl. CPC A61K 31/4545; A61K 31/4985; A61K 31/519; A61P 25/28; A61K 31/5377; A61K 31/498; A61K 31/551; A61K 31/4709

(57) ABSTRACT

A treatment of disorders related to TLR7/8 overexpression or aberrant activation is provided. The disorder may include multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction. A compound of Formula (I) is useful to treat such disorders.

### CROSS-REFERENCE TO RELATED APPLICATIONS

This application is a Continuation of International Application No. PCT/US2018/065112, filed on Dec. 12, 2018, and which claims the benefit of U.S. Provisional Application No. 62/607,406, filed on Dec. 19, 2017, the contents of both of which are hereby incorporated by reference in their entireties.

### REFERENCE TO A SEQUENCE LISTING

The present application is accompanied by an ASCII text file which has been submitted via EFS-Web as a computer readable form containing the sequence listing, titled “2020-05-13-Sequence-listing-as-filed.txt”, created on May 13, 2020, with the file size of 947 bytes, which is incorporated by reference in its entirety.

### BACKGROUND OF THE INVENTION

### Field of the Invention

The present invention provides for the treatment of disorders related to TLR7/8 overexpression or aberrant TLR7/8 activity, such as, multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, Congenital heart block, autoimmune hepatitis, autoimmune pancreatitis, adult onset Still's disease, drug-induced neurological disorders, and substance addiction, using a compound of Formula (I).

### Discussion of the Background

Toll-like receptors (TLR) currently comprising a gene family of 10 receptors with different specificities are part of the cellular pathogen pattern recognition system, which has evolved for defense against a variety of infections (bacteria, virus, fungi). Activation of TLRs leads to cytokine responses, e.g. with release of interferons and activation of specified immune cells. The functional expression of selected TLRs in tissues is highly different. Part of the receptors are located at the cell surface such as TLR4 (stimulated by E. coli lipopolysaccharide LPS), e.g. on epithelial cells, or TLR3, 7, 8 and 9 located at endosomal membranes in specified immune cells. The latter are all activated by nucleic acids, but recognize various types of them. For instance, TLR9 is activated by single stranded DNA containing CpG subsequences, TLR7 and 8 are activated by single stranded RNA, and TLR3 is activated by double-stranded RNA.

TLRs have been implicated in various autoimmune and inflammatory diseases, with the clearest example being the role played by TLR7 in the pathogenesis of systemic lupus erythematosus (Barrat and Coffman, Immunol Rev, 223:271-283, 2008). Additionally, a TLR8 polymorphism has been associated with rheumatoid arthritis (Enevold et al., J Rheumatol, 37:905-10, 2010). Although various TLR7, TLR8 and TLR9 inhibitors have been described, additional TLR inhibitors are desirable. In particular, polynucleotides having inhibitory motifs for one or more of TLR7, TLR8 and TLR9 are needed to precisely inhibit an immune response in a subject (e.g., patient having an autoimmune disease or an inflammatory disorder).

### SUMMARY OF THE INVENTION

In one aspect, the invention provides a method for the treatment of disorders related to TLR7/8 overexpression or TLR7/8 aberrant activation, comprising the step of administering to a patient a compound of Formula (I):

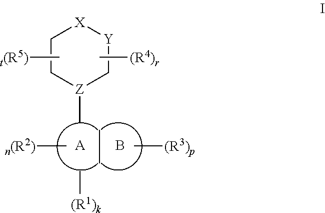

and pharmaceutically acceptable derivatives, solvates, salts, hydrates and stereoisomers thereof.

In another aspect the invention provides a compound of Formula (I) above—or any pharmaceutically acceptable derivative, solvate, salt, hydrate or stereoisomer thereof—for use in the treatment of disorders related to TLR7/8 overexpression or TLR7/8 aberrant activation.

In certain embodiments, the disorder is selected from multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction.

1. A method for the treatment of disorders related to TLR7/8 overexpression or TLR7/8 aberrant activation, comprising the step of administering to a patient a compound of formula I,

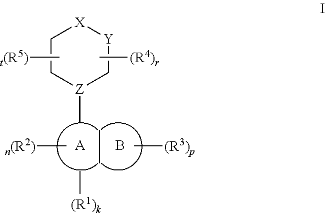

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
each R¹ is independently absent, —H, —CH₃, —CF₃, —CN, —F, —Cl, —OCH₃, —OC₂H₅, or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;

### Z is N or CH;

each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0, 1 or 2;
n is 0, 1, or 2;
p is 0, 1, or 2;
r is 0, 1, or 2; and
t is 0, 1, or 2.

2. The method of embodiment 1, wherein the disorder is selected from multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction.

3. The method of any previous embodiment, wherein the compound is a compound of formula II,

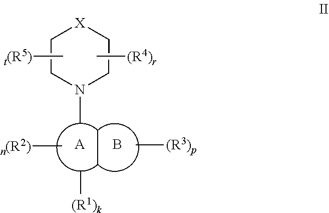

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is absent, —H, —CHF₂, —CF₃, —OMe, —OC₂H₅, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

4. The method of any previous embodiment, wherein the compound is a compound of formula III,

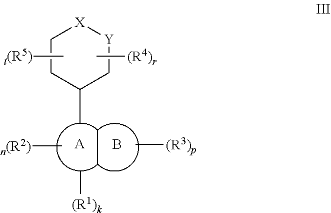

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is heteroaryl having 1-4 heteroatoms independently selected from nitrogen, oxygen, or sulfur; each of which is optionally substituted;
R¹ is —H, —CH₃, —CF₃, —CN, —F, —Cl, —OCH₃, or —OCF₃;
each R² is independently —H, —R, halogen, -haloalkyl, —R, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
   each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
   two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
   k is 1 or 2;
   n is 0, 1, or 2;
   p is 0, 1, or 2;
   r is 0, 1, or 2; and
   t is 0, 1, or 2.

5. The method of any previous embodiment, wherein the compound is selected from Table 1 and Table 2.

6. Compound of Formula I

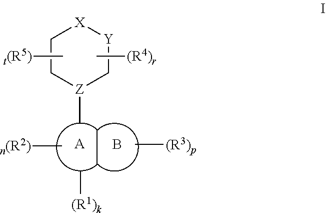

or a pharmaceutically acceptable salt, solvate, hydrate or stereoisomer thereof, for use in the treatment of a disorder related to TLR7/8 overexpression or TLR7/8 aberrant activation, wherein in formula I:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
each R¹ is independently absent, —H, —CH₃, —CF₃, —CN, —F, —Cl, —OCH₃, —OC₂H₅, or —OCF₃;
   each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
   each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
   X is C(R⁴)₂, O, NR⁴, S, S(R⁴), or S(R⁴)₂;
   Y is C(R⁴)₂, O, NR⁴, S, S(R⁴), or S(R⁴)₂;

### Z is N or CH;

each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0, 1 or 2;
n is 0, 1, or 2;
p is 0, 1, or 2;
r is 0, 1, or 2; and
t is 0, 1, or 2.

7. The compound for use of embodiment 6, wherein the disorder is selected from multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction.

8. The compound for use of any of embodiments 6 and 7, wherein the compound is a compound of formula II,

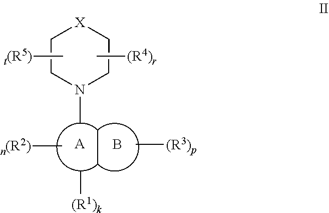

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is absent, —H, —CHF₂, —CF₃, —OMe, —OC₂H₅, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —R, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

9. The compound for use of any of embodiments 6 to 8, wherein the compound is a compound of formula III,

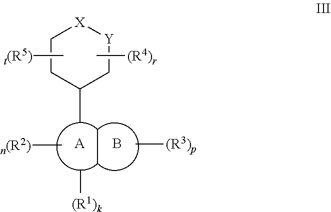

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is heteroaryl having 1-4 heteroatoms independently selected from nitrogen, oxygen, or sulfur; each of which is optionally substituted;
R¹ is —H, —CH₃, —CF₃, —CN, —F, —Cl, —OCH₃, or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 1 or 2;
n is 0, 1, or 2;
p is 0, 1, or 2;
r is 0, 1, or 2; and
t is 0, 1, or 2.

10. The compound for use of any of embodiments 6 to 9, wherein the compound is selected from Table 1 and Table 2.

### BRIEF DESCRIPTION OF THE DRAWINGS

FIG. 1 shows the effect of miRNA treatment on the level of cytokine IL-6 in human peripheral blood lymphocytes.

FIG. 2 shows the effect of miRNA treatment on the level of cytokine INFα in human peripheral blood lymphocytes.

FIG. 3 shows the effect of a TLR7/8 inhibitor on the level of cytokine IL-6 in human peripheral blood lymphocytes.

FIG. 4 shows the effect of a TLR7/8 inhibitor on the level of cytokine INFα in human peripheral blood lymphocytes.

FIG. 5 shows the effect of a TLR7/8 inhibitor on the level of cytokine IL-6 in human peripheral blood lymphocytes pretreated with LL37 protein.

FIG. 6 shows the effect of a TLR7/8 inhibitor on the level of cytokine INFα in human peripheral blood lymphocytes pretreated with LL37 protein.

### DETAILED DESCRIPTION OF THE INVENTION

### 1. Compounds and Definitions

Compounds of this invention include those described generally above, and are further illustrated by the classes, subclasses, and species disclosed herein. Without being limited thereto they include compounds disclosed in International Patent Applications published as WO 2017/106607 A1 and WO 2018/031434 A1. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th}Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Fused rings, as described herein, are described by embodiments for each ring; Ring A and Ring B. Together, Ring A and Ring B form a fused heteroaryl ring as allowed by valence

(e.g., when Ring A is

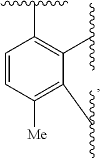 [SMILES: *c1ccc(C)c(*)c1*]

### and Ring B is

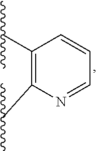 [SMILES: *c1cccnc1*]

then together Ring A and Ring B is

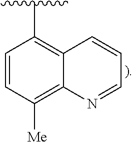 [SMILES: *c1ccc(C)c2ncccc12]

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

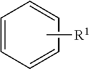

refers to at least

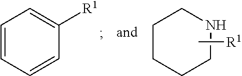

refers to at least

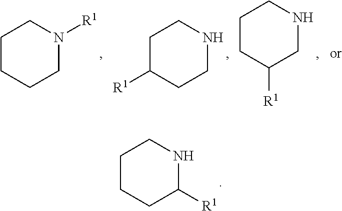

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{∘}; —(CH₂)₀₋₄₀R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which are optionally substituted with R^{∘}; —(CH₂)₀₋₄O(CH₂)₀₋₁Ph which is optionally substituted with R^{∘}; —CH═CHPh, which is optionally substituted with R^{∘}; —(CH₂)₀₋₄O(CH₂)₀₋₁-pyridyl which is optionally substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} is optionally substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{∘} (or the ring formed by taking two independent occurrences of R^{∘} together with their intervening atoms), are independently deuterium, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R¹ is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R*, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:

—F, —Cl, —Br, —I, deuterium,

—OH, protected hydroxy, alkoxy, oxo, thiooxo,

—NO₂, —CN, CF₃, N₃,

—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,

—O— alkyl, —O— alkenyl, —O— alkynyl, —O— cycloalkyl, —O-aryl, —O-heteroaryl, —O-heterocyclic,

—C(O)— alkyl, —C(O)— alkenyl, —C(O)— alkynyl, —C(O)— carbocyclyl, —C(O)-aryl, —C(O)— heteroaryl, —C(O)-heterocyclyl,

—CONH₂, —CONH— alkyl, —CONH— alkenyl, —CONH— alkynyl, —CONH-carbocyclyl, —CONH-aryl, —CONH-heteroaryl, —CONH-heterocyclyl,

—OCO₂-alkyl, —OCO₂-alkenyl, —OCO₂-alkynyl, —OCO₂-carbocyclyl, —OCO₂-aryl, —OCO₂-heteroaryl, —OCO₂-heterocyclyl, μ-OCONH₂, —OCONH— alkyl, —OCONH— alkenyl, —OCONH— alkynyl, —OCONH— carbocyclyl, —OCONH-aryl, —OCONH— heteroaryl, —OCONH— heterocyclyl,

—NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂-alkyl, —NHCO₂-alkenyl, —NHCO₂-alkynyl, —NHCO₂-carbocyclyl, —NHCO₂-aryl, —NHCO₂-heteroaryl, —NHCO₂-heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH-heteroaryl, —NHC(O)NH— heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH-alkenyl, —NHC(NH)NH— alkenyl, —NHC(NH)NH— carbocyclyl, —NHC(NH)NH-aryl, —NHC(NH)NH-heteroaryl, —NHC(NH)NH— heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl,

—C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl,

—S(O)— alkyl, —S(O)-alkenyl, —S(O)-alkynyl, —S(O)-carbocyclyl, —S(O)-aryl, —S(O)-heteroaryl, —S(O)-heterocyclyl —SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl,

—NHSO₂-alkyl, —NHSO₂-alkenyl, —NHSO₂-alkynyl, —NHSO₂-carbocyclyl, —NHSO₂-aryl, —NHSO₂-heteroaryl, —NHSO₂-heterocyclyl,

—CH₂NH₂, —CH₂SO₂CH₃,

-mono-, di-, or tri-alkyl silyl,

-alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

There is furthermore intended that a compound of the formula I includes isotope-labeled forms thereof. An isotope-labeled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the formula I by well-known methods include isotopes of hydrogen, carbon, nitrogen, oxygen, phos-phorus, fluo-rine and chlorine, for example ²H, ³H, ¹³C, ¹⁴C, ¹⁵N, ¹⁸O, ¹⁷O, ³¹P, ³²P, ³⁵S, ¹⁸F and ³⁶CI, respectively. A compound of the formula I, a prodrug, thereof or a pharmaceutically acceptable salt of either which contains one or more of the above-mentioned isotopes and/or other isotopes of other atoms is intended to be part of the present invention. An isotope-labeled compound of the formula I can be used in a number of beneficial ways. For example, an isotope-labeled compound of the formula I into which, for example, a radioisotope, such as ³H or ¹⁴C, has been incorporated, is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the formula I has therapeutic advantages owing to the higher metabolic stability of this isotope-labeled compound. Higher metabolic stability translates directly into an increased in vivo half-life or lower dosages, which under most circumstances would represent a preferred embodiment of the present invention. An isotope-labeled compound of the formula I can usually be prepared by carrying out the procedures disclosed in the synthesis schemes and the related description, in the example part and in the preparation part in the present text, replacing a non-isotope-labeled reactant by a readily available isotope-labeled reactant.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a com-pound of the formula I that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative metabolism. Significant improvements in the pharmacokinetic profiles of compounds of the formula I are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

The following is intended to illustrate the above: a compound of the formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound of the formula I can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

As used herein, the term “modulator” is defined as a compound that binds to and/or inhibits the target with measurable affinity. In certain embodiments, a modulator has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in TLR7/8 activity between a sample comprising a compound of the present invention, or composition thereof, and TLR7/8, and an equivalent sample comprising TLR7/8, in the absence of said compound, or composition thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### 2. Description of the Invention

According to one aspect, the present invention provides a method for the treatment of disorders related to TLR7/8 overexpression or TLR7/8 aberrant activation, comprising the step of administering to a patient a compound of formula I,

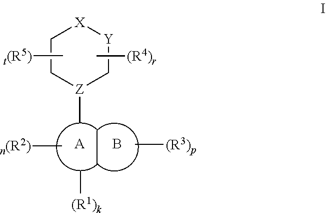

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
each R¹ is independently absent, —H, —CH₃, —CF₃, —CN, —F, —Cl, —OCH₃, —OC₂H or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;
Z is N or CH;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0, 1 or 2;
n is 0, 1, or 2;
p is 0, 1, or 2;
r is 0, 1, or 2; and
t is 0, 1, or 2.

According to another aspect, the present invention provides a compound of Formula (I) as defined above—or a pharmaceutically acceptable derivative, solvate, salt, hydrate or stereoisomer thereof—for use in the treatment of disorders related to TLR7/8 overexpression or TLR7/8 aberrant activation. Further it is understood that wherever in this specification or in the accompanying claims it is referred to or claimed a method of treatment of a disorder or disorders by making use or administering a compound of any of the Formulas specified herein this disclosure refers as well to the respective compound of the Formula specified for use in the treatment of such a disorder or such disorders.

In certain embodiments, the disorder is selected from multiple sclerosis, Alzheimer's Disease, myositis, stroke, ischemia, CNS neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction.

In certain embodiments, the present invention provides the method as described above, wherein the compound is a compound of formula H,

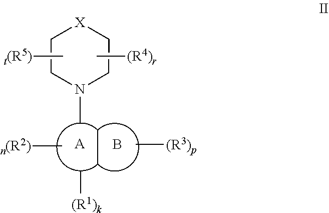

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is absent, —H, —CHF₂, —CF₃, —OMe, —OC₂H₅, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R), or S(R)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

In certain embodiments, R¹ is absent.

In certain embodiments, R¹ is, —H.

In certain embodiments, R¹ is —CHF₂.

In certain embodiments, R¹ is —CF₃.

In certain embodiments, R¹ is —OMe.

In certain embodiments, R¹ is —OC₂H₅.

In certain embodiments, R¹ is —CN.

In certain embodiments, Ring A is C₆ aryl or a 6 membered monocyclic heteroaryl having 1-4 heteroatoms independently selected from nitrogen, oxygen, or sulfur; each of which is optionally substituted.

In certain embodiments, Ring A is phenyl, pyridyl, pyrimidinyl, pyrazinyl, pyridazinyl, or triazinyl; each of which is optionally substituted.

In certain embodiments, Ring A is phenyl, pyridyl, or pyrimidinyl; each of which is optionally substituted.

In certain embodiments, Ring A is

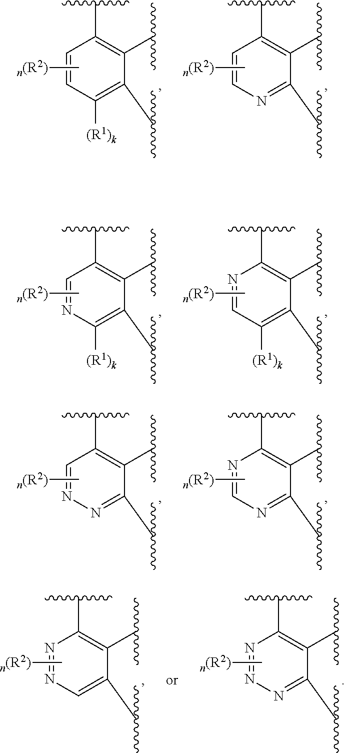

In certain embodiments, Ring A is

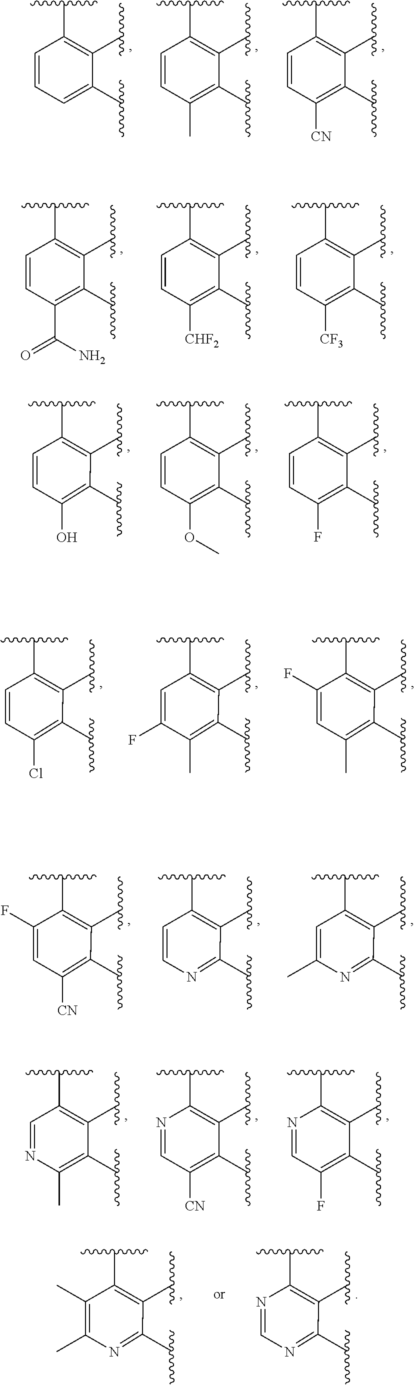

In certain embodiments, Ring B is C₆ aryl or a 5-6 membered monocyclic heteroaryl having 1-4 heteroatoms independently selected from nitrogen, oxygen, or sulfur; each of which is optionally substituted.

In certain embodiments, Ring B is phenyl, pyridyl, pyrimidinyl, pyrazinyl, pyridazinyl, triazinyl, pyrrole, imidazole, isoxazole, oxazole, or thiazole; each of which is optionally substituted.

In certain embodiments, Ring B is

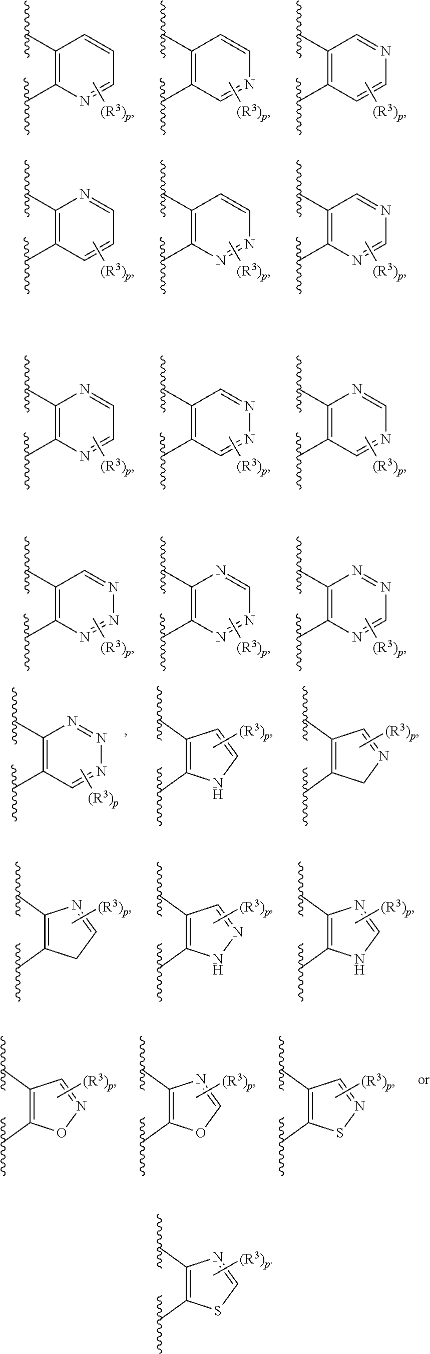

In certain embodiments Ring B is

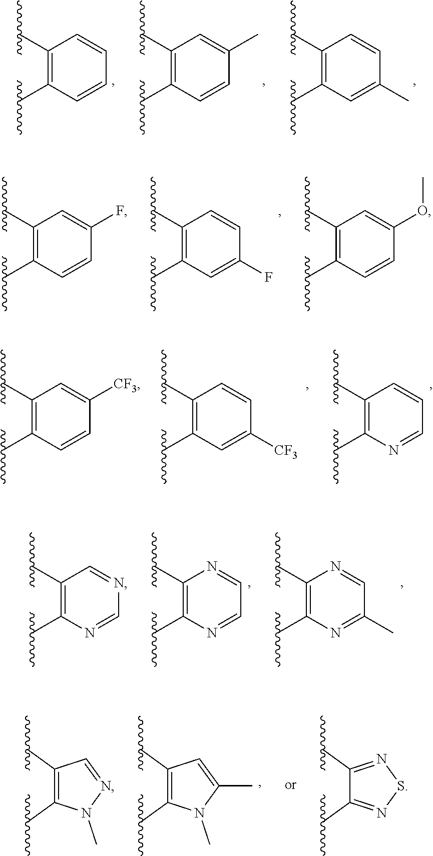

In certain embodiments, each R² is independently —H.

In certain embodiments, each R² is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R² is independently methyl, ethyl, ethyl, propyl, i-propyl, butyl, s-butyl, t-butyl, straight or branched pentyl, or straight or branched hexyl; each of which is optionally substituted.

In certain embodiments, each R² is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R² is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R³ is independently —H.

In certain embodiments, each R³ is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R³ is independently methyl, ethyl, ethyl, propyl, i-propyl, butyl, s-butyl, t-butyl, straight or branched pentyl, or straight or branched hexyl; each of which is optionally substituted.

In certain embodiments, each R³ is independently methyl.

In certain embodiments, each R³ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R³ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, X is C(R⁴)₂ or O.

In certain embodiments, X is C(R⁴)₂. In certain embodiments, X is CH₂.

In certain embodiments, X is O.

In certain embodiments, each R⁴ is independently —H.

In certain embodiments, each R⁴ is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁴ is independently methyl, ethyl, ethyl, propyl, i-propyl, butyl, s-butyl, t-butyl, straight or branched pentyl, or straight or branched hexyl; each of which is optionally substituted.

In certain embodiments, each R⁴ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁴ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R⁴ is independently —H, C₁₋₆ aliphatic, —OR, —C(O)R, —CO₂R, —C(O)N(R)₂, —NRC(O)R, —NRC(O)N(R)₂, —NRSO₂R, or —N(R)₂; each of which is optionally substituted.

In certain embodiments, each R⁴ is independently —H, C₁₋₆ aliphatic, —C(O)N(R)₂, —NRC(O)R, or —N(R)₂, each of which is optionally substituted.

In certain embodiments, each R⁴ is independently

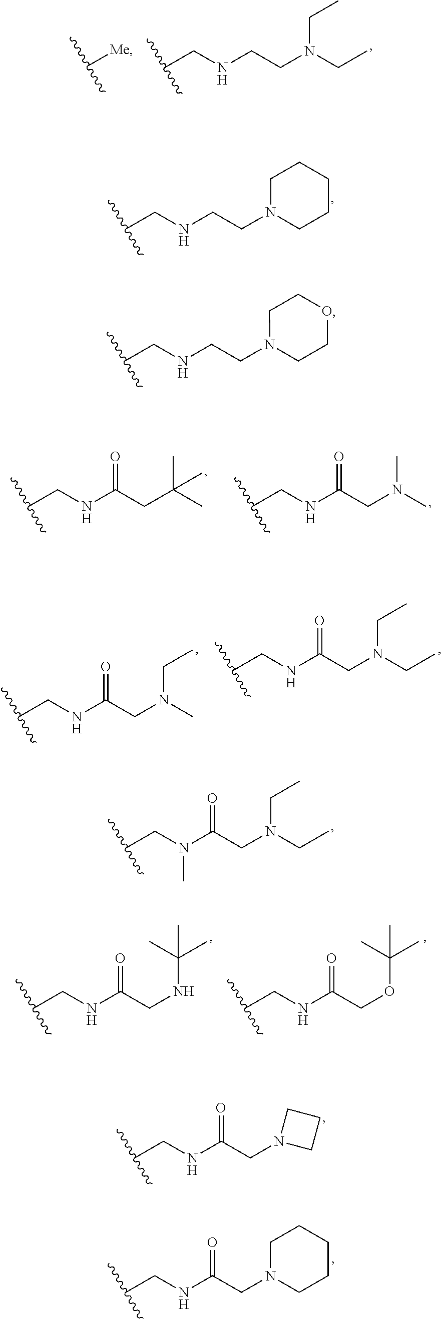

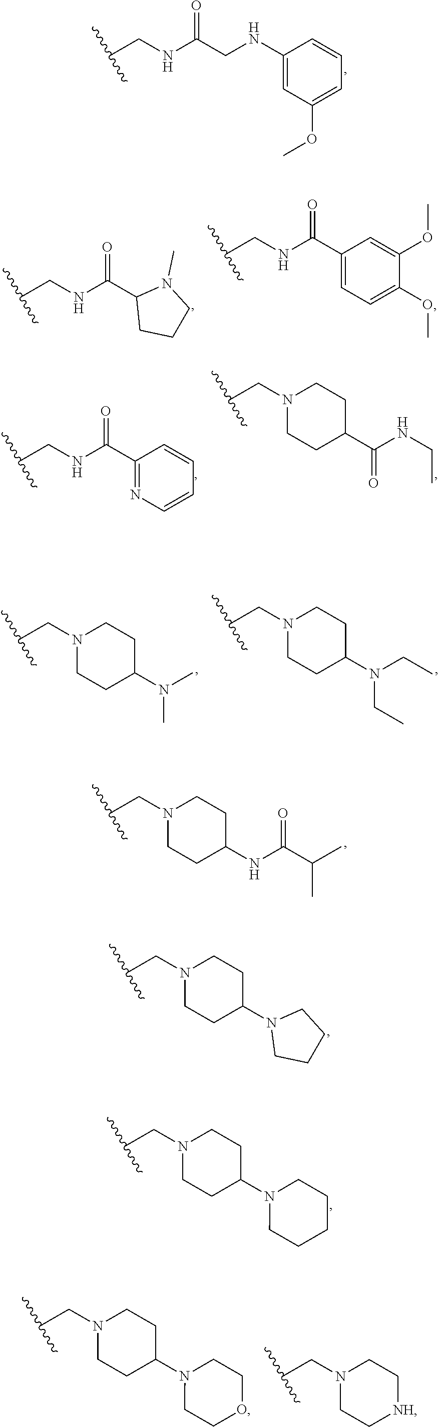

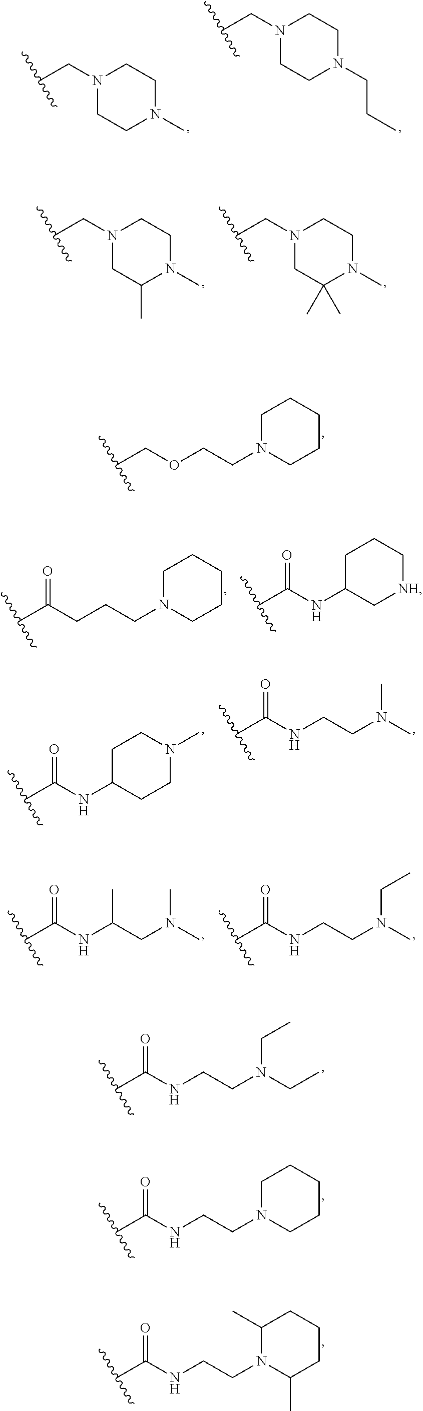

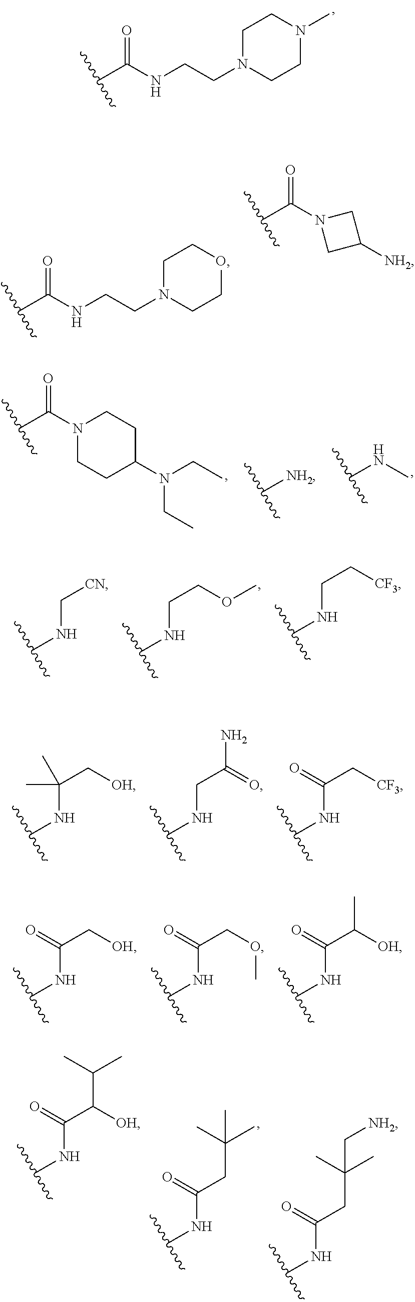

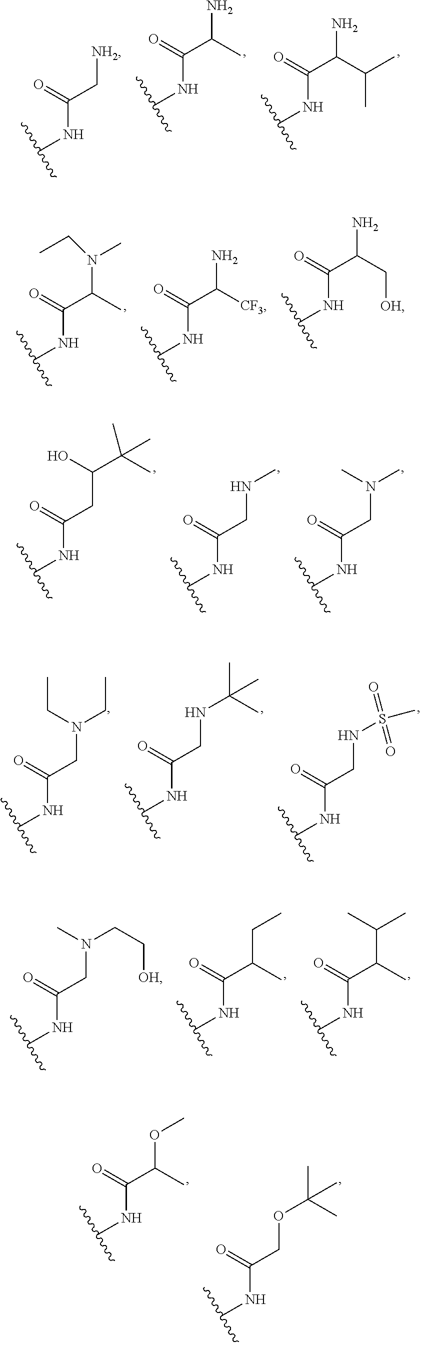

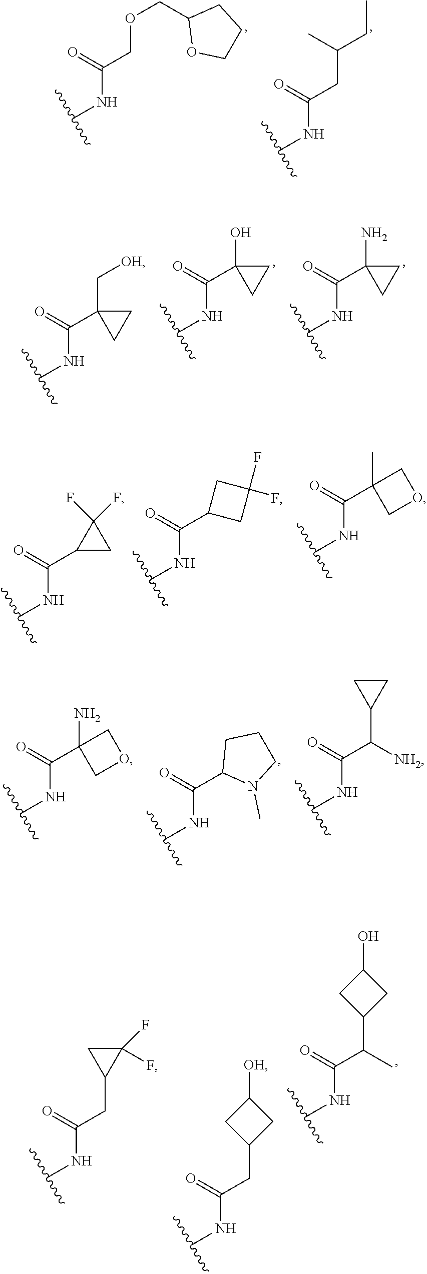

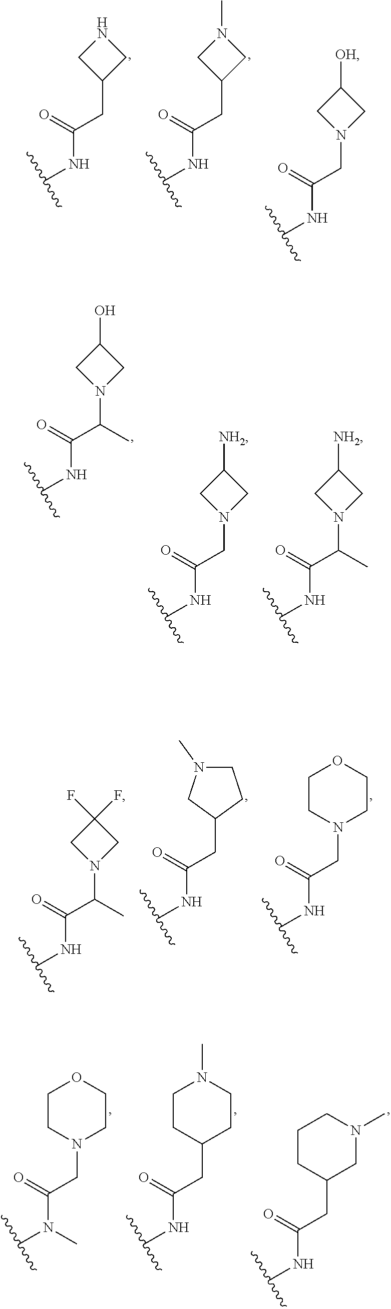

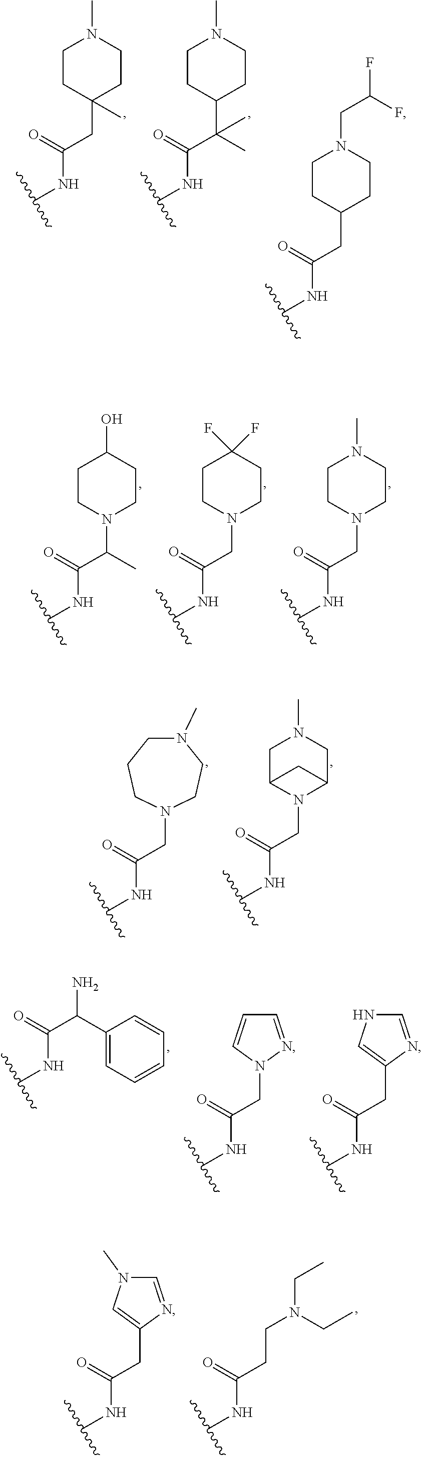

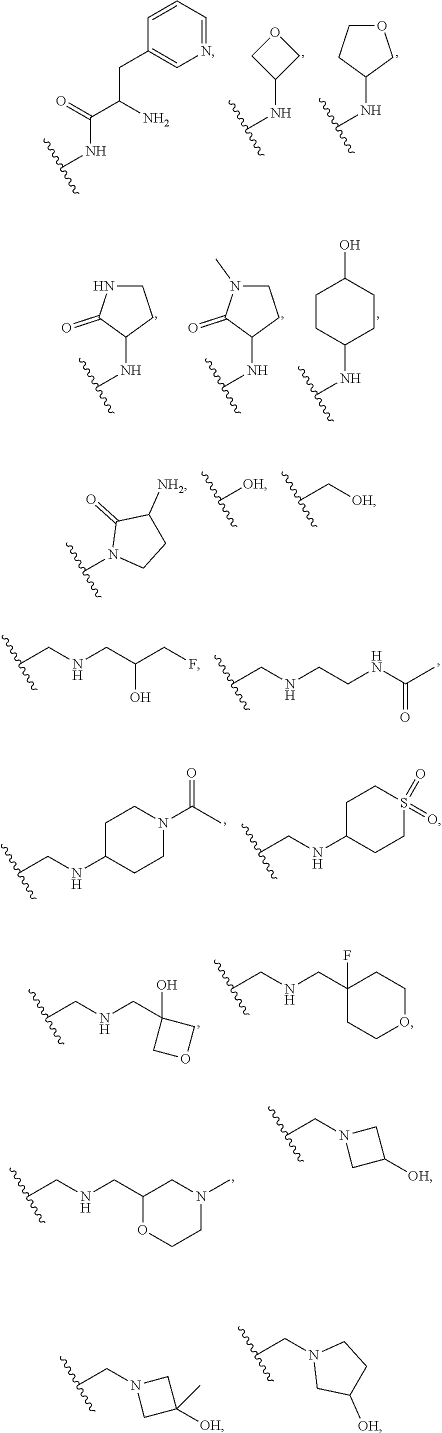

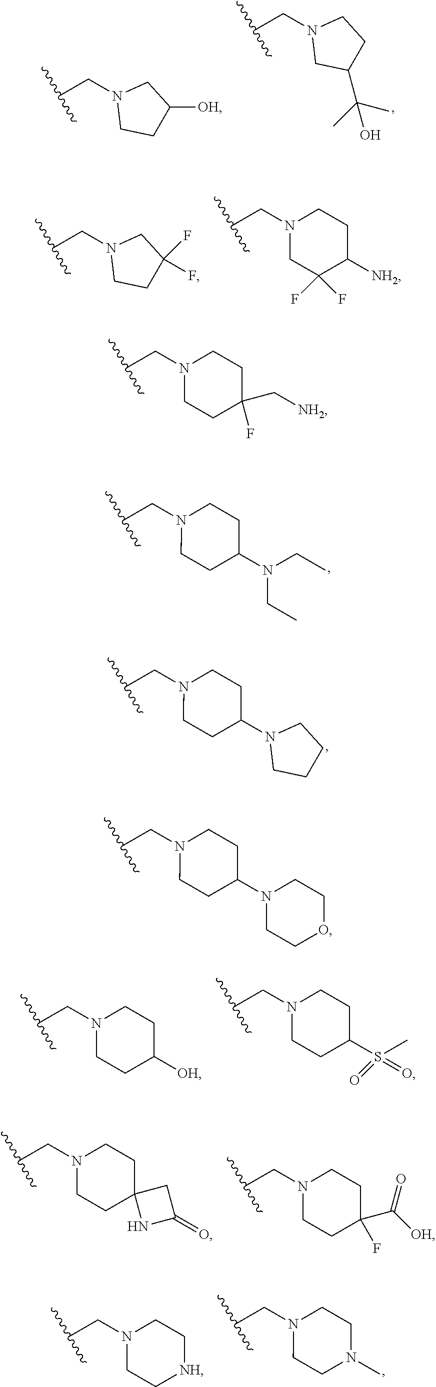

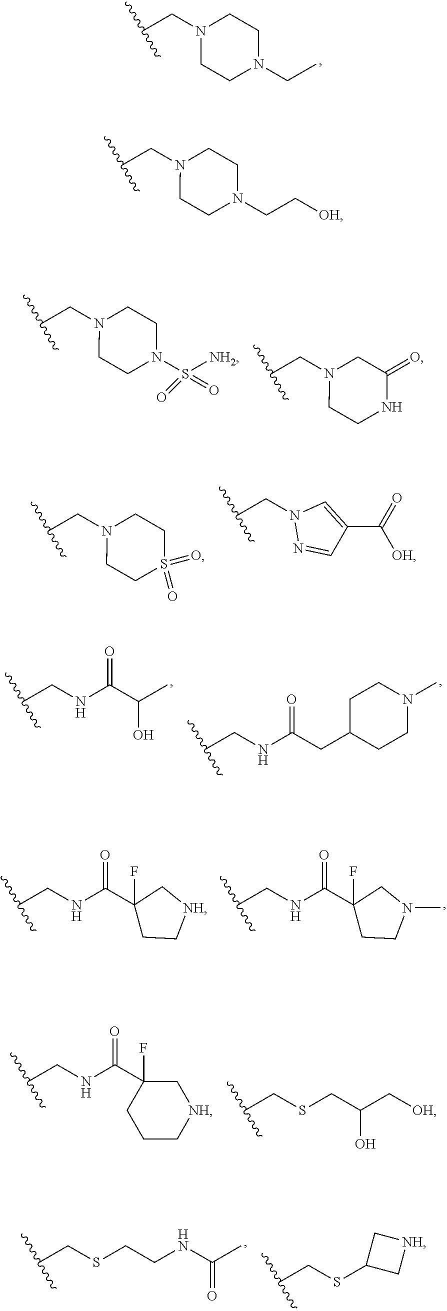

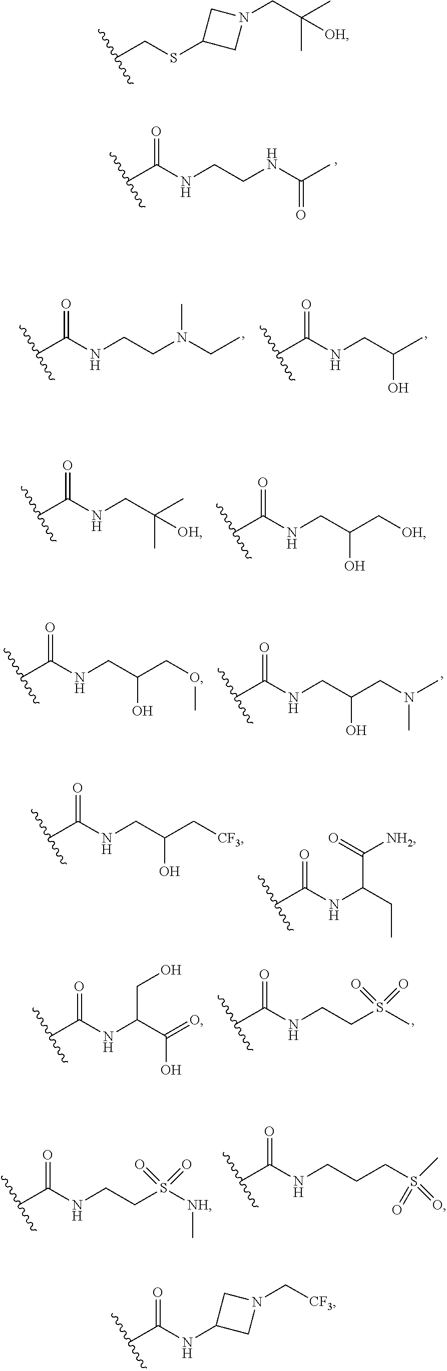

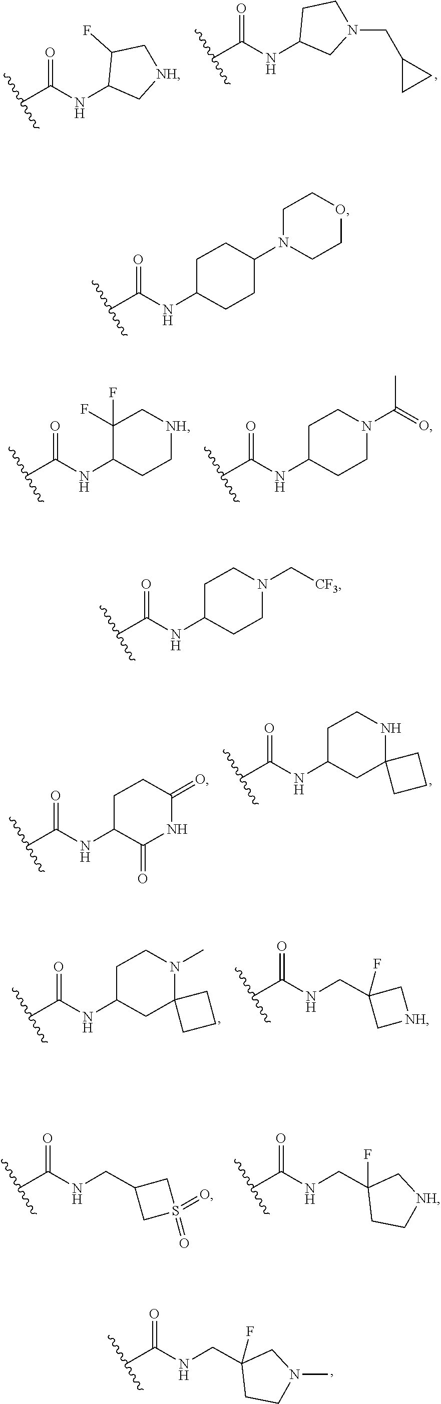

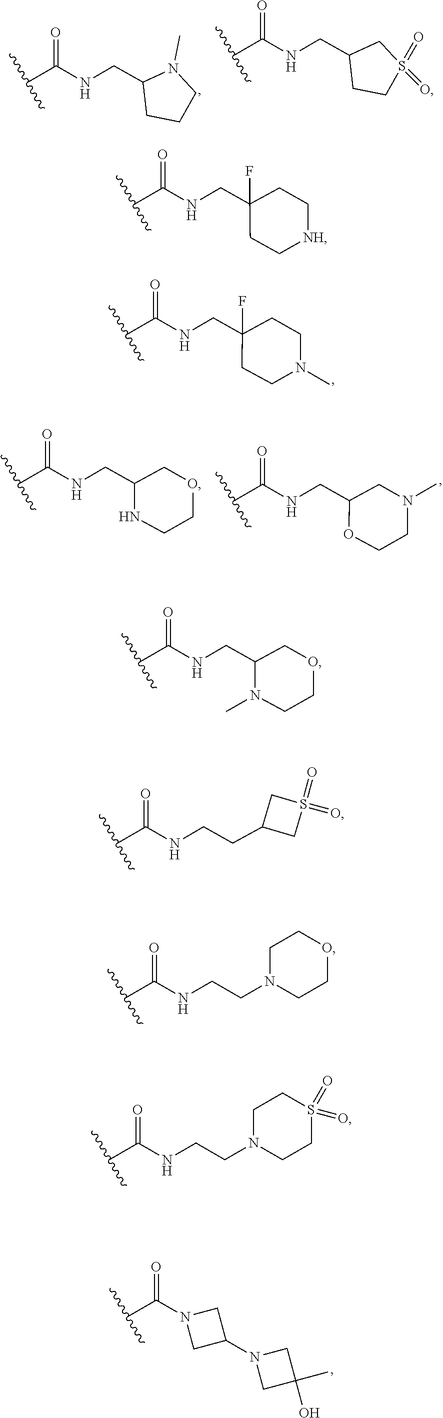

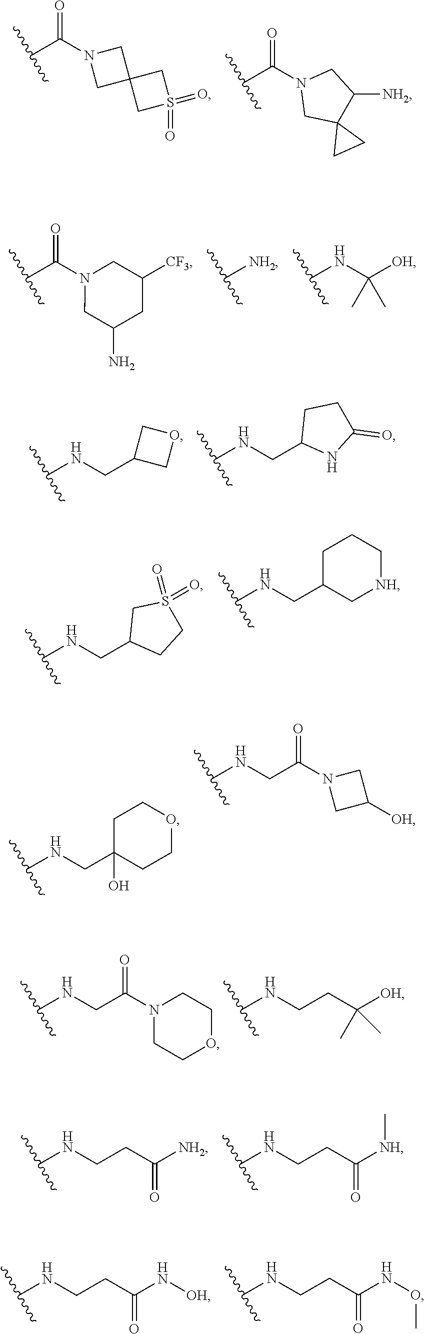

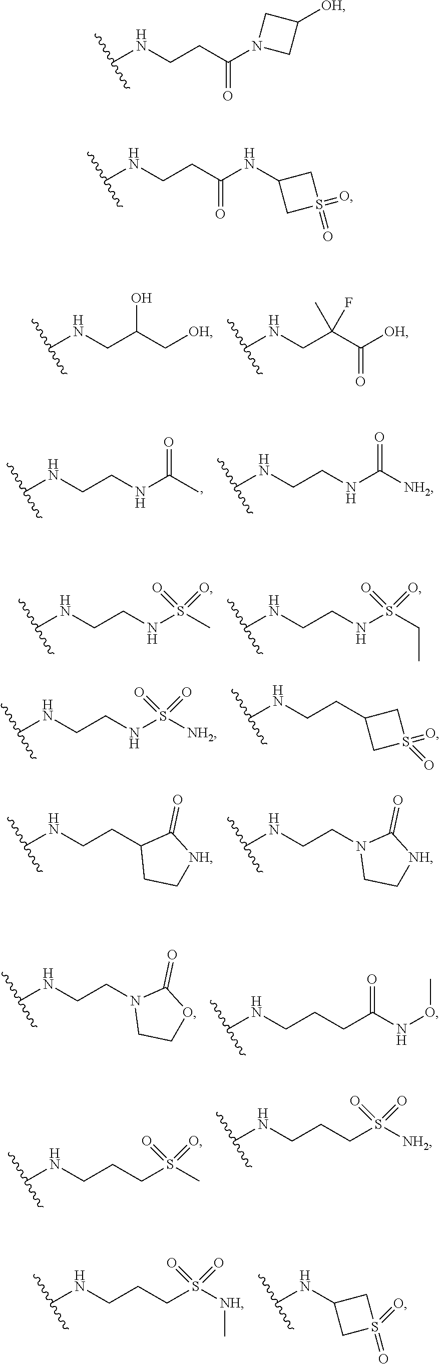

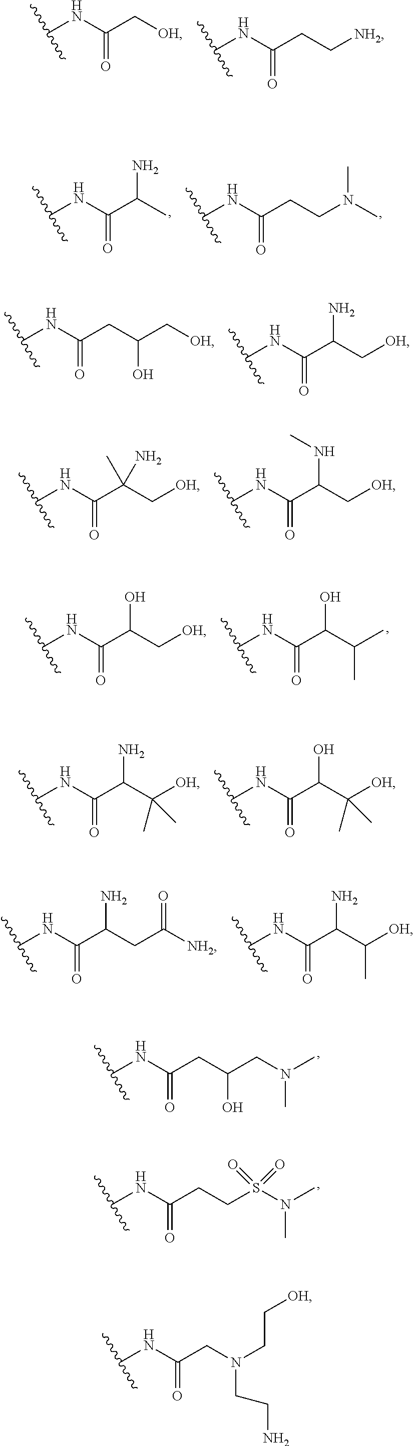

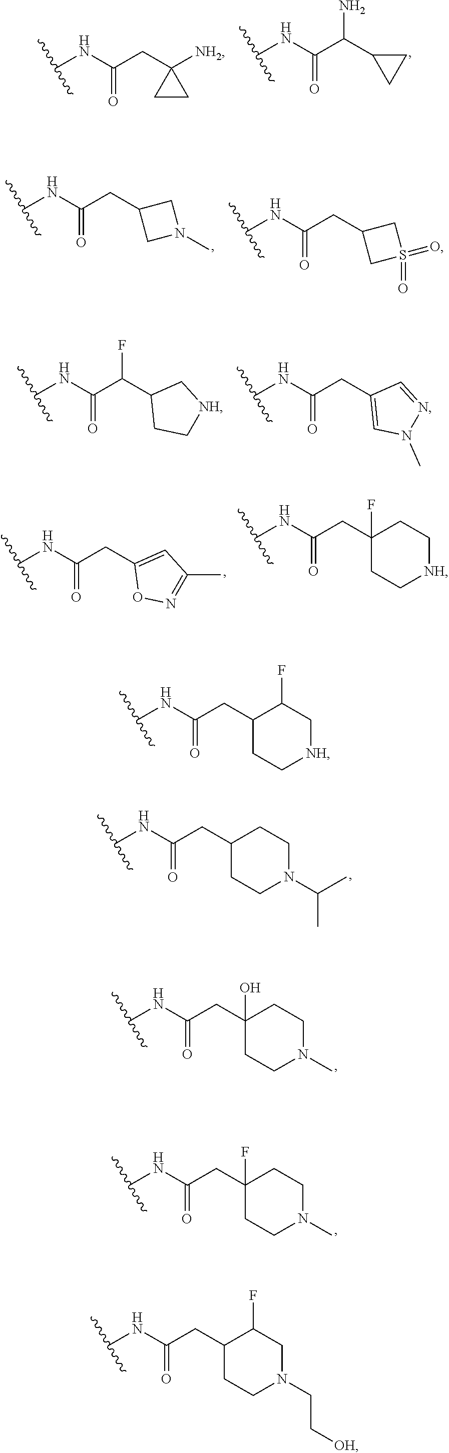

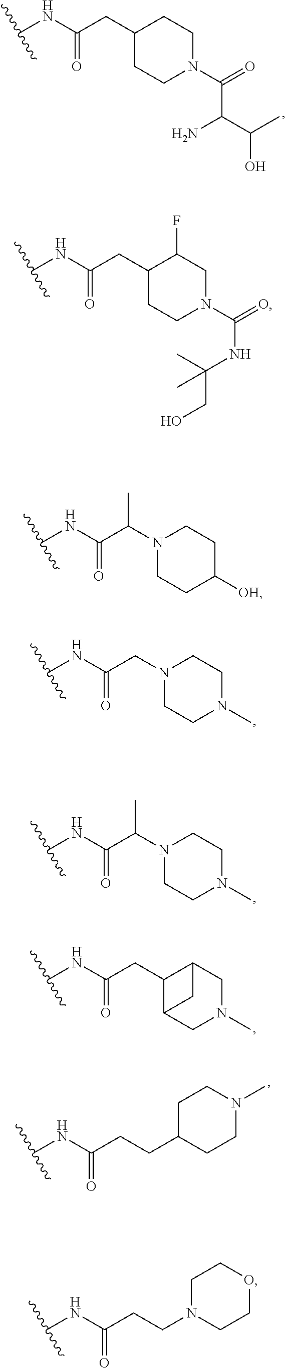

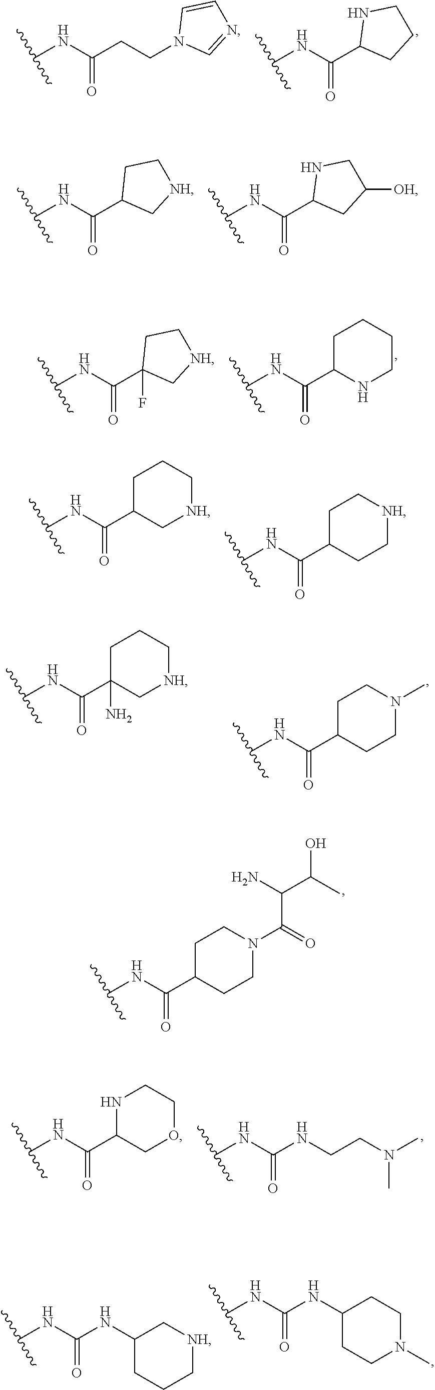

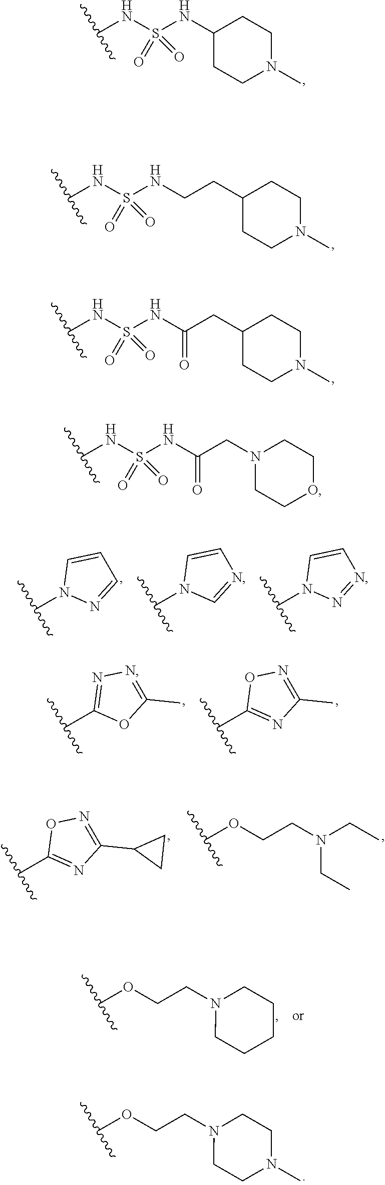

In certain embodiments, each R⁴ is independently

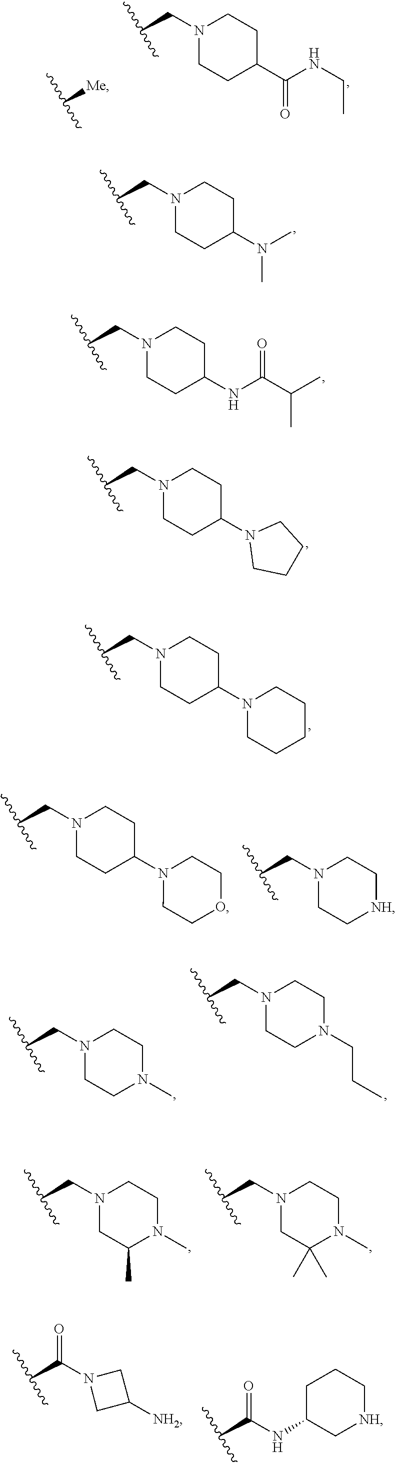

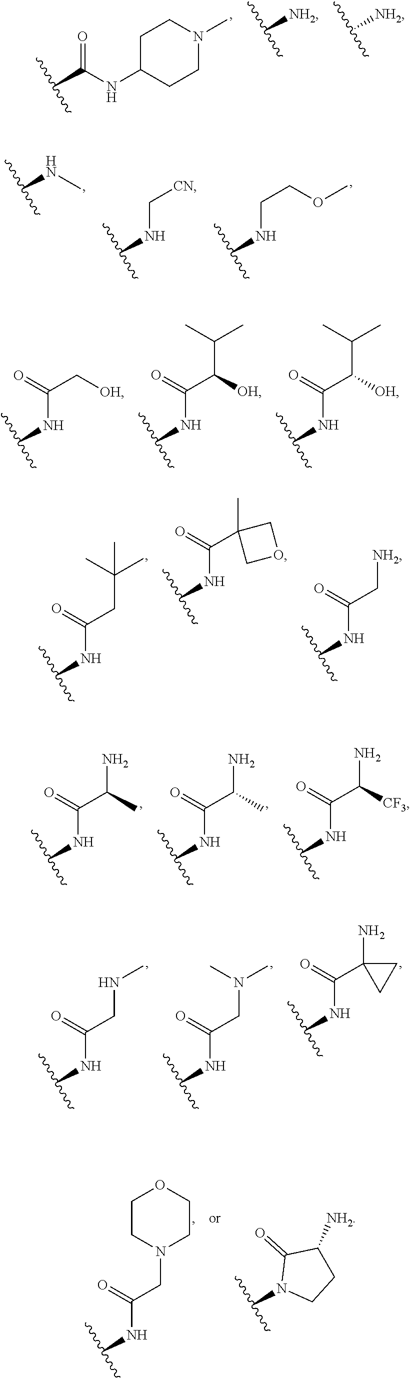

In certain embodiments, each R⁵ is independently —H.

In certain embodiments, each R⁵ is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁵ is independently methyl, ethyl, ethyl, propyl, i-propyl, butyl, s-butyl, t-butyl, straight or branched pentyl, or straight or branched hexyl; each of which is optionally substituted.

In certain embodiments, each R⁵ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁵ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R⁵ is independently methyl, cyclopropyl, —F, or —CF₃.

In certain embodiments, each R⁵ is independently

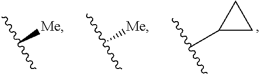

### —F, or —CF₃.

In certain embodiments, each of X, Ring A, Ring B, R¹, R², R³, R⁴, R⁵, k, m, n, p, r, and t, is as defined above and described in embodiments, classes and subclasses above and herein, singly or in combination.

In certain embodiments, the present invention provides the method as described above, wherein the compound is a compound of formula II-a,

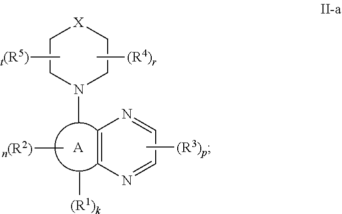

or a pharmaceutically acceptable salt thereof, wherein each of X, Ring A, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-b,

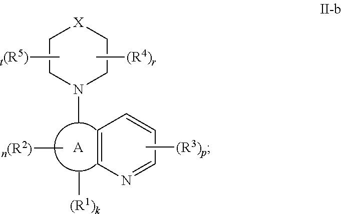

or a pharmaceutically acceptable salt thereof, wherein each of X, Ring A, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-c,

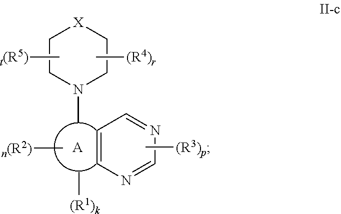

or a pharmaceutically acceptable salt thereof, wherein each of X, Ring A, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-d,

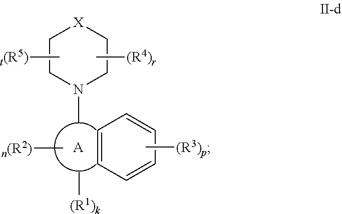

or a pharmaceutically acceptable salt thereof, wherein each of X, Ring A, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-e,

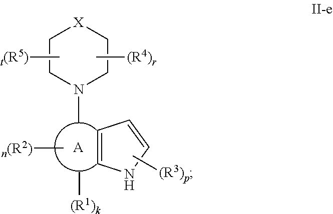

or a pharmaceutically acceptable salt thereof, wherein each of X, Ring A, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-f,

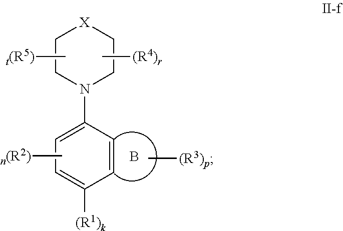

or a pharmaceutically acceptable salt thereof, wherein each of X, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-g,

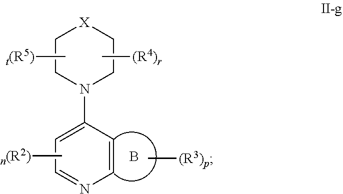

or a pharmaceutically acceptable salt thereof, wherein each of X, Ring B, R², R³, R⁴, R⁵, n, p, r, and t, is as defined above and described in embodiments, classes and subclasses above and herein, singly or in combination.

In certain embodiments, the present invention provides the method as described above, wherein the compound is a compound of formula II-h,

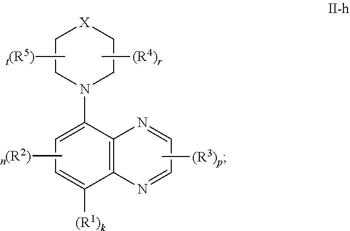

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-j,

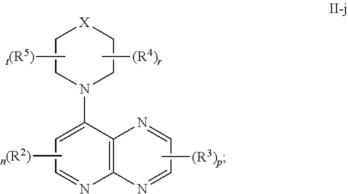

or a pharmaceutically acceptable salt thereof, wherein each of X, R², R³, R⁴, R⁵, n, p, r, and t, is as defined above and described in embodiments, classes and subclasses above and herein, singly or in combination.

In certain embodiments, the present invention provides the method as described above, wherein the compound is a compound of formula II-m,

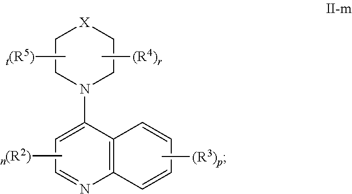

or a pharmaceutically acceptable salt thereof, wherein each of X, R², R³, R⁴, R⁵, n, p, r, and t, is as defined above and described in embodiments, classes and subclasses above and herein, singly or in combination.

In certain embodiments, the present invention provides the method as described above, wherein the compound is a compound of formula II-n,

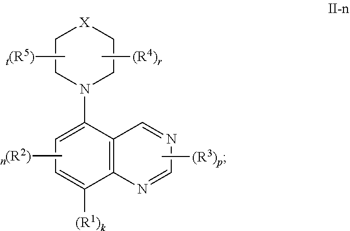

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-p,

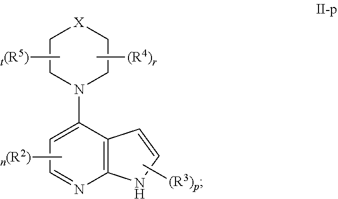

or a pharmaceutically acceptable salt thereof, wherein each of X, R², R³, R⁴, R⁵, n, p, r, and t, is as defined above and described in embodiments, classes and subclasses above and herein, singly or in combination.

In certain embodiments, the present invention provides the method as described above, wherein the compound is a compound of formula II-q,

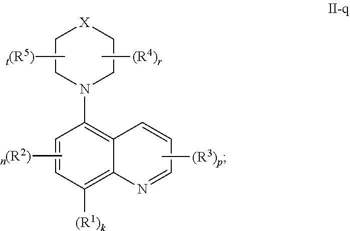

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-r,

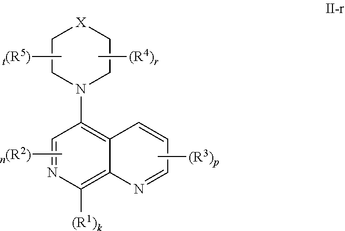

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-s,

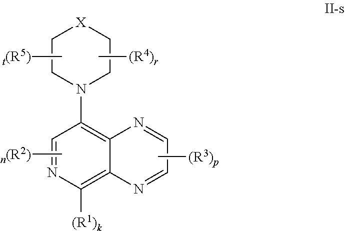

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula II-t,

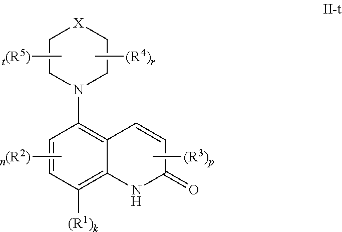

or a pharmaceutically acceptable salt thereof, wherein each of X, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III,

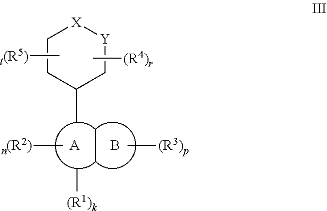

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is heteroaryl having 1-4 heteroatoms independently selected from nitrogen, oxygen, or sulfur; each of which is optionally substituted:
R¹ is —H, —CH₃, —CF₃, —CN, —F, —Cl, —OCH₃, or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂,
X is C(R⁴)₂, O, NR⁴, S, S(R⁴), or S(R⁴)₂;
Y is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂,
each R¹ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 1 or 2;
n is 0, 1, or 2;
p is 0, 1, or 2;
r is 0, 1, or 2; and
t is 0, 1, or 2.

In certain embodiments, R¹ is —H.

In certain embodiments, R¹ is —CH₃.

In certain embodiments, R¹ is —CF₃.

In certain embodiments, R¹ is —CN.

In certain embodiments, R¹ is —F.

In certain embodiments, R¹ is —Cl.

In certain embodiments, R¹ is —OCH₃.

In certain embodiments, R¹ is —OCF₃.

In certain embodiments, Ring A is phenyl or a 5-6 membered monocyclic heteroaryl having 1-4 heteroatoms independently selected from nitrogen, oxygen, or sulfur.

In certain embodiments, Ring A is phenyl or a 6 membered monocyclic heteroaryl having 1-4 heteroatoms independently selected from nitrogen, oxygen, or sulfur.

In certain embodiments, Ring A is phenyl, pyridyl, pyrimidinyl, pyrazinyl, pyridazinyl, or triazinyl.

In certain embodiments, Ring A is phenyl or pyridyl.

In certain embodiments, Ring A is

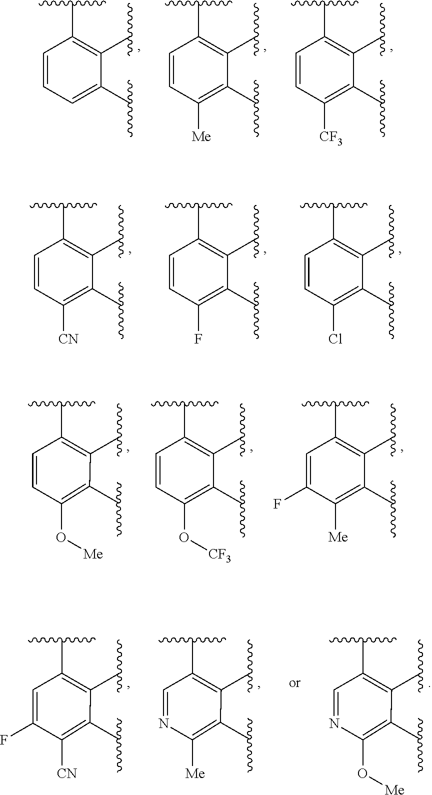

In certain embodiments, Ring A is

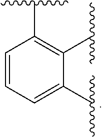 [SMILES: *c1cccc(*)c1*]

In certain embodiments, Ring A is

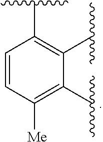 [SMILES: *c1ccc(C)c(*)c1*]

In certain embodiments, Ring A is

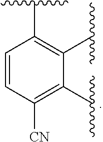 [SMILES: *c1ccc(C#N)c(*)c1*]

In certain embodiments, Ring A is

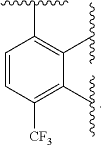 [SMILES: *c1ccc(C(F)(F)F)c(*)c1*]

In certain embodiments, Ring A is

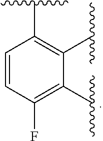 [SMILES: *c1ccc(F)c(*)c1*]

In certain embodiments, Ring A is

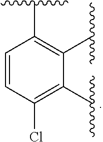 [SMILES: *c1ccc(Cl)c(*)c1*]

In certain embodiments, Ring A is

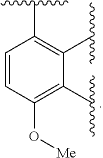 [SMILES: *c1ccc(OC)c(*)c1*]

In certain embodiments, Ring A is

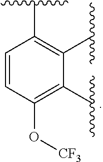 [SMILES: *c1ccc(OC(F)(F)F)c(*)c1*]

In certain embodiments, Ring A is

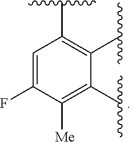 [SMILES: *c1cc(F)c(C)c(*)c1*]

In certain embodiments, Ring A is

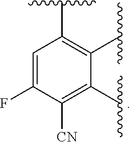 [SMILES: *c1cc(F)c(C#N)c(*)c1*]

In certain embodiments, Ring A is

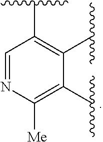 [SMILES: *c1cnc(C)c(*)c1*]

In certain embodiments, Ring A is

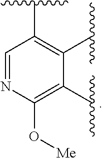 [SMILES: *c1cnc(OC)c(*)c1*]

In certain embodiments, Ring B is a 5-6 membered monocyclic heteroaryl having 1-4 heteroatoms independently selected from nitrogen, oxygen, or sulfur.

In certain embodiments, Ring B is pyridyl, pyrimidinyl, pyrazinyl, pyridazinyl, triazinyl, pyrrole, imidazole, isoxazole, oxazole, or thiazole; each of which is optionally substituted.

In certain embodiments, Ring B is

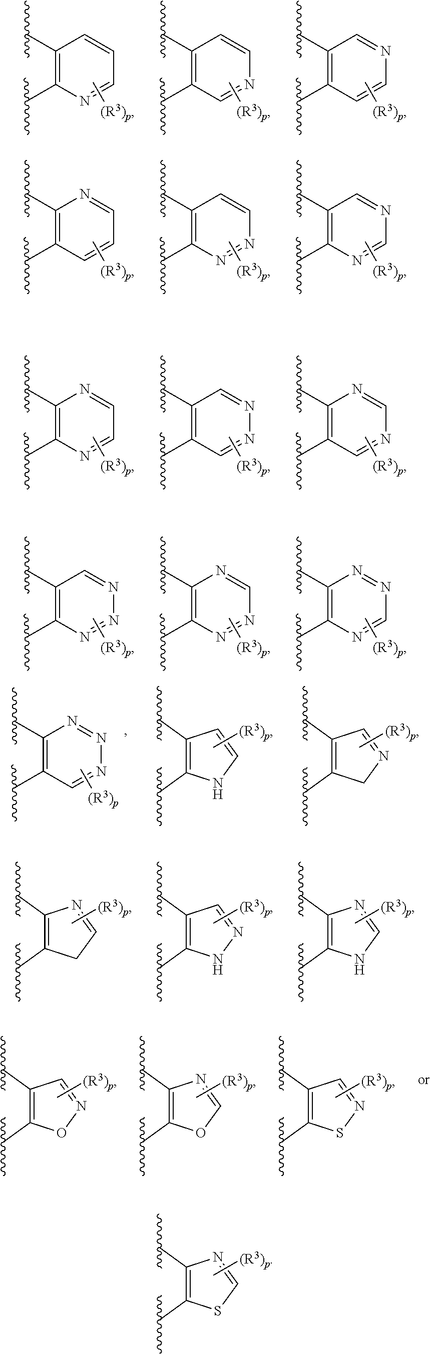

In certain embodiments, Ring B is

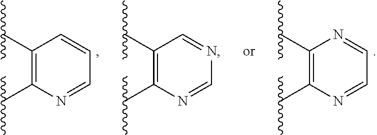

In certain embodiments, Ring B is

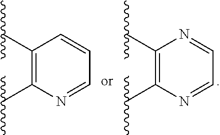

In certain embodiments, each R² is independently —H.

In certain embodiments, each R² is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R² is independently methyl, ethyl, ethyl, propyl, i-propyl, butyl, s-butyl, t-butyl, straight or branched pentyl, or straight or branched hexyl; each of which is optionally substituted.

In certain embodiments, each R² is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R² is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R² is independently F.

In certain embodiments, each R³ is independently —H.

In certain embodiments, each R³ is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R³ is independently methyl, ethyl, ethyl, propyl, i-propyl, butyl, s-butyl, t-butyl, straight or branched pentyl, or straight or branched hexyl; each of which is optionally substituted.

In certain embodiments, each R³ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R³ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, X is C(R⁴)₂. In certain embodiments, X is CH₂.

In certain embodiments, Y is C(R⁴)₂ or NR⁴. In certain embodiments, Y is CH₂. In certain embodiments, Y is NR⁴.

In certain embodiments, each R⁴ is independently —H.

In certain embodiments, each R⁴ is independently C₁₋₆ aliphatic, halogen, -haloalkyl, —OR, —SR, —CN, —NO₂, —SO₂R, —SOR, —C(O)R, —CO₂R, —C(O)N(R)₂, —C(NH)R, —C(NH)NR₂,—NRC(O)R, —NRC(O)N(R)₂, —NRSO₂R, —N(R)₂, or 5-6 membered monocyclic heteroaryl ring having 1-4 heteroatoms independently selected from nitrogen, oxygen, or sulfur.

In certain embodiments, each R⁴ is independently —H, C₁₋₆ aliphatic, —OR, —C(O)R, —CO₂R, —C(O)N(R)₂, —C(NH)R, —C(NH)NR₂,—NRC(O)R, —NRC(O)N(R)₂, —NRSO₂R, —N(R)₂; or 5-6 membered monocyclic heteroaryl ring having 1-4 heteroatoms independently selected from nitrogen, oxygen, or sulfur; each of which is optionally substituted.

In certain embodiments, each R⁴ is independently C₁₋₆ aliphatic, —C(O)R, —C(NH)NR₂, —NRC(O)R, —N(R)₂; or 5-6 membered monocyclic heteroaryl ring having 1-4 heteroatoms independently selected from nitrogen, oxygen, or sulfur; each of which is optionally substituted.

In certain embodiments, each R⁴ is independently

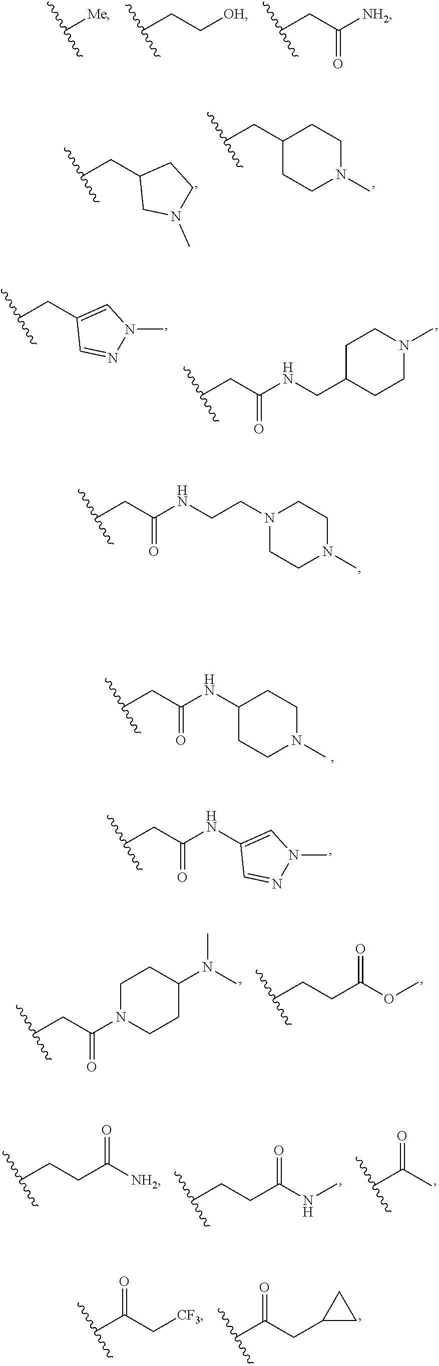

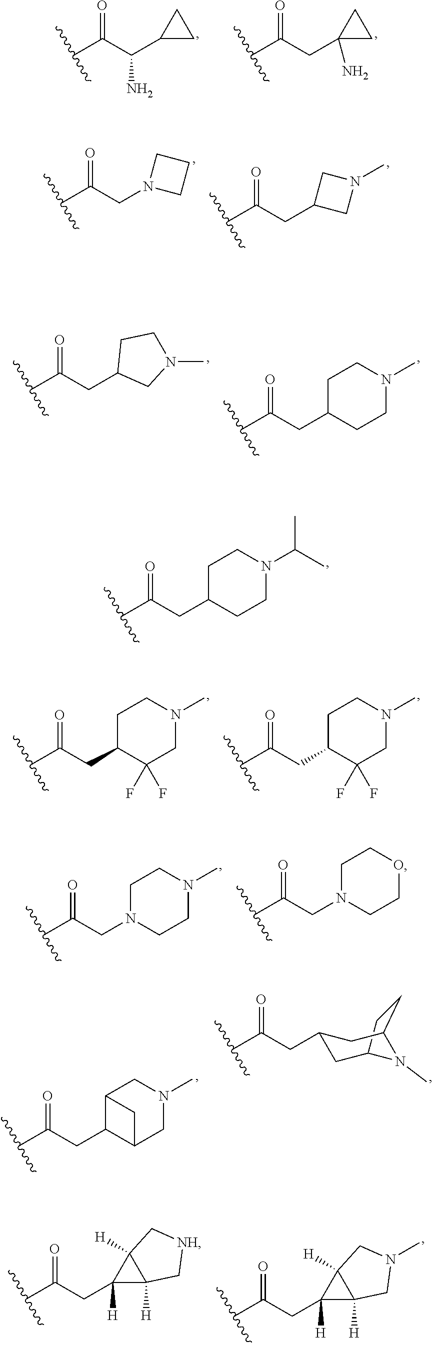

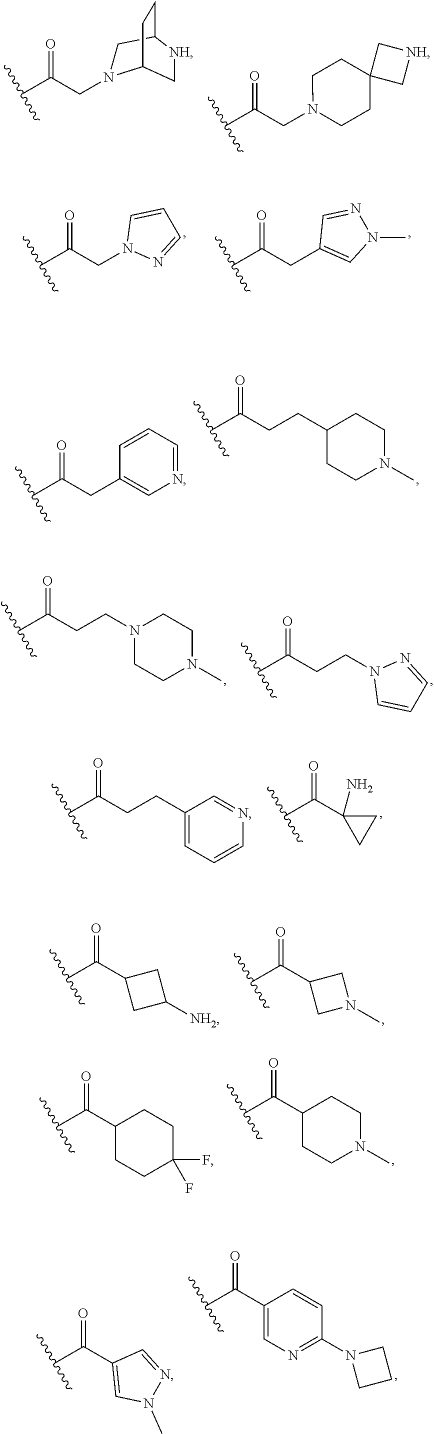

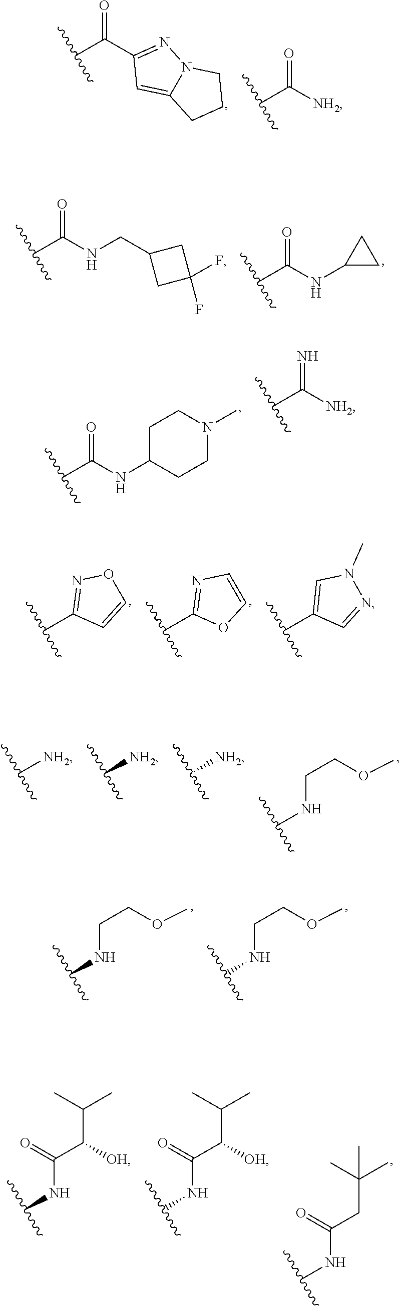

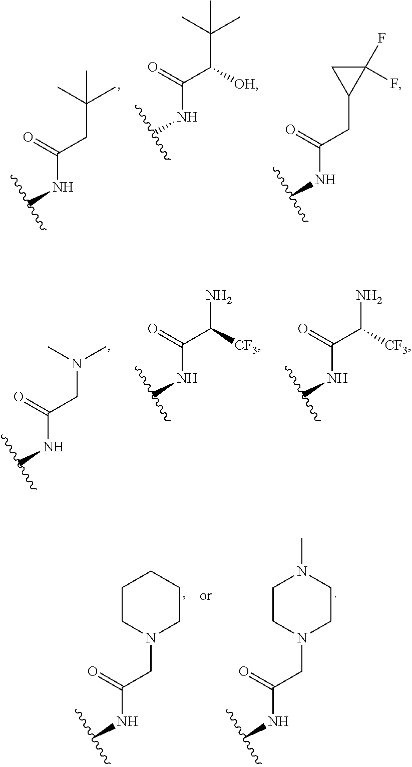

In certain embodiments, each R⁵ is independently —H.

In certain embodiments, each R⁵ is independently C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁵ is independently methyl, ethyl, ethyl, propyl, i-propyl, butyl, s-butyl, t-butyl, straight or branched pentyl, or straight or branched hexyl; each of which is optionally substituted.

In certain embodiments, each R⁵ is independently

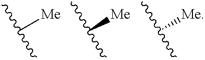

In certain embodiments, each of X, Y, Ring A, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-a,

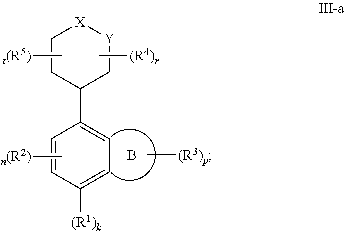

or a pharmaceutically acceptable salt thereof, wherein each of X, Y, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-b,

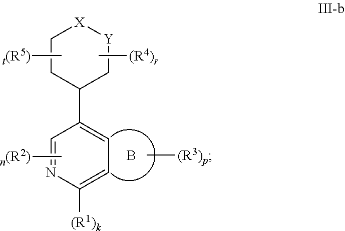

or a pharmaceutically acceptable salt thereof, wherein each of X, Y, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-c,

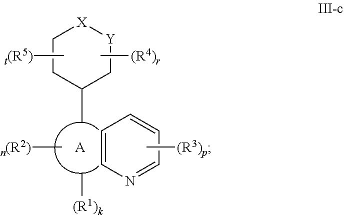

or a pharmaceutically acceptable salt thereof, wherein each of X, Y, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-d,

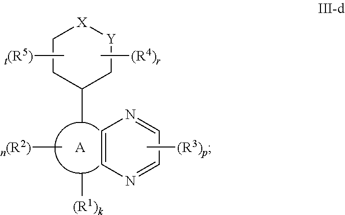

or a pharmaceutically acceptable salt thereof, wherein each of X, Y, Ring B, R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-e,

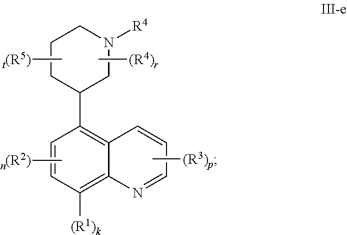

or a pharmaceutically acceptable salt thereof, wherein each of R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-f,

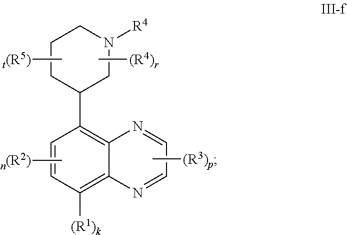

or a pharmaceutically acceptable salt thereof, wherein each of R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-g,

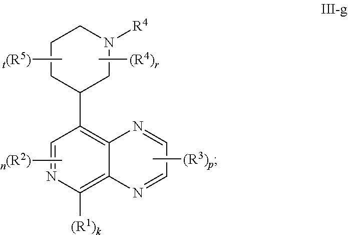

or a pharmaceutically acceptable salt thereof, wherein each of R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-h,

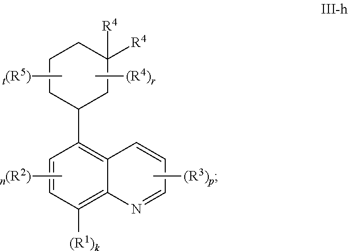

or a pharmaceutically acceptable salt thereof, wherein each of R¹, R², R³, R⁴, R⁵, k, n, p, r, and t, is as defined above and described in embodiments, classes and subclasses above and herein, singly or in combination.

In certain embodiments, the present invention provides the method as described above, wherein the compound is a compound of formula III-j,

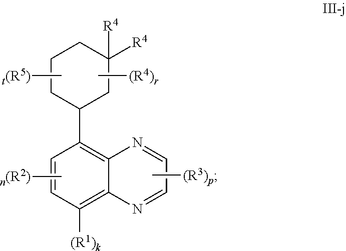

or a pharmaceutically acceptable salt thereof, wherein each of R¹, R², R³, R⁴, R⁵, k, n, p, r, and t, is as defined above and described in embodiments, classes and subclasses above and herein, singly or in combination.

In certain embodiments, the invention provides the method as described above, wherein the compound selected from Table 1:

TABLE 1

| | |
|---|---|
| 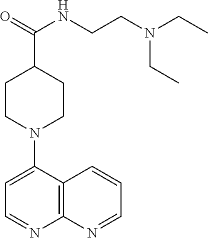 [SMILES: CCN(CC)CCNC(=O)C1CCN(c2ccnc3ncccc23)CC1] | 1 |
| 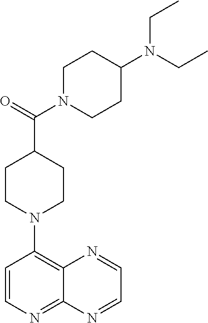 [SMILES: CCN(CC)C1CCN(C(=O)C2CCN(c3ccnc4nccnc34)CC2)CC1] | 2 |
| 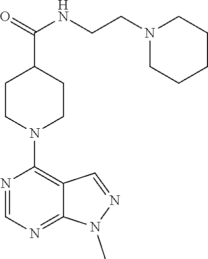 [SMILES: Cn1ncc2c(N3CCC(C(=O)NCCN4CCCCC4)CC3)ncnc21] | 3 |
| 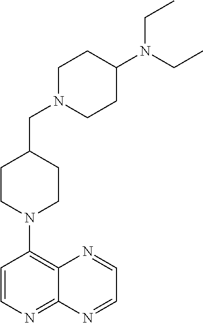 [SMILES: CCN(CC)C1CCN(CC2CCN(c3ccnc4nccnc34)CC2)CC1] | 4 |
| 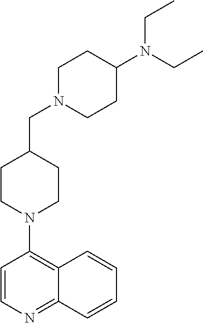 [SMILES: CCN(CC)C1CCN(CC2CCN(c3ccnc4ccccc34)CC2)CC1] | 5 |
| 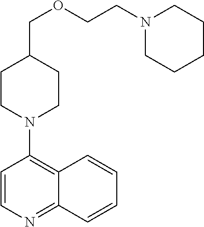 [SMILES: c1ccc2c(N3CCC(COCCN4CCCCC4)CC3)ccnc2c1] | 6 |
| 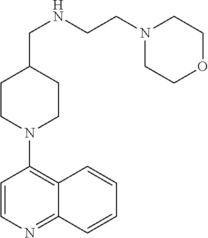 [SMILES: c1ccc2c(N3CCC(CNCCN4CCOCC4)CC3)ccnc2c1] | 7 |
| 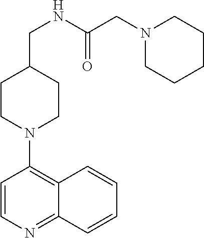 [SMILES: O=C(CN1CCCCC1)NCC1CCN(c2ccnc3ccccc23)CC1] | 8 |
| 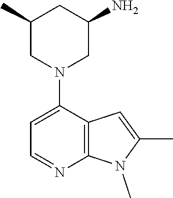 | 9 |
| 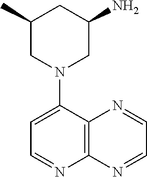 [SMILES: C[C@H]1C[C@@H](N)CN(c2ccnc3nccnc23)C1] | 10 |
| 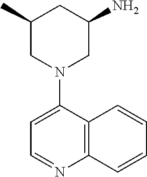 [SMILES: C[C@H]1C[C@@H](N)CN(c2ccnc3ccccc23)C1] | 11 |
| 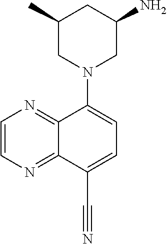 [SMILES: C[C@H]1C[C@@H](N)CN(c2ccc(C#N)c3nccnc23)C1] | 12 |
| 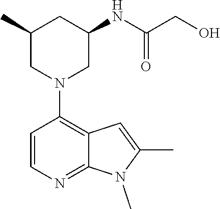 [SMILES: Cc1cc2c(N3C[C@@H](C)C[C@@H](NC(=O)CO)C3)ccnc2n1C] | 13 |
| 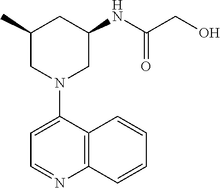 [SMILES: C[C@H]1C[C@@H](NC(=O)CO)CN(c2ccnc3ccccc23)C1] | 14 |
| 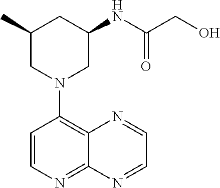 [SMILES: C[C@H]1C[C@@H](NC(=O)CO)CN(c2ccnc3nccnc23)C1] | 15 |
| 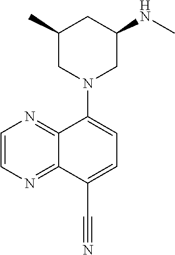 [SMILES: CN[C@@H]1C[C@H](C)CN(c2ccc(C#N)c3nccnc23)C1] | 16 |
| 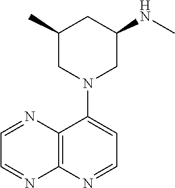 [SMILES: CN[C@@H]1C[C@H](C)CN(c2ccnc3nccnc23)C1] | 17 |
| 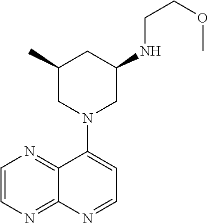 [SMILES: COCCN[C@@H]1C[C@H](C)CN(c2ccnc3nccnc23)C1] | 18 |
| 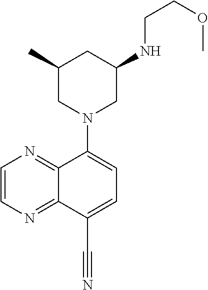 [SMILES: COCCN[C@@H]1C[C@H](C)CN(c2ccc(C#N)c3nccnc23)C1] | 19 |
| 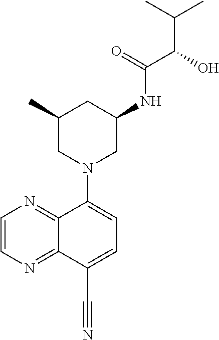 [SMILES: CC(C)[C@H](O)C(=O)N[C@@H]1C[C@H](C)CN(c2ccc(C#N)c3nccnc23)C1] | 20 |
| 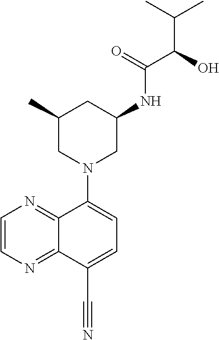 [SMILES: CC(C)[C@@H](O)C(=O)N[C@@H]1C[C@H](C)CN(c2ccc(C#N)c3nccnc23)C1] | 21 |
| 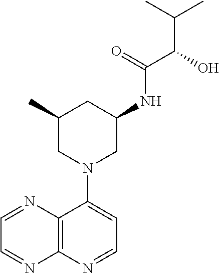 [SMILES: CC(C)[C@H](O)C(=O)N[C@@H]1C[C@H](C)CN(c2ccnc3nccnc23)C1] | 22 |
| 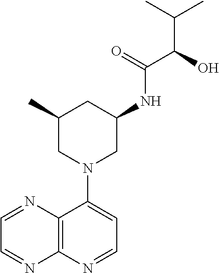 [SMILES: CC(C)[C@@H](O)C(=O)N[C@@H]1C[C@H](C)CN(c2ccnc3nccnc23)C1] | 23 |
| 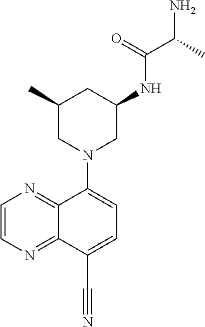 [SMILES: C[C@H]1C[C@@H](NC(=O)[C@@H](C)N)CN(c2ccc(C#N)c3nccnc23)C1] | 24 |
| 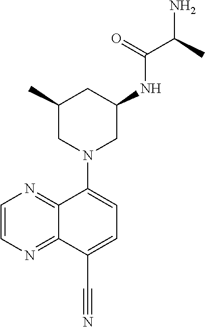 [SMILES: C[C@H]1C[C@@H](NC(=O)[C@H](C)N)CN(c2ccc(C#N)c3nccnc23)C1] | 25 |
| 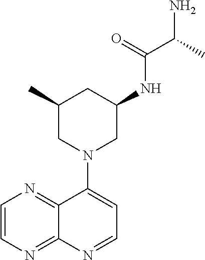 [SMILES: C[C@H]1C[C@@H](NC(=O)[C@@H](C)N)CN(c2ccnc3nccnc23)C1] | 26 |
| 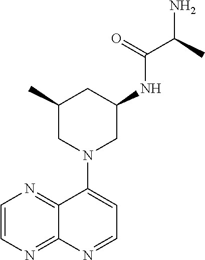 [SMILES: C[C@H]1C[C@@H](NC(=O)[C@H](C)N)CN(c2ccnc3nccnc23)C1] | 27 |
| 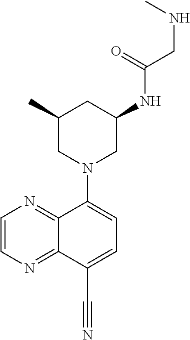 [SMILES: CNCC(=O)N[C@@H]1C[C@H](C)CN(c2ccc(C#N)c3nccnc23)C1] | 28 |
| 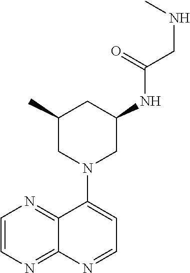 [SMILES: CNCC(=O)N[C@@H]1C[C@H](C)CN(c2ccnc3nccnc23)C1] | 29 |
| 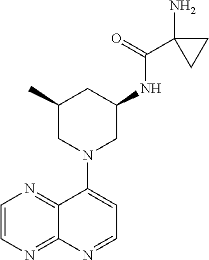 [SMILES: C[C@H]1C[C@@H](NC(=O)C2(N)CC2)CN(c2ccnc3nccnc23)C1] | 30 |
| 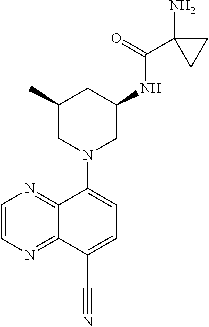 [SMILES: C[C@H]1C[C@@H](NC(=O)C2(N)CC2)CN(c2ccc(C#N)c3nccnc23)C1] | 31 |
| 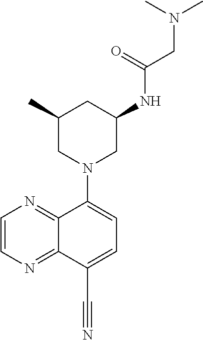 [SMILES: C[C@H]1C[C@@H](NC(=O)CN(C)C)CN(c2ccc(C#N)c3nccnc23)C1] | 32 |
| 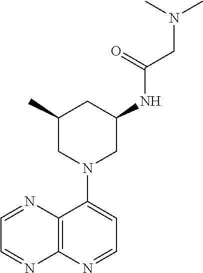 [SMILES: C[C@H]1C[C@@H](NC(=O)CN(C)C)CN(c2ccnc3nccnc23)C1] | 33 |
| 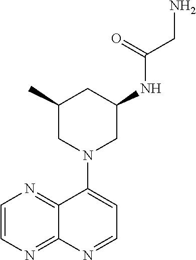 [SMILES: C[C@H]1C[C@@H](NC(=O)CN)CN(c2ccnc3nccnc23)C1] | 34 |
| 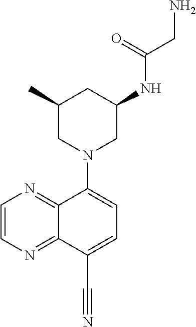 [SMILES: C[C@H]1C[C@@H](NC(=O)CN)CN(c2ccc(C#N)c3nccnc23)C1] | 35 |
| 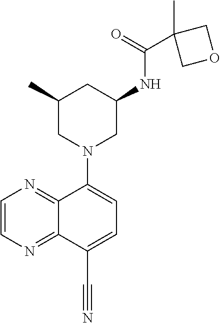 [SMILES: C[C@H]1C[C@@H](NC(=O)C2(C)COC2)CN(c2ccc(C#N)c3nccnc23)C1] | 36 |
| 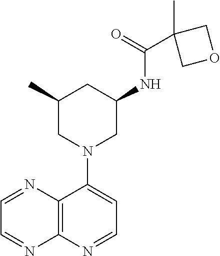 [SMILES: C[C@H]1C[C@@H](NC(=O)C2(C)COC2)CN(c2ccnc3nccnc23)C1] | 37 |
| 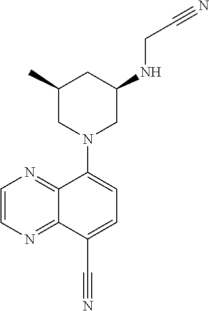 [SMILES: C[C@H]1C[C@@H](NCC#N)CN(c2ccc(C#N)c3nccnc23)C1] | 38 |
| 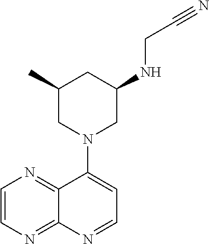 [SMILES: C[C@H]1C[C@@H](NCC#N)CN(c2ccnc3nccnc23)C1] | 39 |
| 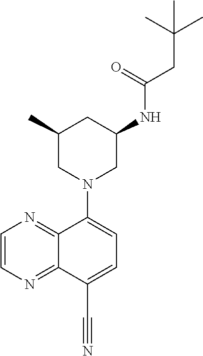 [SMILES: C[C@H]1C[C@@H](NC(=O)CC(C)(C)C)CN(c2ccc(C#N)c3nccnc23)C1] | 40 |
| 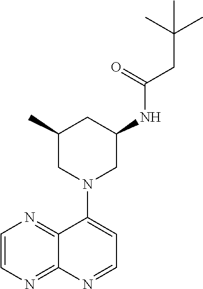 [SMILES: C[C@H]1C[C@@H](NC(=O)CC(C)(C)C)CN(c2ccnc3nccnc23)C1] | 41 |
| 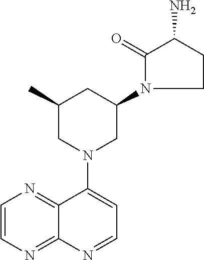 [SMILES: C[C@H]1C[C@@H](N2CC[C@@H](N)C2=O)CN(c2ccnc3nccnc23)C1] | 42 |
| 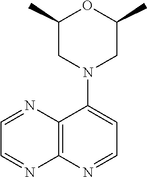 [SMILES: C[C@@H]1CN(c2ccnc3nccnc23)C[C@H](C)O1] | 43 |
| 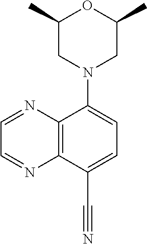 [SMILES: C[C@@H]1CN(c2ccc(C#N)c3nccnc23)C[C@H](C)O1] | 44 |
| 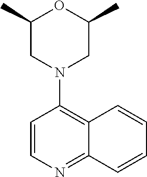 [SMILES: C[C@@H]1CN(c2ccnc3ccccc23)C[C@H](C)O1] | 45 |
| 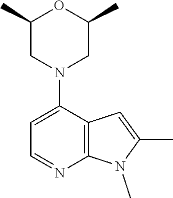 | 46 |
| 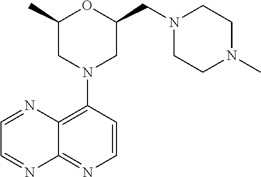 [SMILES: C[C@@H]1CN(c2ccnc3nccnc23)C[C@H](CN2CCN(C)CC2)O1] | 47 |
| 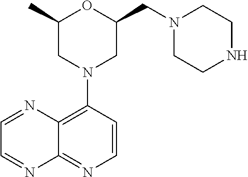 [SMILES: C[C@@H]1CN(c2ccnc3nccnc23)C[C@H](CN2CCNCC2)O1] | 48 |
| 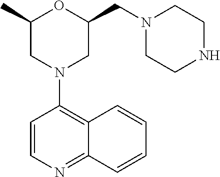 [SMILES: C[C@@H]1CN(c2ccnc3ccccc23)C[C@H](CN2CCNCC2)O1] | 49 |
| 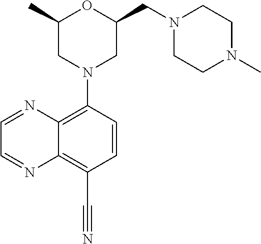 [SMILES: C[C@@H]1CN(c2ccc(C#N)c3nccnc23)C[C@H](CN2CCN(C)CC2)O1] | 50 |
| 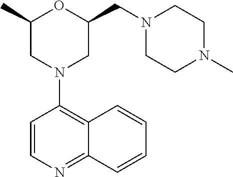 | 51 |
| 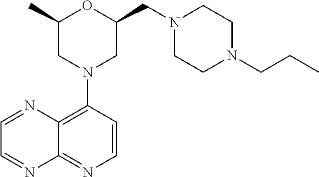 [SMILES: CCCN1CCN(C[C@H]2CN(c3ccnc4nccnc34)C[C@@H](C)O2)CC1] | 52 |
| 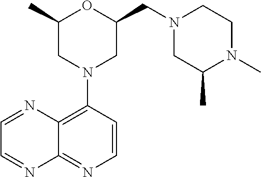 [SMILES: C[C@@H]1CN(c2ccnc3nccnc23)C[C@H](CN2CCN(C)[C@@H](C)C2)O1] | 53 |
| 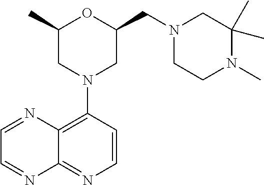 [SMILES: C[C@@H]1CN(c2ccnc3nccnc23)C[C@H](CN2CCN(C)C(C)(C)C2)O1] | 54 |
| 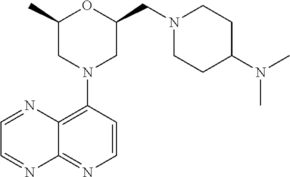 [SMILES: C[C@@H]1CN(c2ccnc3nccnc23)C[C@H](CN2CCC(N(C)C)CC2)O1] | 55 |
| 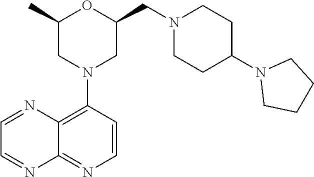 [SMILES: C[C@@H]1CN(c2ccnc3nccnc23)C[C@H](CN2CCC(N3CCCC3)CC2)O1] | 56 |
| 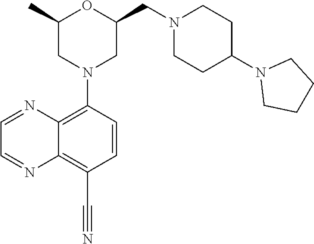 [SMILES: C[C@@H]1CN(c2ccc(C#N)c3nccnc23)C[C@H](CN2CCC(N3CCCC3)CC2)O1] | 57 |
| 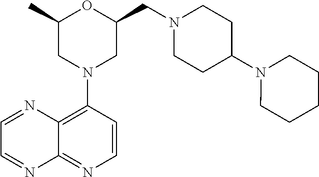 [SMILES: C[C@@H]1CN(c2ccnc3nccnc23)C[C@H](CN2CCC(N3CCCCC3)CC2)O1] | 58 |
| 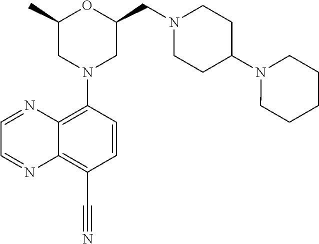 [SMILES: C[C@@H]1CN(c2ccc(C#N)c3nccnc23)C[C@H](CN2CCC(N3CCCCC3)CC2)O1] | 59 |
| 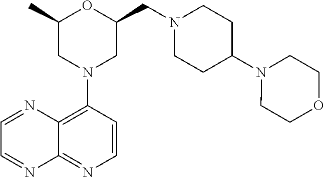 [SMILES: C[C@@H]1CN(c2ccnc3nccnc23)C[C@H](CN2CCC(N3CCOCC3)CC2)O1] | 60 |
| 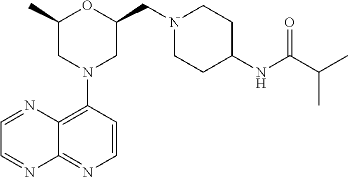 [SMILES: CC(C)C(=O)NC1CCN(C[C@H]2CN(c3ccnc4nccnc34)C[C@@H](C)O2)CC1] | 61 |
| 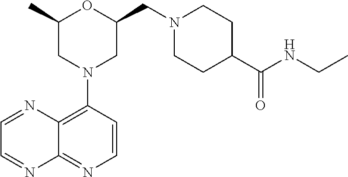 [SMILES: CCNC(=O)C1CCN(C[C@H]2CN(c3ccnc4nccnc34)C[C@@H](C)O2)CC1] | 62 |
| 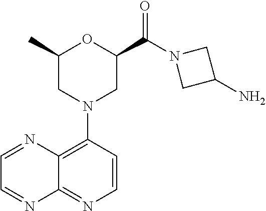 [SMILES: C[C@@H]1CN(c2ccnc3nccnc23)C[C@H](C(=O)N2CC(N)C2)O1] | 63 |
| 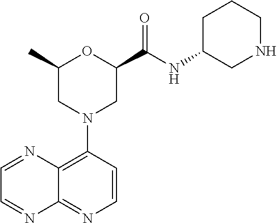 [SMILES: C[C@@H]1CN(c2ccnc3nccnc23)C[C@H](C(=O)N[C@@H]2CCCNC2)O1] | 64 |
| 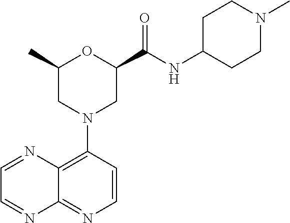 [SMILES: C[C@@H]1CN(c2ccnc3nccnc23)C[C@H](C(=O)NC2CCN(C)CC2)O1] | 65 |
| 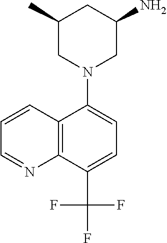 [SMILES: C[C@H]1C[C@@H](N)CN(c2ccc(C(F)(F)F)c3ncccc23)C1] | 66 |
| 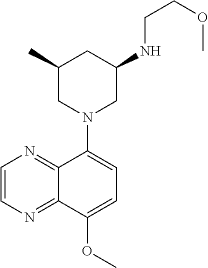 [SMILES: COCCN[C@@H]1C[C@H](C)CN(c2ccc(OC)c3nccnc23)C1] | 67 |
| 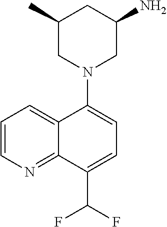 [SMILES: C[C@H]1C[C@@H](N)CN(c2ccc(C(F)F)c3ncccc23)C1] | 68 |
| 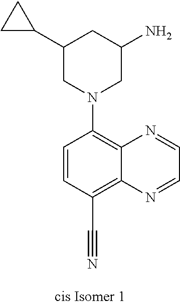 [SMILES: N#Cc1ccc(N2CC(N)CC(C3CC3)C2)c2nccnc12] | 69 |
| 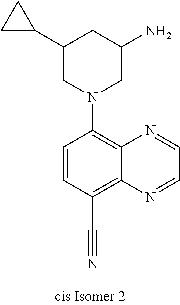 [SMILES: N#Cc1ccc(N2CC(N)CC(C3CC3)C2)c2nccnc12] | 70 |
| 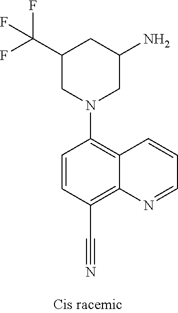 [SMILES: N#Cc1ccc(N2CC(N)CC(C(F)(F)F)C2)c2cccnc12] | 71 |
| 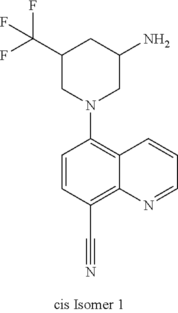 [SMILES: N#Cc1ccc(N2CC(N)CC(C(F)(F)F)C2)c2cccnc12] | 72 |
| 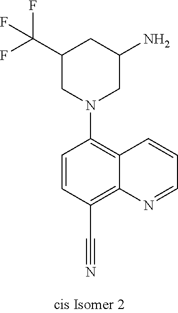 [SMILES: N#Cc1ccc(N2CC(N)CC(C(F)(F)F)C2)c2cccnc12] | 73 |
| 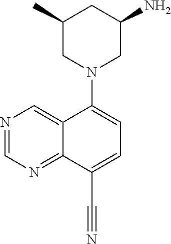 [SMILES: C[C@H]1C[C@@H](N)CN(c2ccc(C#N)c3ncncc23)C1] | 74 |
| 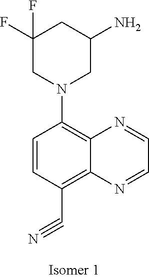 [SMILES: N#Cc1ccc(N2CC(N)CC(F)(F)C2)c2nccnc12] | 75 |
| 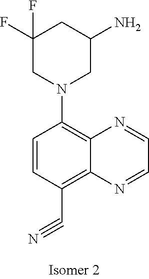 [SMILES: N#Cc1ccc(N2CC(N)CC(F)(F)C2)c2nccnc12] | 76 |
| 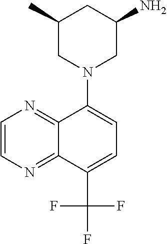 [SMILES: C[C@H]1C[C@@H](N)CN(c2ccc(C(F)(F)F)c3nccnc23)C1] | 77 |
| 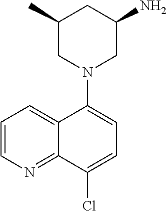 [SMILES: C[C@H]1C[C@@H](N)CN(c2ccc(Cl)c3ncccc23)C1] | 78 |
| 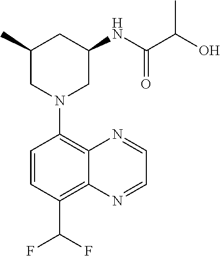 [SMILES: CC(O)C(=O)N[C@@H]1C[C@H](C)CN(c2ccc(C(F)F)c3nccnc23)C1] | 79 |
| 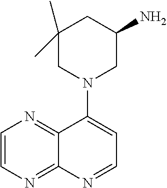 [SMILES: CC1(C)C[C@@H](N)CN(c2ccnc3nccnc23)C1] | 80 |
| 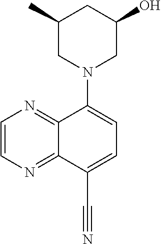 [SMILES: C[C@H]1C[C@@H](O)CN(c2ccc(C#N)c3nccnc23)C1] | 81 |
| 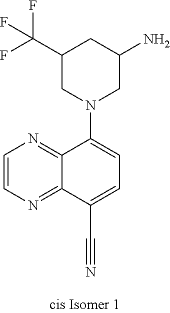 [SMILES: N#Cc1ccc(N2CC(N)CC(C(F)(F)F)C2)c2nccnc12] | 82 |
| 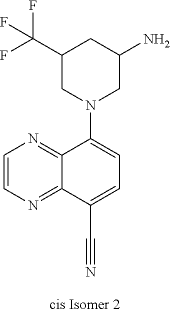 [SMILES: N#Cc1ccc(N2CC(N)CC(C(F)(F)F)C2)c2nccnc12] | 83 |
| 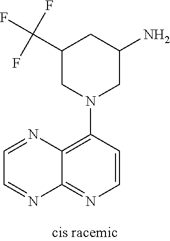 [SMILES: NC1CC(C(F)(F)F)CN(c2ccnc3nccnc23)C1] | 84 |
| 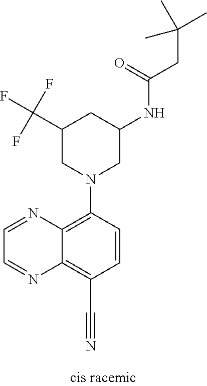 [SMILES: CC(C)(C)CC(=O)NC1CC(C(F)(F)F)CN(c2ccc(C#N)c3nccnc23)C1] | 85 |
| 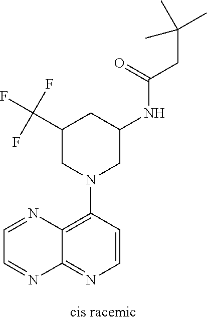 [SMILES: CC(C)(C)CC(=O)NC1CC(C(F)(F)F)CN(c2ccnc3nccnc23)C1] | 86 |
| 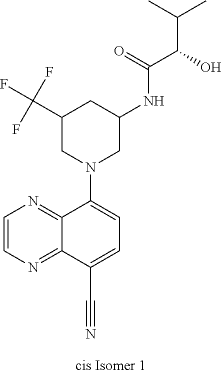 [SMILES: CC(C)[C@H](O)C(=O)NC1CC(C(F)(F)F)CN(c2ccc(C#N)c3nccnc23)C1] | 87 |
| 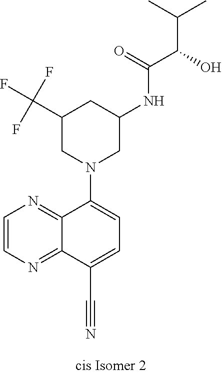 [SMILES: CC(C)[C@H](O)C(=O)NC1CC(C(F)(F)F)CN(c2ccc(C#N)c3nccnc23)C1] | 88 |
| 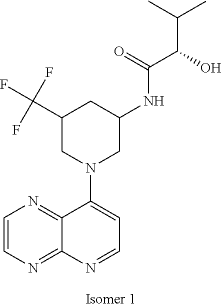 [SMILES: CC(C)[C@H](O)C(=O)NC1CC(C(F)(F)F)CN(c2ccnc3nccnc23)C1] | 89 |
| 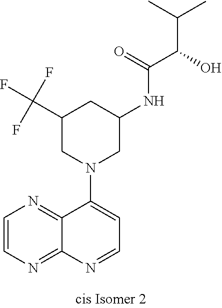 [SMILES: CC(C)[C@H](O)C(=O)NC1CC(C(F)(F)F)CN(c2ccnc3nccnc23)C1] | 90 |
| 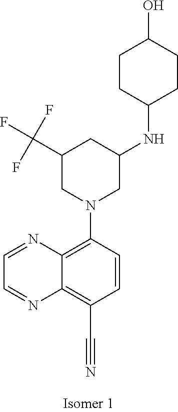 [SMILES: N#Cc1ccc(N2CC(NC3CCC(O)CC3)CC(C(F)(F)F)C2)c2nccnc12] | 91 |
| 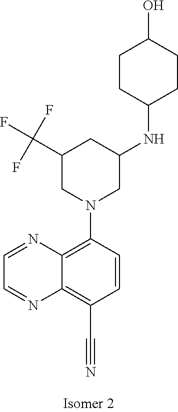 [SMILES: N#Cc1ccc(N2CC(NC3CCC(O)CC3)CC(C(F)(F)F)C2)c2nccnc12] | 92 |
| 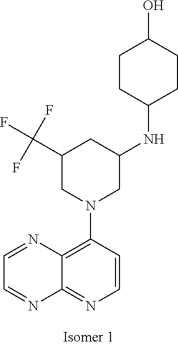 [SMILES: OC1CCC(NC2CC(C(F)(F)F)CN(c3ccnc4nccnc34)C2)CC1] | 93 |
| 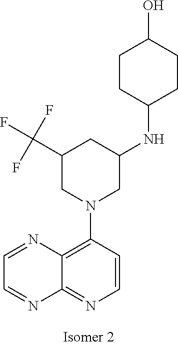 [SMILES: OC1CCC(NC2CC(C(F)(F)F)CN(c3ccnc4nccnc34)C2)CC1] | 94 |
| 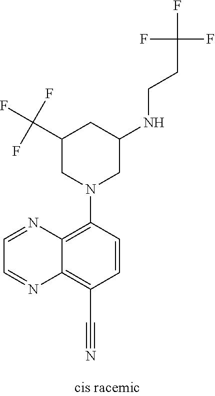 [SMILES: N#Cc1ccc(N2CC(NCCC(F)(F)F)CC(C(F)(F)F)C2)c2nccnc12] | 95 |
| 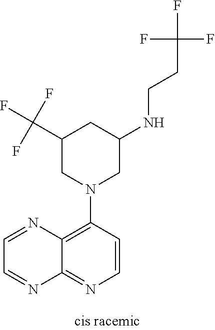 [SMILES: FC(F)(F)CCNC1CC(C(F)(F)F)CN(c2ccnc3nccnc23)C1] | 96 |
| 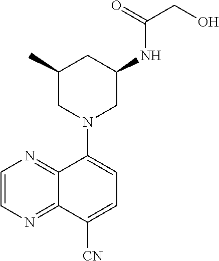 [SMILES: C[C@H]1C[C@@H](NC(=O)CO)CN(c2ccc(C#N)c3nccnc23)C1] | 97 |
| 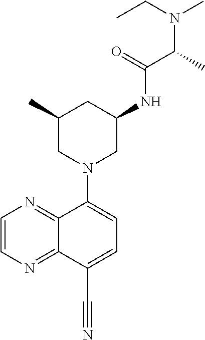 [SMILES: CCN(C)[C@H](C)C(=O)N[C@@H]1C[C@H](C)CN(c2ccc(C#N)c3nccnc23)C1] | 98 |
| 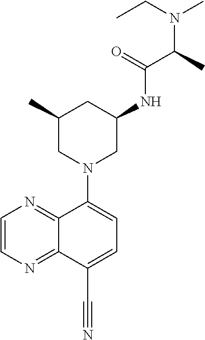 [SMILES: CCN(C)[C@@H](C)C(=O)N[C@@H]1C[C@H](C)CN(c2ccc(C#N)c3nccnc23)C1] | 99 |
| 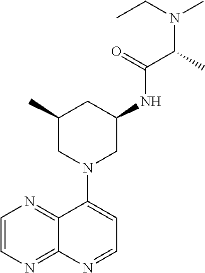 [SMILES: CCN(C)[C@H](C)C(=O)N[C@@H]1C[C@H](C)CN(c2ccnc3nccnc23)C1] | 100 |
| 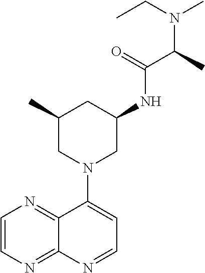 [SMILES: CCN(C)[C@@H](C)C(=O)N[C@@H]1C[C@H](C)CN(c2ccnc3nccnc23)C1] | 101 |
| 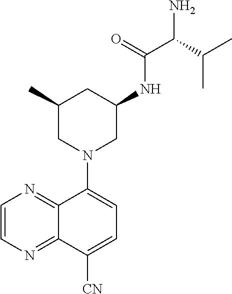 [SMILES: CC(C)[C@@H](N)C(=O)N[C@@H]1C[C@H](C)CN(c2ccc(C#N)c3nccnc23)C1] | 102 |
| 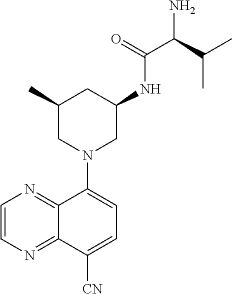 [SMILES: CC(C)[C@H](N)C(=O)N[C@@H]1C[C@H](C)CN(c2ccc(C#N)c3nccnc23)C1] | 103 |
| 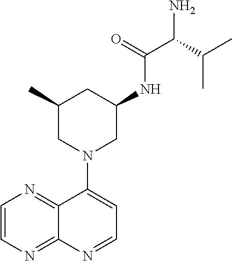 [SMILES: CC(C)[C@@H](N)C(=O)N[C@@H]1C[C@H](C)CN(c2ccnc3nccnc23)C1] | 104 |
| 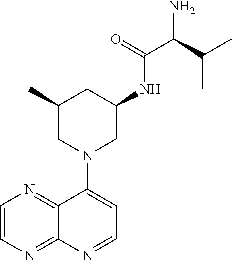 [SMILES: CC(C)[C@H](N)C(=O)N[C@@H]1C[C@H](C)CN(c2ccnc3nccnc23)C1] | 105 |
| 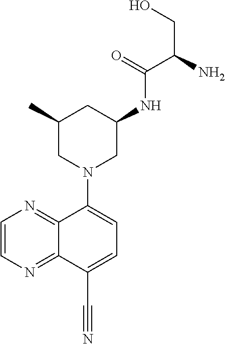 [SMILES: C[C@H]1C[C@@H](NC(=O)[C@H](N)CO)CN(c2ccc(C#N)c3nccnc23)C1] | 106 |
| 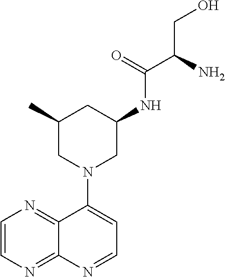 [SMILES: C[C@H]1C[C@@H](NC(=O)[C@H](N)CO)CN(c2ccnc3nccnc23)C1] | 107 |
| 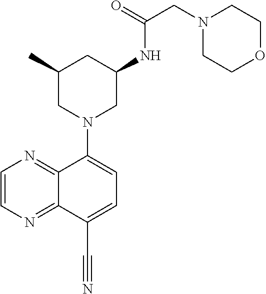 [SMILES: C[C@H]1C[C@@H](NC(=O)CN2CCOCC2)CN(c2ccc(C#N)c3nccnc23)C1] | 108 |
| 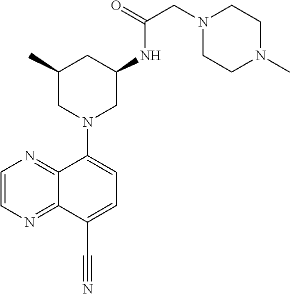 [SMILES: C[C@H]1C[C@@H](NC(=O)CN2CCN(C)CC2)CN(c2ccc(C#N)c3nccnc23)C1] | 109 |
| 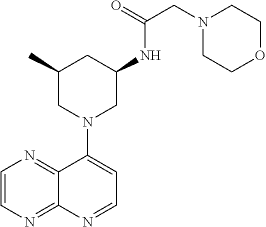 [SMILES: C[C@H]1C[C@@H](NC(=O)CN2CCOCC2)CN(c2ccnc3nccnc23)C1] | 110 |
| 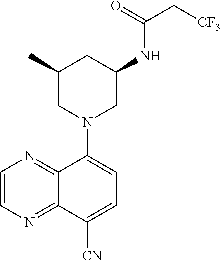 [SMILES: C[C@H]1C[C@@H](NC(=O)CC(F)(F)F)CN(c2ccc(C#N)c3nccnc23)C1] | 111 |
| 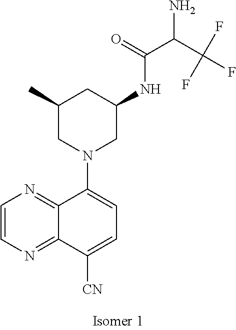 [SMILES: C[C@H]1C[C@@H](NC(=O)C(N)C(F)(F)F)CN(c2ccc(C#N)c3nccnc23)C1] | 112 |
| 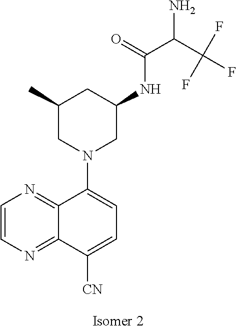 [SMILES: C[C@H]1C[C@@H](NC(=O)C(N)C(F)(F)F)CN(c2ccc(C#N)c3nccnc23)C1] | 113 |
| 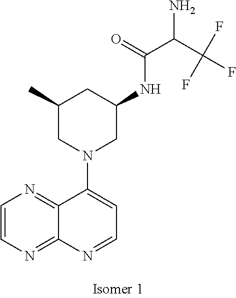 [SMILES: C[C@H]1C[C@@H](NC(=O)C(N)C(F)(F)F)CN(c2ccnc3nccnc23)C1] | 114 |
| 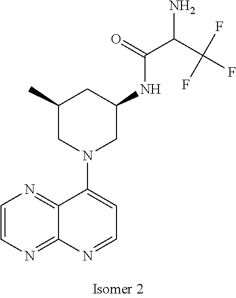 [SMILES: C[C@H]1C[C@@H](NC(=O)C(N)C(F)(F)F)CN(c2ccnc3nccnc23)C1] | 115 |
| 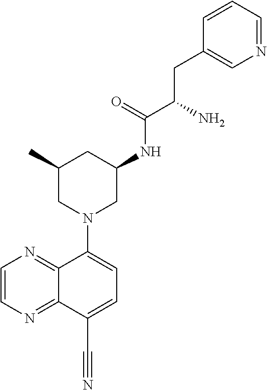 [SMILES: C[C@H]1C[C@@H](NC(=O)[C@@H](N)Cc2cccnc2)CN(c2ccc(C#N)c3nccnc23)C1] | 116 |
| 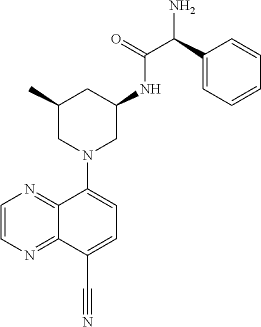 [SMILES: C[C@H]1C[C@@H](NC(=O)[C@@H](N)c2ccccc2)CN(c2ccc(C#N)c3nccnc23)C1] | 117 |
| 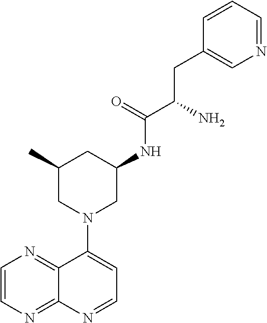 [SMILES: C[C@H]1C[C@@H](NC(=O)[C@@H](N)Cc2cccnc2)CN(c2ccnc3nccnc23)C1] | 118 |
| 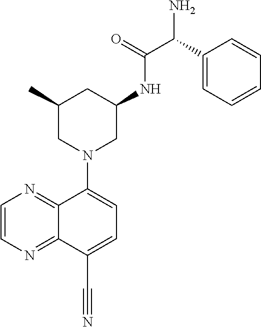 [SMILES: C[C@H]1C[C@@H](NC(=O)[C@H](N)c2ccccc2)CN(c2ccc(C#N)c3nccnc23)C1] | 119 |
| 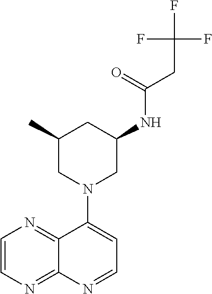 [SMILES: C[C@H]1C[C@@H](NC(=O)CC(F)(F)F)CN(c2ccnc3nccnc23)C1] | 120 |
| 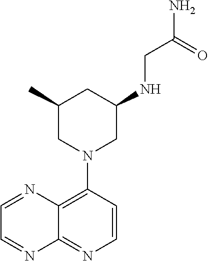 [SMILES: C[C@H]1C[C@@H](NCC(N)=O)CN(c2ccnc3nccnc23)C1] | 121 |
| 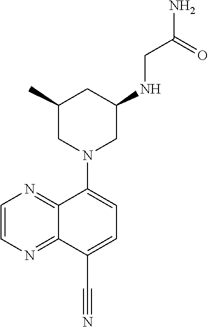 [SMILES: C[C@H]1C[C@@H](NCC(N)=O)CN(c2ccc(C#N)c3nccnc23)C1] | 122 |
| 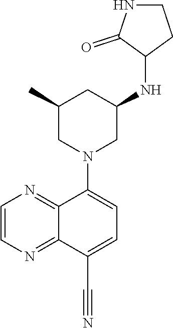 [SMILES: C[C@H]1C[C@@H](NC2CCNC2=O)CN(c2ccc(C#N)c3nccnc23)C1] | 123 |
| 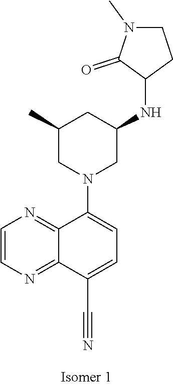 [SMILES: C[C@H]1C[C@@H](NC2CCN(C)C2=O)CN(c2ccc(C#N)c3nccnc23)C1] | 124 |
| 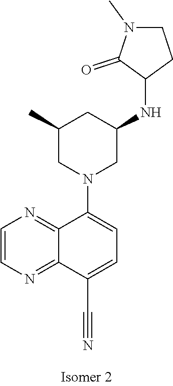 [SMILES: C[C@H]1C[C@@H](NC2CCN(C)C2=O)CN(c2ccc(C#N)c3nccnc23)C1] | 125 |
| 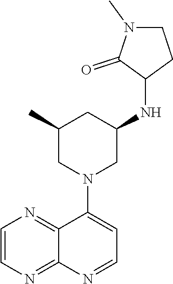 [SMILES: C[C@H]1C[C@@H](NC2CCN(C)C2=O)CN(c2ccnc3nccnc23)C1] | 126 |
| 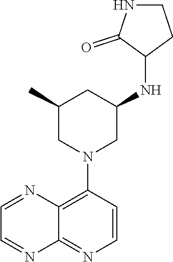 [SMILES: C[C@H]1C[C@@H](NC2CCNC2=O)CN(c2ccnc3nccnc23)C1] | 127 |
| 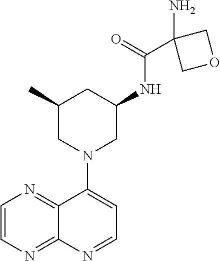 [SMILES: C[C@H]1C[C@@H](NC(=O)C2(N)COC2)CN(c2ccnc3nccnc23)C1] | 128 |
| 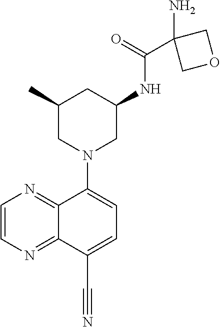 [SMILES: C[C@H]1C[C@@H](NC(=O)C2(N)COC2)CN(c2ccc(C#N)c3nccnc23)C1] | 129 |
| 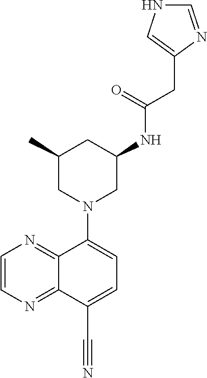 [SMILES: C[C@H]1C[C@@H](NC(=O)Cc2c[nH]cn2)CN(c2ccc(C#N)c3nccnc23)C1] | 130 |
| 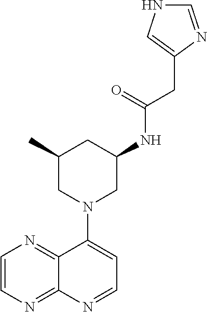 [SMILES: C[C@H]1C[C@@H](NC(=O)Cc2c[nH]cn2)CN(c2ccnc3nccnc23)C1] | 131 |
| 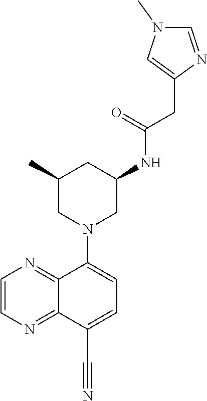 [SMILES: C[C@H]1C[C@@H](NC(=O)Cc2cn(C)cn2)CN(c2ccc(C#N)c3nccnc23)C1] | 132 |
| 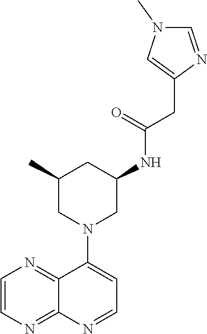 [SMILES: C[C@H]1C[C@@H](NC(=O)Cc2cn(C)cn2)CN(c2ccnc3nccnc23)C1] | 133 |
| 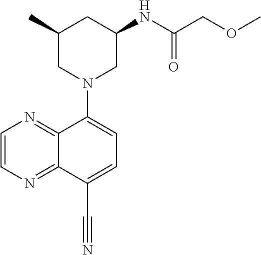 [SMILES: COCC(=O)N[C@@H]1C[C@H](C)CN(c2ccc(C#N)c3nccnc23)C1] | 134 |
| 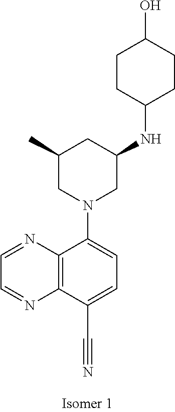 [SMILES: C[C@H]1C[C@@H](NC2CCC(O)CC2)CN(c2ccc(C#N)c3nccnc23)C1] | 135 |
| 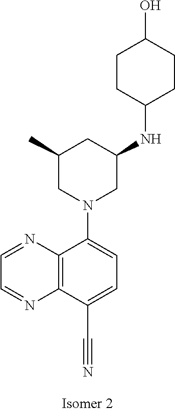 [SMILES: C[C@H]1C[C@@H](NC2CCC(O)CC2)CN(c2ccc(C#N)c3nccnc23)C1] | 136 |
| 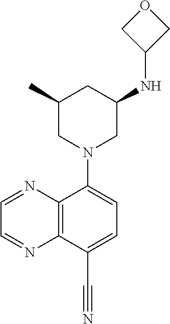 [SMILES: C[C@H]1C[C@@H](NC2COC2)CN(c2ccc(C#N)c3nccnc23)C1] | 137 |
| 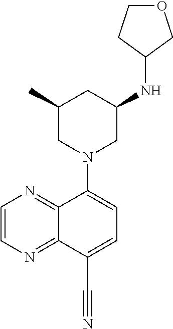 [SMILES: C[C@H]1C[C@@H](NC2CCOC2)CN(c2ccc(C#N)c3nccnc23)C1] | 138 |
| 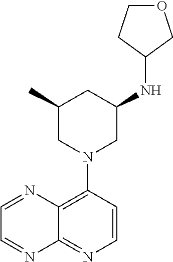 [SMILES: C[C@H]1C[C@@H](NC2CCOC2)CN(c2ccnc3nccnc23)C1] | 139 |
| 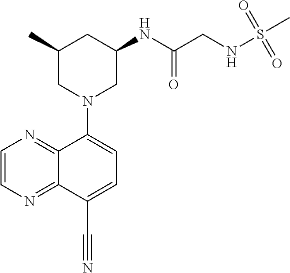 [SMILES: C[C@H]1C[C@@H](NC(=O)CNS(C)(=O)=O)CN(c2ccc(C#N)c3nccnc23)C1] | 140 |
| 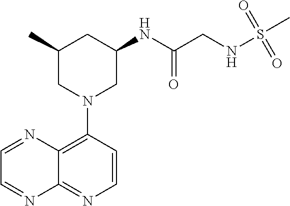 [SMILES: C[C@H]1C[C@@H](NC(=O)CNS(C)(=O)=O)CN(c2ccnc3nccnc23)C1] | 141 |
| 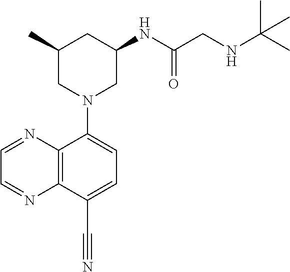 [SMILES: C[C@H]1C[C@@H](NC(=O)CNC(C)(C)C)CN(c2ccc(C#N)c3nccnc23)C1] | 142 |
| 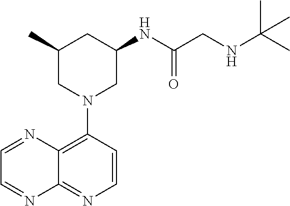 [SMILES: C[C@H]1C[C@@H](NC(=O)CNC(C)(C)C)CN(c2ccnc3nccnc23)C1] | 143 |
| 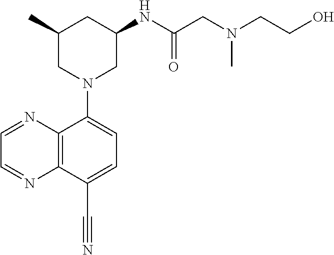 [SMILES: C[C@H]1C[C@@H](NC(=O)CN(C)CCO)CN(c2ccc(C#N)c3nccnc23)C1] | 144 |
| 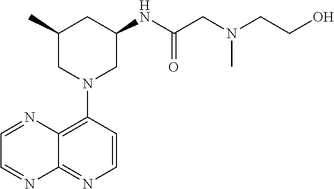 [SMILES: C[C@H]1C[C@@H](NC(=O)CN(C)CCO)CN(c2ccnc3nccnc23)C1] | 145 |
| 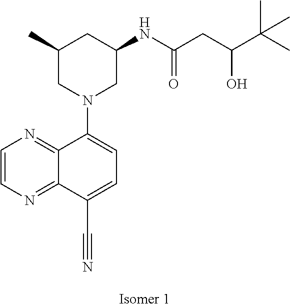 [SMILES: C[C@H]1C[C@@H](NC(=O)CC(O)C(C)(C)C)CN(c2ccc(C#N)c3nccnc23)C1] | 146 |
| 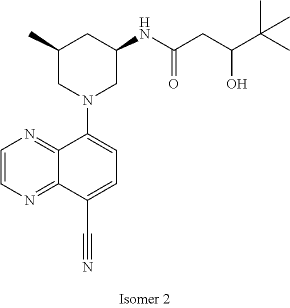 [SMILES: C[C@H]1C[C@@H](NC(=O)CC(O)C(C)(C)C)CN(c2ccc(C#N)c3nccnc23)C1] | 147 |
| 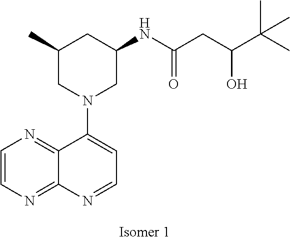 [SMILES: C[C@H]1C[C@@H](NC(=O)CC(O)C(C)(C)C)CN(c2ccnc3nccnc23)C1] | 148 |
| 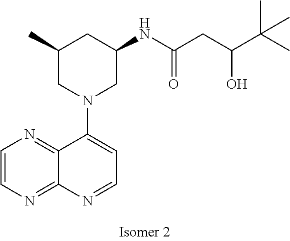 [SMILES: C[C@H]1C[C@@H](NC(=O)CC(O)C(C)(C)C)CN(c2ccnc3nccnc23)C1] | 149 |
| 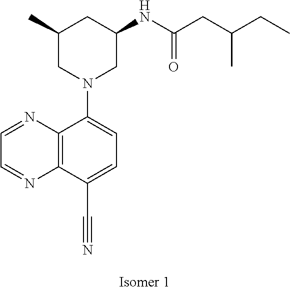 [SMILES: CCC(C)CC(=O)N[C@@H]1C[C@H](C)CN(c2ccc(C#N)c3nccnc23)C1] | 150 |
| 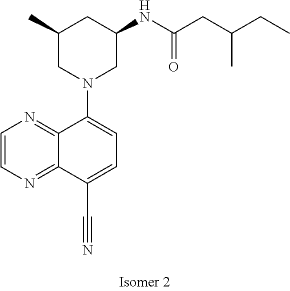 [SMILES: CCC(C)CC(=O)N[C@@H]1C[C@H](C)CN(c2ccc(C#N)c3nccnc23)C1] | 151 |
| 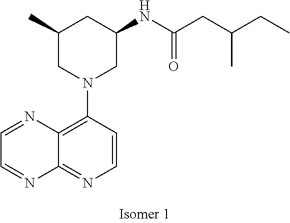 [SMILES: CCC(C)CC(=O)N[C@@H]1C[C@H](C)CN(c2ccnc3nccnc23)C1] | 152 |
| 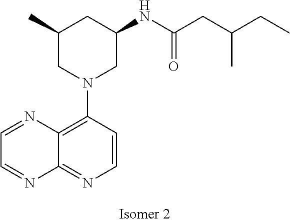 [SMILES: CCC(C)CC(=O)N[C@@H]1C[C@H](C)CN(c2ccnc3nccnc23)C1] | 153 |
| 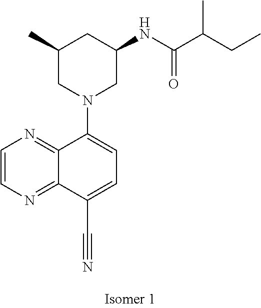 [SMILES: CCC(C)C(=O)N[C@@H]1C[C@H](C)CN(c2ccc(C#N)c3nccnc23)C1] | 154 |
| 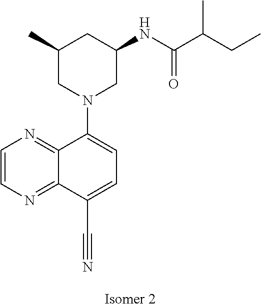 [SMILES: CCC(C)C(=O)N[C@@H]1C[C@H](C)CN(c2ccc(C#N)c3nccnc23)C1] | 155 |
| 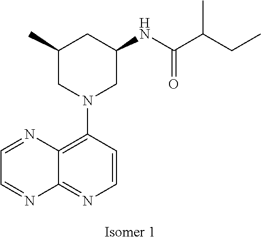 [SMILES: CCC(C)C(=O)N[C@@H]1C[C@H](C)CN(c2ccnc3nccnc23)C1] | 156 |
| 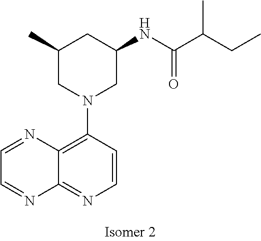 [SMILES: CCC(C)C(=O)N[C@@H]1C[C@H](C)CN(c2ccnc3nccnc23)C1] | 157 |
| 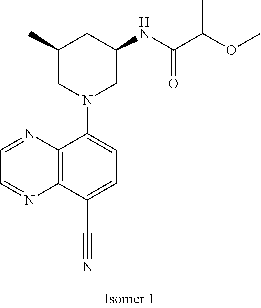 [SMILES: COC(C)C(=O)N[C@@H]1C[C@H](C)CN(c2ccc(C#N)c3nccnc23)C1] | 158 |
| 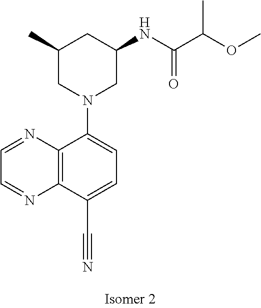 [SMILES: COC(C)C(=O)N[C@@H]1C[C@H](C)CN(c2ccc(C#N)c3nccnc23)C1] | 159 |
| 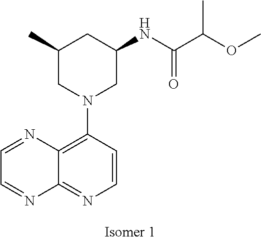 [SMILES: COC(C)C(=O)N[C@@H]1C[C@H](C)CN(c2ccnc3nccnc23)C1] | 160 |
| 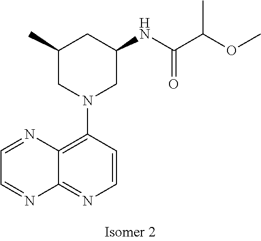 [SMILES: COC(C)C(=O)N[C@@H]1C[C@H](C)CN(c2ccnc3nccnc23)C1] | 161 |
| 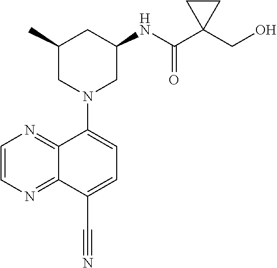 [SMILES: C[C@H]1C[C@@H](NC(=O)C2(CO)CC2)CN(c2ccc(C#N)c3nccnc23)C1] | 162 |
| 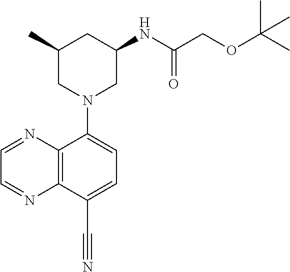 [SMILES: C[C@H]1C[C@@H](NC(=O)COC(C)(C)C)CN(c2ccc(C#N)c3nccnc23)C1] | 163 |
| 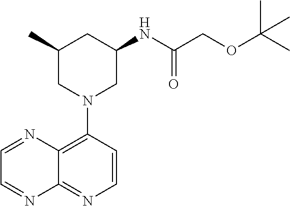 [SMILES: C[C@H]1C[C@@H](NC(=O)COC(C)(C)C)CN(c2ccnc3nccnc23)C1] | 164 |
| 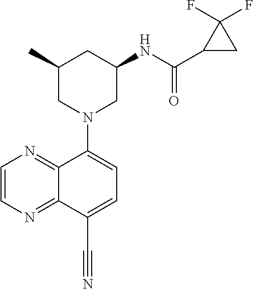 [SMILES: C[C@H]1C[C@@H](NC(=O)C2CC2(F)F)CN(c2ccc(C#N)c3nccnc23)C1] | 165 |
| 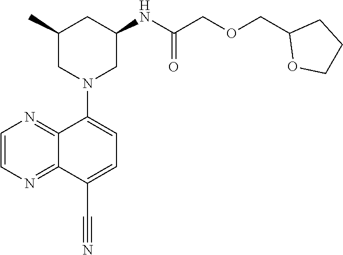 [SMILES: C[C@H]1C[C@@H](NC(=O)COCC2CCCO2)CN(c2ccc(C#N)c3nccnc23)C1] | 166 |
| 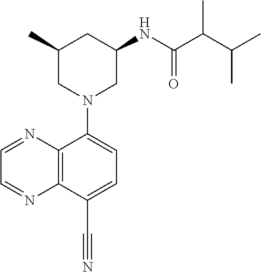 [SMILES: CC(C)C(C)C(=O)N[C@@H]1C[C@H](C)CN(c2ccc(C#N)c3nccnc23)C1] | 167 |
| 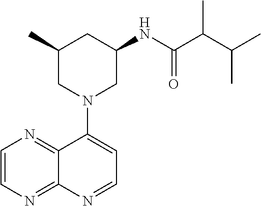 [SMILES: CC(C)C(C)C(=O)N[C@@H]1C[C@H](C)CN(c2ccnc3nccnc23)C1] | 168 |
| 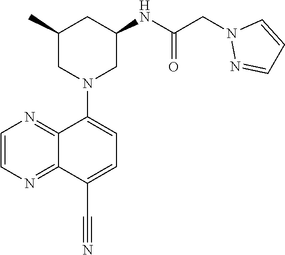 [SMILES: C[C@H]1C[C@@H](NC(=O)Cn2cccn2)CN(c2ccc(C#N)c3nccnc23)C1] | 169 |
| 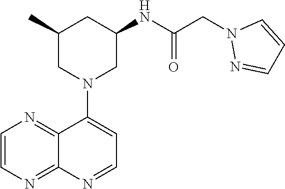 [SMILES: C[C@H]1C[C@@H](NC(=O)Cn2cccn2)CN(c2ccnc3nccnc23)C1] | 170 |
| 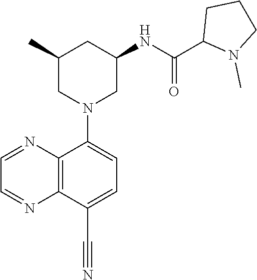 [SMILES: C[C@H]1C[C@@H](NC(=O)C2CCCN2C)CN(c2ccc(C#N)c3nccnc23)C1] | 171 |
| 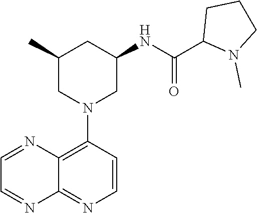 [SMILES: C[C@H]1C[C@@H](NC(=O)C2CCCN2C)CN(c2ccnc3nccnc23)C1] | 172 |
| 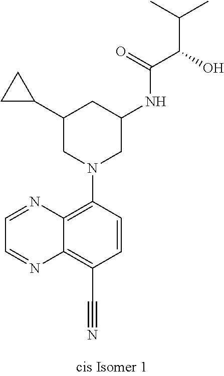 [SMILES: CC(C)[C@H](O)C(=O)NC1CC(C2CC2)CN(c2ccc(C#N)c3nccnc23)C1] | 173 |
| 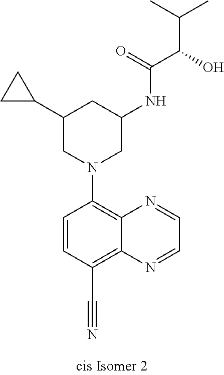 | 174 |
| 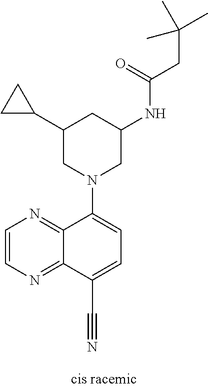 [SMILES: CC(C)(C)CC(=O)NC1CC(C2CC2)CN(c2ccc(C#N)c3nccnc23)C1] | 175 |
| 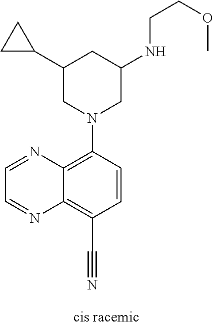 [SMILES: COCCNC1CC(C2CC2)CN(c2ccc(C#N)c3nccnc23)C1] | 176 |
| 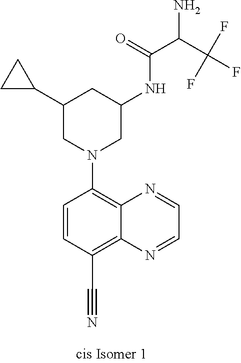 | 177 |
| 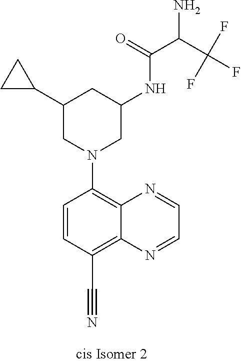 | 178 |
| 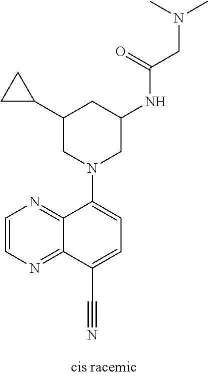 [SMILES: CN(C)CC(=O)NC1CC(C2CC2)CN(c2ccc(C#N)c3nccnc23)C1] | 179 |
| 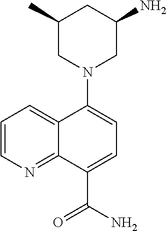 [SMILES: C[C@H]1C[C@@H](N)CN(c2ccc(C(N)=O)c3ncccc23)C1] | 180 |
| 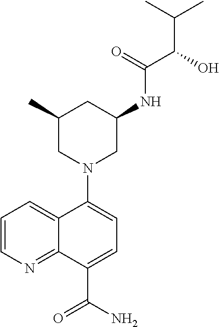 [SMILES: CC(C)[C@H](O)C(=O)N[C@@H]1C[C@H](C)CN(c2ccc(C(N)=O)c3ncccc23)C1] | 181 |
| 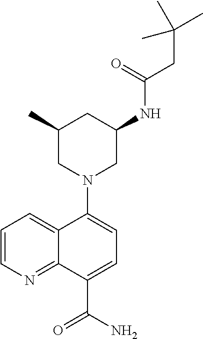 [SMILES: C[C@H]1C[C@@H](NC(=O)CC(C)(C)C)CN(c2ccc(C(N)=O)c3ncccc23)C1] | 182 |
| 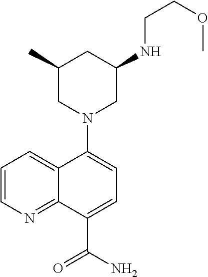 [SMILES: COCCN[C@@H]1C[C@H](C)CN(c2ccc(C(N)=O)c3ncccc23)C1] | 183 |
| 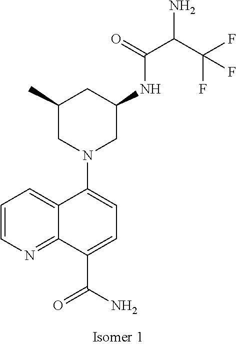 [SMILES: C[C@H]1C[C@@H](NC(=O)C(N)C(F)(F)F)CN(c2ccc(C(N)=O)c3ncccc23)C1] | 184 |
| 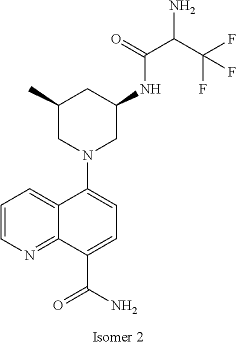 [SMILES: C[C@H]1C[C@@H](NC(=O)C(N)C(F)(F)F)CN(c2ccc(C(N)=O)c3ncccc23)C1] | 185 |
| 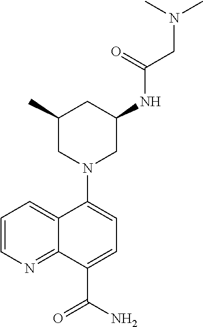 [SMILES: C[C@H]1C[C@@H](NC(=O)CN(C)C)CN(c2ccc(C(N)=O)c3ncccc23)C1] | 186 |
| 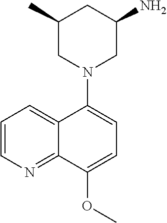 [SMILES: COc1ccc(N2C[C@@H](C)C[C@@H](N)C2)c2cccnc12] | 187 |
| 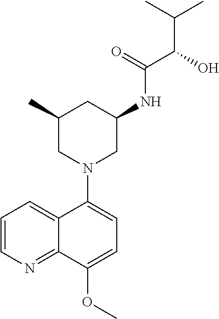 [SMILES: COc1ccc(N2C[C@@H](C)C[C@@H](NC(=O)[C@@H](O)C(C)C)C2)c2cccnc12] | 188 |
| 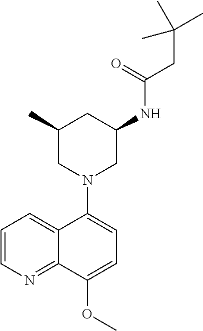 [SMILES: COc1ccc(N2C[C@@H](C)C[C@@H](NC(=O)CC(C)(C)C)C2)c2cccnc12] | 189 |
| 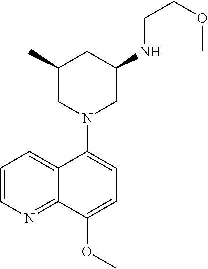 [SMILES: COCCN[C@@H]1C[C@H](C)CN(c2ccc(OC)c3ncccc23)C1] | 190 |
| 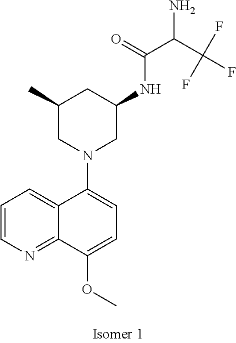 [SMILES: COc1ccc(N2C[C@@H](C)C[C@@H](NC(=O)C(N)C(F)(F)F)C2)c2cccnc12] | 191 |
| 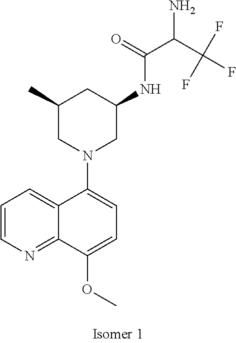 [SMILES: COc1ccc(N2C[C@@H](C)C[C@@H](NC(=O)C(N)C(F)(F)F)C2)c2cccnc12] | 192 |
| 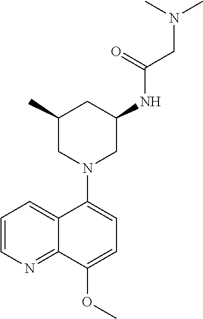 [SMILES: COc1ccc(N2C[C@@H](C)C[C@@H](NC(=O)CN(C)C)C2)c2cccnc12] | 193 |
| 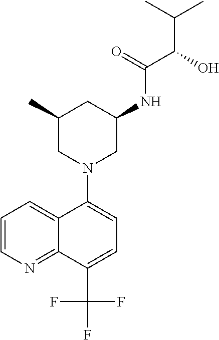 [SMILES: CC(C)[C@H](O)C(=O)N[C@@H]1C[C@H](C)CN(c2ccc(C(F)(F)F)c3ncccc23)C1] | 194 |
| 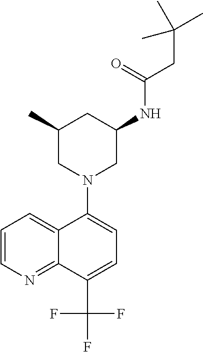 [SMILES: C[C@H]1C[C@@H](NC(=O)CC(C)(C)C)CN(c2ccc(C(F)(F)F)c3ncccc23)C1] | 195 |
| 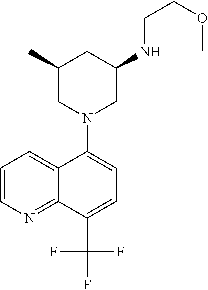 [SMILES: COCCN[C@@H]1C[C@H](C)CN(c2ccc(C(F)(F)F)c3ncccc23)C1] | 196 |
| 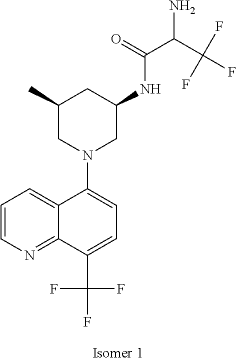 [SMILES: C[C@H]1C[C@@H](NC(=O)C(N)C(F)(F)F)CN(c2ccc(C(F)(F)F)c3ncccc23)C1] | 197 |
| 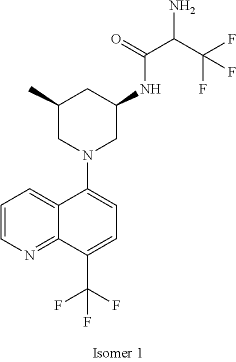 [SMILES: C[C@H]1C[C@@H](NC(=O)C(N)C(F)(F)F)CN(c2ccc(C(F)(F)F)c3ncccc23)C1] | 198 |
| 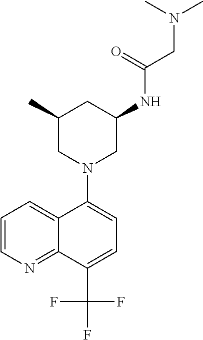 [SMILES: C[C@H]1C[C@@H](NC(=O)CN(C)C)CN(c2ccc(C(F)(F)F)c3ncccc23)C1] | 199 |
| 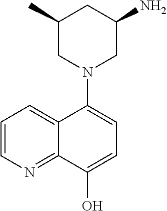 [SMILES: C[C@H]1C[C@@H](N)CN(c2ccc(O)c3ncccc23)C1] | 200 |
| 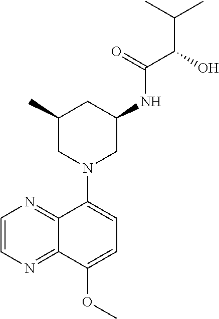 [SMILES: COc1ccc(N2C[C@@H](C)C[C@@H](NC(=O)[C@@H](O)C(C)C)C2)c2nccnc12] | 201 |
| 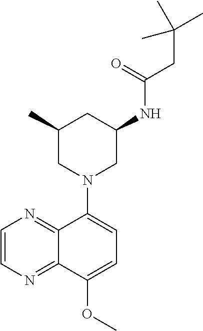 [SMILES: COc1ccc(N2C[C@@H](C)C[C@@H](NC(=O)CC(C)(C)C)C2)c2nccnc12] | 202 |
| 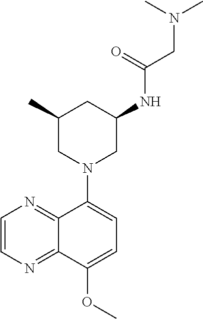 [SMILES: COc1ccc(N2C[C@@H](C)C[C@@H](NC(=O)CN(C)C)C2)c2nccnc12] | 203 |
| 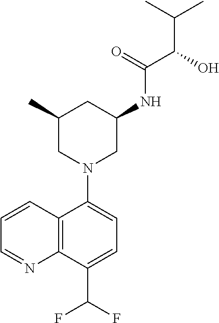 [SMILES: CC(C)[C@H](O)C(=O)N[C@@H]1C[C@H](C)CN(c2ccc(C(F)F)c3ncccc23)C1] | 204 |
| 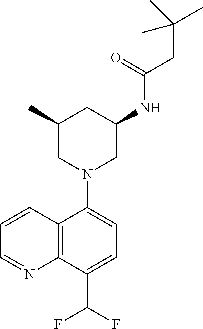 [SMILES: C[C@H]1C[C@@H](NC(=O)CC(C)(C)C)CN(c2ccc(C(F)F)c3ncccc23)C1] | 205 |
| 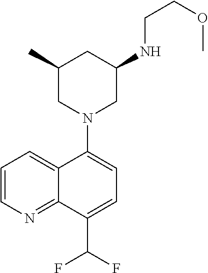 [SMILES: COCCN[C@@H]1C[C@H](C)CN(c2ccc(C(F)F)c3ncccc23)C1] | 206 |
| 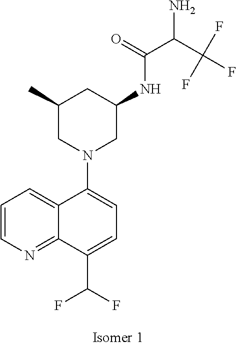 [SMILES: C[C@H]1C[C@@H](NC(=O)C(N)C(F)(F)F)CN(c2ccc(C(F)F)c3ncccc23)C1] | 207 |
| 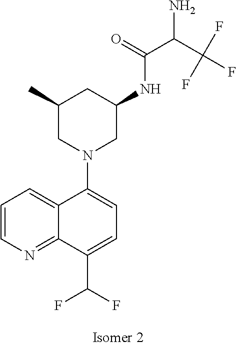 [SMILES: C[C@H]1C[C@@H](NC(=O)C(N)C(F)(F)F)CN(c2ccc(C(F)F)c3ncccc23)C1] | 208 |
| 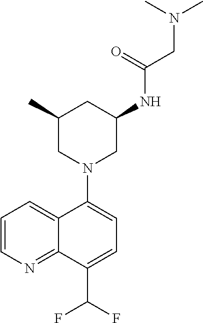 [SMILES: C[C@H]1C[C@@H](NC(=O)CN(C)C)CN(c2ccc(C(F)F)c3ncccc23)C1] | 209 |
| 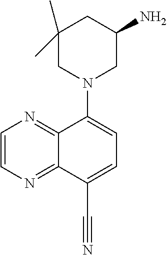 [SMILES: CC1(C)C[C@@H](N)CN(c2ccc(C#N)c3nccnc23)C1] | 210 |
| 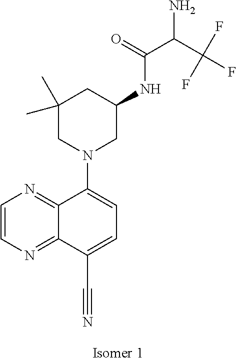 [SMILES: CC1(C)C[C@@H](NC(=O)C(N)C(F)(F)F)CN(c2ccc(C#N)c3nccnc23)C1] | 211 |
| 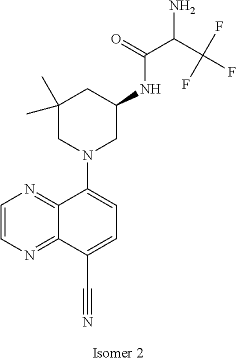 [SMILES: CC1(C)C[C@@H](NC(=O)C(N)C(F)(F)F)CN(c2ccc(C#N)c3nccnc23)C1] | 212 |
| 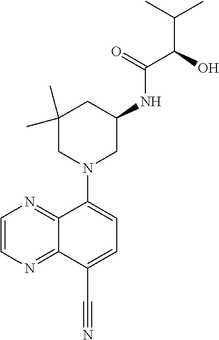 [SMILES: CC(C)[C@@H](O)C(=O)N[C@H]1CN(c2ccc(C#N)c3nccnc23)CC(C)(C)C1] | 213 |
| 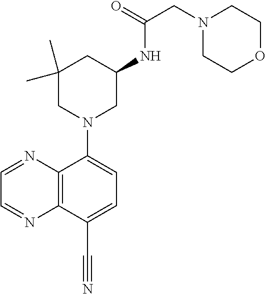 [SMILES: CC1(C)C[C@@H](NC(=O)CN2CCOCC2)CN(c2ccc(C#N)c3nccnc23)C1] | 214 |
| 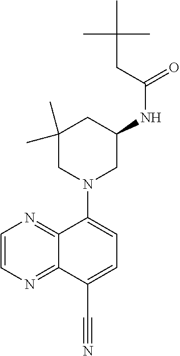 [SMILES: CC(C)(C)CC(=O)N[C@H]1CN(c2ccc(C#N)c3nccnc23)CC(C)(C)C1] | 215 |
| 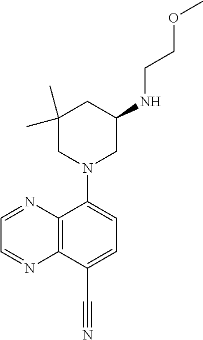 [SMILES: COCCN[C@H]1CN(c2ccc(C#N)c3nccnc23)CC(C)(C)C1] | 216 |
| 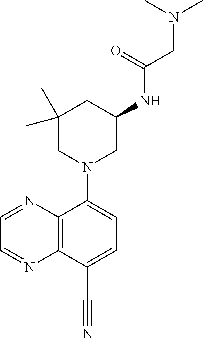 [SMILES: CN(C)CC(=O)N[C@H]1CN(c2ccc(C#N)c3nccnc23)CC(C)(C)C1] | 217 |
| 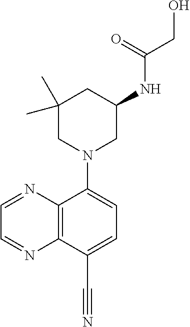 [SMILES: CC1(C)C[C@@H](NC(=O)CO)CN(c2ccc(C#N)c3nccnc23)C1] | 218 |
| 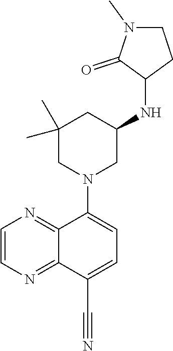 [SMILES: CN1CCC(N[C@H]2CN(c3ccc(C#N)c4nccnc34)CC(C)(C)C2)C1=O] | 219 |
| 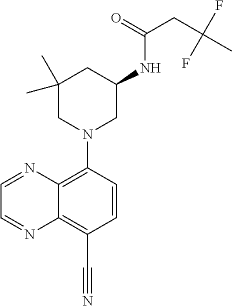 [SMILES: CC(F)(F)CC(=O)N[C@H]1CN(c2ccc(C#N)c3nccnc23)CC(C)(C)C1] | 220 |
| 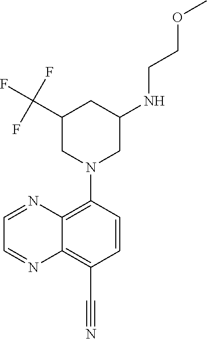 [SMILES: COCCNC1CC(C(F)(F)F)CN(c2ccc(C#N)c3nccnc23)C1] | 221 |
| 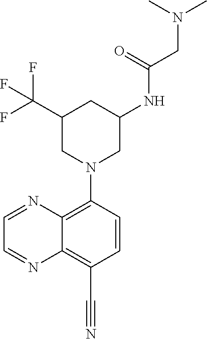 [SMILES: CN(C)CC(=O)NC1CC(C(F)(F)F)CN(c2ccc(C#N)c3nccnc23)C1] | 222 |
| 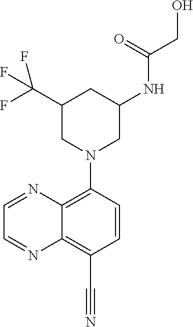 [SMILES: N#Cc1ccc(N2CC(NC(=O)CO)CC(C(F)(F)F)C2)c2nccnc12] | 223 |
| 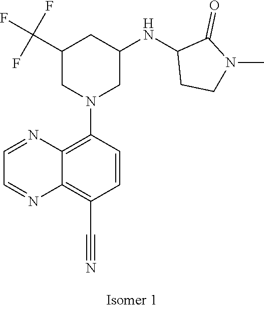 [SMILES: CN1CCC(NC2CC(C(F)(F)F)CN(c3ccc(C#N)c4nccnc34)C2)C1=O] | 224 |
| 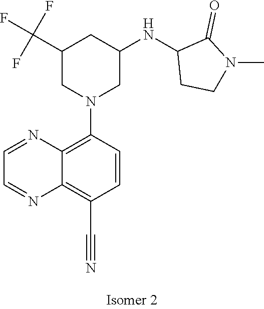 [SMILES: CN1CCC(NC2CC(C(F)(F)F)CN(c3ccc(C#N)c4nccnc34)C2)C1=O] | 225 |
| 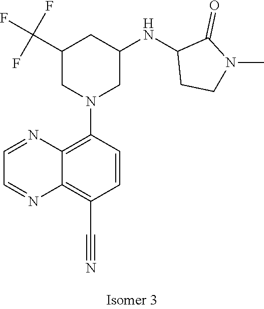 [SMILES: CN1CCC(NC2CC(C(F)(F)F)CN(c3ccc(C#N)c4nccnc34)C2)C1=O] | 226 |
| 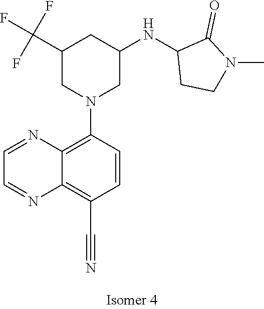 [SMILES: CN1CCC(NC2CC(C(F)(F)F)CN(c3ccc(C#N)c4nccnc34)C2)C1=O] | 227 |
| 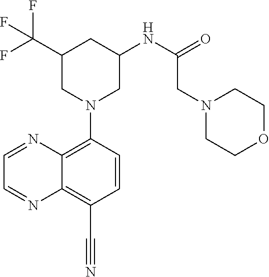 [SMILES: N#Cc1ccc(N2CC(NC(=O)CN3CCOCC3)CC(C(F)(F)F)C2)c2nccnc12] | 228 |
| 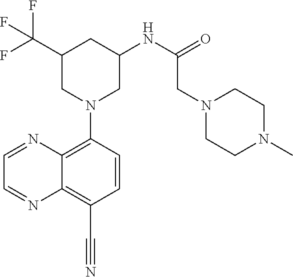 [SMILES: CN1CCN(CC(=O)NC2CC(C(F)(F)F)CN(c3ccc(C#N)c4nccnc34)C2)CC1] | 229 |
| 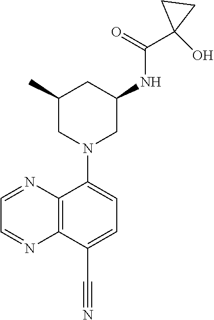 [SMILES: C[C@H]1C[C@@H](NC(=O)C2(O)CC2)CN(c2ccc(C#N)c3nccnc23)C1] | 230 |
| 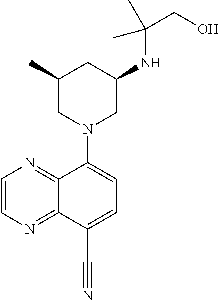 [SMILES: C[C@H]1C[C@@H](NC(C)(C)CO)CN(c2ccc(C#N)c3nccnc23)C1] | 231 |
| 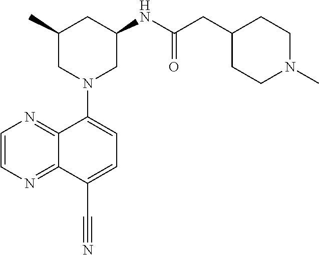 [SMILES: C[C@H]1C[C@@H](NC(=O)CC2CCN(C)CC2)CN(c2ccc(C#N)c3nccnc23)C1] | 232 |
| 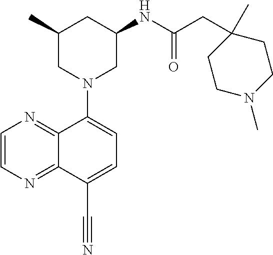 [SMILES: C[C@H]1C[C@@H](NC(=O)CC2(C)CCN(C)CC2)CN(c2ccc(C#N)c3nccnc23)C1] | 233 |
| 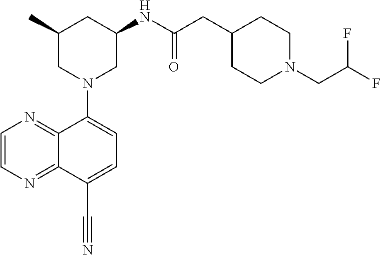 [SMILES: C[C@H]1C[C@@H](NC(=O)CC2CCN(CC(F)F)CC2)CN(c2ccc(C#N)c3nccnc23)C1] | 234 |
| 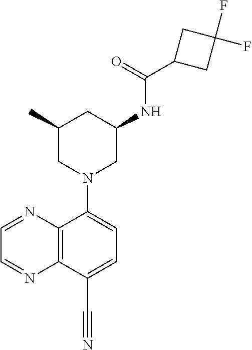 [SMILES: C[C@H]1C[C@@H](NC(=O)C2CC(F)(F)C2)CN(c2ccc(C#N)c3nccnc23)C1] | 235 |
| 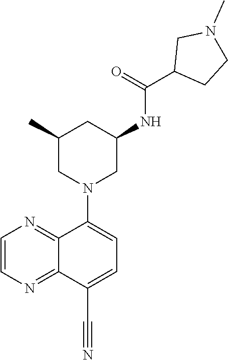 [SMILES: C[C@H]1C[C@@H](NC(=O)C2CCN(C)C2)CN(c2ccc(C#N)c3nccnc23)C1] | 236 |
| 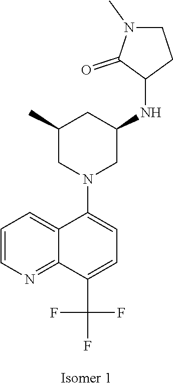 [SMILES: C[C@H]1C[C@@H](NC2CCN(C)C2=O)CN(c2ccc(C(F)(F)F)c3ncccc23)C1] | 237 |
| 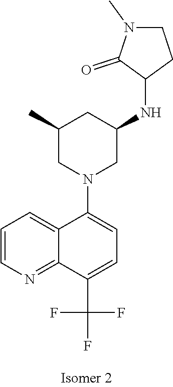 [SMILES: C[C@H]1C[C@@H](NC2CCN(C)C2=O)CN(c2ccc(C(F)(F)F)c3ncccc23)C1] | 238 |
| 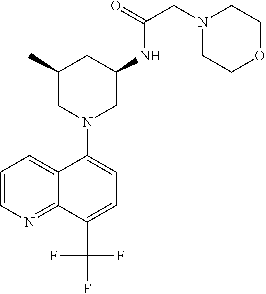 [SMILES: C[C@H]1C[C@@H](NC(=O)CN2CCOCC2)CN(c2ccc(C(F)(F)F)c3ncccc23)C1] | 239 |
| 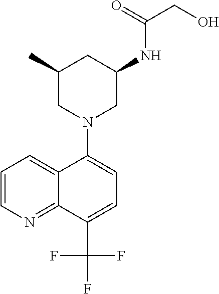 [SMILES: C[C@H]1C[C@@H](NC(=O)CO)CN(c2ccc(C(F)(F)F)c3ncccc23)C1] | 240 |
| 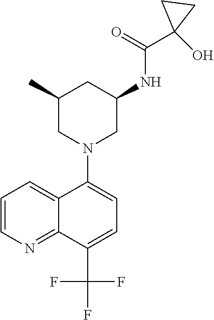 [SMILES: C[C@H]1C[C@@H](NC(=O)C2(O)CC2)CN(c2ccc(C(F)(F)F)c3ncccc23)C1] | 241 |
| 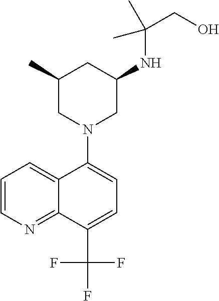 [SMILES: C[C@H]1C[C@@H](NC(C)(C)CO)CN(c2ccc(C(F)(F)F)c3ncccc23)C1] | 242 |
| 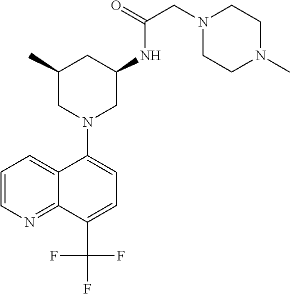 [SMILES: C[C@H]1C[C@@H](NC(=O)CN2CCN(C)CC2)CN(c2ccc(C(F)(F)F)c3ncccc23)C1] | 243 |
| 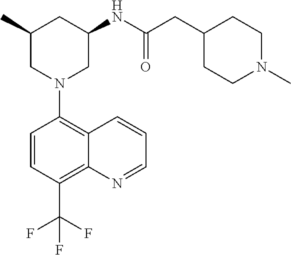 | 244 |
| 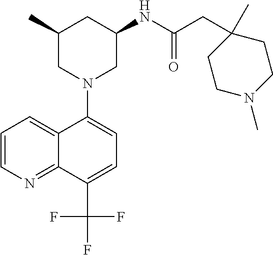 [SMILES: C[C@H]1C[C@@H](NC(=O)CC2(C)CCN(C)CC2)CN(c2ccc(C(F)(F)F)c3ncccc23)C1] | 245 |
| 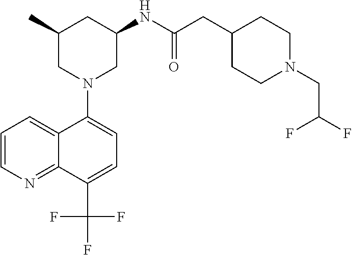 [SMILES: C[C@H]1C[C@@H](NC(=O)CC2CCN(CC(F)F)CC2)CN(c2ccc(C(F)(F)F)c3ncccc23)C1] | 246 |
| 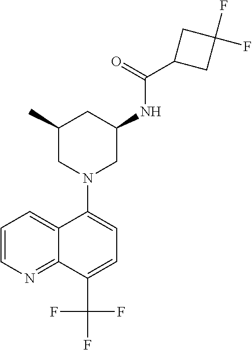 [SMILES: C[C@H]1C[C@@H](NC(=O)C2CC(F)(F)C2)CN(c2ccc(C(F)(F)F)c3ncccc23)C1] | 247 |
| 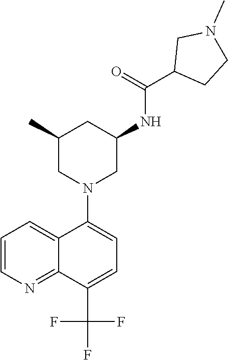 [SMILES: C[C@H]1C[C@@H](NC(=O)C2CCN(C)C2)CN(c2ccc(C(F)(F)F)c3ncccc23)C1] | 248 |
| 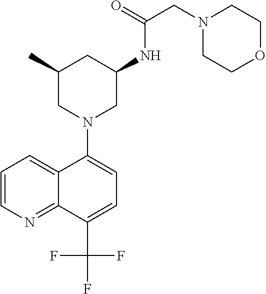 [SMILES: C[C@H]1C[C@@H](NC(=O)CN2CCOCC2)CN(c2ccc(C(F)(F)F)c3ncccc23)C1] | 249 |
| 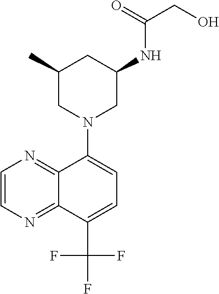 [SMILES: C[C@H]1C[C@@H](NC(=O)CO)CN(c2ccc(C(F)(F)F)c3nccnc23)C1] | 250 |
| 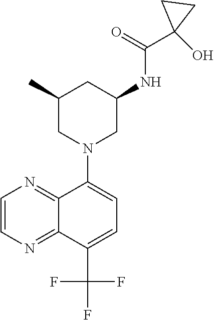 [SMILES: C[C@H]1C[C@@H](NC(=O)C2(O)CC2)CN(c2ccc(C(F)(F)F)c3nccnc23)C1] | 251 |
| 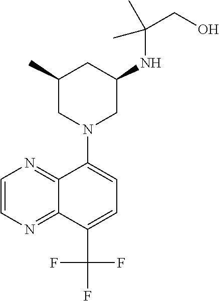 [SMILES: C[C@H]1C[C@@H](NC(C)(C)CO)CN(c2ccc(C(F)(F)F)c3nccnc23)C1] | 252 |
| 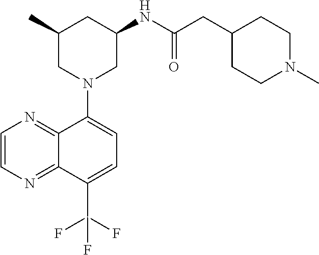 | 253 |
| 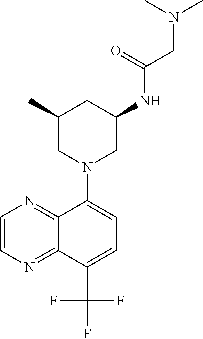 [SMILES: C[C@H]1C[C@@H](NC(=O)CN(C)C)CN(c2ccc(C(F)(F)F)c3nccnc23)C1] | 254 |
| 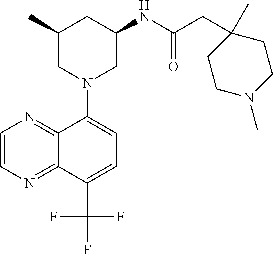 [SMILES: C[C@H]1C[C@@H](NC(=O)CC2(C)CCN(C)CC2)CN(c2ccc(C(F)(F)F)c3nccnc23)C1] | 255 |
| 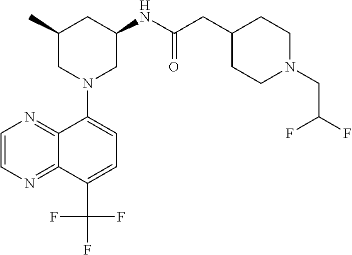 [SMILES: C[C@H]1C[C@@H](NC(=O)CC2CCN(CC(F)F)CC2)CN(c2ccc(C(F)(F)F)c3nccnc23)C1] | 256 |
| 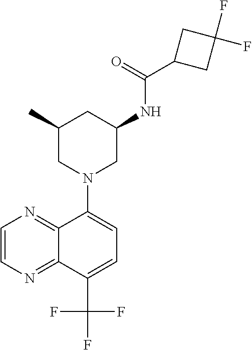 [SMILES: C[C@H]1C[C@@H](NC(=O)C2CC(F)(F)C2)CN(c2ccc(C(F)(F)F)c3nccnc23)C1] | 257 |
| 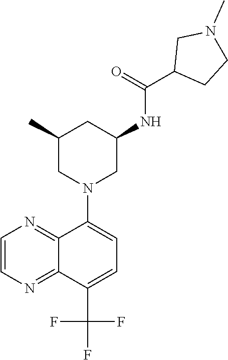 [SMILES: C[C@H]1C[C@@H](NC(=O)C2CCN(C)C2)CN(c2ccc(C(F)(F)F)c3nccnc23)C1] | 258 |
| 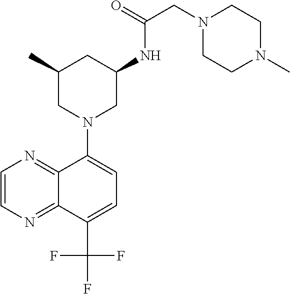 [SMILES: C[C@H]1C[C@@H](NC(=O)CN2CCN(C)CC2)CN(c2ccc(C(F)(F)F)c3nccnc23)C1] | 259 |
| 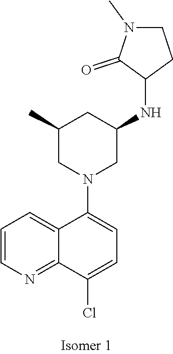 [SMILES: C[C@H]1C[C@@H](NC2CCN(C)C2=O)CN(c2ccc(Cl)c3ncccc23)C1] | 260 |
| 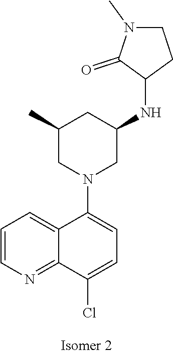 [SMILES: C[C@H]1C[C@@H](NC2CCN(C)C2=O)CN(c2ccc(Cl)c3ncccc23)C1] | 261 |
| 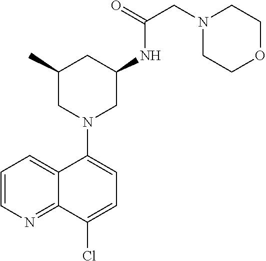 [SMILES: C[C@H]1C[C@@H](NC(=O)CN2CCOCC2)CN(c2ccc(Cl)c3ncccc23)C1] | 262 |
| 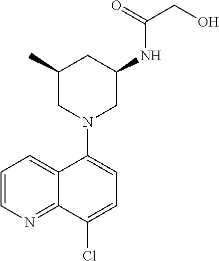 [SMILES: C[C@H]1C[C@@H](NC(=O)CO)CN(c2ccc(Cl)c3ncccc23)C1] | 263 |
| 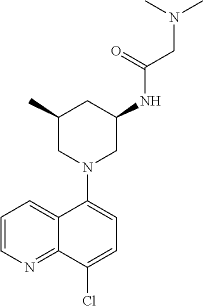 [SMILES: C[C@H]1C[C@@H](NC(=O)CN(C)C)CN(c2ccc(Cl)c3ncccc23)C1] | 264 |
| 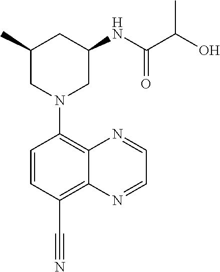 [SMILES: CC(O)C(=O)N[C@@H]1C[C@H](C)CN(c2ccc(C#N)c3nccnc23)C1] | 265 |
| 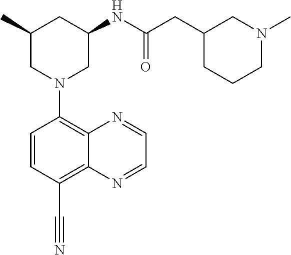 | 266 |
| 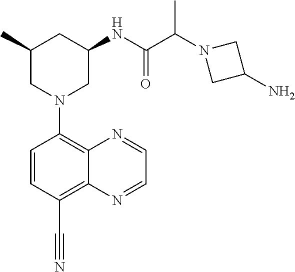 [SMILES: CC(C(=O)N[C@@H]1C[C@H](C)CN(c2ccc(C#N)c3nccnc23)C1)N1CC(N)C1] | 267 |
| 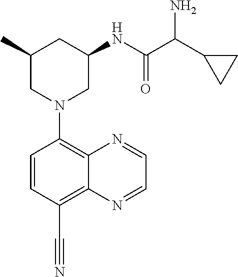 [SMILES: C[C@H]1C[C@@H](NC(=O)C(N)C2CC2)CN(c2ccc(C#N)c3nccnc23)C1] | 268 |
| 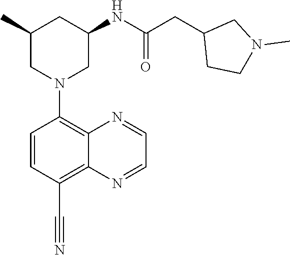 | 269 |
| 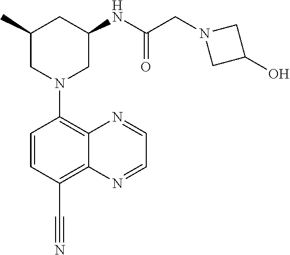 [SMILES: C[C@H]1C[C@@H](NC(=O)CN2CC(O)C2)CN(c2ccc(C#N)c3nccnc23)C1] | 270 |
| 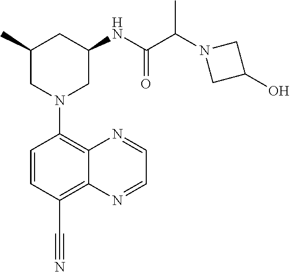 [SMILES: CC(C(=O)N[C@@H]1C[C@H](C)CN(c2ccc(C#N)c3nccnc23)C1)N1CC(O)C1] | 271 |
| 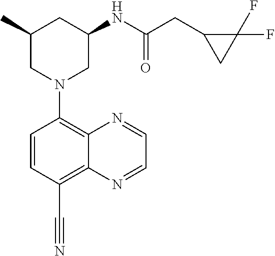 [SMILES: C[C@H]1C[C@@H](NC(=O)CC2CC2(F)F)CN(c2ccc(C#N)c3nccnc23)C1] | 272 |
| 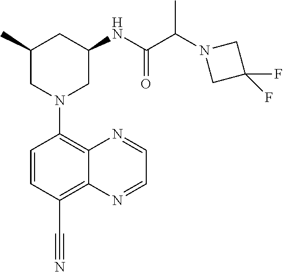 [SMILES: CC(C(=O)N[C@@H]1C[C@H](C)CN(c2ccc(C#N)c3nccnc23)C1)N1CC(F)(F)C1] | 273 |
| 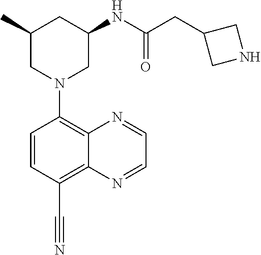 [SMILES: C[C@H]1C[C@@H](NC(=O)CC2CNC2)CN(c2ccc(C#N)c3nccnc23)C1] | 274 |
| 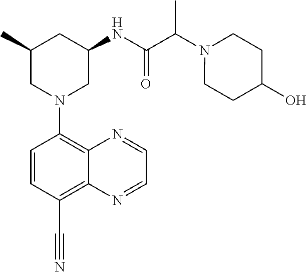 [SMILES: CC(C(=O)N[C@@H]1C[C@H](C)CN(c2ccc(C#N)c3nccnc23)C1)N1CCC(O)CC1] | 275 |
| 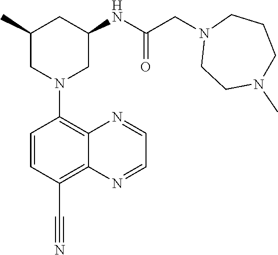 | 276 |
| 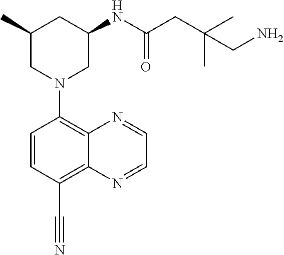 [SMILES: C[C@H]1C[C@@H](NC(=O)CC(C)(C)CN)CN(c2ccc(C#N)c3nccnc23)C1] | 277 |
| 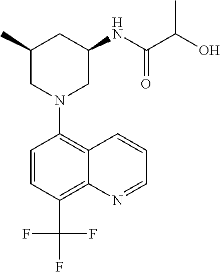 [SMILES: CC(O)C(=O)N[C@@H]1C[C@H](C)CN(c2ccc(C(F)(F)F)c3ncccc23)C1] | 278 |
| 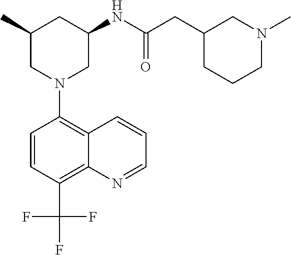 | 279 |
| 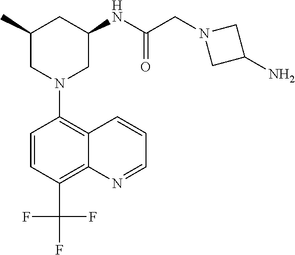 [SMILES: C[C@H]1C[C@@H](NC(=O)CN2CC(N)C2)CN(c2ccc(C(F)(F)F)c3ncccc23)C1] | 280 |
| 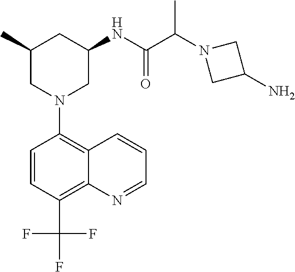 [SMILES: CC(C(=O)N[C@@H]1C[C@H](C)CN(c2ccc(C(F)(F)F)c3ncccc23)C1)N1CC(N)C1] | 281 |
| 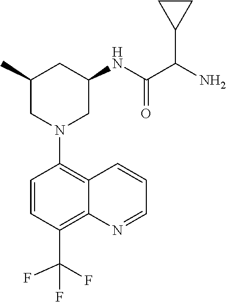 [SMILES: C[C@H]1C[C@@H](NC(=O)C(N)C2CC2)CN(c2ccc(C(F)(F)F)c3ncccc23)C1] | 282 |
| 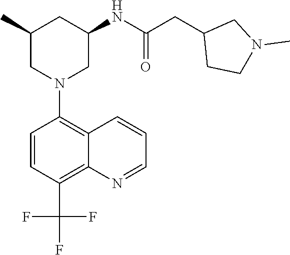 | 283 |
| 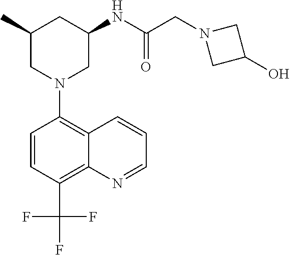 [SMILES: C[C@H]1C[C@@H](NC(=O)CN2CC(O)C2)CN(c2ccc(C(F)(F)F)c3ncccc23)C1] | 284 |
| 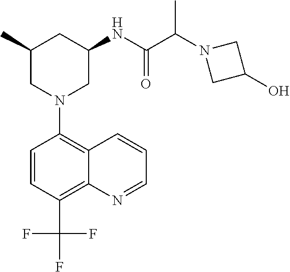 [SMILES: CC(C(=O)N[C@@H]1C[C@H](C)CN(c2ccc(C(F)(F)F)c3ncccc23)C1)N1CC(O)C1] | 285 |
| 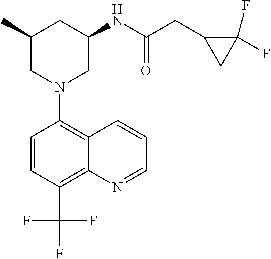 [SMILES: C[C@H]1C[C@@H](NC(=O)CC2CC2(F)F)CN(c2ccc(C(F)(F)F)c3ncccc23)C1] | 286 |
| 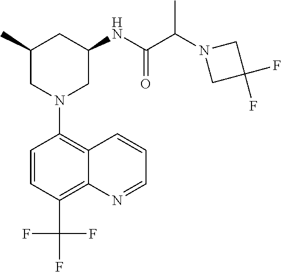 [SMILES: CC(C(=O)N[C@@H]1C[C@H](C)CN(c2ccc(C(F)(F)F)c3ncccc23)C1)N1CC(F)(F)C1] | 287 |
| 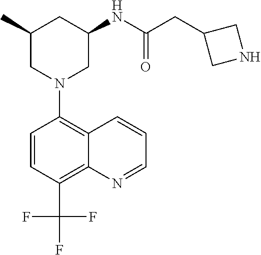 [SMILES: C[C@H]1C[C@@H](NC(=O)CC2CNC2)CN(c2ccc(C(F)(F)F)c3ncccc23)C1] | 288 |
| 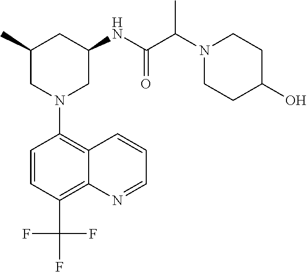 [SMILES: CC(C(=O)N[C@@H]1C[C@H](C)CN(c2ccc(C(F)(F)F)c3ncccc23)C1)N1CCC(O)CC1] | 289 |
| 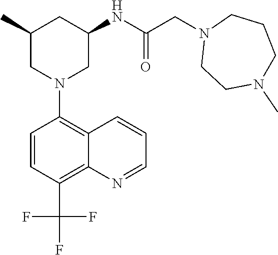 | 290 |
| 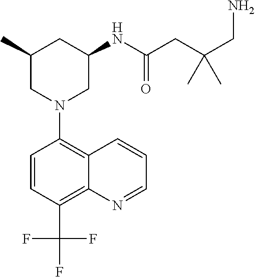 [SMILES: C[C@H]1C[C@@H](NC(=O)CC(C)(C)CN)CN(c2ccc(C(F)(F)F)c3ncccc23)C1] | 291 |
| 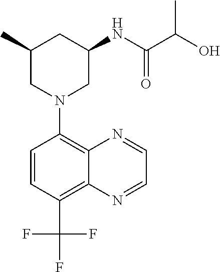 [SMILES: CC(O)C(=O)N[C@@H]1C[C@H](C)CN(c2ccc(C(F)(F)F)c3nccnc23)C1] | 292 |
| 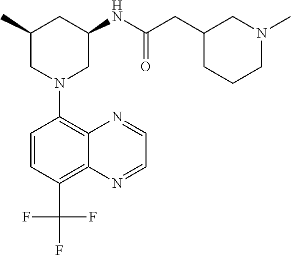 | 293 |
| 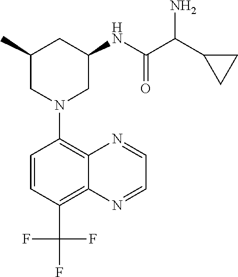 [SMILES: C[C@H]1C[C@@H](NC(=O)C(N)C2CC2)CN(c2ccc(C(F)(F)F)c3nccnc23)C1] | 294 |
| 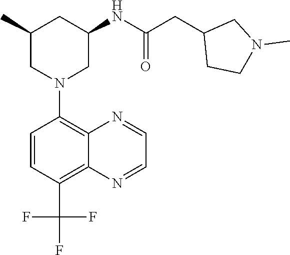 | 295 |
| 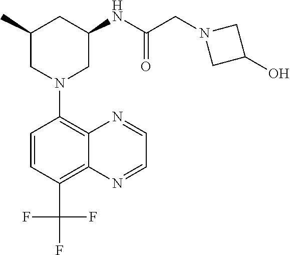 [SMILES: C[C@H]1C[C@@H](NC(=O)CN2CC(O)C2)CN(c2ccc(C(F)(F)F)c3nccnc23)C1] | 296 |
| 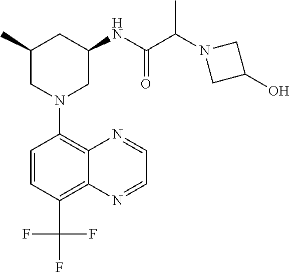 [SMILES: CC(C(=O)N[C@@H]1C[C@H](C)CN(c2ccc(C(F)(F)F)c3nccnc23)C1)N1CC(O)C1] | 297 |
| 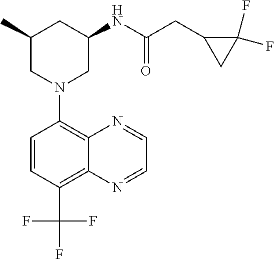 [SMILES: C[C@H]1C[C@@H](NC(=O)CC2CC2(F)F)CN(c2ccc(C(F)(F)F)c3nccnc23)C1] | 298 |
| 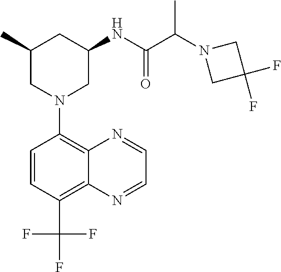 [SMILES: CC(C(=O)N[C@@H]1C[C@H](C)CN(c2ccc(C(F)(F)F)c3nccnc23)C1)N1CC(F)(F)C1] | 299 |
| 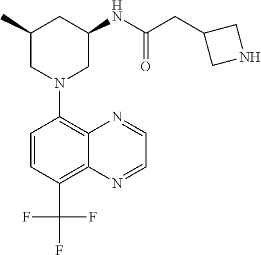 [SMILES: C[C@H]1C[C@@H](NC(=O)CC2CNC2)CN(c2ccc(C(F)(F)F)c3nccnc23)C1] | 300 |
| 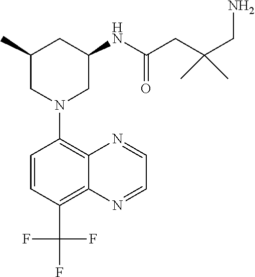 [SMILES: C[C@H]1C[C@@H](NC(=O)CC(C)(C)CN)CN(c2ccc(C(F)(F)F)c3nccnc23)C1] | 301 |
| 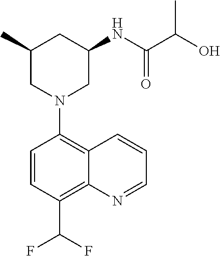 [SMILES: CC(O)C(=O)N[C@@H]1C[C@H](C)CN(c2ccc(C(F)F)c3ncccc23)C1] | 302 |
| 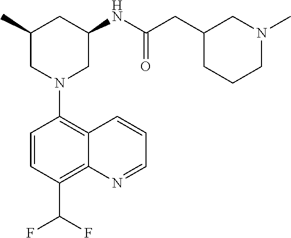 | 303 |
| 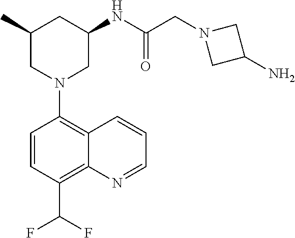 [SMILES: C[C@H]1C[C@@H](NC(=O)CN2CC(N)C2)CN(c2ccc(C(F)F)c3ncccc23)C1] | 304 |
| 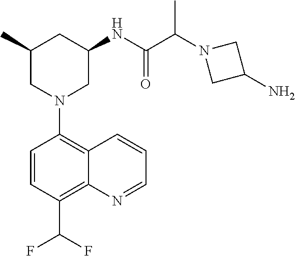 [SMILES: CC(C(=O)N[C@@H]1C[C@H](C)CN(c2ccc(C(F)F)c3ncccc23)C1)N1CC(N)C1] | 305 |
| 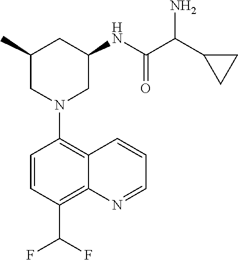 [SMILES: C[C@H]1C[C@@H](NC(=O)C(N)C2CC2)CN(c2ccc(C(F)F)c3ncccc23)C1] | 306 |
| 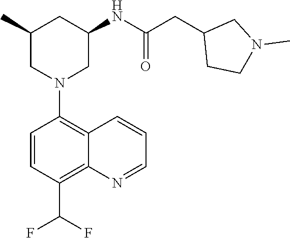 | 307 |
| 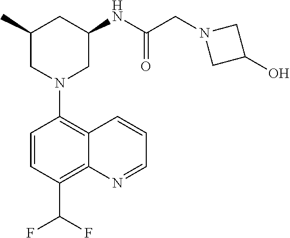 [SMILES: C[C@H]1C[C@@H](NC(=O)CN2CC(O)C2)CN(c2ccc(C(F)F)c3ncccc23)C1] | 308 |
| 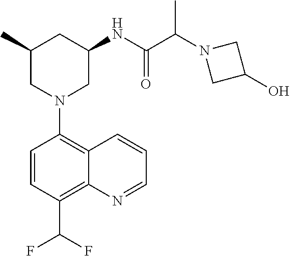 [SMILES: CC(C(=O)N[C@@H]1C[C@H](C)CN(c2ccc(C(F)F)c3ncccc23)C1)N1CC(O)C1] | 309 |
| 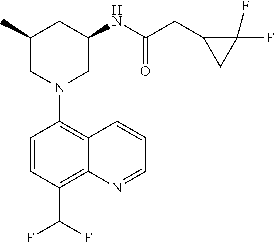 [SMILES: C[C@H]1C[C@@H](NC(=O)CC2CC2(F)F)CN(c2ccc(C(F)F)c3ncccc23)C1] | 310 |
| 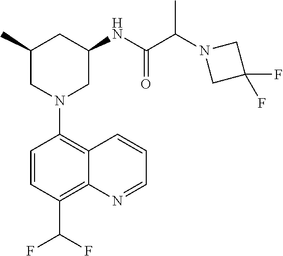 [SMILES: CC(C(=O)N[C@@H]1C[C@H](C)CN(c2ccc(C(F)F)c3ncccc23)C1)N1CC(F)(F)C1] | 311 |
| 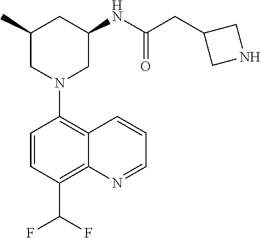 [SMILES: C[C@H]1C[C@@H](NC(=O)CC2CNC2)CN(c2ccc(C(F)F)c3ncccc23)C1] | 312 |
| 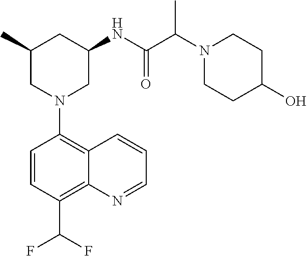 [SMILES: CC(C(=O)N[C@@H]1C[C@H](C)CN(c2ccc(C(F)F)c3ncccc23)C1)N1CCC(O)CC1] | 313 |
| 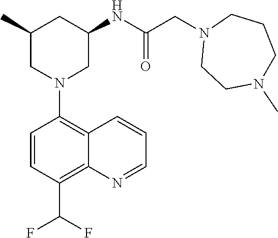 | 314 |
| 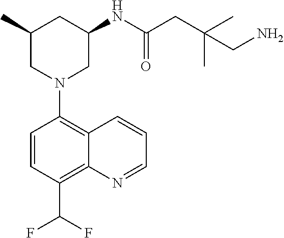 [SMILES: C[C@H]1C[C@@H](NC(=O)CC(C)(C)CN)CN(c2ccc(C(F)F)c3ncccc23)C1] | 315 |
| 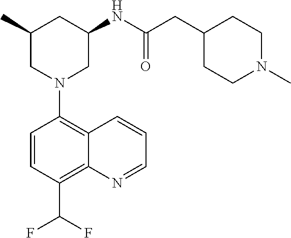 | 316 |
| 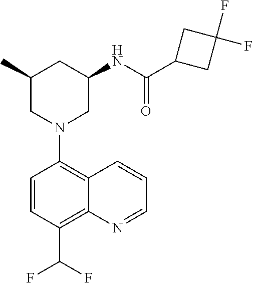 [SMILES: C[C@H]1C[C@@H](NC(=O)C2CC(F)(F)C2)CN(c2ccc(C(F)F)c3ncccc23)C1] | 317 |
| 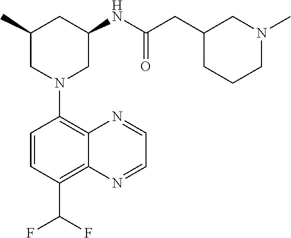 | 318 |
| 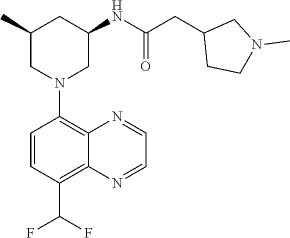 | 319 |
| 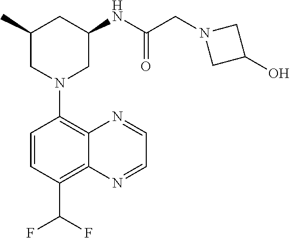 [SMILES: C[C@H]1C[C@@H](NC(=O)CN2CC(O)C2)CN(c2ccc(C(F)F)c3nccnc23)C1] | 320 |
| 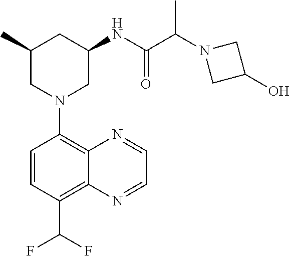 [SMILES: CC(C(=O)N[C@@H]1C[C@H](C)CN(c2ccc(C(F)F)c3nccnc23)C1)N1CC(O)C1] | 321 |
| 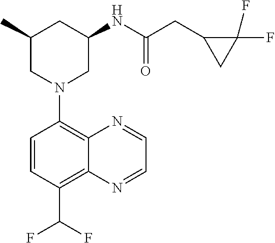 [SMILES: C[C@H]1C[C@@H](NC(=O)CC2CC2(F)F)CN(c2ccc(C(F)F)c3nccnc23)C1] | 322 |
| 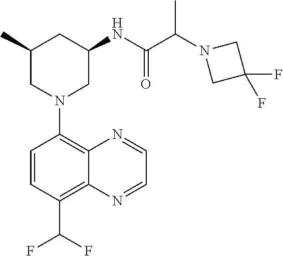 [SMILES: CC(C(=O)N[C@@H]1C[C@H](C)CN(c2ccc(C(F)F)c3nccnc23)C1)N1CC(F)(F)C1] | 323 |
| 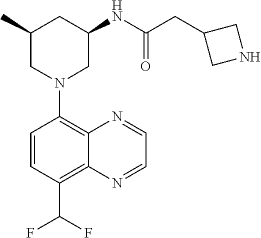 [SMILES: C[C@H]1C[C@@H](NC(=O)CC2CNC2)CN(c2ccc(C(F)F)c3nccnc23)C1] | 324 |
| 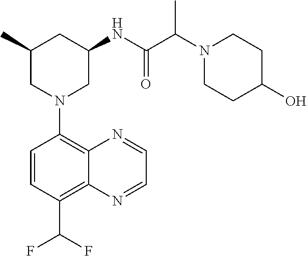 [SMILES: CC(C(=O)N[C@@H]1C[C@H](C)CN(c2ccc(C(F)F)c3nccnc23)C1)N1CCC(O)CC1] | 325 |
| 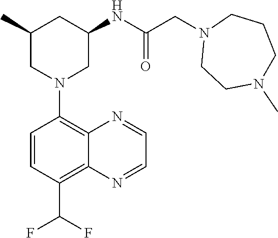 | 326 |
| 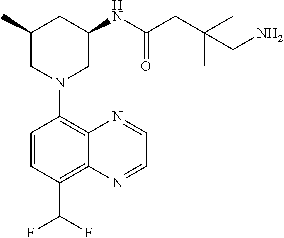 [SMILES: C[C@H]1C[C@@H](NC(=O)CC(C)(C)CN)CN(c2ccc(C(F)F)c3nccnc23)C1] | 327 |
| 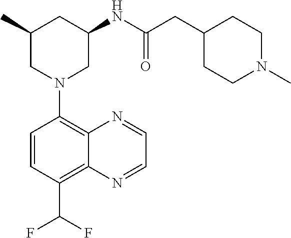 | 328 |
| 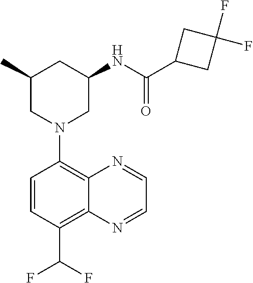 [SMILES: C[C@H]1C[C@@H](NC(=O)C2CC(F)(F)C2)CN(c2ccc(C(F)F)c3nccnc23)C1] | 329 |
| 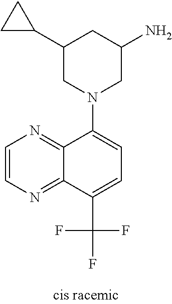 [SMILES: NC1CC(C2CC2)CN(c2ccc(C(F)(F)F)c3nccnc23)C1] | 330 |
| 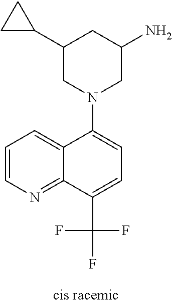 [SMILES: NC1CC(C2CC2)CN(c2ccc(C(F)(F)F)c3ncccc23)C1] | 331 |
| 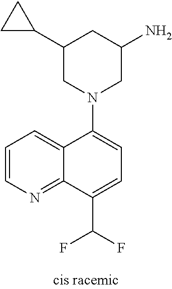 [SMILES: NC1CC(C2CC2)CN(c2ccc(C(F)F)c3ncccc23)C1] | 332 |
| 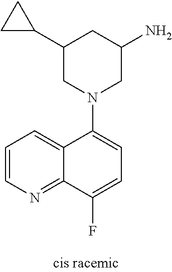 [SMILES: NC1CC(C2CC2)CN(c2ccc(F)c3ncccc23)C1] | 333 |
| 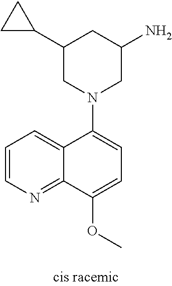 [SMILES: COc1ccc(N2CC(N)CC(C3CC3)C2)c2cccnc12] | 334 |
| 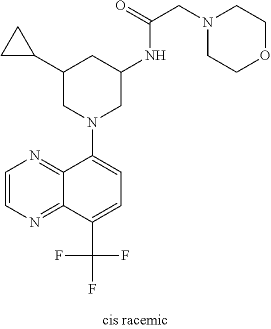 [SMILES: O=C(CN1CCOCC1)NC1CC(C2CC2)CN(c2ccc(C(F)(F)F)c3nccnc23)C1] | 335 |
| 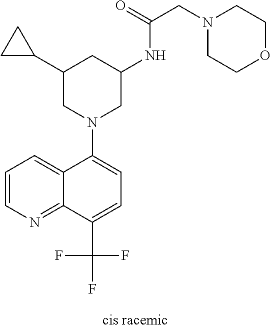 [SMILES: O=C(CN1CCOCC1)NC1CC(C2CC2)CN(c2ccc(C(F)(F)F)c3ncccc23)C1] | 336 |
| 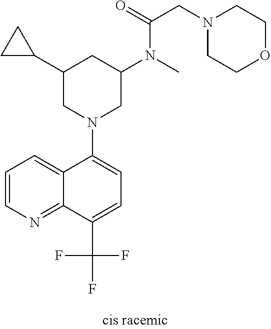 [SMILES: CN(C(=O)CN1CCOCC1)C1CC(C2CC2)CN(c2ccc(C(F)(F)F)c3ncccc23)C1] | 337 |
| 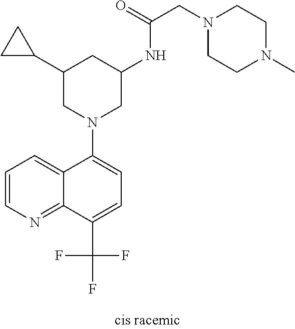 [SMILES: CN1CCN(CC(=O)NC2CC(C3CC3)CN(c3ccc(C(F)(F)F)c4ncccc34)C2)CC1] | 338 |
| 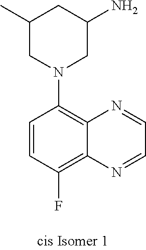 [SMILES: CC1CC(N)CN(c2ccc(F)c3nccnc23)C1] | 339 |
| 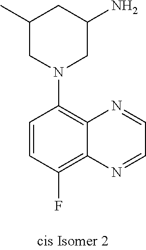 [SMILES: CC1CC(N)CN(c2ccc(F)c3nccnc23)C1] | 340 |
| 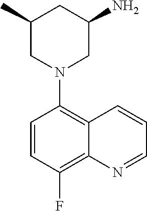 [SMILES: C[C@H]1C[C@@H](N)CN(c2ccc(F)c3ncccc23)C1] | 341 |
| 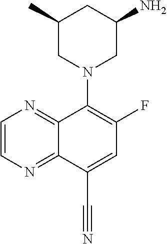 [SMILES: C[C@H]1C[C@@H](N)CN(c2c(F)cc(C#N)c3nccnc23)C1] | 342 |
| 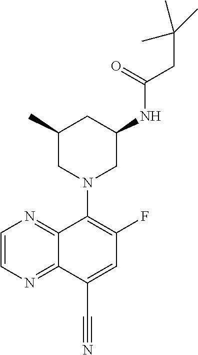 [SMILES: C[C@H]1C[C@@H](NC(=O)CC(C)(C)C)CN(c2c(F)cc(C#N)c3nccnc23)C1] | 343 |
| 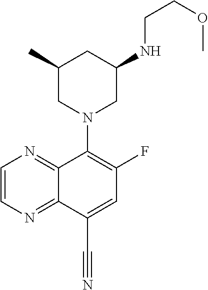 [SMILES: COCCN[C@@H]1C[C@H](C)CN(c2c(F)cc(C#N)c3nccnc23)C1] | 344 |
| 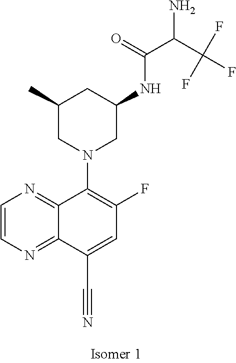 [SMILES: C[C@H]1C[C@@H](NC(=O)C(N)C(F)(F)F)CN(c2c(F)cc(C#N)c3nccnc23)C1] | 345 |
| 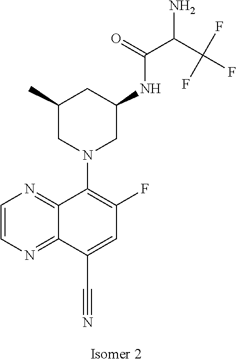 [SMILES: C[C@H]1C[C@@H](NC(=O)C(N)C(F)(F)F)CN(c2c(F)cc(C#N)c3nccnc23)C1] | 346 |
| 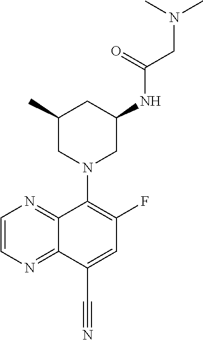 [SMILES: C[C@H]1C[C@@H](NC(=O)CN(C)C)CN(c2c(F)cc(C#N)c3nccnc23)C1] | 347 |
| 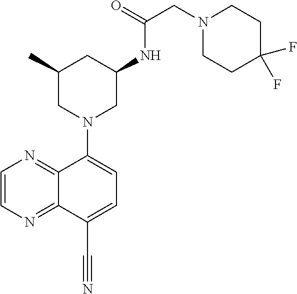 [SMILES: C[C@H]1C[C@@H](NC(=O)CN2CCC(F)(F)CC2)CN(c2ccc(C#N)c3nccnc23)C1] | 348 |
| 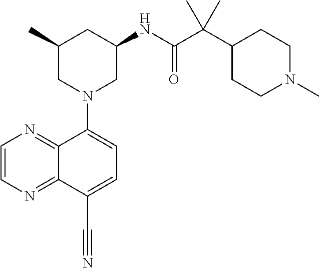 [SMILES: C[C@H]1C[C@@H](NC(=O)C(C)(C)C2CCN(C)CC2)CN(c2ccc(C#N)c3nccnc23)C1] | 349 |
| 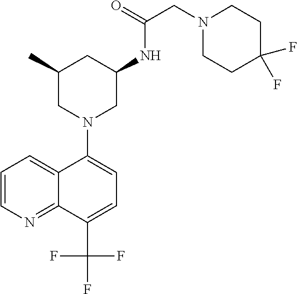 [SMILES: C[C@H]1C[C@@H](NC(=O)CN2CCC(F)(F)CC2)CN(c2ccc(C(F)(F)F)c3ncccc23)C1] | 350 |
| 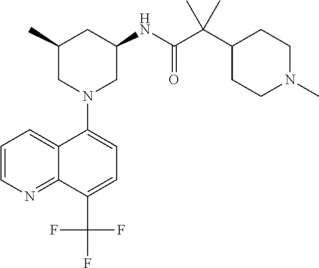 [SMILES: C[C@H]1C[C@@H](NC(=O)C(C)(C)C2CCN(C)CC2)CN(c2ccc(C(F)(F)F)c3ncccc23)C1] | 351 |
| 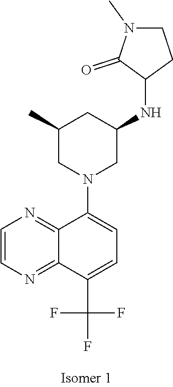 [SMILES: C[C@H]1C[C@@H](NC2CCN(C)C2=O)CN(c2ccc(C(F)(F)F)c3nccnc23)C1] | 352 |
| 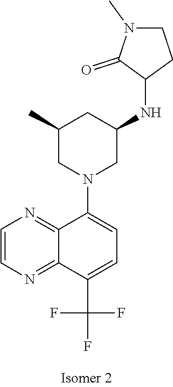 [SMILES: C[C@H]1C[C@@H](NC2CCN(C)C2=O)CN(c2ccc(C(F)(F)F)c3nccnc23)C1] | 353 |
| 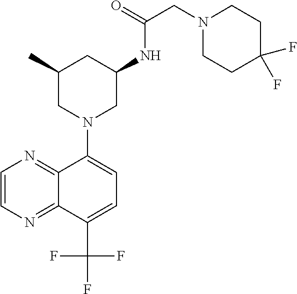 [SMILES: C[C@H]1C[C@@H](NC(=O)CN2CCC(F)(F)CC2)CN(c2ccc(C(F)(F)F)c3nccnc23)C1] | 354 |
| 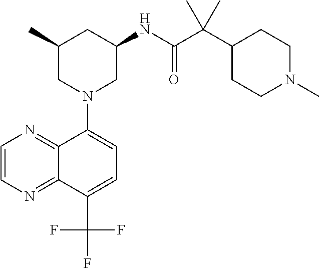 [SMILES: C[C@H]1C[C@@H](NC(=O)C(C)(C)C2CCN(C)CC2)CN(c2ccc(C(F)(F)F)c3nccnc23)C1] | 355 |
| 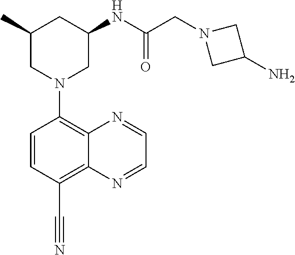 [SMILES: C[C@H]1C[C@@H](NC(=O)CN2CC(N)C2)CN(c2ccc(C#N)c3nccnc23)C1] | 356 |
| 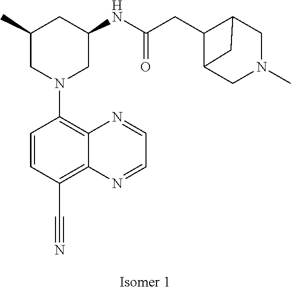 | 357 |
| 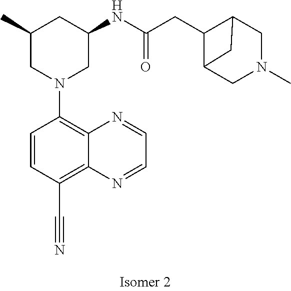 | 358 |
| 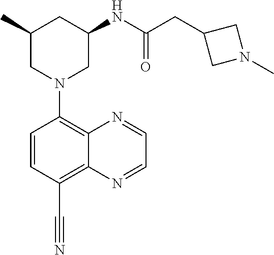 | 359 |
| 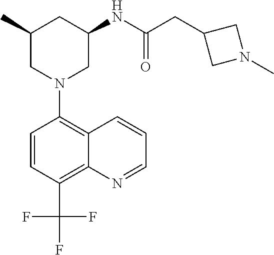 | 360 |
| 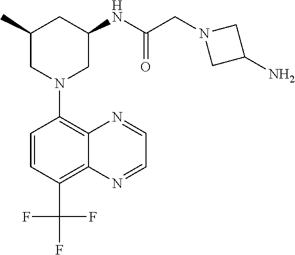 [SMILES: C[C@H]1C[C@@H](NC(=O)CN2CC(N)C2)CN(c2ccc(C(F)(F)F)c3nccnc23)C1] | 361 |
| 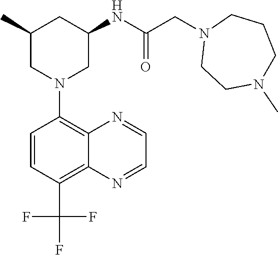 | 362 |
| 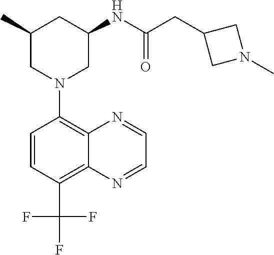 | 363 |
| 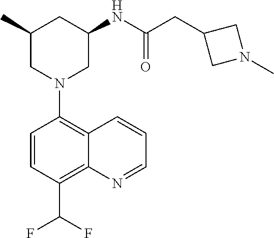 | 364 |
| 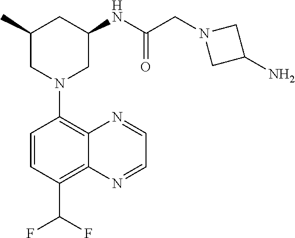 [SMILES: C[C@H]1C[C@@H](NC(=O)CN2CC(N)C2)CN(c2ccc(C(F)F)c3nccnc23)C1] | 365 |
| 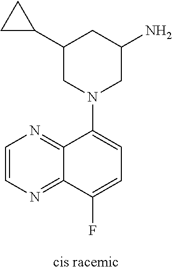 [SMILES: NC1CC(C2CC2)CN(c2ccc(F)c3nccnc23)C1] | 366 |
| 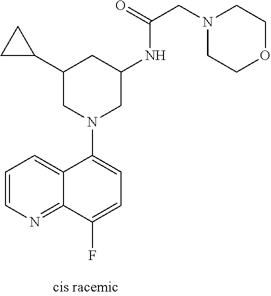 [SMILES: O=C(CN1CCOCC1)NC1CC(C2CC2)CN(c2ccc(F)c3ncccc23)C1] | 367 |
| 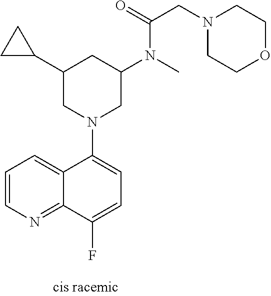 [SMILES: CN(C(=O)CN1CCOCC1)C1CC(C2CC2)CN(c2ccc(F)c3ncccc23)C1] | 368 |
| 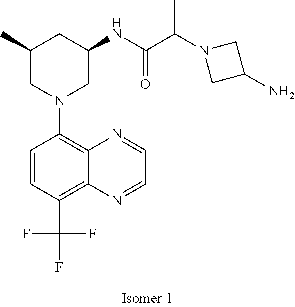 [SMILES: CC(C(=O)N[C@@H]1C[C@H](C)CN(c2ccc(C(F)(F)F)c3nccnc23)C1)N1CC(N)C1] | 369 |
| 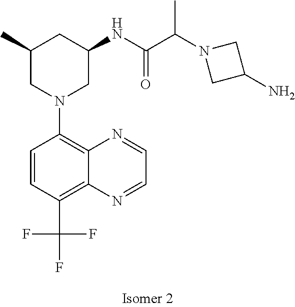 [SMILES: CC(C(=O)N[C@@H]1C[C@H](C)CN(c2ccc(C(F)(F)F)c3nccnc23)C1)N1CC(N)C1] | 370 |
| 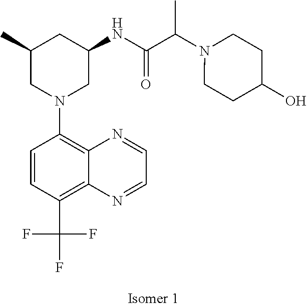 [SMILES: CC(C(=O)N[C@@H]1C[C@H](C)CN(c2ccc(C(F)(F)F)c3nccnc23)C1)N1CCC(O)CC1] | 371 |
| 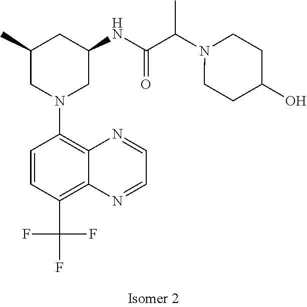 [SMILES: CC(C(=O)N[C@@H]1C[C@H](C)CN(c2ccc(C(F)(F)F)c3nccnc23)C1)N1CCC(O)CC1] | 372 |
| 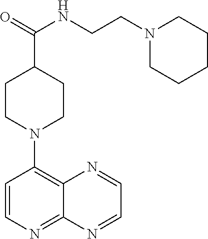 [SMILES: O=C(NCCN1CCCCC1)C1CCN(c2ccnc3nccnc23)CC1] | 373 |
| 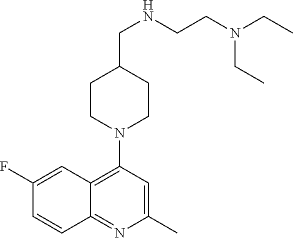 [SMILES: CCN(CC)CCNCC1CCN(c2cc(C)nc3ccc(F)cc23)CC1] | 374 |
| 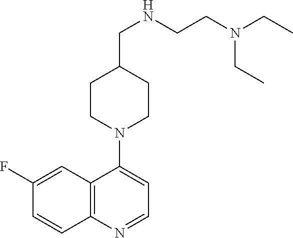 [SMILES: CCN(CC)CCNCC1CCN(c2ccnc3ccc(F)cc23)CC1] | 375 |
| 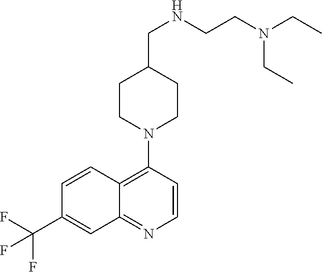 [SMILES: CCN(CC)CCNCC1CCN(c2ccnc3cc(C(F)(F)F)ccc23)CC1] | 376 |
| 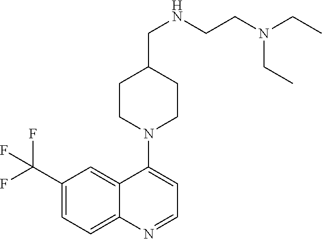 [SMILES: CCN(CC)CCNCC1CCN(c2ccnc3ccc(C(F)(F)F)cc23)CC1] | 377 |
| 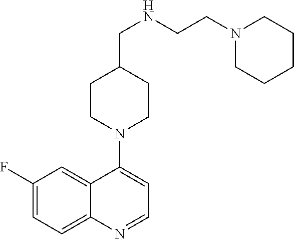 [SMILES: Fc1ccc2nccc(N3CCC(CNCCN4CCCCC4)CC3)c2c1] | 378 |
| 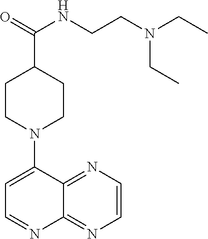 [SMILES: CCN(CC)CCNC(=O)C1CCN(c2ccnc3nccnc23)CC1] | 379 |
| 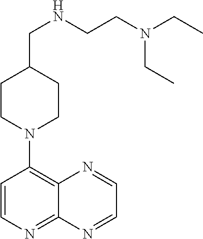 [SMILES: CCN(CC)CCNCC1CCN(c2ccnc3nccnc23)CC1] | 380 |
| 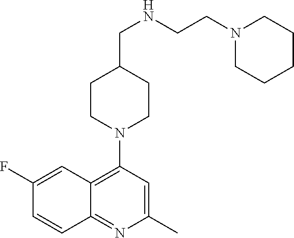 [SMILES: Cc1cc(N2CCC(CNCCN3CCCCC3)CC2)c2cc(F)ccc2n1] | 381 |
| 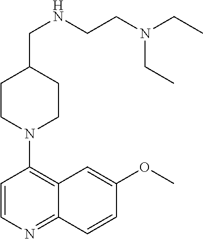 [SMILES: CCN(CC)CCNCC1CCN(c2ccnc3ccc(OC)cc23)CC1] | 382 |
| 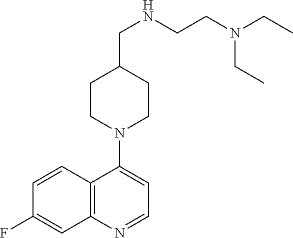 [SMILES: CCN(CC)CCNCC1CCN(c2ccnc3cc(F)ccc23)CC1] | 383 |
| 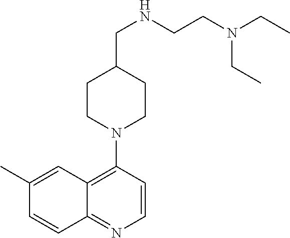 [SMILES: CCN(CC)CCNCC1CCN(c2ccnc3ccc(C)cc23)CC1] | 384 |
| 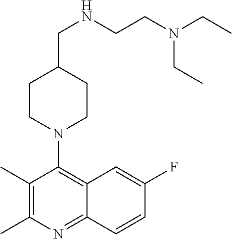 [SMILES: CCN(CC)CCNCC1CCN(c2c(C)c(C)nc3ccc(F)cc23)CC1] | 385 |
| 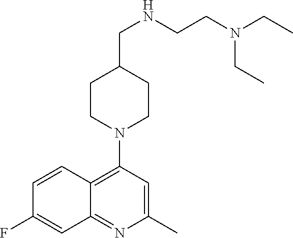 [SMILES: CCN(CC)CCNCC1CCN(c2cc(C)nc3cc(F)ccc23)CC1] | 386 |
| 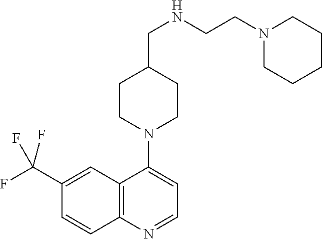 [SMILES: FC(F)(F)c1ccc2nccc(N3CCC(CNCCN4CCCCC4)CC3)c2c1] | 387 |
| 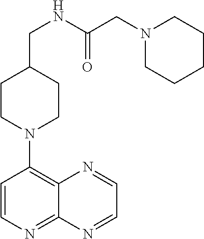 [SMILES: O=C(CN1CCCCC1)NCC1CCN(c2ccnc3nccnc23)CC1] | 388 |
| 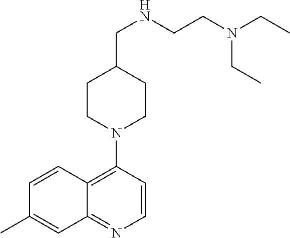 [SMILES: CCN(CC)CCNCC1CCN(c2ccnc3cc(C)ccc23)CC1] | 389 |
| 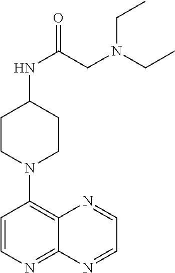 [SMILES: CCN(CC)CC(=O)NC1CCN(c2ccnc3nccnc23)CC1] | 390 |
| 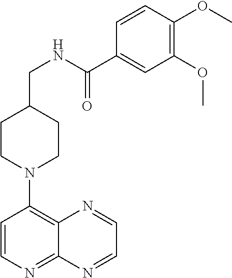 [SMILES: COc1ccc(C(=O)NCC2CCN(c3ccnc4nccnc34)CC2)cc1OC] | 391 |
| 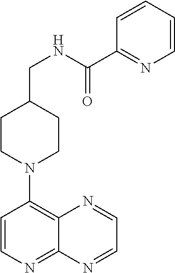 [SMILES: O=C(NCC1CCN(c2ccnc3nccnc23)CC1)c1ccccn1] | 392 |
| 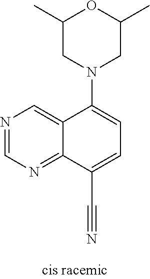 [SMILES: CC1CN(c2ccc(C#N)c3ncncc23)CC(C)O1] | 393 |
| 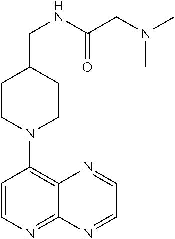 [SMILES: CN(C)CC(=O)NCC1CCN(c2ccnc3nccnc23)CC1] | 394 |
| 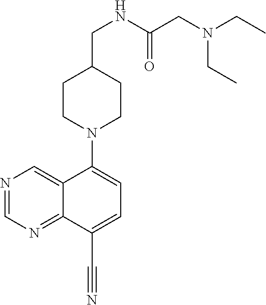 [SMILES: CCN(CC)CC(=O)NCC1CCN(c2ccc(C#N)c3ncncc23)CC1] | 395 |
| 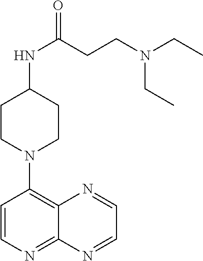 [SMILES: CCN(CC)CCC(=O)NC1CCN(c2ccnc3nccnc23)CC1] | 396 |
| 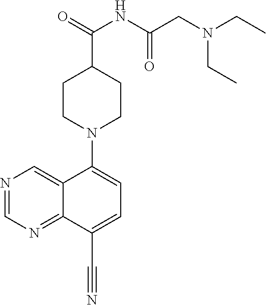 [SMILES: CCN(CC)CC(=O)NC(=O)C1CCN(c2ccc(C#N)c3ncncc23)CC1] | 397 |
| 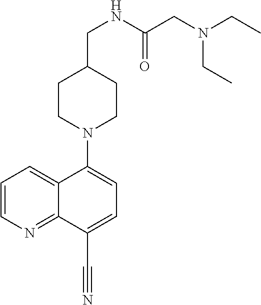 [SMILES: CCN(CC)CC(=O)NCC1CCN(c2ccc(C#N)c3ncccc23)CC1] | 398 |
| 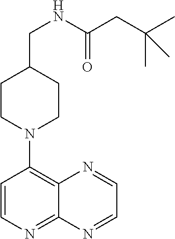 [SMILES: CC(C)(C)CC(=O)NCC1CCN(c2ccnc3nccnc23)CC1] | 399 |
| 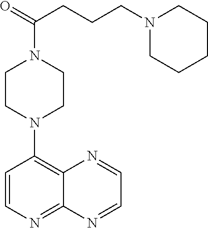 [SMILES: O=C(CCCN1CCCCC1)N1CCN(c2ccnc3nccnc23)CC1] | 400 |
| 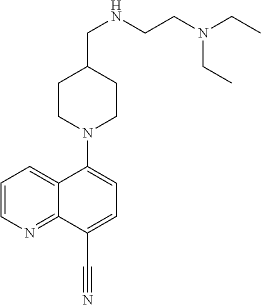 [SMILES: CCN(CC)CCNCC1CCN(c2ccc(C#N)c3ncccc23)CC1] | 401 |
| 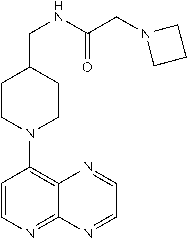 [SMILES: O=C(CN1CCC1)NCC1CCN(c2ccnc3nccnc23)CC1] | 402 |
| 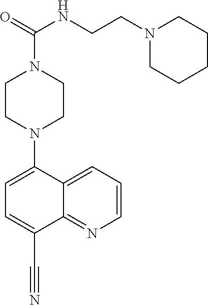 [SMILES: N#Cc1ccc(N2CCN(C(=O)NCCN3CCCCC3)CC2)c2cccnc12] | 403 |
| 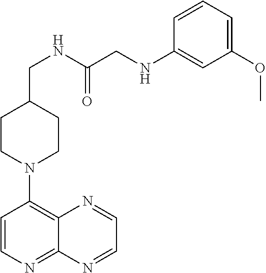 [SMILES: COc1cccc(NCC(=O)NCC2CCN(c3ccnc4nccnc34)CC2)c1] | 404 |
| 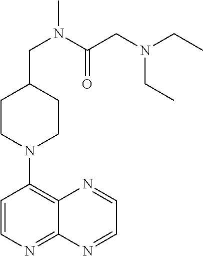 [SMILES: CCN(CC)CC(=O)N(C)CC1CCN(c2ccnc3nccnc23)CC1] | 405 |
| 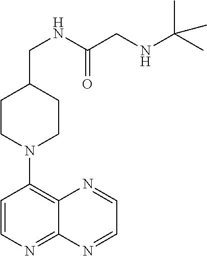 [SMILES: CC(C)(C)NCC(=O)NCC1CCN(c2ccnc3nccnc23)CC1] | 406 |
| 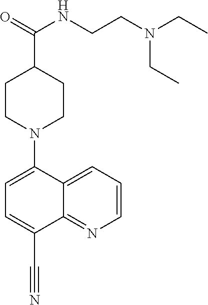 [SMILES: CCN(CC)CCNC(=O)C1CCN(c2ccc(C#N)c3ncccc23)CC1] | 407 |
| 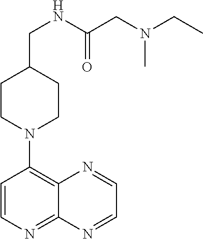 [SMILES: CCN(C)CC(=O)NCC1CCN(c2ccnc3nccnc23)CC1] | 408 |
| 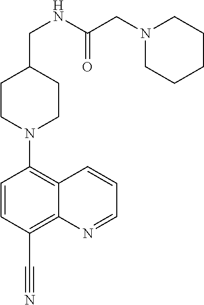 [SMILES: N#Cc1ccc(N2CCC(CNC(=O)CN3CCCCC3)CC2)c2cccnc12] | 409 |
| 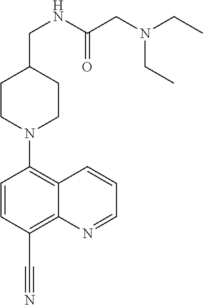 [SMILES: CCN(CC)CC(=O)NCC1CCN(c2ccc(C#N)c3ncccc23)CC1] | 410 |
| 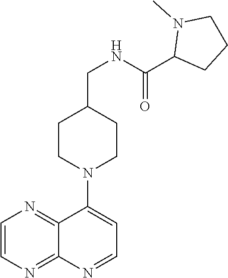 [SMILES: CN1CCCC1C(=O)NCC1CCN(c2ccnc3nccnc23)CC1] | 411 |
| 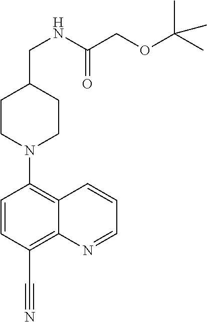 [SMILES: CC(C)(C)OCC(=O)NCC1CCN(c2ccc(C#N)c3ncccc23)CC1] | 412 |
| 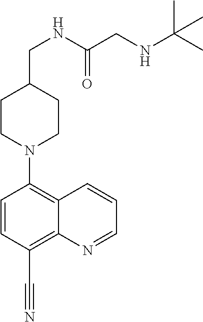 [SMILES: CC(C)(C)NCC(=O)NCC1CCN(c2ccc(C#N)c3ncccc23)CC1] | 413 |
| 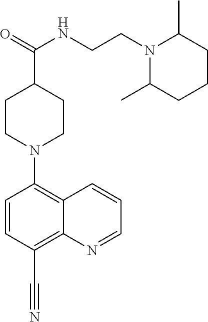 [SMILES: CC1CCCC(C)N1CCNC(=O)C1CCN(c2ccc(C#N)c3ncccc23)CC1] | 414 |
| 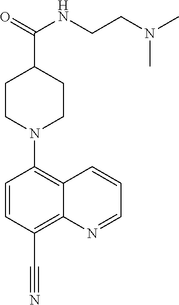 [SMILES: CN(C)CCNC(=O)C1CCN(c2ccc(C#N)c3ncccc23)CC1] | 415 |
| 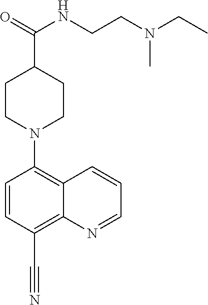 [SMILES: CCN(C)CCNC(=O)C1CCN(c2ccc(C#N)c3ncccc23)CC1] | 416 |
| 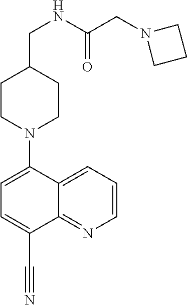 [SMILES: N#Cc1ccc(N2CCC(CNC(=O)CN3CCC3)CC2)c2cccnc12] | 417 |
| 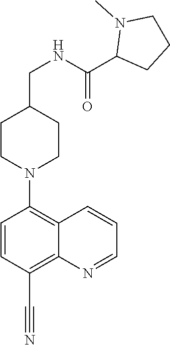 [SMILES: CN1CCCC1C(=O)NCC1CCN(c2ccc(C#N)c3ncccc23)CC1] | 418 |
| 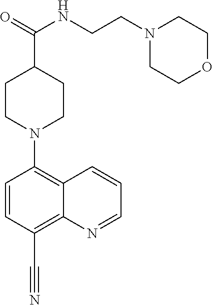 [SMILES: N#Cc1ccc(N2CCC(C(=O)NCCN3CCOCC3)CC2)c2cccnc12] | 419 |
| 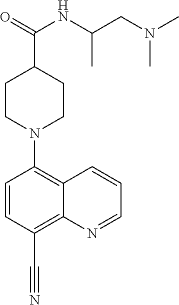 [SMILES: CC(CN(C)C)NC(=O)C1CCN(c2ccc(C#N)c3ncccc23)CC1] | 420 |
| 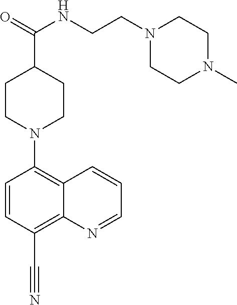 [SMILES: CN1CCN(CCNC(=O)C2CCN(c3ccc(C#N)c4ncccc34)CC2)CC1] | 421 |
| 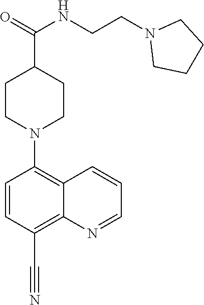 [SMILES: N#Cc1ccc(N2CCC(C(=O)NCCN3CCCC3)CC2)c2cccnc12] | 422 |
| 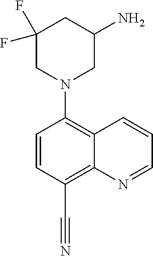 [SMILES: N#Cc1ccc(N2CC(N)CC(F)(F)C2)c2cccnc12] | 423 |
| 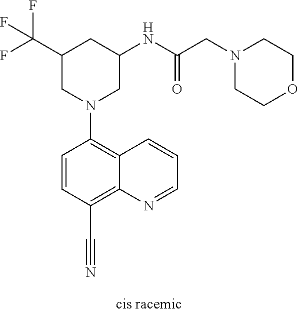 [SMILES: N#Cc1ccc(N2CC(NC(=O)CN3CCOCC3)CC(C(F)(F)F)C2)c2cccnc12] | 424 |
| 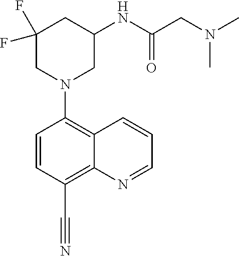 [SMILES: CN(C)CC(=O)NC1CN(c2ccc(C#N)c3ncccc23)CC(F)(F)C1] | 425 |
| 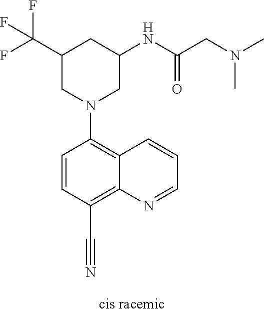 [SMILES: CN(C)CC(=O)NC1CC(C(F)(F)F)CN(c2ccc(C#N)c3ncccc23)C1] | 426 |
| 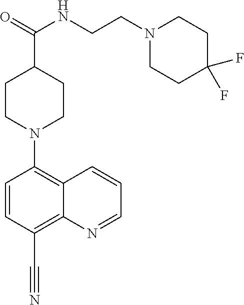 [SMILES: N#Cc1ccc(N2CCC(C(=O)NCCN3CCC(F)(F)CC3)CC2)c2cccnc12] | 427 |
| 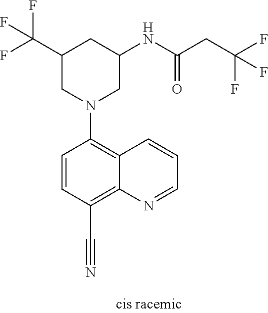 [SMILES: N#Cc1ccc(N2CC(NC(=O)CC(F)(F)F)CC(C(F)(F)F)C2)c2cccnc12] | 428 |
| 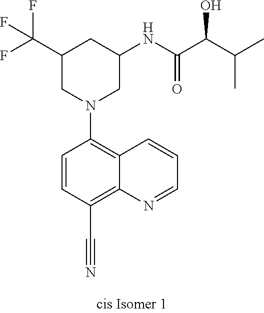 [SMILES: CC(C)[C@H](O)C(=O)NC1CC(C(F)(F)F)CN(c2ccc(C#N)c3ncccc23)C1] | 429 |
| 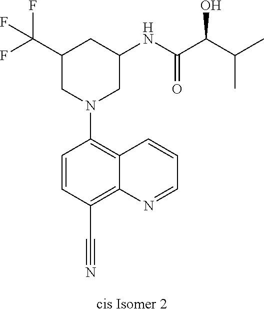 [SMILES: CC(C)[C@H](O)C(=O)NC1CC(C(F)(F)F)CN(c2ccc(C#N)c3ncccc23)C1] | 430 |
| 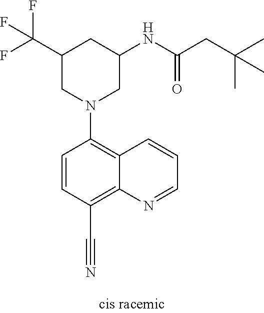 [SMILES: CC(C)(C)CC(=O)NC1CC(C(F)(F)F)CN(c2ccc(C#N)c3ncccc23)C1] | 431 |
| 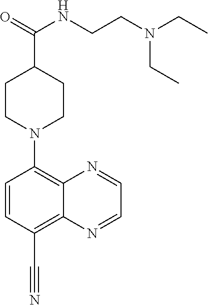 [SMILES: CCN(CC)CCNC(=O)C1CCN(c2ccc(C#N)c3nccnc23)CC1] | 432 |
| 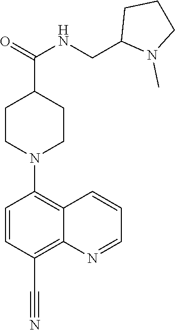 [SMILES: CN1CCCC1CNC(=O)C1CCN(c2ccc(C#N)c3ncccc23)CC1] | 433 |
| 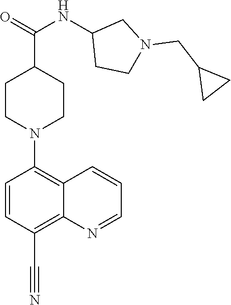 [SMILES: N#Cc1ccc(N2CCC(C(=O)NC3CCN(CC4CC4)C3)CC2)c2cccnc12] | 434 |
| 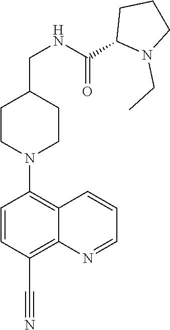 [SMILES: CCN1CCC[C@H]1C(=O)NCC1CCN(c2ccc(C#N)c3ncccc23)CC1] | 435 |
| 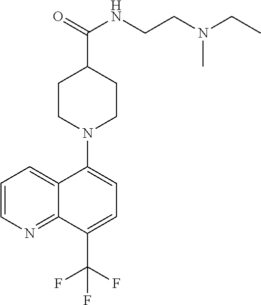 [SMILES: CCN(C)CCNC(=O)C1CCN(c2ccc(C(F)(F)F)c3ncccc23)CC1] | 436 |
| 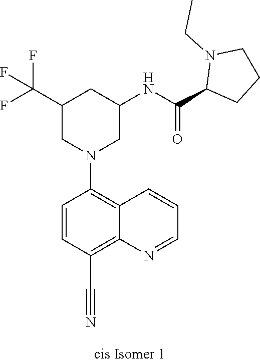 [SMILES: CCN1CCC[C@H]1C(=O)NC1CC(C(F)(F)F)CN(c2ccc(C#N)c3ncccc23)C1] | 437 |
| 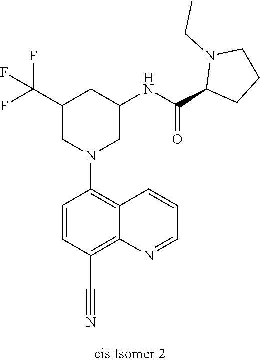 [SMILES: CCN1CCC[C@H]1C(=O)NC1CC(C(F)(F)F)CN(c2ccc(C#N)c3ncccc23)C1] | 438 |
| 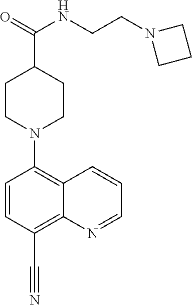 [SMILES: N#Cc1ccc(N2CCC(C(=O)NCCN3CCC3)CC2)c2cccnc12] | 439 |
| 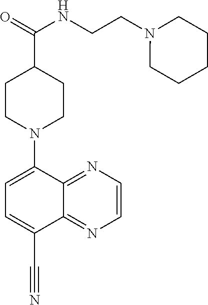 [SMILES: N#Cc1ccc(N2CCC(C(=O)NCCN3CCCCC3)CC2)c2nccnc12] | 440 |
| 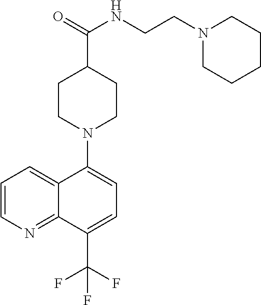 [SMILES: O=C(NCCN1CCCCC1)C1CCN(c2ccc(C(F)(F)F)c3ncccc23)CC1] | 441 |
| 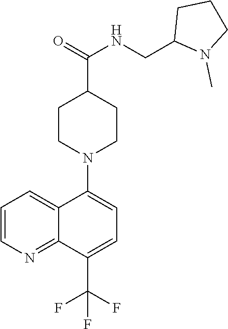 [SMILES: CN1CCCC1CNC(=O)C1CCN(c2ccc(C(F)(F)F)c3ncccc23)CC1] | 442 |
| 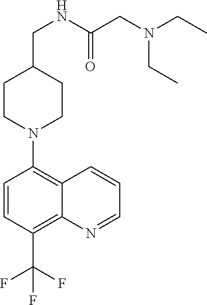 [SMILES: CCN(CC)CC(=O)NCC1CCN(c2ccc(C(F)(F)F)c3ncccc23)CC1] | 443 |
| 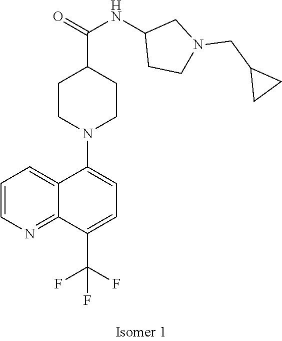 [SMILES: O=C(NC1CCN(CC2CC2)C1)C1CCN(c2ccc(C(F)(F)F)c3ncccc23)CC1] | 444 |
| 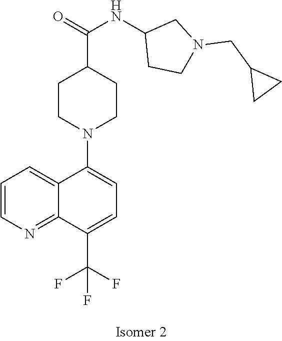 [SMILES: O=C(NC1CCN(CC2CC2)C1)C1CCN(c2ccc(C(F)(F)F)c3ncccc23)CC1] | 445 |
| 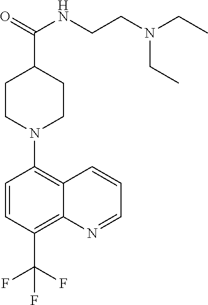 [SMILES: CCN(CC)CCNC(=O)C1CCN(c2ccc(C(F)(F)F)c3ncccc23)CC1] | 446 |
| 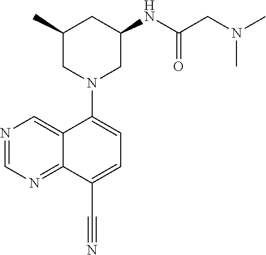 [SMILES: C[C@H]1C[C@@H](NC(=O)CN(C)C)CN(c2ccc(C#N)c3ncncc23)C1] | 447 |
| 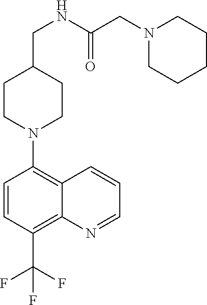 [SMILES: O=C(CN1CCCCC1)NCC1CCN(c2ccc(C(F)(F)F)c3ncccc23)CC1] | 448 |
| 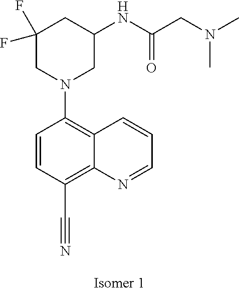 [SMILES: CN(C)CC(=O)NC1CN(c2ccc(C#N)c3ncccc23)CC(F)(F)C1] | 449 |
| 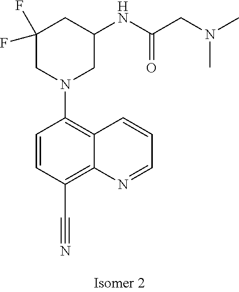 [SMILES: CN(C)CC(=O)NC1CN(c2ccc(C#N)c3ncccc23)CC(F)(F)C1] | 450 |
| 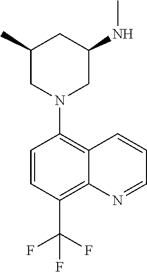 [SMILES: CN[C@@H]1C[C@H](C)CN(c2ccc(C(F)(F)F)c3ncccc23)C1] | 451 |
| 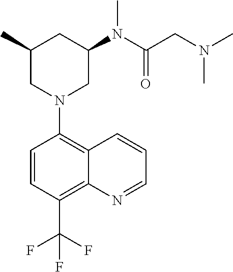 | 452 |
| 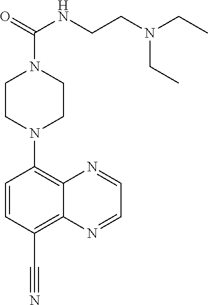 [SMILES: CCN(CC)CCNC(=O)N1CCN(c2ccc(C#N)c3nccnc23)CC1] | 453 |
| 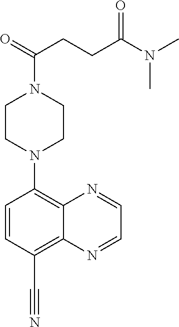 [SMILES: CN(C)C(=O)CCC(=O)N1CCN(c2ccc(C#N)c3nccnc23)CC1] | 454 |
| 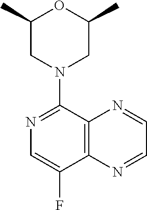 [SMILES: C[C@@H]1CN(c2ncc(F)c3nccnc23)C[C@H](C)O1] | 455 |
| 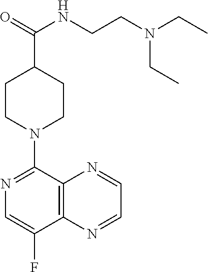 [SMILES: CCN(CC)CCNC(=O)C1CCN(c2ncc(F)c3nccnc23)CC1] | 456 |
| 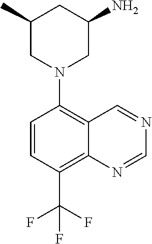 [SMILES: C[C@H]1C[C@@H](N)CN(c2ccc(C(F)(F)F)c3ncncc23)C1] | 457 |
| 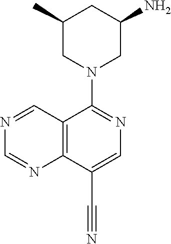 [SMILES: C[C@H]1C[C@@H](N)CN(c2ncc(C#N)c3ncncc23)C1] | 458 |
| 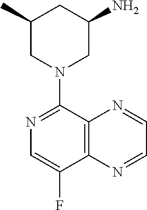 [SMILES: C[C@H]1C[C@@H](N)CN(c2ncc(F)c3nccnc23)C1] | 459 |
| 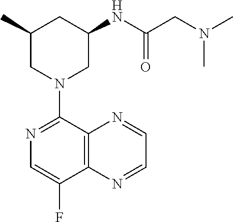 | 460 |
| 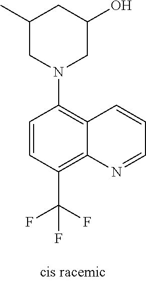 [SMILES: CC1CC(O)CN(c2ccc(C(F)(F)F)c3ncccc23)C1] | 461 |
| 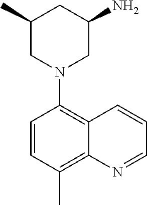 [SMILES: Cc1ccc(N2C[C@@H](C)C[C@@H](N)C2)c2cccnc12] | 462 |
| 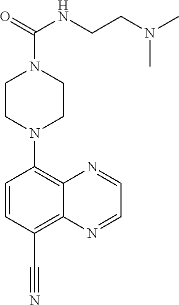 [SMILES: CN(C)CCNC(=O)N1CCN(c2ccc(C#N)c3nccnc23)CC1] | 463 |
| 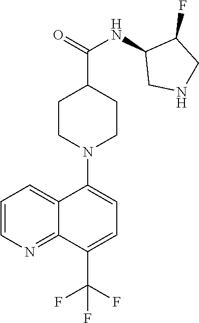 [SMILES: O=C(N[C@@H]1CNC[C@@H]1F)C1CCN(c2ccc(C(F)(F)F)c3ncccc23)CC1] | 464 |
| 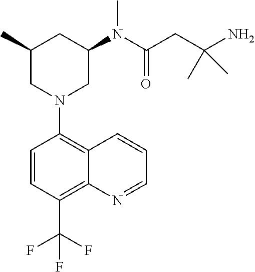 [SMILES: C[C@H]1C[C@@H](N(C)C(=O)CC(C)(C)N)CN(c2ccc(C(F)(F)F)c3ncccc23)C1] | 465 |
| 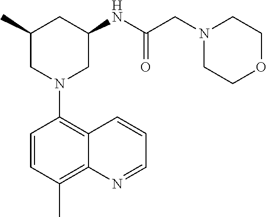 [SMILES: Cc1ccc(N2C[C@@H](C)C[C@@H](NC(=O)CN3CCOCC3)C2)c2cccnc12] | 466 |
| 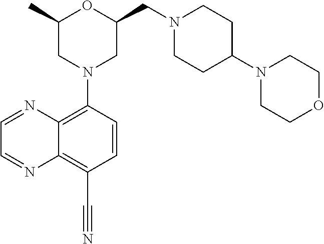 [SMILES: C[C@@H]1CN(c2ccc(C#N)c3nccnc23)C[C@H](CN2CCC(N3CCOCC3)CC2)O1] | 467 |
| 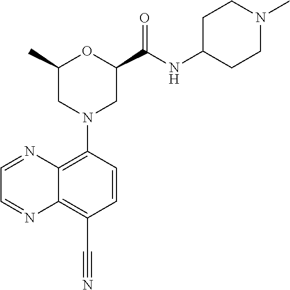 [SMILES: C[C@@H]1CN(c2ccc(C#N)c3nccnc23)C[C@H](C(=O)NC2CCN(C)CC2)O1] | 468 |
| 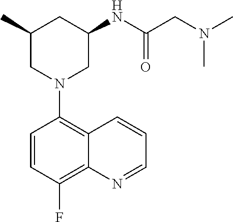 | 469 |
| 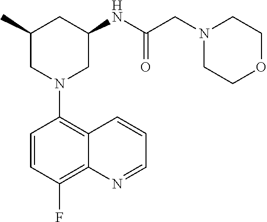 [SMILES: C[C@H]1C[C@@H](NC(=O)CN2CCOCC2)CN(c2ccc(F)c3ncccc23)C1] | 470 |
| 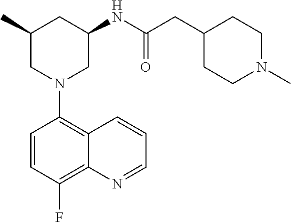 | 471 |
| 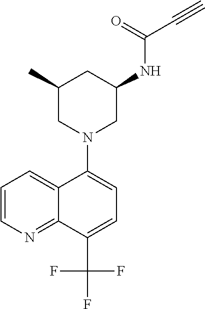 [SMILES: C#CC(=O)N[C@@H]1C[C@H](C)CN(c2ccc(C(F)(F)F)c3ncccc23)C1] | 472 |
| 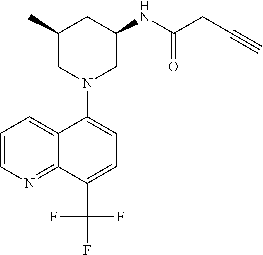 [SMILES: C#CCC(=O)N[C@@H]1C[C@H](C)CN(c2ccc(C(F)(F)F)c3ncccc23)C1] | 473 |
| 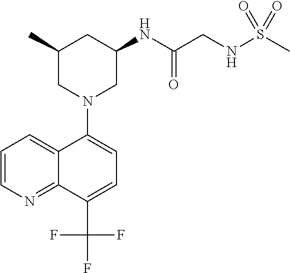 [SMILES: C[C@H]1C[C@@H](NC(=O)CNS(C)(=O)=O)CN(c2ccc(C(F)(F)F)c3ncccc23)C1] | 474 |
| 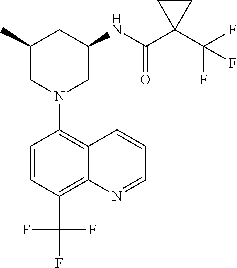 | 475 |
| 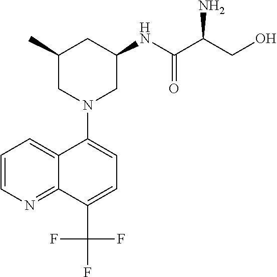 [SMILES: C[C@H]1C[C@@H](NC(=O)[C@@H](N)CO)CN(c2ccc(C(F)(F)F)c3ncccc23)C1] | 476 |
| 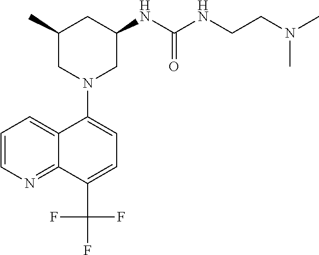 [SMILES: C[C@H]1C[C@@H](NC(=O)NCCN(C)C)CN(c2ccc(C(F)(F)F)c3ncccc23)C1] | 477 |
| 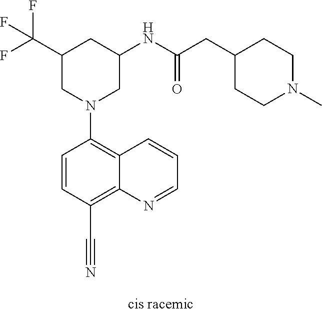 [SMILES: CN1CCC(CC(=O)NC2CC(C(F)(F)F)CN(c3ccc(C#N)c4ncccc34)C2)CC1] | 478 |
| 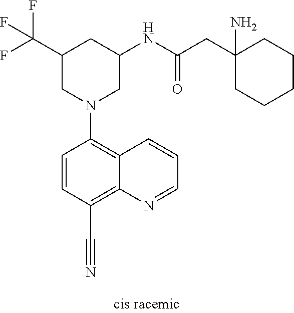 [SMILES: N#Cc1ccc(N2CC(NC(=O)CC3(N)CCCCC3)CC(C(F)(F)F)C2)c2cccnc12] | 479 |
| 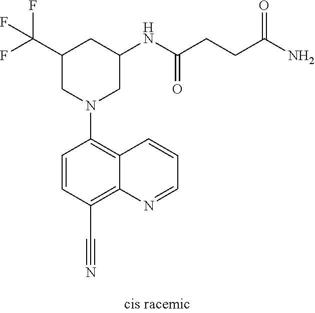 [SMILES: N#Cc1ccc(N2CC(NC(=O)CCC(N)=O)CC(C(F)(F)F)C2)c2cccnc12] | 480 |
| 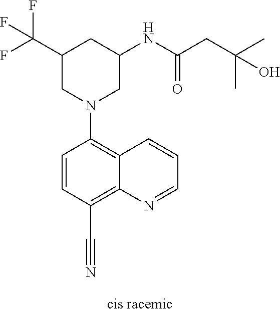 [SMILES: CC(C)(O)CC(=O)NC1CC(C(F)(F)F)CN(c2ccc(C#N)c3ncccc23)C1] | 481 |
| 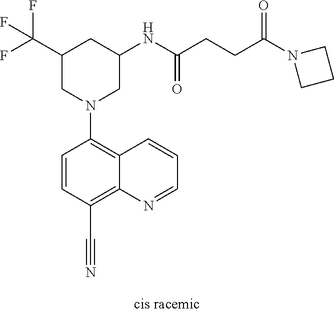 [SMILES: N#Cc1ccc(N2CC(NC(=O)CCC(=O)N3CCC3)CC(C(F)(F)F)C2)c2cccnc12] | 482 |
| 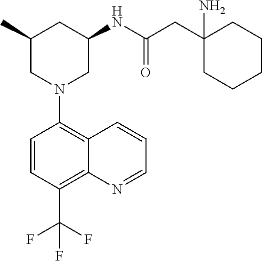 [SMILES: C[C@H]1C[C@@H](NC(=O)CC2(N)CCCCC2)CN(c2ccc(C(F)(F)F)c3ncccc23)C1] | 483 |
| 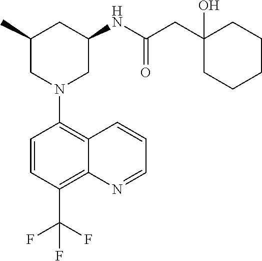 [SMILES: C[C@H]1C[C@@H](NC(=O)CC2(O)CCCCC2)CN(c2ccc(C(F)(F)F)c3ncccc23)C1] | 484 |
| 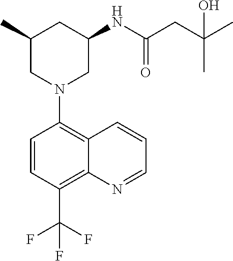 [SMILES: C[C@H]1C[C@@H](NC(=O)CC(C)(C)O)CN(c2ccc(C(F)(F)F)c3ncccc23)C1] | 485 |
| 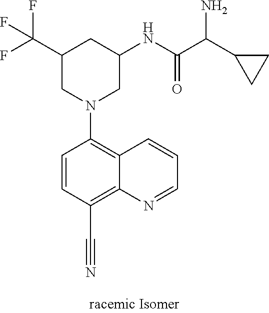 [SMILES: N#Cc1ccc(N2CC(NC(=O)C(N)C3CC3)CC(C(F)(F)F)C2)c2cccnc12] | 486 |
| 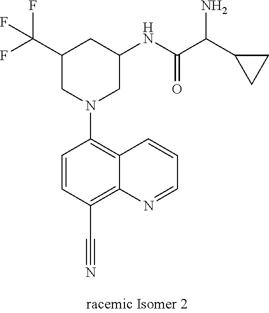 [SMILES: N#Cc1ccc(N2CC(NC(=O)C(N)C3CC3)CC(C(F)(F)F)C2)c2cccnc12] | 487 |
| 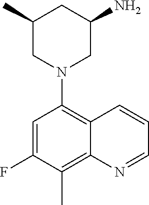 [SMILES: Cc1c(F)cc(N2C[C@@H](C)C[C@@H](N)C2)c2cccnc12] | 488 |
| 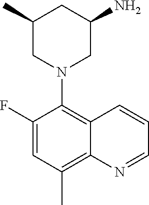 [SMILES: Cc1cc(F)c(N2C[C@@H](C)C[C@@H](N)C2)c2cccnc12] | 489 |
| 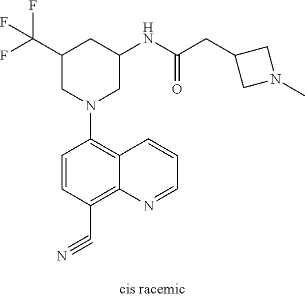 [SMILES: CN1CC(CC(=O)NC2CC(C(F)(F)F)CN(c3ccc(C#N)c4ncccc34)C2)C1] | 490 |
| 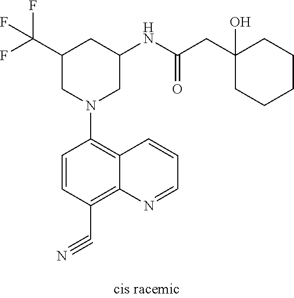 [SMILES: N#Cc1ccc(N2CC(NC(=O)CC3(O)CCCCC3)CC(C(F)(F)F)C2)c2cccnc12] | 491 |
| 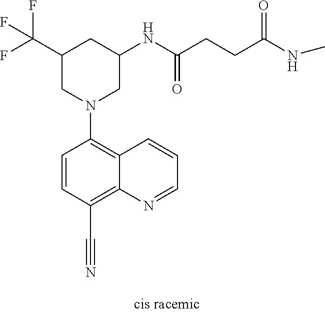 [SMILES: CNC(=O)CCC(=O)NC1CC(C(F)(F)F)CN(c2ccc(C#N)c3ncccc23)C1] | 492 |
| 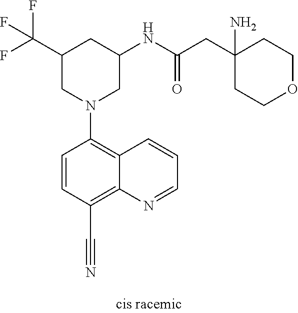 [SMILES: N#Cc1ccc(N2CC(NC(=O)CC3(N)CCOCC3)CC(C(F)(F)F)C2)c2cccnc12] | 493 |
| 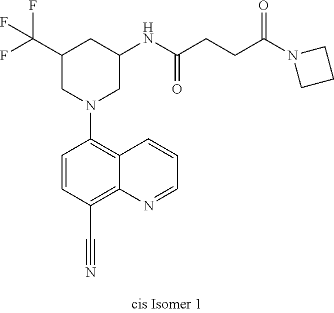 [SMILES: N#Cc1ccc(N2CC(NC(=O)CCC(=O)N3CCC3)CC(C(F)(F)F)C2)c2cccnc12] | 494 |
| 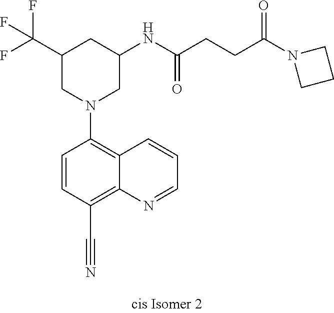 [SMILES: N#Cc1ccc(N2CC(NC(=O)CCC(=O)N3CCC3)CC(C(F)(F)F)C2)c2cccnc12] | 495 |
| 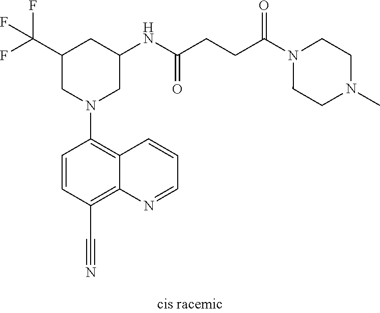 [SMILES: CN1CCN(C(=O)CCC(=O)NC2CC(C(F)(F)F)CN(c3ccc(C#N)c4ncccc34)C2)CC1] | 496 |
| 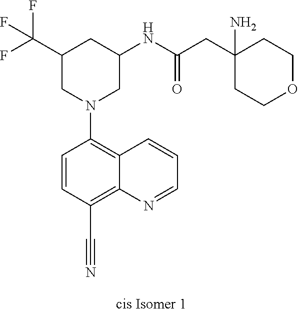 [SMILES: N#Cc1ccc(N2CC(NC(=O)CC3(N)CCOCC3)CC(C(F)(F)F)C2)c2cccnc12] | 497 |
| 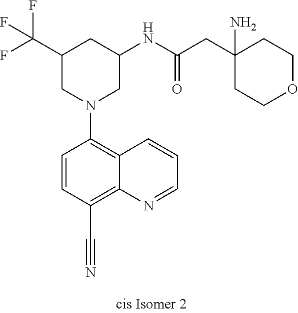 [SMILES: N#Cc1ccc(N2CC(NC(=O)CC3(N)CCOCC3)CC(C(F)(F)F)C2)c2cccnc12] | 498 |
| 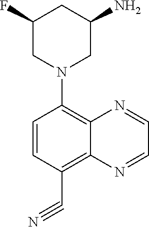 [SMILES: N#Cc1ccc(N2C[C@H](N)C[C@H](F)C2)c2nccnc12] | 499 |
| 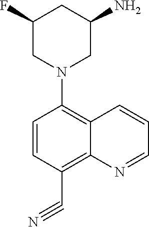 [SMILES: N#Cc1ccc(N2C[C@H](N)C[C@H](F)C2)c2cccnc12] | 500 |
| 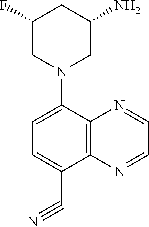 [SMILES: N#Cc1ccc(N2C[C@@H](N)C[C@@H](F)C2)c2nccnc12] | 501 |
| 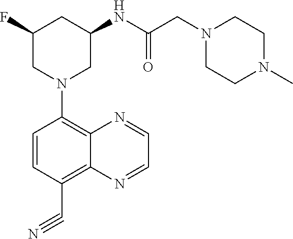 [SMILES: CN1CCN(CC(=O)N[C@@H]2C[C@H](F)CN(c3ccc(C#N)c4nccnc34)C2)CC1] | 502 |
| 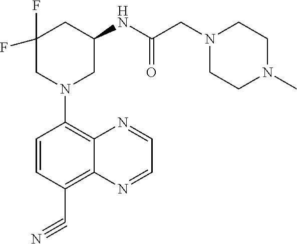 [SMILES: CN1CCN(CC(=O)N[C@H]2CN(c3ccc(C#N)c4nccnc34)CC(F)(F)C2)CC1] | 503 |
| 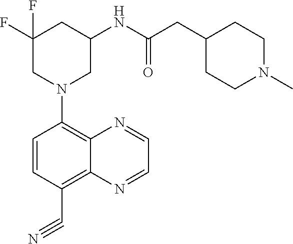 [SMILES: CN1CCC(CC(=O)NC2CN(c3ccc(C#N)c4nccnc34)CC(F)(F)C2)CC1] | 504 |
| 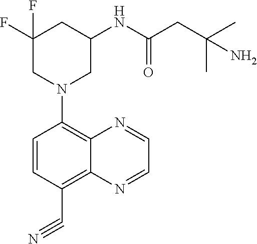 [SMILES: CC(C)(N)CC(=O)NC1CN(c2ccc(C#N)c3nccnc23)CC(F)(F)C1] | 505 |
| 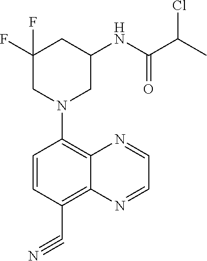 [SMILES: CC(Cl)C(=O)NC1CN(c2ccc(C#N)c3nccnc23)CC(F)(F)C1] | 506 |
| 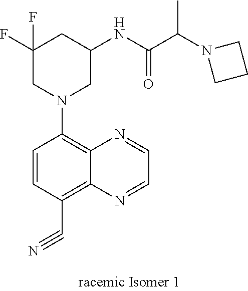 [SMILES: CC(C(=O)NC1CN(c2ccc(C#N)c3nccnc23)CC(F)(F)C1)N1CCC1] | 507 |
| 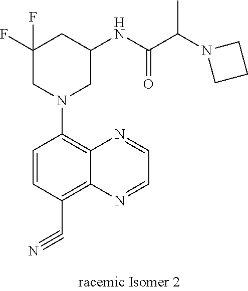 [SMILES: CC(C(=O)NC1CN(c2ccc(C#N)c3nccnc23)CC(F)(F)C1)N1CCC1] | 508 |
| 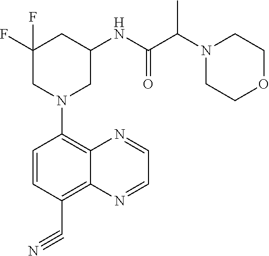 [SMILES: CC(C(=O)NC1CN(c2ccc(C#N)c3nccnc23)CC(F)(F)C1)N1CCOCC1] | 509 |
| 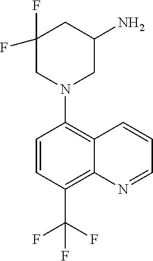 [SMILES: NC1CN(c2ccc(C(F)(F)F)c3ncccc23)CC(F)(F)C1] | 510 |
| 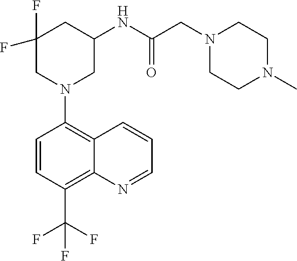 [SMILES: CN1CCN(CC(=O)NC2CN(c3ccc(C(F)(F)F)c4ncccc34)CC(F)(F)C2)CC1] | 511 |
| 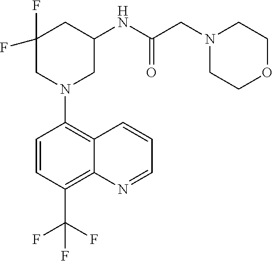 [SMILES: O=C(CN1CCOCC1)NC1CN(c2ccc(C(F)(F)F)c3ncccc23)CC(F)(F)C1] | 512 |
| 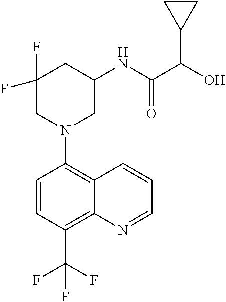 [SMILES: O=C(NC1CN(c2ccc(C(F)(F)F)c3ncccc23)CC(F)(F)C1)C(O)C1CC1] | 513 |
| 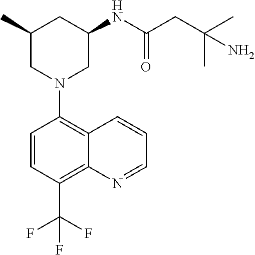 [SMILES: C[C@H]1C[C@@H](NC(=O)CC(C)(C)N)CN(c2ccc(C(F)(F)F)c3ncccc23)C1] | 514 |
| 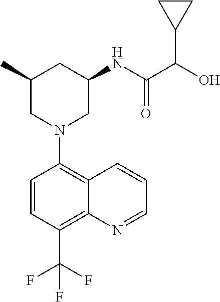 [SMILES: C[C@H]1C[C@@H](NC(=O)C(O)C2CC2)CN(c2ccc(C(F)(F)F)c3ncccc23)C1] | 515 |
| 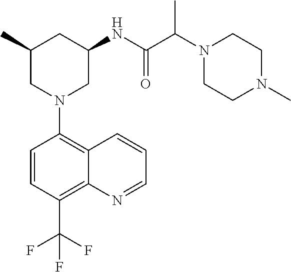 | 516 |
| 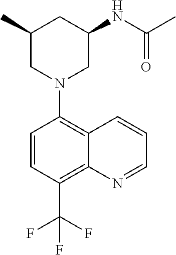 [SMILES: CC(=O)N[C@@H]1C[C@H](C)CN(c2ccc(C(F)(F)F)c3ncccc23)C1] | 517 |
| 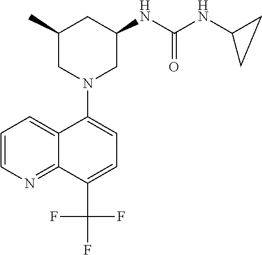 [SMILES: C[C@H]1C[C@@H](NC(=O)NC2CC2)CN(c2ccc(C(F)(F)F)c3ncccc23)C1] | 518 |
| 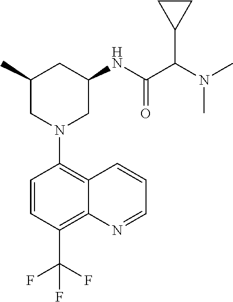 | 519 |
| 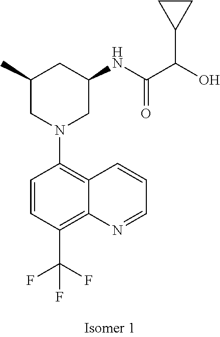 [SMILES: C[C@H]1C[C@@H](NC(=O)C(O)C2CC2)CN(c2ccc(C(F)(F)F)c3ncccc23)C1] | 520 |
| 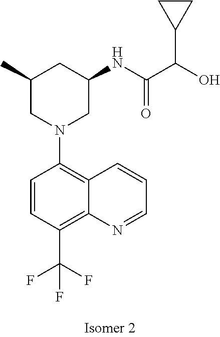 [SMILES: C[C@H]1C[C@@H](NC(=O)C(O)C2CC2)CN(c2ccc(C(F)(F)F)c3ncccc23)C1] | 521 |
| 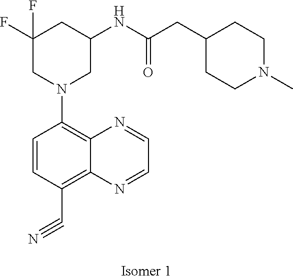 [SMILES: CN1CCC(CC(=O)NC2CN(c3ccc(C#N)c4nccnc34)CC(F)(F)C2)CC1] | 522 |
| 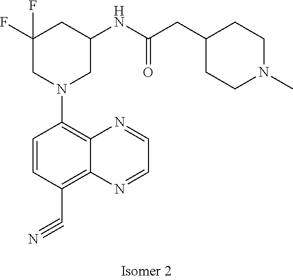 [SMILES: CN1CCC(CC(=O)NC2CN(c3ccc(C#N)c4nccnc34)CC(F)(F)C2)CC1] | 523 |
| 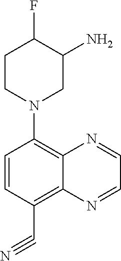 [SMILES: N#Cc1ccc(N2CCC(F)C(N)C2)c2nccnc12] | 524 |
| 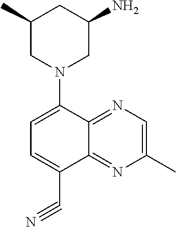 | 525 |
| 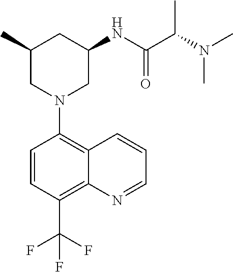 | 526 |
| 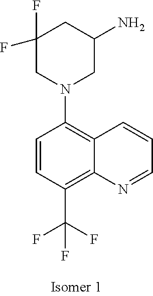 [SMILES: NC1CN(c2ccc(C(F)(F)F)c3ncccc23)CC(F)(F)C1] | 527 |
| 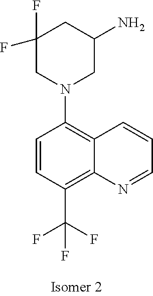 [SMILES: NC1CN(c2ccc(C(F)(F)F)c3ncccc23)CC(F)(F)C1] | 528 |
| 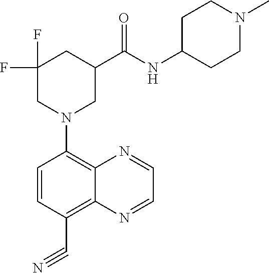 [SMILES: CN1CCC(NC(=O)C2CN(c3ccc(C#N)c4nccnc34)CC(F)(F)C2)CC1] | 529 |
| 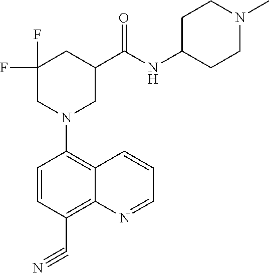 [SMILES: CN1CCC(NC(=O)C2CN(c3ccc(C#N)c4ncccc34)CC(F)(F)C2)CC1] | 530 |
| 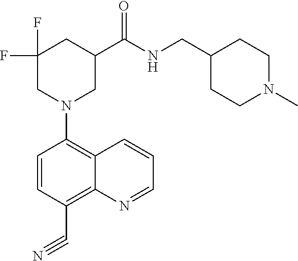 [SMILES: CN1CCC(CNC(=O)C2CN(c3ccc(C#N)c4ncccc34)CC(F)(F)C2)CC1] | 531 |
| 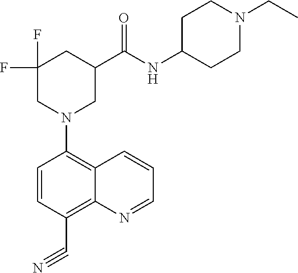 [SMILES: CCN1CCC(NC(=O)C2CN(c3ccc(C#N)c4ncccc34)CC(F)(F)C2)CC1] | 532 |
| 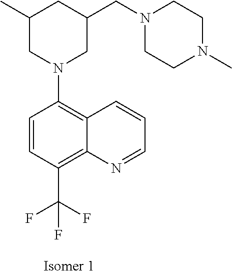 | 533 |
| 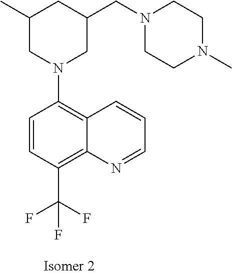 | 534 |
| 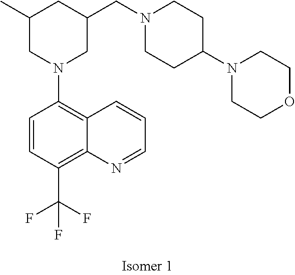 [SMILES: CC1CC(CN2CCC(N3CCOCC3)CC2)CN(c2ccc(C(F)(F)F)c3ncccc23)C1] | 535 |
| 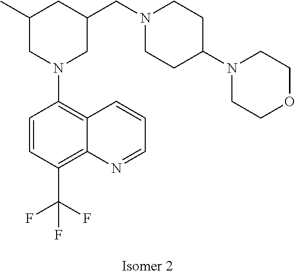 [SMILES: CC1CC(CN2CCC(N3CCOCC3)CC2)CN(c2ccc(C(F)(F)F)c3ncccc23)C1] | 536 |
| 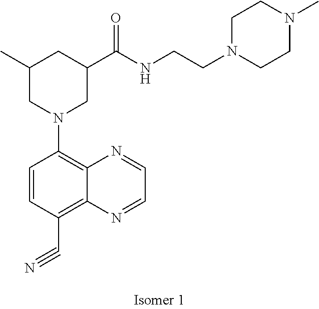 | 537 |
| 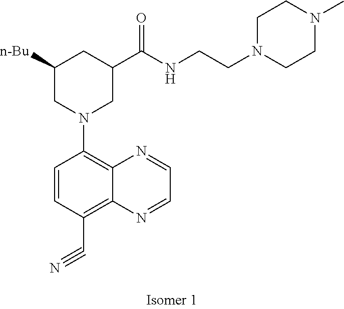 | 538 |
| 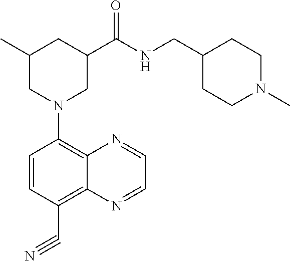 | 539 |
| 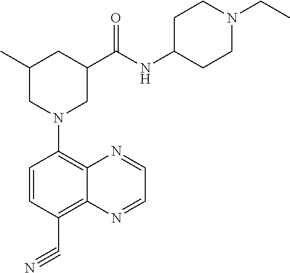 [SMILES: CCN1CCC(NC(=O)C2CC(C)CN(c3ccc(C#N)c4nccnc34)C2)CC1] | 540 |
| 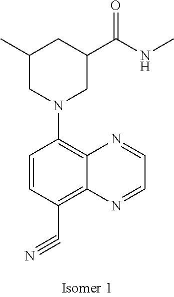 | 541 |
| 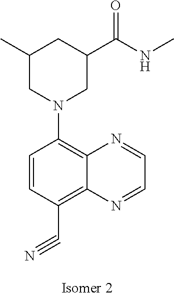 | 542 |
| 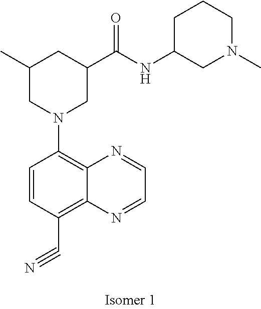 | 543 |
| 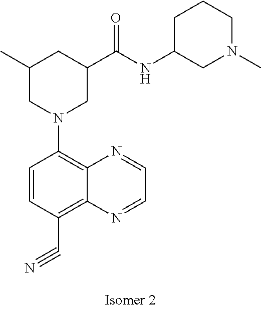 | 544 |
| 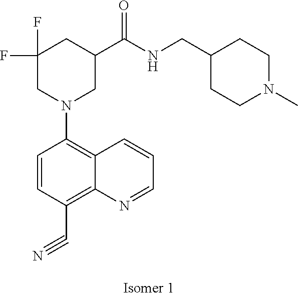 [SMILES: CN1CCC(CNC(=O)C2CN(c3ccc(C#N)c4ncccc34)CC(F)(F)C2)CC1] | 545 |
| 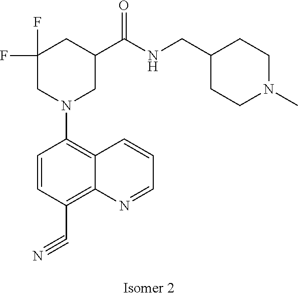 [SMILES: CN1CCC(CNC(=O)C2CN(c3ccc(C#N)c4ncccc34)CC(F)(F)C2)CC1] | 546 |
| 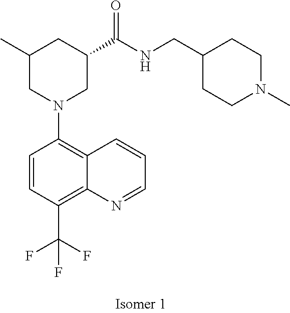 | 547 |
| 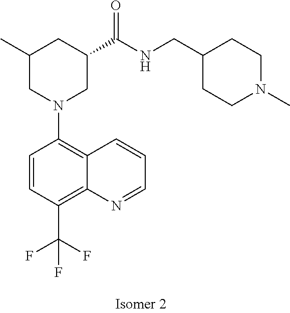 | 548 |
| 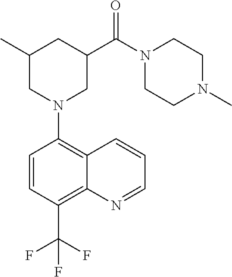 | 549 |
| 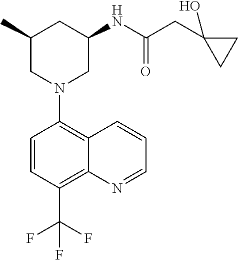 [SMILES: C[C@H]1C[C@@H](NC(=O)CC2(O)CC2)CN(c2ccc(C(F)(F)F)c3ncccc23)C1] | 550 |
| 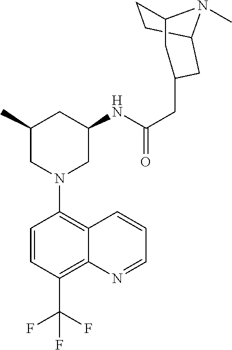 | 551 |
| 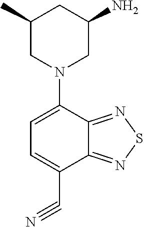 [SMILES: C[C@H]1C[C@@H](N)CN(c2ccc(C#N)c3nsnc23)C1] | 552 |
| 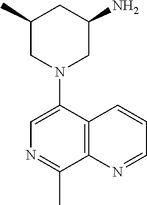 [SMILES: Cc1ncc(N2C[C@@H](C)C[C@@H](N)C2)c2cccnc12] | 553 |
| 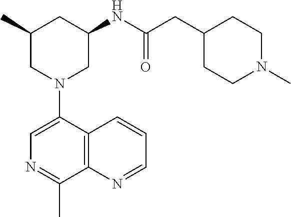 | 554 |
| 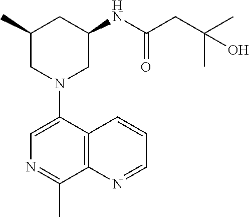 [SMILES: Cc1ncc(N2C[C@@H](C)C[C@@H](NC(=O)CC(C)(C)O)C2)c2cccnc12] | 555 |
| 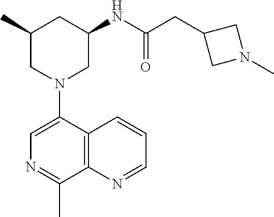 | 556 |
| 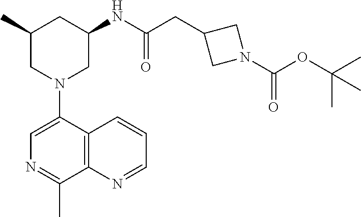 [SMILES: Cc1ncc(N2C[C@@H](C)C[C@@H](NC(=O)CC3CN(C(=O)OC(C)(C)C)C3)C2)c2cccnc12] | 557 |
| 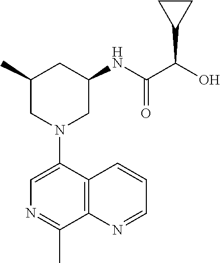 [SMILES: Cc1ncc(N2C[C@@H](C)C[C@@H](NC(=O)[C@H](O)C3CC3)C2)c2cccnc12] | 558 |
| 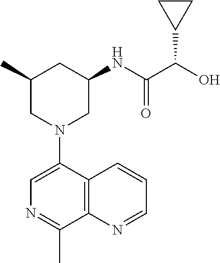 [SMILES: Cc1ncc(N2C[C@@H](C)C[C@@H](NC(=O)[C@@H](O)C3CC3)C2)c2cccnc12] | 559 |
| 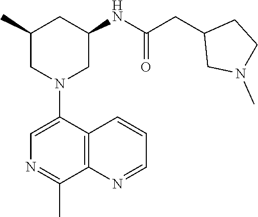 [SMILES: Cc1ncc(N2C[C@@H](C)C[C@@H](NC(=O)CC3CCN(C)C3)C2)c2cccnc12] | 560 |
| 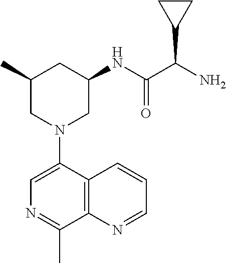 [SMILES: Cc1ncc(N2C[C@@H](C)C[C@@H](NC(=O)[C@H](N)C3CC3)C2)c2cccnc12] | 561 |
| 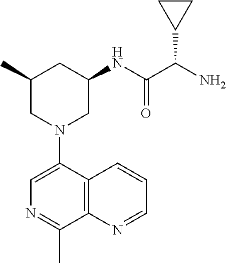 [SMILES: Cc1ncc(N2C[C@@H](C)C[C@@H](NC(=O)[C@@H](N)C3CC3)C2)c2cccnc12] | 562 |
| 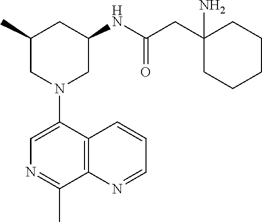 [SMILES: Cc1ncc(N2C[C@@H](C)C[C@@H](NC(=O)CC3(N)CCCCC3)C2)c2cccnc12] | 563 |
| 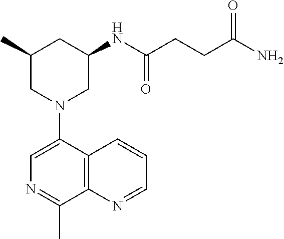 [SMILES: Cc1ncc(N2C[C@@H](C)C[C@@H](NC(=O)CCC(N)=O)C2)c2cccnc12] | 564 |
| 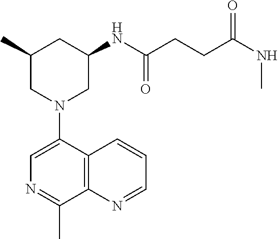 | 565 |
| 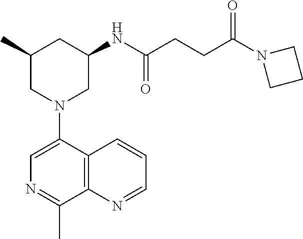 [SMILES: Cc1ncc(N2C[C@@H](C)C[C@@H](NC(=O)CCC(=O)N3CCC3)C2)c2cccnc12] | 566 |
| 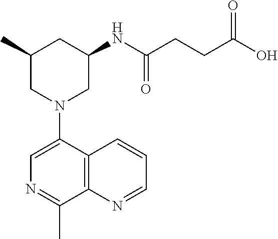 [SMILES: Cc1ncc(N2C[C@@H](C)C[C@@H](NC(=O)CCC(=O)O)C2)c2cccnc12] | 567 |
| 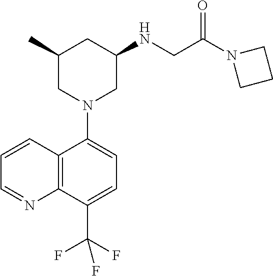 [SMILES: C[C@H]1C[C@@H](NCC(=O)N2CCC2)CN(c2ccc(C(F)(F)F)c3ncccc23)C1] | 568 |
| 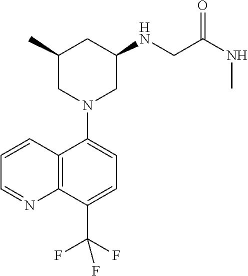 [SMILES: CNC(=O)CN[C@@H]1C[C@H](C)CN(c2ccc(C(F)(F)F)c3ncccc23)C1] | 569 |
| 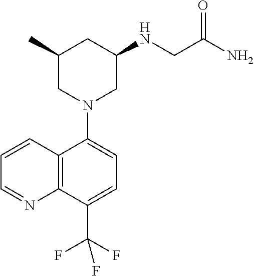 [SMILES: C[C@H]1C[C@@H](NCC(N)=O)CN(c2ccc(C(F)(F)F)c3ncccc23)C1] | 570 |
| 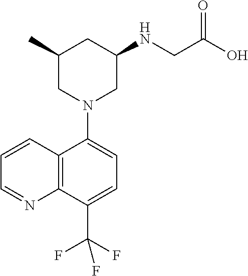 [SMILES: C[C@H]1C[C@@H](NCC(=O)O)CN(c2ccc(C(F)(F)F)c3ncccc23)C1] | 571 |
| 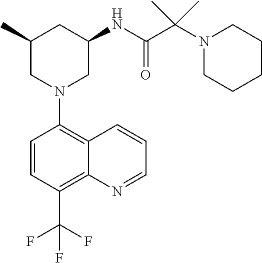 [SMILES: C[C@H]1C[C@@H](NC(=O)C(C)(C)N2CCCCC2)CN(c2ccc(C(F)(F)F)c3ncccc23)C1] | 572 |
| 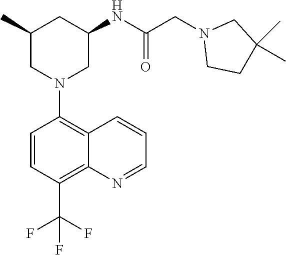 | 573 |
| 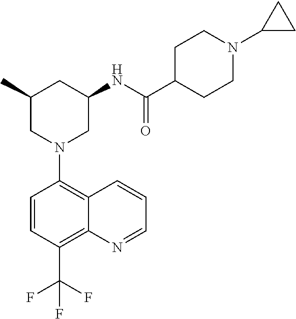 [SMILES: C[C@H]1C[C@@H](NC(=O)C2CCN(C3CC3)CC2)CN(c2ccc(C(F)(F)F)c3ncccc23)C1] | 574 |
| 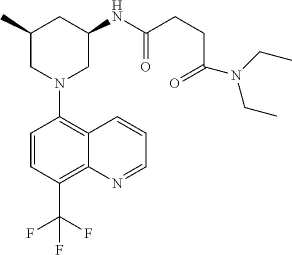 [SMILES: CCN(CC)C(=O)CCC(=O)N[C@@H]1C[C@H](C)CN(c2ccc(C(F)(F)F)c3ncccc23)C1] | 575 |
| 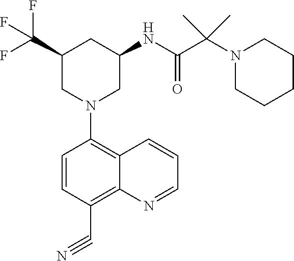 [SMILES: CC(C)(C(=O)N[C@@H]1C[C@H](C(F)(F)F)CN(c2ccc(C#N)c3ncccc23)C1)N1CCCCC1] | 576 |
| 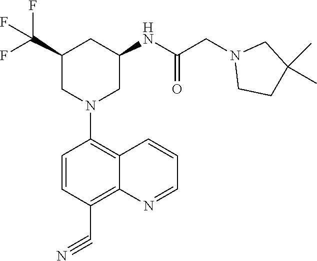 [SMILES: CC1(C)CCN(CC(=O)N[C@@H]2C[C@H](C(F)(F)F)CN(c3ccc(C#N)c4ncccc34)C2)C1] | 577 |
| 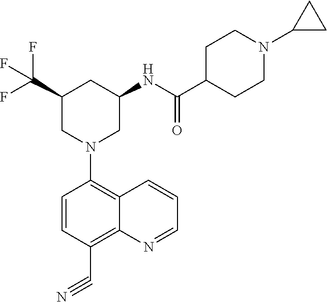 [SMILES: N#Cc1ccc(N2C[C@H](NC(=O)C3CCN(C4CC4)CC3)C[C@H](C(F)(F)F)C2)c2cccnc12] | 578 |
| 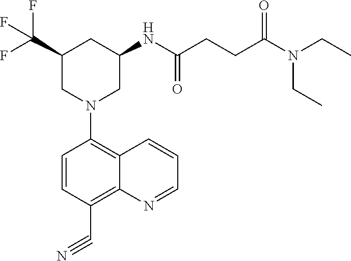 [SMILES: CCN(CC)C(=O)CCC(=O)N[C@@H]1C[C@H](C(F)(F)F)CN(c2ccc(C#N)c3ncccc23)C1] | 579 |
| 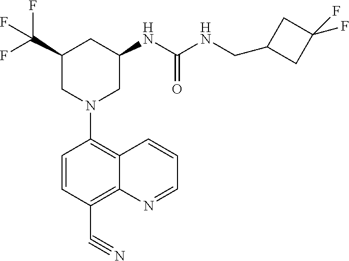 [SMILES: N#Cc1ccc(N2C[C@H](NC(=O)NCC3CC(F)(F)C3)C[C@H](C(F)(F)F)C2)c2cccnc12] | 580 |
| 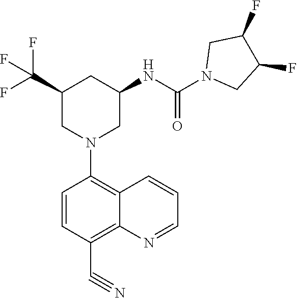 [SMILES: N#Cc1ccc(N2C[C@H](NC(=O)N3C[C@@H](F)[C@@H](F)C3)C[C@H](C(F)(F)F)C2)c2cccnc12] | 581 |
| 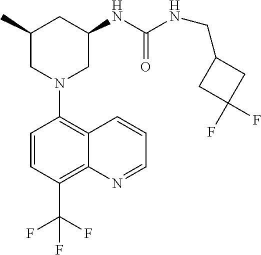 [SMILES: C[C@H]1C[C@@H](NC(=O)NCC2CC(F)(F)C2)CN(c2ccc(C(F)(F)F)c3ncccc23)C1] | 582 |
| 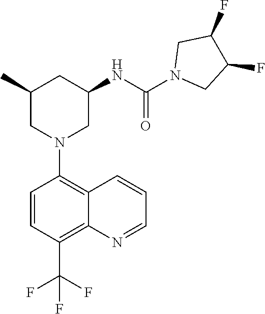 [SMILES: C[C@H]1C[C@@H](NC(=O)N2C[C@@H](F)[C@@H](F)C2)CN(c2ccc(C(F)(F)F)c3ncccc23)C1] | 583 |
| 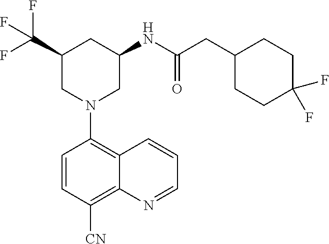 [SMILES: N#Cc1ccc(N2C[C@H](NC(=O)CC3CCC(F)(F)CC3)C[C@H](C(F)(F)F)C2)c2cccnc12] | 584 |
| 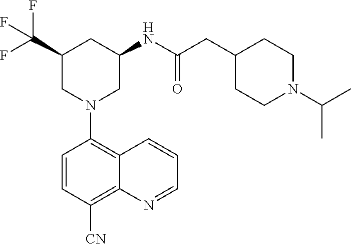 [SMILES: CC(C)N1CCC(CC(=O)N[C@@H]2C[C@H](C(F)(F)F)CN(c3ccc(C#N)c4ncccc34)C2)CC1] | 585 |
| 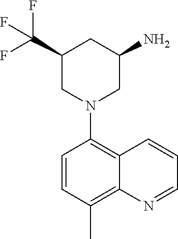 [SMILES: Cc1ccc(N2C[C@H](N)C[C@H](C(F)(F)F)C2)c2cccnc12] | 586 |
| 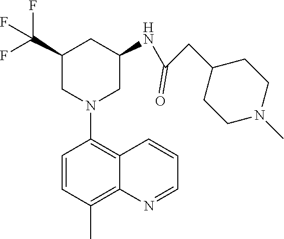 [SMILES: Cc1ccc(N2C[C@H](NC(=O)CC3CCN(C)CC3)C[C@H](C(F)(F)F)C2)c2cccnc12] | 587 |
| 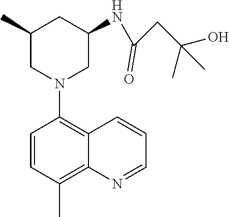 [SMILES: Cc1ccc(N2C[C@@H](C)C[C@@H](NC(=O)CC(C)(C)O)C2)c2cccnc12] | 588 |
| 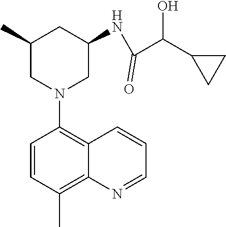 [SMILES: Cc1ccc(N2C[C@@H](C)C[C@@H](NC(=O)C(O)C3CC3)C2)c2cccnc12] | 589 |
| 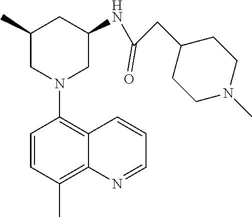 | 590 |
| 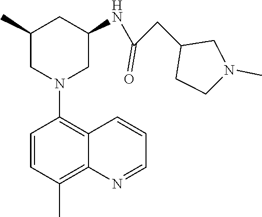 | 591 |

In certain embodiments, the invention provides the method as described above, wherein the compound selected from Table 2:

TABLE 2

| | |
|---|---|
| 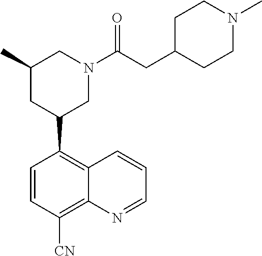 | Compound 1 |
| 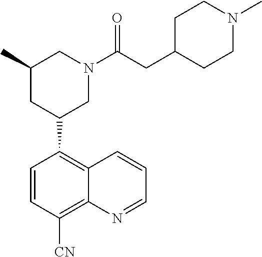 | Compound 2 |
| 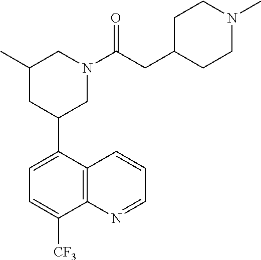 | Compound 3 |
| 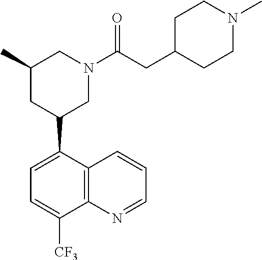 | Compound 4 |
| 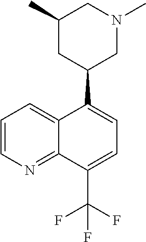 [SMILES: C[C@@H]1C[C@H](c2ccc(C(F)(F)F)c3ncccc23)CN(C)C1] | Compound 5 |
| 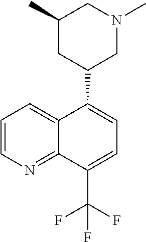 | Compound 6 |
| 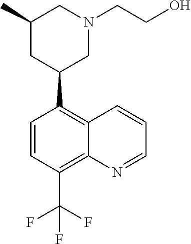 [SMILES: C[C@@H]1C[C@H](c2ccc(C(F)(F)F)c3ncccc23)CN(CCO)C1] | Compound 7 |
| 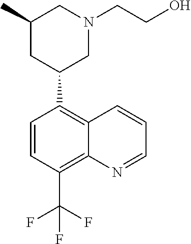 [SMILES: C[C@@H]1C[C@@H](c2ccc(C(F)(F)F)c3ncccc23)CN(CCO)C1] | Compound 8 |
| 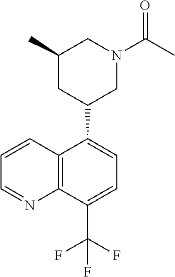 | Compound 9 |
| 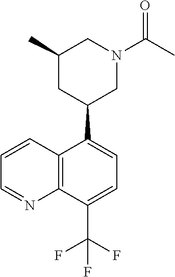 [SMILES: CC(=O)N1C[C@H](C)C[C@H](c2ccc(C(F)(F)F)c3ncccc23)C1] | Compound 10 |
| 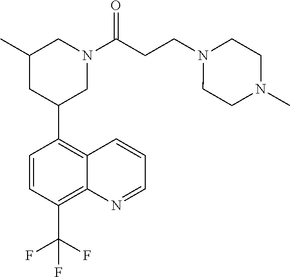 | Compound 11 |
| 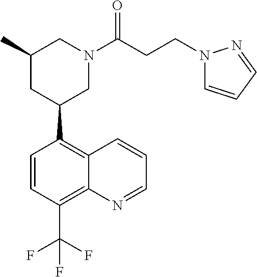 [SMILES: C[C@@H]1C[C@H](c2ccc(C(F)(F)F)c3ncccc23)CN(C(=O)CCn2cccn2)C1] | Compound 12 |
| 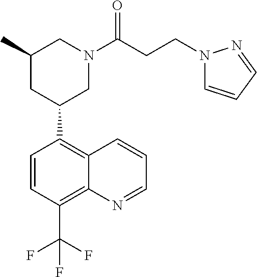 [SMILES: C[C@@H]1C[C@@H](c2ccc(C(F)(F)F)c3ncccc23)CN(C(=O)CCn2cccn2)C1] | Compound 13 |
| 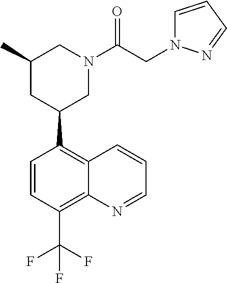 [SMILES: C[C@@H]1C[C@H](c2ccc(C(F)(F)F)c3ncccc23)CN(C(=O)Cn2cccn2)C1] | Compound 14 |
| 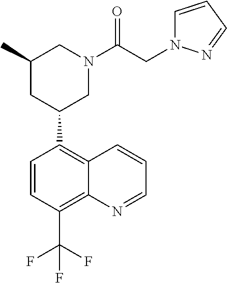 [SMILES: C[C@@H]1C[C@@H](c2ccc(C(F)(F)F)c3ncccc23)CN(C(=O)Cn2cccn2)C1] | Compound 15 |
| 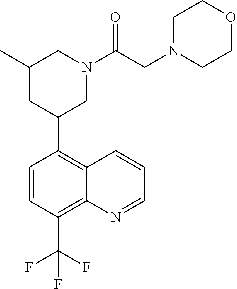 [SMILES: CC1CC(c2ccc(C(F)(F)F)c3ncccc23)CN(C(=O)CN2CCOCC2)C1] | Compound 16 |
| 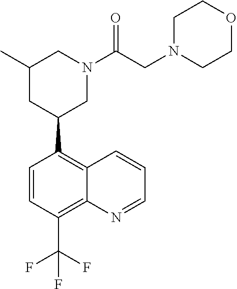 [SMILES: CC1C[C@H](c2ccc(C(F)(F)F)c3ncccc23)CN(C(=O)CN2CCOCC2)C1] | Compound 17 |
| 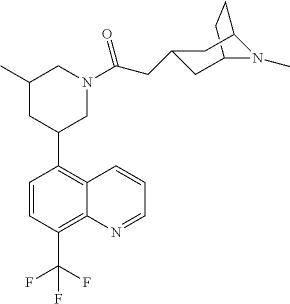 | Compound 18 |
| 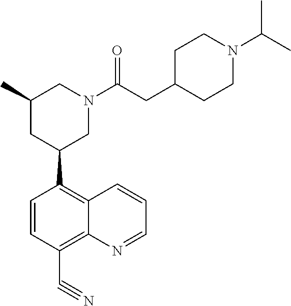 [SMILES: CC(C)N1CCC(CC(=O)N2C[C@H](C)C[C@H](c3ccc(C#N)c4ncccc34)C2)CC1] | Compound 19 |
| 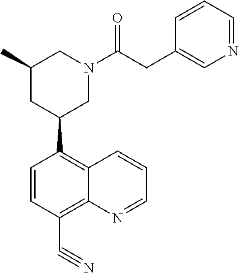 [SMILES: C[C@@H]1C[C@H](c2ccc(C#N)c3ncccc23)CN(C(=O)Cc2cccnc2)C1] | Compound 20 |
| 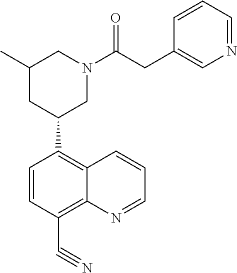 [SMILES: CC1C[C@@H](c2ccc(C#N)c3ncccc23)CN(C(=O)Cc2cccnc2)C1] | Compound 21 |
| 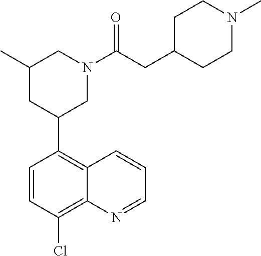 | Compound 22 |
| 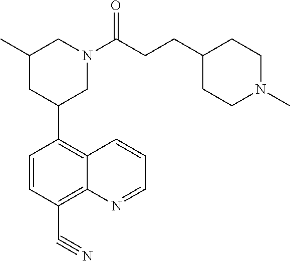 | Compound 23 |
| 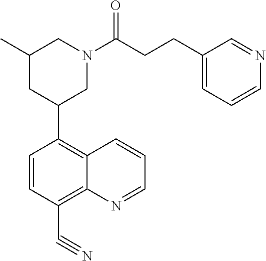 [SMILES: CC1CC(c2ccc(C#N)c3ncccc23)CN(C(=O)CCc2cccnc2)C1] | Compound 24 |
| 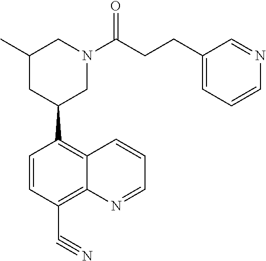 [SMILES: CC1C[C@H](c2ccc(C#N)c3ncccc23)CN(C(=O)CCc2cccnc2)C1] | Compound 25 |
| 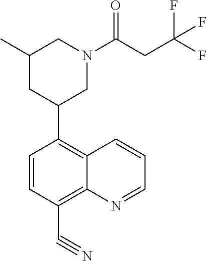 [SMILES: CC1CC(c2ccc(C#N)c3ncccc23)CN(C(=O)CC(F)(F)F)C1] | Compound 26 |
| 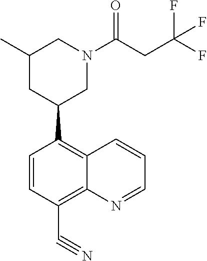 [SMILES: CC1C[C@H](c2ccc(C#N)c3ncccc23)CN(C(=O)CC(F)(F)F)C1] | Compound 27 |
| 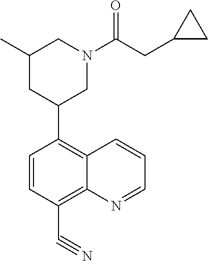 [SMILES: CC1CC(c2ccc(C#N)c3ncccc23)CN(C(=O)CC2CC2)C1] | Compound 28 |
| 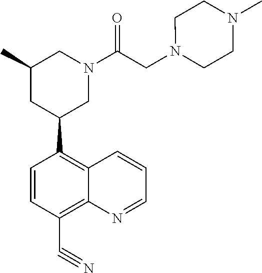 | Compound 29 |
| 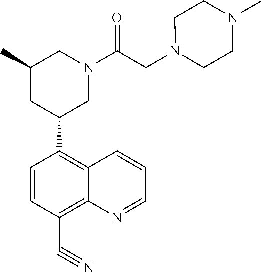 | Compound 30 |
| 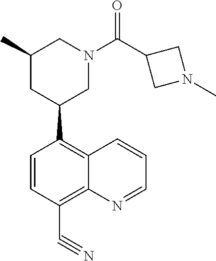 | Compound 31 |
| 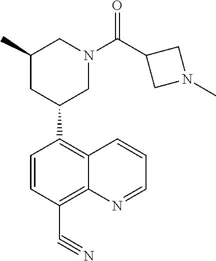 | Compound 32 |
| 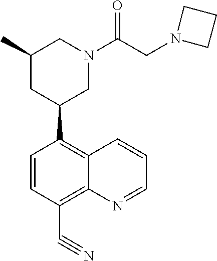 [SMILES: C[C@@H]1C[C@H](c2ccc(C#N)c3ncccc23)CN(C(=O)CN2CCC2)C1] | Compound 33 |
| 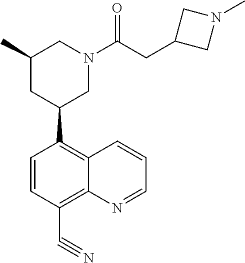 | Compound 34 |
| 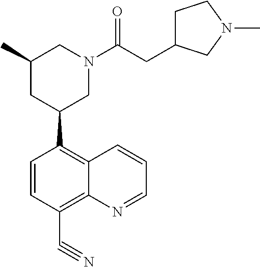 | Compound 35 |
| 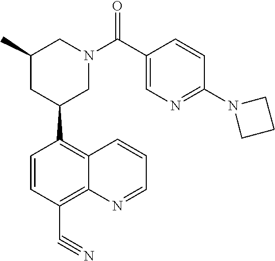 [SMILES: C[C@@H]1C[C@H](c2ccc(C#N)c3ncccc23)CN(C(=O)c2ccc(N3CCC3)nc2)C1] | Compound 36 |
| 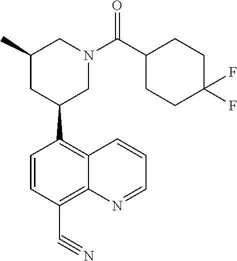 [SMILES: C[C@@H]1C[C@H](c2ccc(C#N)c3ncccc23)CN(C(=O)C2CCC(F)(F)CC2)C1] | Compound 37 |
| 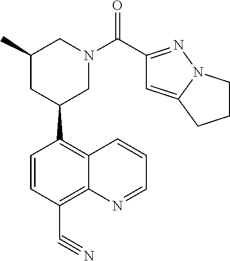 [SMILES: C[C@@H]1C[C@H](c2ccc(C#N)c3ncccc23)CN(C(=O)c2cc3n(n2)CCC3)C1] | Compound 38 |
| 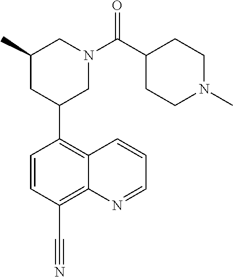 | Compound 39 |
| 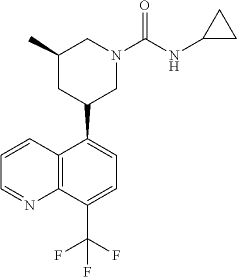 | Compound 40 |
| 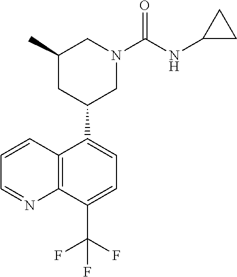 [SMILES: C[C@@H]1C[C@@H](c2ccc(C(F)(F)F)c3ncccc23)CN(C(=O)NC2CC2)C1] | Compound 41 |
| 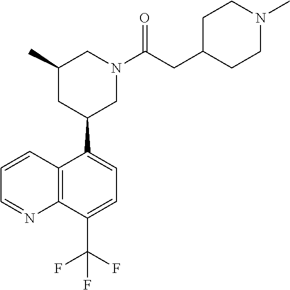 [SMILES: C[C@@H]1C[C@H](c2ccc(C(F)(F)F)c3ncccc23)CN(C(=O)CC2CCN(C)CC2)C1] | Compound 42 |
| 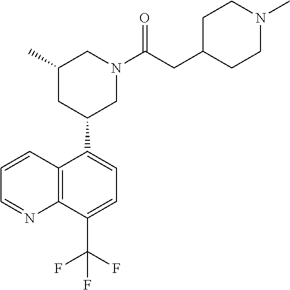 [SMILES: C[C@H]1C[C@@H](c2ccc(C(F)(F)F)c3ncccc23)CN(C(=O)CC2CCN(C)CC2)C1] | Compound 43 |
| 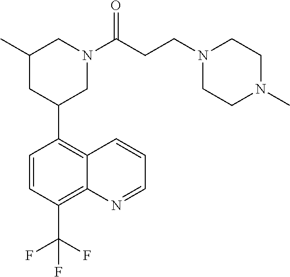 | Compound 44 |
| 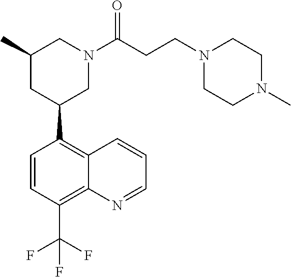 | Compound 45 |
| 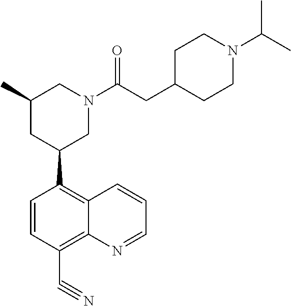 [SMILES: CC(C)N1CCC(CC(=O)N2C[C@H](C)C[C@H](c3ccc(C#N)c4ncccc34)C2)CC1] | Compound 46 |
| 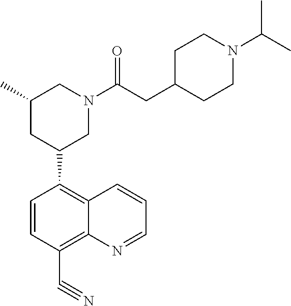 | Compound 47 |
| 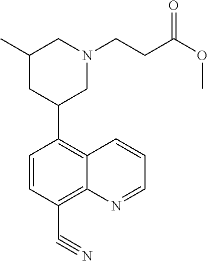 | Compound 48 |
| 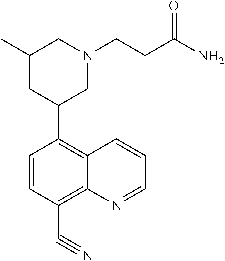 [SMILES: CC1CC(c2ccc(C#N)c3ncccc23)CN(CCC(N)=O)C1] | Compound 49 |
| 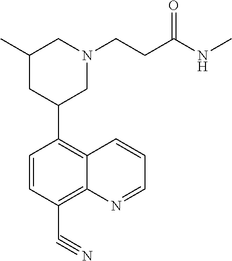 | Compound 50 |
| 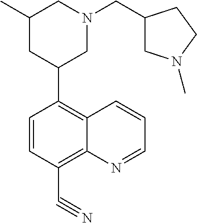 [SMILES: CC1CC(c2ccc(C#N)c3ncccc23)CN(CC2CCN(C)C2)C1] | Compound 51 |
| 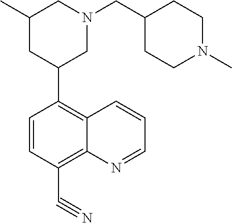 | Compound 52 |
| 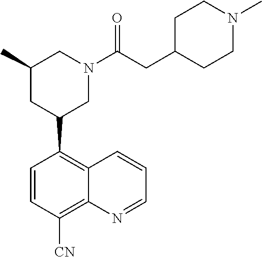 | Compound 53 |
| 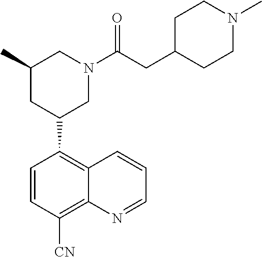 | Compound 54 |
| 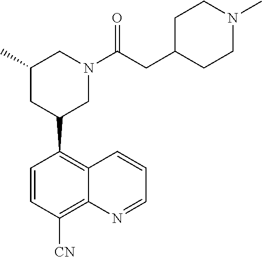 | Compound 55 |
| 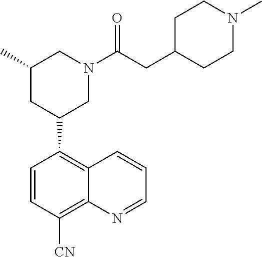 | Compound 56 |
| 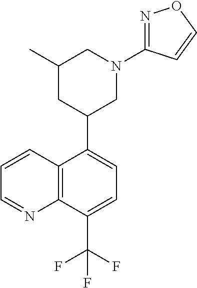 [SMILES: CC1CC(c2ccc(C(F)(F)F)c3ncccc23)CN(c2ccon2)C1] | Compound 57 |
| 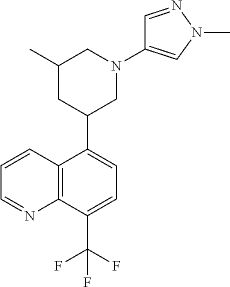 [SMILES: CC1CC(c2ccc(C(F)(F)F)c3ncccc23)CN(c2cnn(C)c2)C1] | Compound 58 |
| 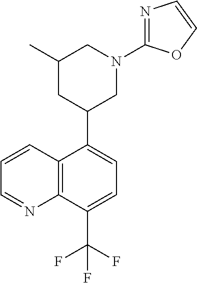 [SMILES: CC1CC(c2ccc(C(F)(F)F)c3ncccc23)CN(c2ncco2)C1] | Compound 59 |
| 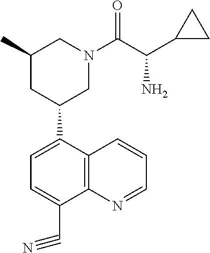 [SMILES: C[C@@H]1C[C@@H](c2ccc(C#N)c3ncccc23)CN(C(=O)[C@@H](N)C2CC2)C1] | Compound 60 |
| 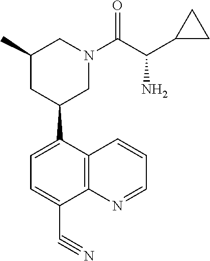 [SMILES: C[C@@H]1C[C@H](c2ccc(C#N)c3ncccc23)CN(C(=O)[C@@H](N)C2CC2)C1] | Compound 61 |
| 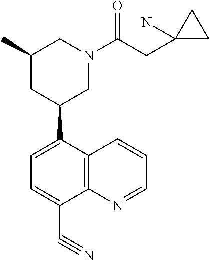 [SMILES: C[C@@H]1C[C@H](c2ccc(C#N)c3ncccc23)CN(C(=O)CC2(N)CC2)C1] | Compound 62 |
| 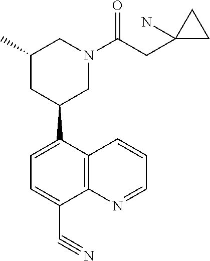 [SMILES: C[C@H]1C[C@H](c2ccc(C#N)c3ncccc23)CN(C(=O)CC2(N)CC2)C1] | Compound 63 |
| 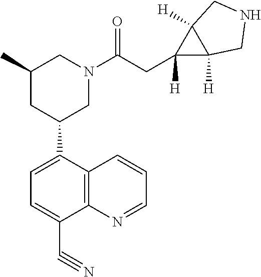 [SMILES: C[C@@H]1C[C@@H](c2ccc(C#N)c3ncccc23)CN(C(=O)C[C@H]2[C@@H]3CNC[C@@H]32)C1] | Compound 64 |
| 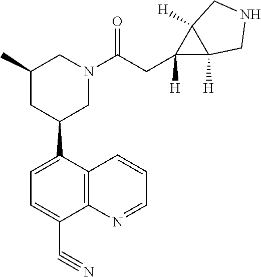 [SMILES: C[C@@H]1C[C@H](c2ccc(C#N)c3ncccc23)CN(C(=O)C[C@H]2[C@@H]3CNC[C@@H]32)C1] | Compound 65 |
| 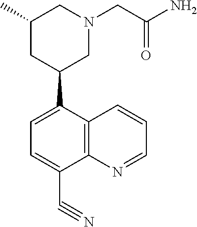 [SMILES: C[C@H]1C[C@H](c2ccc(C#N)c3ncccc23)CN(CC(N)=O)C1] | Compound 66 |
| 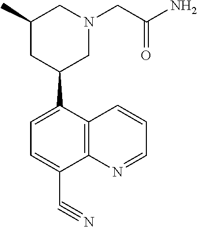 [SMILES: C[C@@H]1C[C@H](c2ccc(C#N)c3ncccc23)CN(CC(N)=O)C1] | Compound 67 |
| 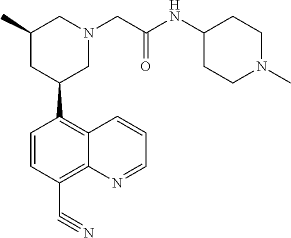 | Compound 68 |
| 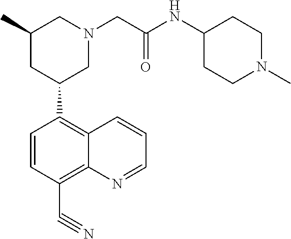 | Compound 69 |
| 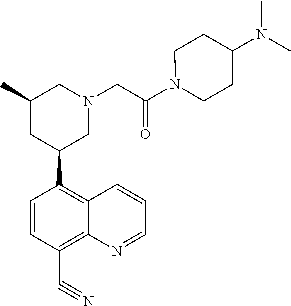 | Compound 70 |
| 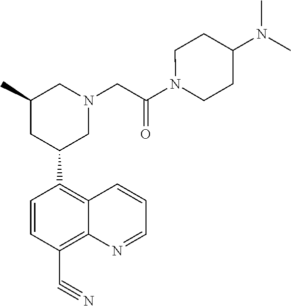 | Compound 71 |
| 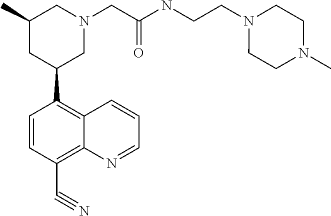 | Compound 72 |
| 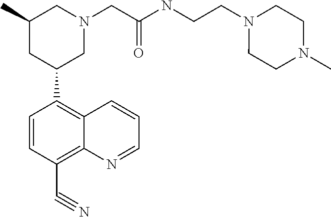 | Compound 73 |
| 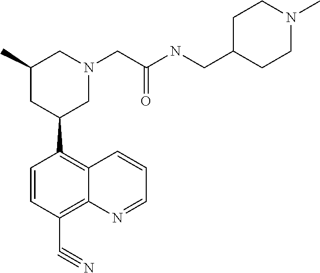 | Compound 74 |
| 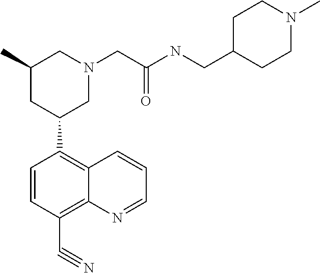 | Compound 75 |
| 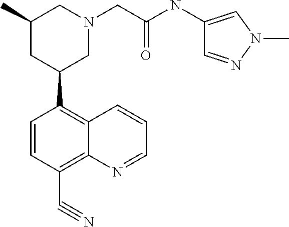 | Compound 76 |
| 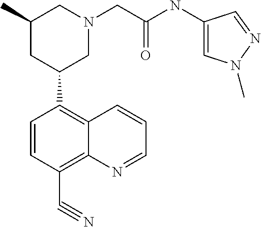 [SMILES: C[C@@H]1C[C@@H](c2ccc(C#N)c3ncccc23)CN(CC(=O)Nc2cnn(C)c2)C1] | Compound 77 |
| 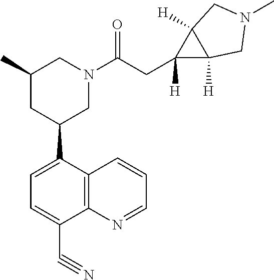 | Compound 78 |
| 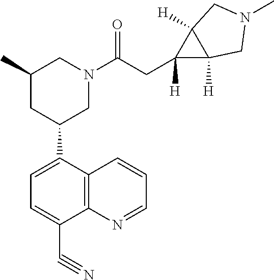 | Compound 79 |
| 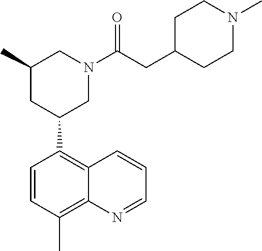 | Compound 80 |
| 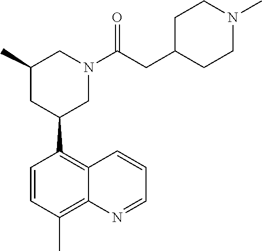 | Compound 81 |
| 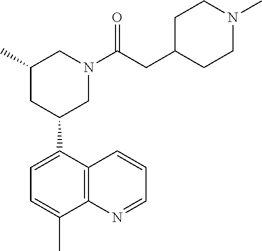 | Compound 82 |
| 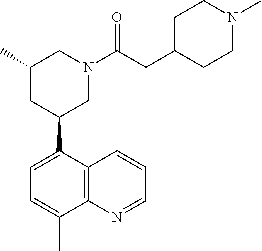 | Compound 83 |
| 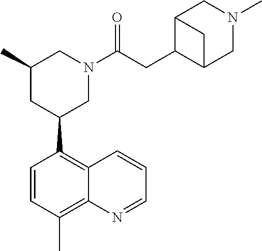 | Compound 84 |
| 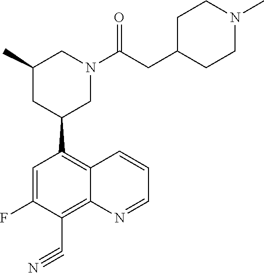 | Compound 85 |
| 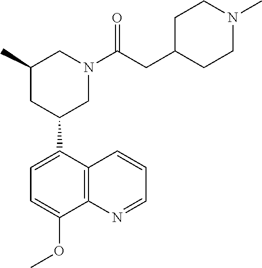 | Compound 86 |
| 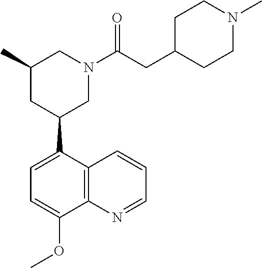 | Compound 87 |
| 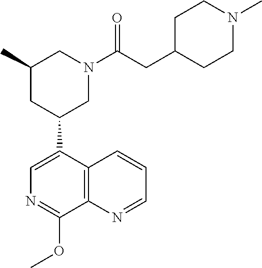 | Compound 88 |
| 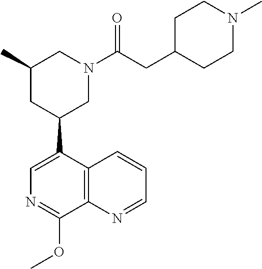 | Compound 89 |
| 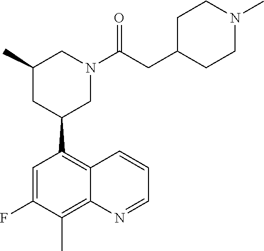 | Compound 90 |
| 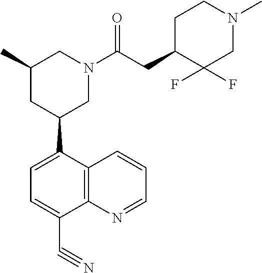 | Compound 91 |
| 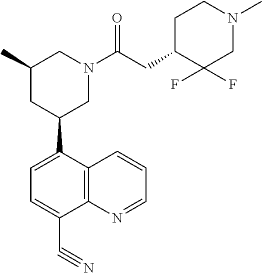 | Compound 92 |
| 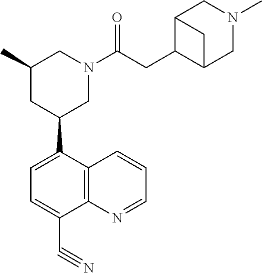 | Compound 93 |
| 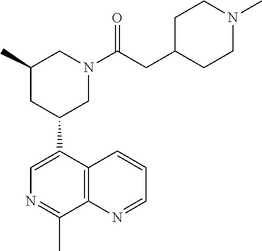 | Compound 94 |
| 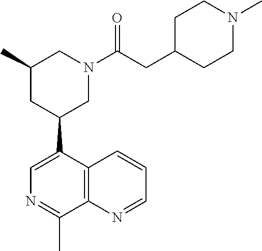 | Compound 95 |
| 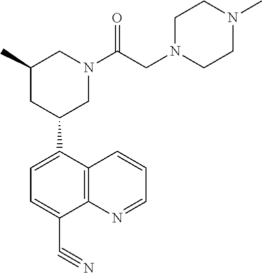 | Compound 96 |
| 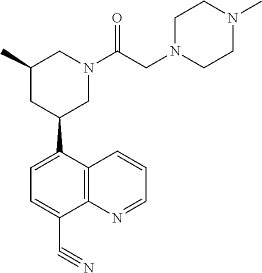 | Compound 97 |
| 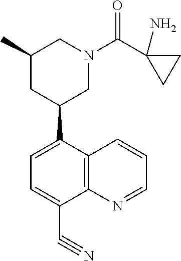 [SMILES: C[C@@H]1C[C@H](c2ccc(C#N)c3ncccc23)CN(C(=O)C2(N)CC2)C1] | Compound 98 |
| 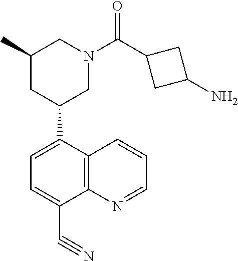 [SMILES: C[C@@H]1C[C@@H](c2ccc(C#N)c3ncccc23)CN(C(=O)C2CC(N)C2)C1] | Compound 99 |
| 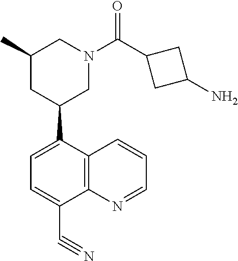 [SMILES: C[C@@H]1C[C@H](c2ccc(C#N)c3ncccc23)CN(C(=O)C2CC(N)C2)C1] | Compound 100 |
| 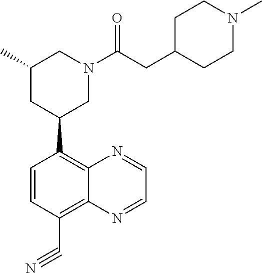 | Compound 101 |
| 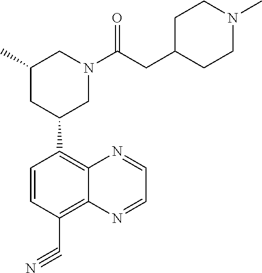 | Compound 102 |
| 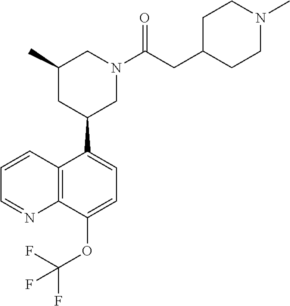 [SMILES: C[C@@H]1C[C@H](c2ccc(OC(F)(F)F)c3ncccc23)CN(C(=O)CC2CCN(C)CC2)C1] | Compound 103 |
| 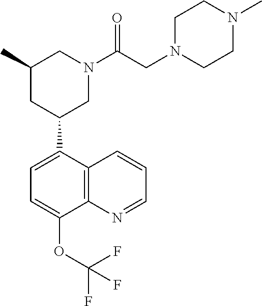 | Compound 104 |
| 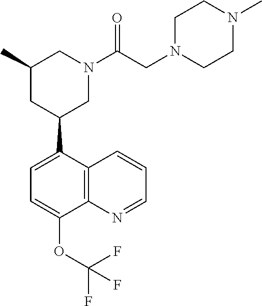 | Compound 105 |
| 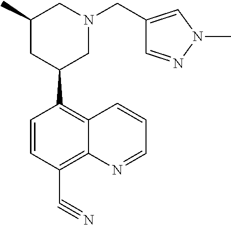 | Compound 106 |
| 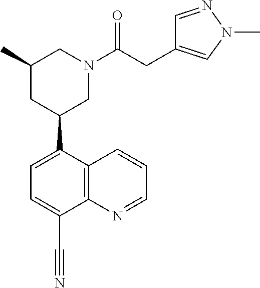 | Compound 107 |
| 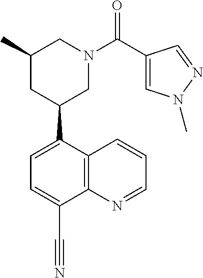 [SMILES: C[C@@H]1C[C@H](c2ccc(C#N)c3ncccc23)CN(C(=O)c2cnn(C)c2)C1] | Compound 108 |
| 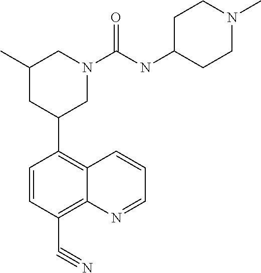 | Compound 109 |
| 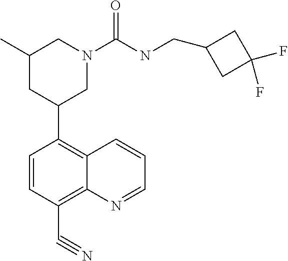 [SMILES: CC1CC(c2ccc(C#N)c3ncccc23)CN(C(=O)NCC2CC(F)(F)C2)C1] | Compound 110 |
| 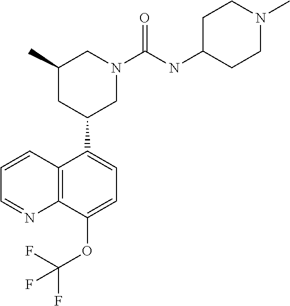 [SMILES: C[C@@H]1C[C@@H](c2ccc(OC(F)(F)F)c3ncccc23)CN(C(=O)NC2CCN(C)CC2)C1] | Compound 111 |
| 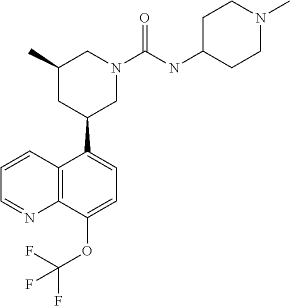 [SMILES: C[C@@H]1C[C@H](c2ccc(OC(F)(F)F)c3ncccc23)CN(C(=O)NC2CCN(C)CC2)C1] | Compound 112 |
| 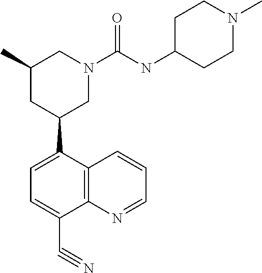 | Compound 113 |
| 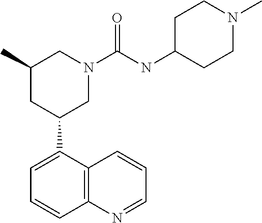 | Compound 114 |
| 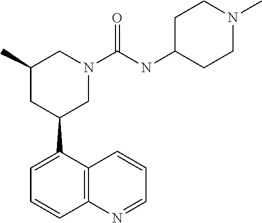 | Compound 115 |
| 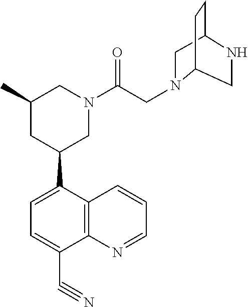 [SMILES: C[C@@H]1C[C@H](c2ccc(C#N)c3ncccc23)CN(C(=O)CN2CC3CCC2CN3)C1] | Compound 116 |
| 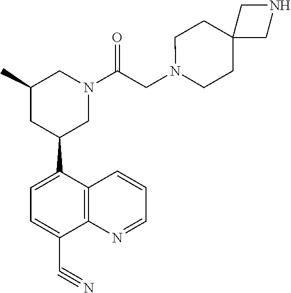 [SMILES: C[C@@H]1C[C@H](c2ccc(C#N)c3ncccc23)CN(C(=O)CN2CCC3(CC2)CNC3)C1] | Compound 117 |
| 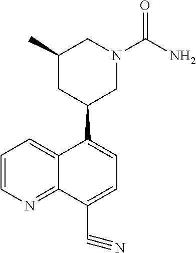 [SMILES: C[C@@H]1C[C@H](c2ccc(C#N)c3ncccc23)CN(C(N)=O)C1] | Compound 118 |
| 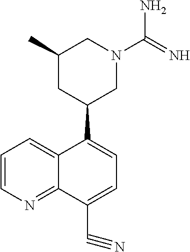 [SMILES: C[C@@H]1C[C@H](c2ccc(C#N)c3ncccc23)CN(C(=N)N)C1] | Compound 119 |
| 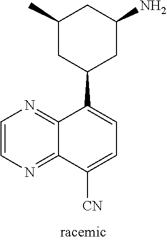 [SMILES: C[C@H]1C[C@@H](N)C[C@@H](c2ccc(C#N)c3nccnc23)C1] | Compound 120 |
| 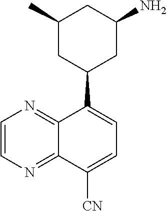 [SMILES: C[C@H]1C[C@@H](N)C[C@@H](c2ccc(C#N)c3nccnc23)C1] | Compound 121 |
| 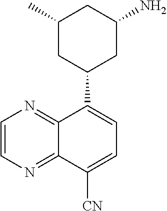 [SMILES: C[C@@H]1C[C@H](N)C[C@H](c2ccc(C#N)c3nccnc23)C1] | Compound 122 |
| 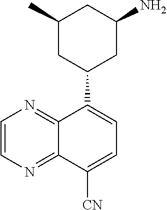 [SMILES: C[C@H]1C[C@@H](N)C[C@H](c2ccc(C#N)c3nccnc23)C1] | Compound 123 |
| 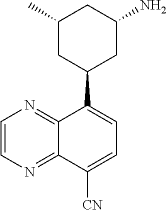 [SMILES: C[C@@H]1C[C@H](N)C[C@@H](c2ccc(C#N)c3nccnc23)C1] | Compound 124 |
| 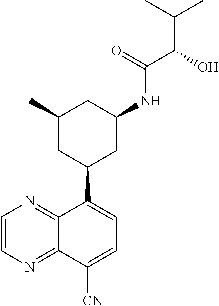 [SMILES: CC(C)[C@H](O)C(=O)N[C@@H]1C[C@H](C)C[C@H](c2ccc(C#N)c3nccnc23)C1] | Compound 125 |
| 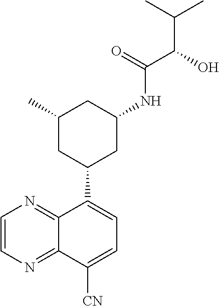 [SMILES: CC(C)[C@H](O)C(=O)N[C@H]1C[C@@H](C)C[C@@H](c2ccc(C#N)c3nccnc23)C1] | Compound 126 |
| 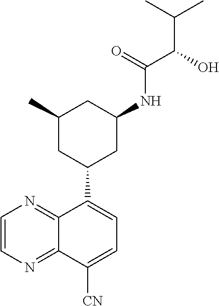 [SMILES: CC(C)[C@H](O)C(=O)N[C@@H]1C[C@H](C)C[C@@H](c2ccc(C#N)c3nccnc23)C1] | Compound 127 |
| 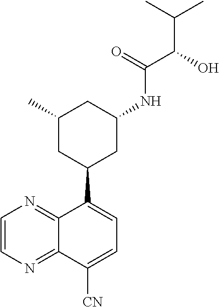 [SMILES: CC(C)[C@H](O)C(=O)N[C@H]1C[C@@H](C)C[C@H](c2ccc(C#N)c3nccnc23)C1] | Compound 128 |
| 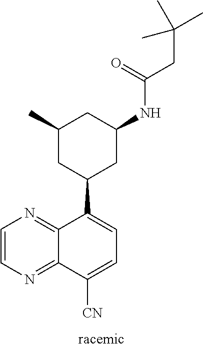 [SMILES: C[C@H]1C[C@@H](NC(=O)CC(C)(C)C)C[C@@H](c2ccc(C#N)c3nccnc23)C1] | Compound 129 |
| 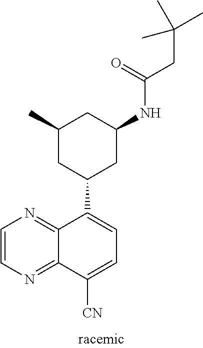 [SMILES: C[C@H]1C[C@@H](NC(=O)CC(C)(C)C)C[C@H](c2ccc(C#N)c3nccnc23)C1] | Compound 130 |
| 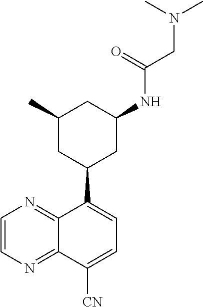 [SMILES: C[C@H]1C[C@@H](NC(=O)CN(C)C)C[C@@H](c2ccc(C#N)c3nccnc23)C1] | Compound 131 |
| 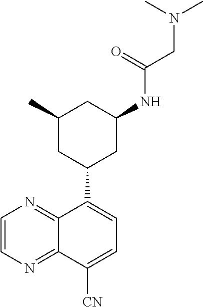 [SMILES: C[C@H]1C[C@@H](NC(=O)CN(C)C)C[C@H](c2ccc(C#N)c3nccnc23)C1] | Compound 132 |
| 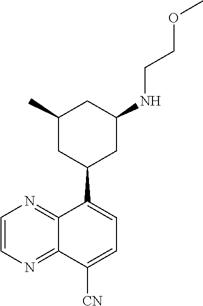 [SMILES: COCCN[C@@H]1C[C@H](C)C[C@H](c2ccc(C#N)c3nccnc23)C1] | Compound 133 |
| 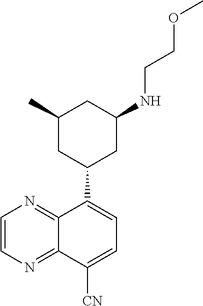 [SMILES: COCCN[C@@H]1C[C@H](C)C[C@@H](c2ccc(C#N)c3nccnc23)C1] | Compound 134 |
| 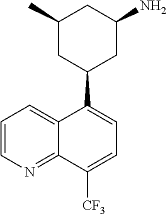 [SMILES: C[C@H]1C[C@@H](N)C[C@@H](c2ccc(C(F)(F)F)c3ncccc23)C1] | Compound 135 |
| 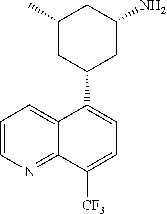 [SMILES: C[C@@H]1C[C@H](N)C[C@H](c2ccc(C(F)(F)F)c3ncccc23)C1] | Compound 136 |
| 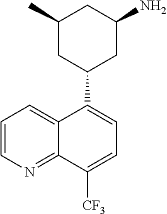 [SMILES: C[C@H]1C[C@@H](N)C[C@H](c2ccc(C(F)(F)F)c3ncccc23)C1] | Compound 137 |
| 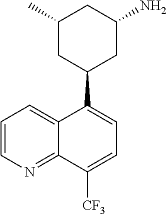 [SMILES: C[C@@H]1C[C@H](N)C[C@@H](c2ccc(C(F)(F)F)c3ncccc23)C1] | Compound 138 |
| 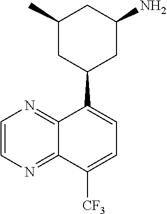 [SMILES: C[C@H]1C[C@@H](N)C[C@@H](c2ccc(C(F)(F)F)c3nccnc23)C1] | Compound 139 |
| 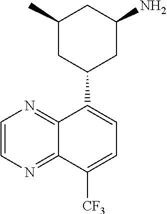 [SMILES: C[C@H]1C[C@@H](N)C[C@H](c2ccc(C(F)(F)F)c3nccnc23)C1] | Compound 140 |
| 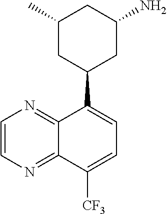 [SMILES: C[C@@H]1C[C@H](N)C[C@@H](c2ccc(C(F)(F)F)c3nccnc23)C1] | Compound 141 |
| 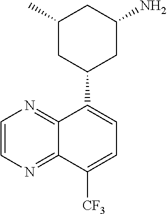 [SMILES: C[C@@H]1C[C@H](N)C[C@H](c2ccc(C(F)(F)F)c3nccnc23)C1] | Compound 142 |
| 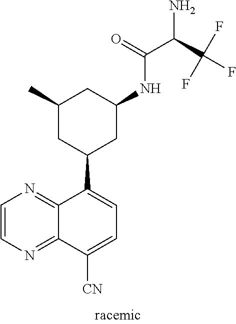 [SMILES: C[C@H]1C[C@@H](NC(=O)[C@@H](N)C(F)(F)F)C[C@@H](c2ccc(C#N)c3nccnc23)C1] | Compound 143 |
| 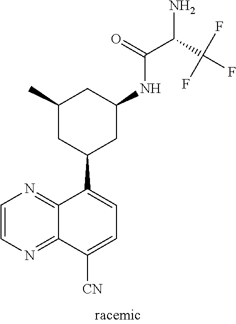 [SMILES: C[C@H]1C[C@@H](NC(=O)[C@H](N)C(F)(F)F)C[C@@H](c2ccc(C#N)c3nccnc23)C1] | Compound 144 |
| 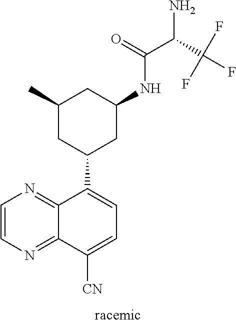 [SMILES: C[C@H]1C[C@@H](NC(=O)[C@H](N)C(F)(F)F)C[C@H](c2ccc(C#N)c3nccnc23)C1] | Compound 145 |
| 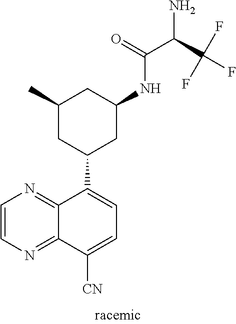 [SMILES: C[C@H]1C[C@@H](NC(=O)[C@@H](N)C(F)(F)F)C[C@H](c2ccc(C#N)c3nccnc23)C1] | Compound 146 |
| 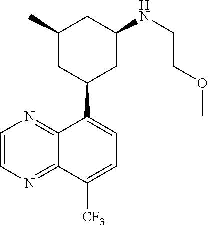 [SMILES: COCCN[C@@H]1C[C@H](C)C[C@H](c2ccc(C(F)(F)F)c3nccnc23)C1] | Compound 147 |
| 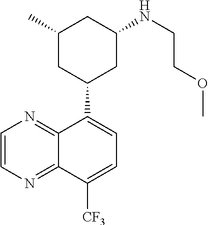 [SMILES: COCCN[C@H]1C[C@@H](C)C[C@@H](c2ccc(C(F)(F)F)c3nccnc23)C1] | Compound 148 |
| 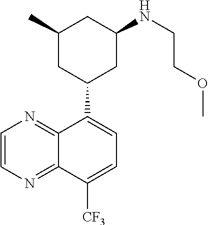 [SMILES: COCCN[C@@H]1C[C@H](C)C[C@@H](c2ccc(C(F)(F)F)c3nccnc23)C1] | Compound 149 |
| 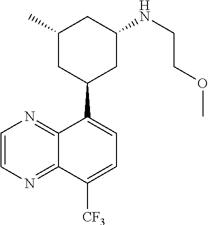 [SMILES: COCCN[C@H]1C[C@@H](C)C[C@H](c2ccc(C(F)(F)F)c3nccnc23)C1] | Compound 150 |
| 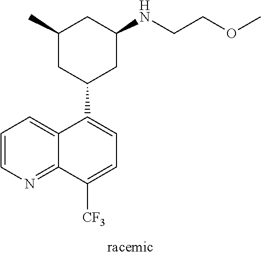 [SMILES: COCCN[C@@H]1C[C@H](C)C[C@@H](c2ccc(C(F)(F)F)c3ncccc23)C1] | Compound 151 |
| 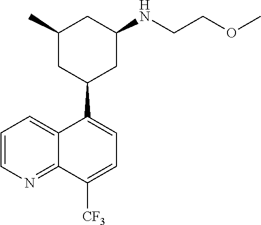 [SMILES: COCCN[C@@H]1C[C@H](C)C[C@H](c2ccc(C(F)(F)F)c3ncccc23)C1] | Compound 152 |
| 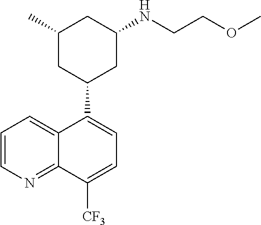 [SMILES: COCCN[C@H]1C[C@@H](C)C[C@@H](c2ccc(C(F)(F)F)c3ncccc23)C1] | Compound 153 |
| 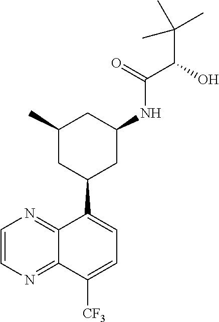 [SMILES: C[C@H]1C[C@@H](NC(=O)[C@@H](O)C(C)(C)C)C[C@@H](c2ccc(C(F)(F)F)c3nccnc23)C1] | Compound 154 |
| 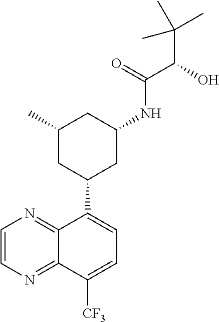 [SMILES: C[C@@H]1C[C@H](NC(=O)[C@@H](O)C(C)(C)C)C[C@H](c2ccc(C(F)(F)F)c3nccnc23)C1] | Compound 155 |
| 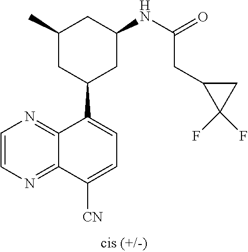 [SMILES: C[C@H]1C[C@@H](NC(=O)CC2CC2(F)F)C[C@@H](c2ccc(C#N)c3nccnc23)C1] | Compound 156 |
| 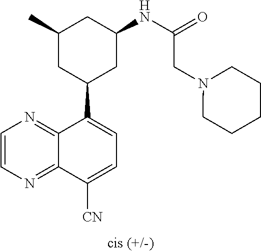 [SMILES: C[C@H]1C[C@@H](NC(=O)CN2CCCCC2)C[C@@H](c2ccc(C#N)c3nccnc23)C1] | Compound 157 |
| 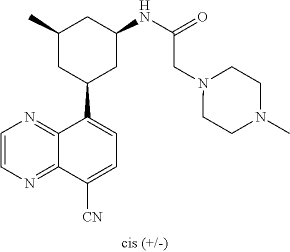 [SMILES: C[C@H]1C[C@@H](NC(=O)CN2CCN(C)CC2)C[C@@H](c2ccc(C#N)c3nccnc23)C1] | Compound 158 |
| 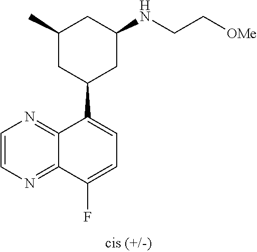 [SMILES: COCCN[C@@H]1C[C@H](C)C[C@H](c2ccc(F)c3nccnc23)C1] | Compound 159 |
| 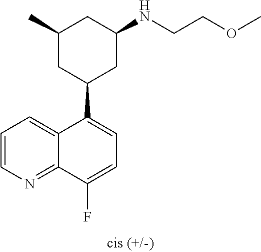 [SMILES: COCCN[C@@H]1C[C@H](C)C[C@H](c2ccc(F)c3ncccc23)C1] | Compound 160 |

In some embodiments, the present invention provides the method as described above, using a compound selected from those depicted above, or a pharmaceutically acceptable salt thereof.

Various structural depictions may show a heteroatom without an attached group, radical, charge, or counterion. Those of ordinary skill in the art are aware that such depictions are meant to indicate that the heteroatom is attached to hydrogen (e.g.,

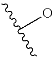 [SMILES: *O]

is understood to be

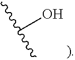 [SMILES: *O]

### 3. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit TLR7/8, or a mutant thereof, in a biological sample or in a patient. In certain embodiments, the amount of compound in compositions of this invention is such that is effective to measurably inhibit TLR7/8, or a mutant thereof, in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In some embodiments of any of the methods involving administration of a TLR inhibitor to an individual, the TLR inhibitor has a therapeutically acceptable safety profile. The TLR inhibitor may for example, have a therapeutically acceptable histological profile including an acceptably low, if any, toxicity of the liver, kidney, pancreas, or other organs. On occasion, polynucleotides have been associated with toxicity to certain organs such as the liver, kidney and pancreas. In some embodiments, the TLR inhibitor has a safety profile that is unexpected and advantageous. In some embodiments, a safety profile includes evaluation of toxicity, histological profile, and/or necrosis (e.g., liver, kidneys and/or heart). In some embodiments, the TLR inhibitor has a therapeutically acceptable level of toxicity. In some embodiments, the TLR inhibitor has a reduced level of toxicity as compared to another TLR inhibitor. In some embodiments, the TLR inhibitor induces a therapeutically acceptable reduction in body weight as compared to the initial body weight of a treated individual. In some embodiments, the TLR inhibitor induces less than 5%, 7.5%, 10%, 12.5, or 15% reduction in total body weight. In some embodiments, the TLR inhibitor has a therapeutically acceptable histology profile. In some embodiments, the TLR inhibitor has a better (e.g., lower severity score) histology profile, for example, as compared to a reference TLR inhibitor. In some embodiments, the TLR inhibitor has a better (e.g., lower severity score) histology profile upon evaluation of the liver, kidneys and/or heart, for example. In some embodiments, the TLR inhibitor has a therapeutically acceptable necrosis score. In some embodiments, the TLR inhibitor has reduced necrosis and/or better (e.g., lower) necrosis score, for example, as compared to a reference TLR inhibitor. In some embodiments, the TLR inhibitor has reduced renal and/or hepatocellular necrosis and/or a better renal and/or hepatocellular necrosis score, for example, as compared to a reference TLR inhibitor.

Accordingly, the invention provides a method of activating TLR7 in an animal, especially a mammal, preferably a human comprising administering an effective amount of a compound of Formula I to the animal. As with all compositions for inhibition of an immune response, the effective amounts and method of administration of the particular TLR inhibitor formulation can vary based on the individual, what condition is to be treated and other factors evident to one skilled in the art. An effective amount of a compound will vary according to factors known in the art but is expected to be a dose of about 0.1 to 10 mg/kg, 0.5 to 10 mg/kg, 1 to 10 mg/kg, 0.1 to 20 mg/kg, 0.1 to 20 mg/kg, or 1 to 20 mg/kg.

In various embodiments, compounds of formula (I), and related formulae exhibit an IC50 for the binding to TLR7/8 of less than about 5 μM, preferably less than about 1 μM and even more preferably less than about 0.100 μM.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to inhibit TLR7/8 activity, usually between about one hour and one week. In-vitro treatment can be carried out using cultivated cells from a biopsy sample or cell line.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

For identification of a signal transduction pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds according to the invention can also be used as reagents for testing TLR7/8-dependent signal transduction pathways in animals and/or cell culture models or in the clinical diseases mentioned in this application.

Moreover, the subsequent teaching of the present specification concerning the use of the compounds according to formula (I) and its derivatives for the production of a medicament for the prophylactic or therapeutic treatment and/or monitoring is considered as valid and applicable without restrictions to the use of the compound for the inhibition of TLR7/8 activity if expedient.

The invention also relates to the use of compounds according to formula (I) and/or physiologically acceptable salts thereof for the prophylactic or therapeutic treatment and/or monitoring of diseases that are caused, mediated and/or propagated by TLR7/8 activity. Furthermore, the invention relates to the use of compounds according to formula (I) and/or physiologically acceptable salts thereof for the production of a medicament for the prophylactic or therapeutic treatment and/or monitoring of diseases that are caused, mediated and/or propagated by TLR7/8 activity. In certain embodiments, the invention provides the use of a compound according to formula I or physiologically acceptable salts thereof, for the production of a medicament for the prophylactic or therapeutic treatment of a TLR7/8-mediated disorder.

Compounds of formula (I) and/or a physiologically acceptable salt thereof can furthermore be employed as intermediate for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

The compounds of formula (I) according to the invention can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease completely. Either the identical compound or different compounds can be applied. The methods of the invention can also be used to reduce the likelihood of developing a disorder or even prevent the initiation of disorders associated with TLR7/8 activity in advance or to treat the arising and continuing symptoms.

In the meaning of the invention, prophylactic treatment is advisable if the subject possesses any preconditions for the aforementioned physiological or pathological conditions, such as a familial disposition, a genetic defect, or a previously incurred disease.

The invention furthermore relates to a medicament comprising at least one compound according to the invention and/or pharmaceutically usable derivatives, salts, solvates and stereoisomers thereof, including mixtures thereof in all ratios. In certain embodiments, the invention relates to a medicament comprising at least one compound according to the invention and/or physiologically acceptable salts thereof.

A “medicament” in the meaning of the invention is any agent in the field of medicine, which comprises one or more compounds of formula (I) or preparations thereof (e.g. a pharmaceutical composition or pharmaceutical formulation) and can be used in prophylaxis, therapy, follow-up or aftercare of patients who suffer from diseases, which are associated with TLR7/8 activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of formula (I) is combined with at least another agent as active ingredient, which is either another compound of formula (I) or a compound of different structural scaffold. The active ingredients can be used either simultaneously or sequentially.

Also provided herein are kits comprising a TLR inhibitor as provided herein, and instructions for use in the methods of inhibiting a TLR7- and/or TLR8-dependent immune response.

The kits may comprise one or more containers comprising a TLR inhibitor (or a formulation comprising a TLR inhibitor) as described herein, and a set of instructions, generally written instructions although electronic storage media (e.g., magnetic diskette or optical disk) containing instructions are also acceptable, relating to the use and dosage of the TLR inhibitor or formulation for the intended treatment. The instructions included with the kit generally include information as to dosage, dosing schedule, and route of administration for the intended treatment. The containers for the TLR inhibitor (or formulations comprising a TLR inhibitor) may be unit doses, bulk packages (e.g., multi-dose packages) or sub-unit doses. The kits may further comprise a container comprising an adjuvant.

In another aspect, the invention provides for a kit consisting of separate packs of an effective amount of a compound according to the invention and/or pharmaceutically acceptable salts, derivatives, solvates and stereoisomers thereof, including mixtures thereof in all ratios, and optionally, an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect.

In certain embodiments, a therapeutically effective amount of a compound of the formula (I), and related formulae and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In certain embodiments, the pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Liquid dosage forms for oral administration include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are formulated according to the known art using suitable dispersing or wetting agents and suspending agents. The sterile injectable preparation are also a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This is accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type are also employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting TLR7/8 activity in a biological sample comprising the step of contacting said biological sample with a compound of this invention, or a composition comprising said compound.

According to another embodiment, the invention relates to a method of inhibiting TLR7/8, or a mutant thereof, activity in a biological sample in a positive manner, comprising the step of contacting said biological sample with a compound of this invention, or a composition comprising said compound.

The compounds of the invention are useful in-vitro as unique tools for understanding the biological role of TLR7/8, including the evaluation of the many factors thought to influence, and be influenced by, the production of TLR7/8 and the interaction of TLR7/8. The present compounds are also useful in the development of other compounds that interact with TLR7/8 since the present compounds provide important structure-activity relationship (SAR) information that facilitate that development. Compounds of the present invention that bind to TLR7/8 can be used as reagents for detecting TLR7/8 in living cells, fixed cells, in biological fluids, in tissue homogenates, in purified, natural biological materials, etc. For example, by labeling such compounds, one can identify cells expressing TLR7/8. In addition, based on their ability to bind TLR7/8, compounds of the present invention can be used in in-situ staining, FACS (fluorescence-activated cell sorting), sodium dodecyl sulfate polyacrylamide gel electrophoresis (SDS-PAGE), ELISA (enzyme-linked immunoadsorptive assay), etc., enzyme purification, or in purifying cells expressing TLR7/8 inside permeabilized cells. The compounds of the invention can also be utilized as commercial research reagents for various medical research and diagnostic uses. Such uses can include but are not limited to: use as a calibration standard for quantifying the activities of candidate TLR7/8 inhibitors in a variety of functional assays: use as blocking reagents in random compound screening, i.e. in looking for new families of TLR7/8 ligands, the compounds can be used to block recovery of the presently claimed TLR7/8 compounds; use in the co-crystallization with TLR7/8, i.e. the compounds of the present invention will allow formation of crystals of the compound bound to TLR7/8, enabling the determination of enzyme/compound structure by x-ray crystallography; other research and diagnostic applications, wherein TLR7/8 is preferably activated or such activation is conveniently calibrated against a known quantity of an TLR7/8 inhibitor, etc.; use in assays as probes for determining the expression of TLR7/8 in cells; and developing assays for detecting compounds which bind to the same site as the TLR7/8 binding ligands.

The compounds of the invention can be applied either themselves and/or in combination with physical measurements for diagnostics of treatment effectiveness. Pharmaceutical compositions containing said compounds and the use of said compounds to treat TLR7/8-mediated conditions is a promising, novel approach for a broad spectrum of therapies causing a direct and immediate improvement in the state of health, whether in human or in animal. The orally bioavailable and active new chemical entities of the invention improve convenience for patients and compliance for physicians.

The compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Modulation of TLR7/8, or a mutant thereof, activity in a biological sample is useful for a variety of purposes that are known to one of skill in the art. Examples of such purposes include, but are not limited to, blood transfusion, organ transplantation, biological specimen storage, and biological assays.

### EXAMPLES

### Example 1. Pharmaceutical Preparations

(A) Injection vials: A solution of 100 g of an active ingredient according to the invention and 5 g of disodium hydrogen phosphate in 31 of bidistilled water is adjusted to pH 6.5 using 2 N hydrochloric acid, sterile filtered, transferred into injection vials, is lyophilized under sterile conditions and is sealed under sterile conditions. Each injection vial contains 5 mg of active ingredient.

(B) Suppositories: A mixture of 20 g of an active ingredient according to the invention is melted with 100 g of soy lecithin and 1400 g of cocoa butter, is poured into moulds and is allowed to cool. Each suppository contains 20 mg of active ingredient.

(C) Solution: A solution is prepared from 1 g of an active ingredient according to the invention, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilized by irradiation. This solution could be used in the form of eye drops.

(D) Ointment: 500 mg of an active ingredient according to the invention is mixed with 99.5 g of Vaseline under aseptic conditions.

(E) Tablets: A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

(F) Coated tablets: Tablets are pressed analogously to Example E and subsequently are coated in a conventional manner with a coating of sucrose, potato starch, talc, tragacanth and dye.

(G) Capsules: 2 kg of an active ingredient according to the invention are introduced into hard gelatin capsules in a conventional manner in such a way that each capsule contains 20 mg of the active ingredient.

(H) Ampoules: A solution of 1 kg of an active ingredient according to the invention in 601 of bidistilled water is sterile filtered, transferred into ampoules, is lyophilized under sterile conditions and is sealed under sterile conditions. Each ampoule contains 10 mg of active ingredient.

(I) Inhalation spray: 14 g of an active ingredient according to the invention are dissolved in 10 l of isotonic NaCl solution, and the solution is transferred into commercially available spray containers with a pump mechanism. The solution could be sprayed into the mouth or nose. One spray shot (about 0.1 ml) corresponds to a dose of about 0.14 mg.

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

A large proportion of lupus patients suffer from neurological complications but no currently used lupus treatment ameliorates these symptoms (Magro-Checa C. et al., Drugs 2016, March; 76(4):459-83)

In recent years evidence has been reported that (over-)activation or overexpression of TLR7 or TLR8 by microRNAs (miRNAs) in the CNS may play a role in the development and progress of certain CNS disorders, in particular of inflammatory or auto-immunological CNS disorders.

Several reports document the increased levels of microRNAs from the let-7 family in the cerebrospinal fluid from Alzheimer's disease patients (Lehmann, S. M., et al., Nat Neurosci. 2012 June; 15(6):827-35). miRNAs regulate gene expression by modulation of mRNA stability or translation, however, in this case the miRNAs were detected outside of the cell in microsomes. In addition, these miRNAs were found to activate TLR7 in neurons of mice when introduced by intrathecal injection. Mice lacking TLR7 were resistant to this effect. Similarly, additional reports describe the effect of the let7 family of miRNAs on neurons via activation of TLR7 that causes defects in neuronal dendritic arborization in mice (Liu, H., et al., Exp Neurol. 2015 July; 269:202-12).

In addition, TLR7 may be involved in mediating pain and itch by detecting miRNAs that are released by injured tissues. In a model of mechanical allodynia, injection of let7 miRNA resulted in pain sensing that was dependent on expression of TLR7 in neurons (Helley, M. P., et al., Neuroscience. 2015 Dec. 3; 310:686-98; Park, C. K., Neuron. 2014 Apr. 2; 82(1):47-54).

While the mechanism of miRNA transport from one cell to another is not completely understood yet, several groups suggested a role of various alarmin proteins, i.e. HMGB1 or LL37 in forming complexes with miRNAs and transducing neighboring cells in a receptor-dependent or independent manner (Coleman, L. G. jr., et al., J Neuroinflammation. 2017 Jan. 25; 14(1):22).

In summary, miRNAs secreted by stressed cells can serve as stress signals that activate cells in vicinity by activating their TLR7/8. The type of response that is driven by recognition of miRNAs is cell-type dependent. Neurons respond by, inter alia, activating pain signaling, shortening their dendrites, demyelination, etc. Thus, TLR7/8 inhibitors that can enter CNS are able to prevent these pathological processes and can be used as therapeutics for treatment of CNS disorders, in particular of systemic lupus erythematosus (SLE), lupus nephritis (LN), Sjogren's syndrome, multiple sclerosis (MS), Alzheimer's disease (AD), and other diseases characterized by CNS disorders.

The experiments exhibited below show that miRNAs of let7 family that were found in the CNS can induce cytokines in human blood cells and that their activity can be blocked using TLR7/8 antagonists described herein.

### Example 2

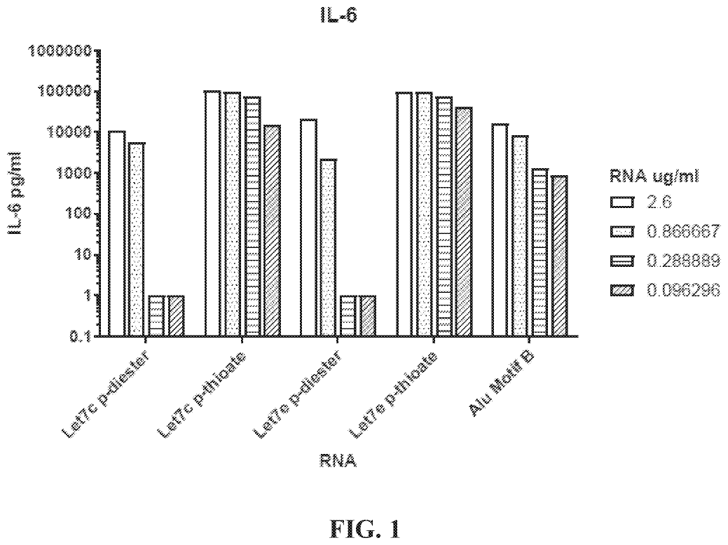

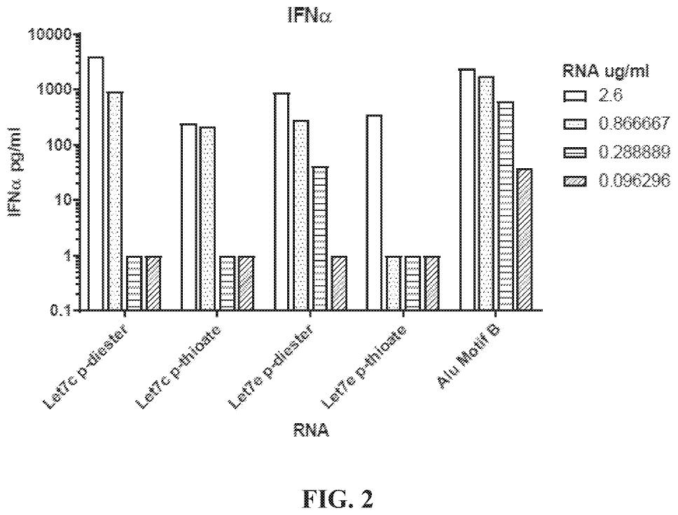

Human peripheral blood lymphocytes were transfected with let-7c and let-7e miRNAs. 24 hours following transfection, cell supernatants were analysed for the presence of IL-6 (FIG. 1) and IFNα (FIG. 2) cytokines. Two versions of the RNA oligos with phosphoester or phosphorothioate bonds were used, respectively.

Hu let-7c
UGAGGUAGUAGGUUGUAUGGUU
(+/− phosphorothioate bonds)
Hu let-7e
UGAGGUAGGAGGUUGUAUAGUU
(+/− phosphorothioate bonds)
Alu motif B
UUUUUUUUUUUUUUUUUUUUUUUUGAGACGGAGUCUCGCUCUGUCGCC
(diester bonds only)

These findings show that delivery of let7 miRNAs into human PBMCs induces production of IFNα and IL-6 in a dose-dependent manner.

### Example 3

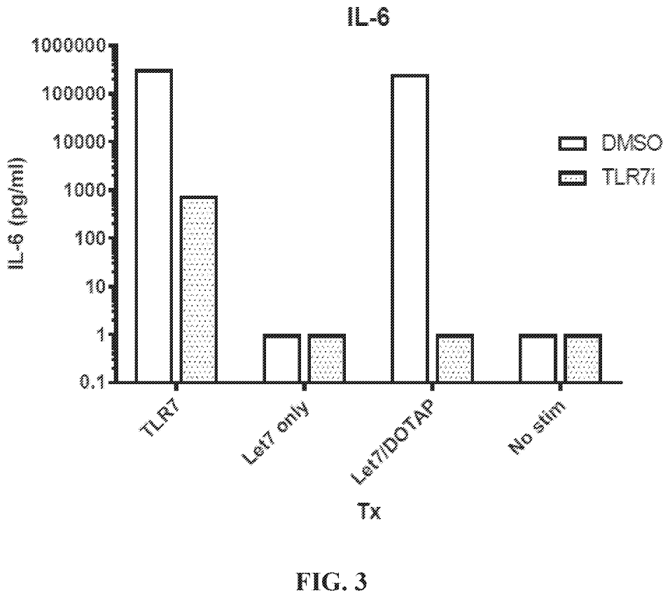

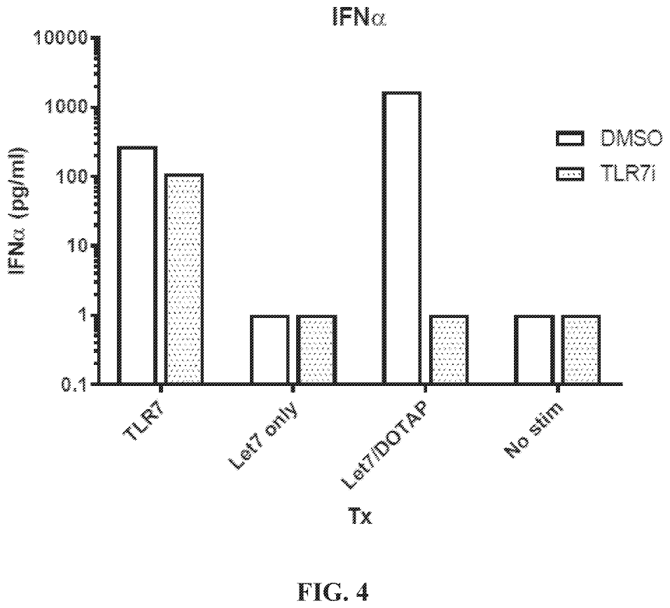

Human PBMCs were treated with TLR7 agonist (TLR7), let7c miRNA or transfected with let7c miRNA (let7/DOTAP) in the presence of TLR7/8 antagonist (Compound 467 in Table 1 above). Levels of IL-6 (FIG. 3) and IFNα (FIG. 4) were measured following overnight incubation.

These findings show that small molecule TLR7/8 antagonist blocks production of cytokines induced by let-7 miRNA in human PBMCs.

### Example 4

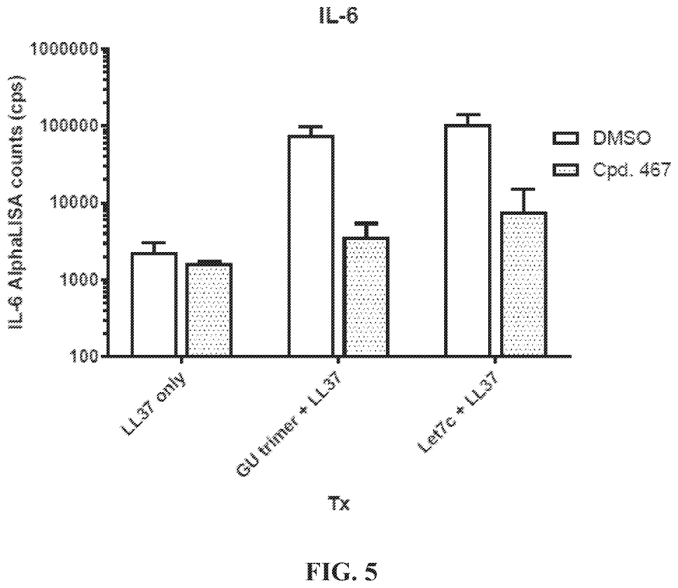

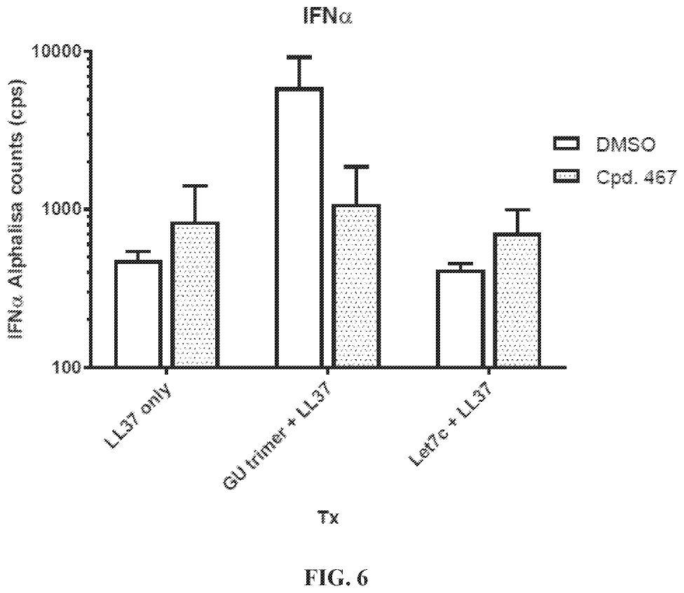

Binding of LL37 to miRNA enables delivery of miRNA to human PBMCs and stimulate TLR7/8-mediated production of cytokines. Human recombinant LL37 protein was used alone, or in a complex with GU trimer or let-7c miRNA to activate human PBMCs in the presence or absence of TLR7/8 inhibitor (Compound 467 in Table 1 above). Levels of IL-6 (FIG. 5) and IFNα (FIG. 6) were measured following overnight incubation.

GU trimer:
G*U*U*G*U*G*U*U*G*U*G*U*U*G*U
(phosphorothioate only)

LL37 can form complexes with RNAs and deliver them inside the cell to activate TLR7/8. In the presence of a TLR7 inhibitor of the invention the activation is suppressed.

We claim:

1. A method for the treatment of disorders related to TLR7/8 overexpression or TLR7/8 aberrant activation, the method comprising:
administering to a patient a compound of formula I, 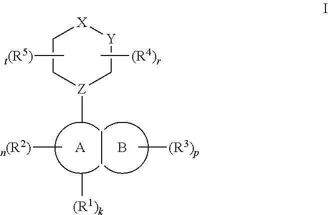
or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
each R¹ is independently absent, —H, —CH₃, —CF₃, —CN, —F, —Cl, —OCH₃, —OC₂H₅, or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;
Z is N or CH;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0, 1 or 2;
n is 0, 1, or 2;
p is 0, 1, or 2;
r is 0, 1, or 2; and
t is 0, 1, or 2.

2. The method of claim 1, wherein the disorder is selected from multiple sclerosis, Alzheimer's Disease, myositis, stroke, ischemia, Central Nervous System (CNS) neuropathies, systemic lupus erythematosus, lupus nephritis, Sjogren's syndrome, Guillain-Barré syndrome, alcoholic hepatitis, non-alcoholic steatohepatitis, congenital heart block, autoimmune hepatitis, autoimmune pancreatitis, adult onset Still's disease, drug-induced neurological disorders, and substance addiction.

3. The method of claim 1, wherein the compound is a compound of formula II, 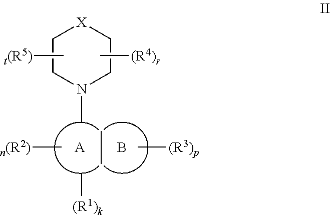
or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is absent, —H, —CHF₂, —CF₃, —OMe, —OC₂H₅, or —CN;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂, X is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SOR, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

4. The method of claim 1, wherein the compound is a compound of formula III, 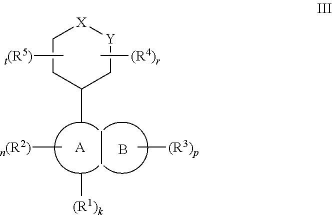
or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is heteroaryl having 1-4 heteroatoms independently selected from nitrogen, oxygen, or sulfur; each of which is optionally substituted;
R¹ is —H, —CH₃, —CF₃, —CN, —F, —Cl, —OCH₃, or —OCF₃;
each R² is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR⁴, S, S(R⁴), or S(R⁴)₂;
Y is C(R⁴)₂, O, NR⁴, S, S(R⁴), or S(R⁴)₂;
each R⁴ is independently —H, —R, halogen, -haloalkyl, —OR, —SR, —CN, —NO₂, —SO₂R, —SOR, —C(O)R, —CO₂R, —C(O)N(R)₂, —C(NH)R, —C(NH)NR₂, —NRC(O)R, —NRC(O)N(R)₂, —NRSO₂R, or —N(R)₂;
each R⁵ is independently —H, —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 1 or 2;
n is 0, 1, or 2;
p is 0, 1, or 2;
r is 0, 1, or 2; and
t is 0, 1, or 2.

5. The method of claim 1, wherein the compound is selected from the group consisting of a compound shown in the following Table:

| | |
|---|---|
| 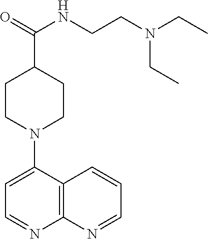 [SMILES: CCN(CC)CCNC(=O)C1CCN(c2ccnc3ncccc23)CC1] | 1 |
| 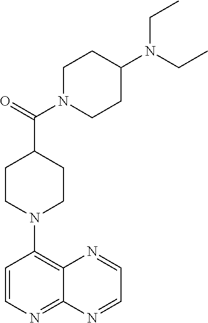 [SMILES: CCN(CC)C1CCN(C(=O)C2CCN(c3ccnc4nccnc34)CC2)CC1] | 2 |
| 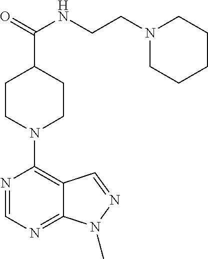 [SMILES: Cn1ncc2c(N3CCC(C(=O)NCCN4CCCCC4)CC3)ncnc21] | 3 |
| 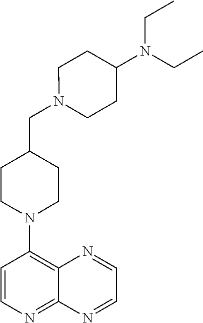 [SMILES: CCN(CC)C1CCN(CC2CCN(c3ccnc4nccnc34)CC2)CC1] | 4 |
| 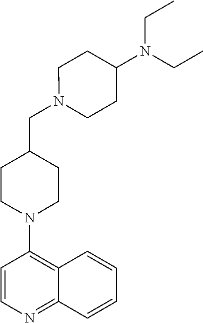 [SMILES: CCN(CC)C1CCN(CC2CCN(c3ccnc4ccccc34)CC2)CC1] | 5 |
| 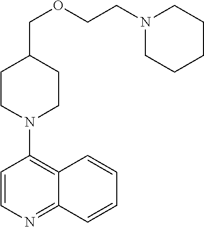 [SMILES: c1ccc2c(N3CCC(COCCN4CCCCC4)CC3)ccnc2c1] | 6 |
| 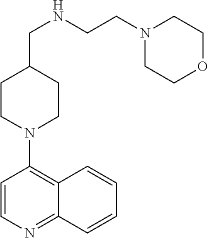 [SMILES: c1ccc2c(N3CCC(CNCCN4CCOCC4)CC3)ccnc2c1] | 7 |
| 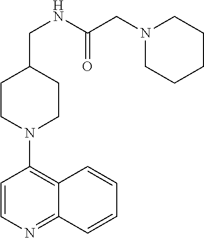 [SMILES: O=C(CN1CCCCC1)NCC1CCN(c2ccnc3ccccc23)CC1] | 8 |
| 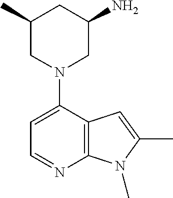 | 9 |
| 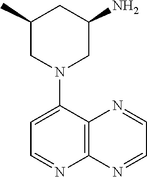 [SMILES: C[C@H]1C[C@@H](N)CN(c2ccnc3nccnc23)C1] | 10 |
| 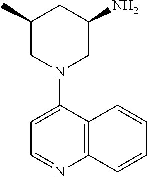 [SMILES: C[C@H]1C[C@@H](N)CN(c2ccnc3ccccc23)C1] | 11 |
| 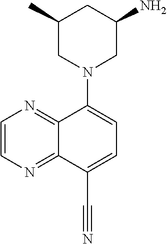 [SMILES: C[C@H]1C[C@@H](N)CN(c2ccc(C#N)c3nccnc23)C1] | 12 |
| 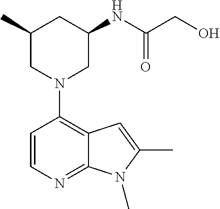 [SMILES: Cc1cc2c(N3C[C@@H](C)C[C@@H](NC(=O)CO)C3)ccnc2n1C] | 13 |
| 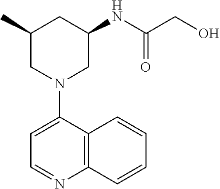 [SMILES: C[C@H]1C[C@@H](NC(=O)CO)CN(c2ccnc3ccccc23)C1] | 14 |
| 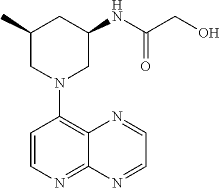 [SMILES: C[C@H]1C[C@@H](NC(=O)CO)CN(c2ccnc3nccnc23)C1] | 15 |
| 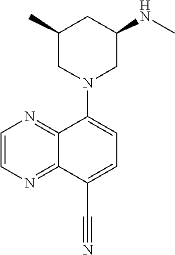 [SMILES: CN[C@@H]1C[C@H](C)CN(c2ccc(C#N)c3nccnc23)C1] | 16 |
| 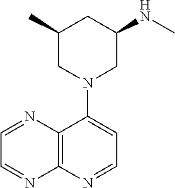 [SMILES: CN[C@@H]1C[C@H](C)CN(c2ccnc3nccnc23)C1] | 17 |
| 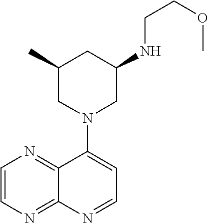 [SMILES: COCCN[C@@H]1C[C@H](C)CN(c2ccnc3nccnc23)C1] | 18 |
| 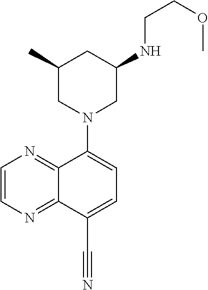 [SMILES: COCCN[C@@H]1C[C@H](C)CN(c2ccc(C#N)c3nccnc23)C1] | 19 |
| 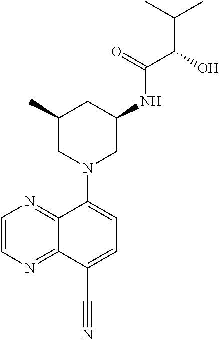 [SMILES: CC(C)[C@H](O)C(=O)N[C@@H]1C[C@H](C)CN(c2ccc(C#N)c3nccnc23)C1] | 20 |
| 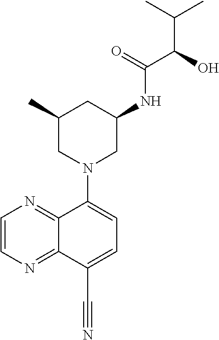 [SMILES: CC(C)[C@@H](O)C(=O)N[C@@H]1C[C@H](C)CN(c2ccc(C#N)c3nccnc23)C1] | 21 |
| 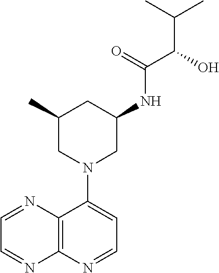 [SMILES: CC(C)[C@H](O)C(=O)N[C@@H]1C[C@H](C)CN(c2ccnc3nccnc23)C1] | 22 |
| 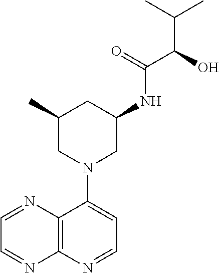 [SMILES: CC(C)[C@@H](O)C(=O)N[C@@H]1C[C@H](C)CN(c2ccnc3nccnc23)C1] | 23 |
| 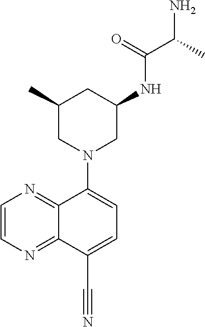 [SMILES: C[C@H]1C[C@@H](NC(=O)[C@@H](C)N)CN(c2ccc(C#N)c3nccnc23)C1] | 24 |
| 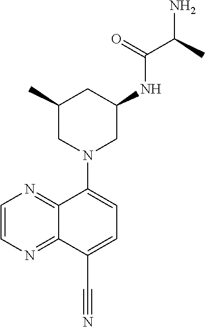 [SMILES: C[C@H]1C[C@@H](NC(=O)[C@H](C)N)CN(c2ccc(C#N)c3nccnc23)C1] | 25 |
| 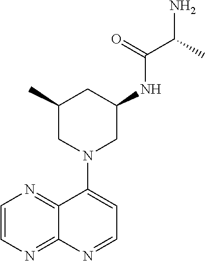 [SMILES: C[C@H]1C[C@@H](NC(=O)[C@@H](C)N)CN(c2ccnc3nccnc23)C1] | 26 |
| 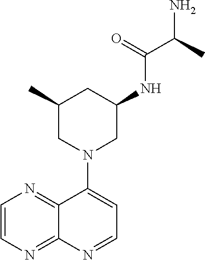 [SMILES: C[C@H]1C[C@@H](NC(=O)[C@H](C)N)CN(c2ccnc3nccnc23)C1] | 27 |
| 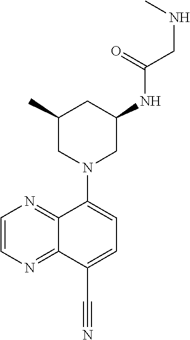 [SMILES: CNCC(=O)N[C@@H]1C[C@H](C)CN(c2ccc(C#N)c3nccnc23)C1] | 28 |
| 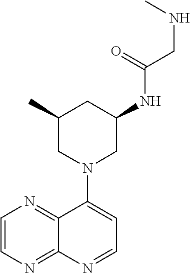 [SMILES: CNCC(=O)N[C@@H]1C[C@H](C)CN(c2ccnc3nccnc23)C1] | 29 |
| 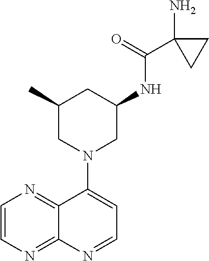 [SMILES: C[C@H]1C[C@@H](NC(=O)C2(N)CC2)CN(c2ccnc3nccnc23)C1] | 30 |
| 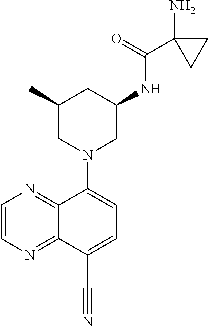 [SMILES: C[C@H]1C[C@@H](NC(=O)C2(N)CC2)CN(c2ccc(C#N)c3nccnc23)C1] | 31 |
| 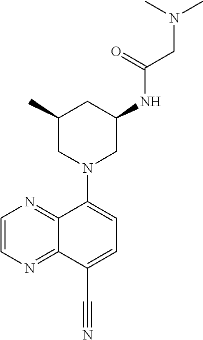 [SMILES: C[C@H]1C[C@@H](NC(=O)CN(C)C)CN(c2ccc(C#N)c3nccnc23)C1] | 32 |
| 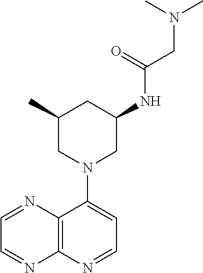 [SMILES: C[C@H]1C[C@@H](NC(=O)CN(C)C)CN(c2ccnc3nccnc23)C1] | 33 |
| 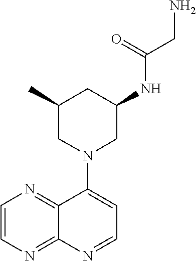 [SMILES: C[C@H]1C[C@@H](NC(=O)CN)CN(c2ccnc3nccnc23)C1] | 34 |
| 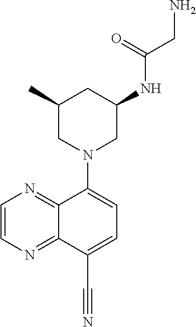 [SMILES: C[C@H]1C[C@@H](NC(=O)CN)CN(c2ccc(C#N)c3nccnc23)C1] | 35 |
| 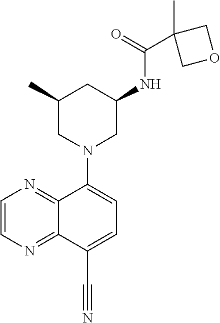 [SMILES: C[C@H]1C[C@@H](NC(=O)C2(C)COC2)CN(c2ccc(C#N)c3nccnc23)C1] | 36 |
| 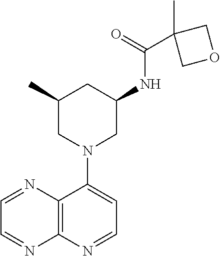 [SMILES: C[C@H]1C[C@@H](NC(=O)C2(C)COC2)CN(c2ccnc3nccnc23)C1] | 37 |
| 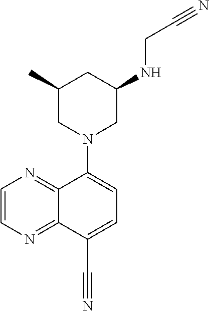 [SMILES: C[C@H]1C[C@@H](NCC#N)CN(c2ccc(C#N)c3nccnc23)C1] | 38 |
| 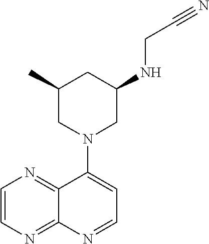 [SMILES: C[C@H]1C[C@@H](NCC#N)CN(c2ccnc3nccnc23)C1] | 39 |
| 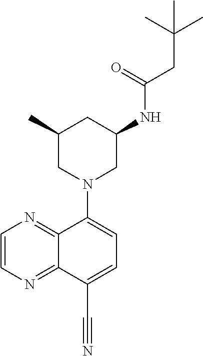 [SMILES: C[C@H]1C[C@@H](NC(=O)CC(C)(C)C)CN(c2ccc(C#N)c3nccnc23)C1] | 40 |
| 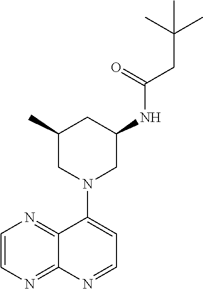 [SMILES: C[C@H]1C[C@@H](NC(=O)CC(C)(C)C)CN(c2ccnc3nccnc23)C1] | 41 |
| 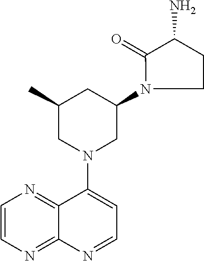 [SMILES: C[C@H]1C[C@@H](N2CC[C@@H](N)C2=O)CN(c2ccnc3nccnc23)C1] | 42 |
| 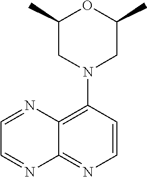 [SMILES: C[C@@H]1CN(c2ccnc3nccnc23)C[C@H](C)O1] | 43 |
| 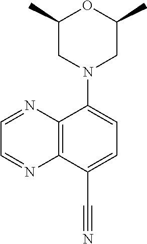 [SMILES: C[C@@H]1CN(c2ccc(C#N)c3nccnc23)C[C@H](C)O1] | 44 |
| 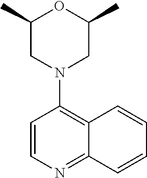 [SMILES: C[C@@H]1CN(c2ccnc3ccccc23)C[C@H](C)O1] | 45 |
| 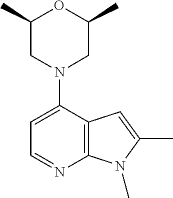 | 46 |
| 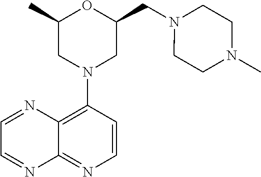 [SMILES: C[C@@H]1CN(c2ccnc3nccnc23)C[C@H](CN2CCN(C)CC2)O1] | 47 |
| 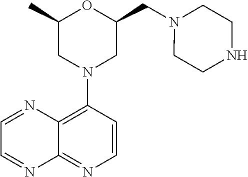 [SMILES: C[C@@H]1CN(c2ccnc3nccnc23)C[C@H](CN2CCNCC2)O1] | 48 |
| 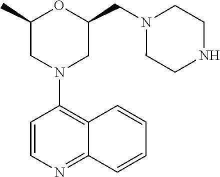 [SMILES: C[C@@H]1CN(c2ccnc3ccccc23)C[C@H](CN2CCNCC2)O1] | 49 |
| 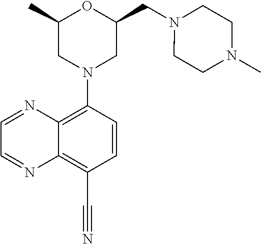 [SMILES: C[C@@H]1CN(c2ccc(C#N)c3nccnc23)C[C@H](CN2CCN(C)CC2)O1] | 50 |
| 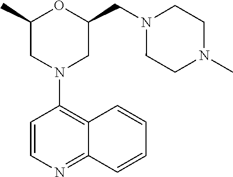 | 51 |
| 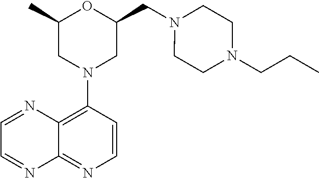 [SMILES: CCCN1CCN(C[C@H]2CN(c3ccnc4nccnc34)C[C@@H](C)O2)CC1] | 52 |
| 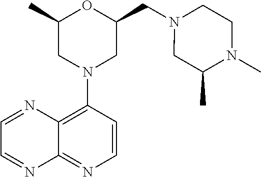 [SMILES: C[C@@H]1CN(c2ccnc3nccnc23)C[C@H](CN2CCN(C)[C@@H](C)C2)O1] | 53 |
| 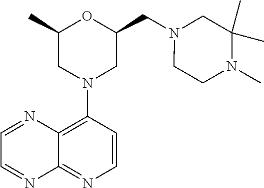 [SMILES: C[C@@H]1CN(c2ccnc3nccnc23)C[C@H](CN2CCN(C)C(C)(C)C2)O1] | 54 |
| 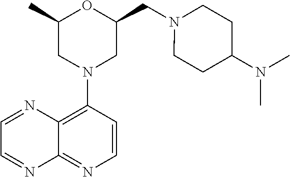 [SMILES: C[C@@H]1CN(c2ccnc3nccnc23)C[C@H](CN2CCC(N(C)C)CC2)O1] | 55 |
| 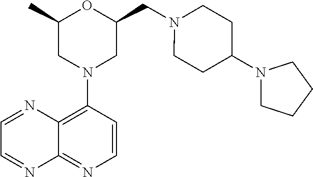 [SMILES: C[C@@H]1CN(c2ccnc3nccnc23)C[C@H](CN2CCC(N3CCCC3)CC2)O1] | 56 |
| 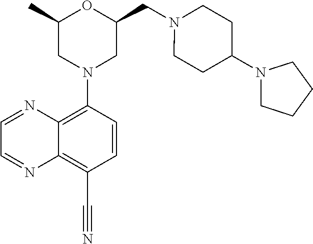 [SMILES: C[C@@H]1CN(c2ccc(C#N)c3nccnc23)C[C@H](CN2CCC(N3CCCC3)CC2)O1] | 57 |
| 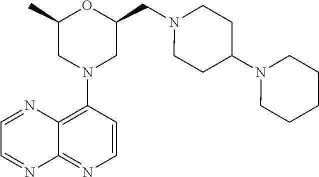 [SMILES: C[C@@H]1CN(c2ccnc3nccnc23)C[C@H](CN2CCC(N3CCCCC3)CC2)O1] | 58 |
| 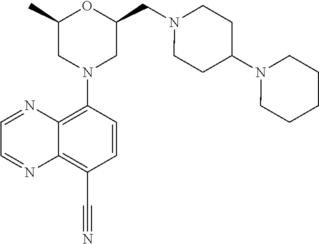 [SMILES: C[C@@H]1CN(c2ccc(C#N)c3nccnc23)C[C@H](CN2CCC(N3CCCCC3)CC2)O1] | 59 |
| 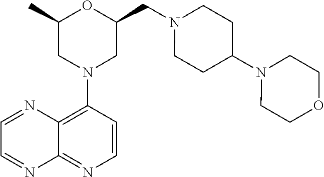 [SMILES: C[C@@H]1CN(c2ccnc3nccnc23)C[C@H](CN2CCC(N3CCOCC3)CC2)O1] | 60 |
| 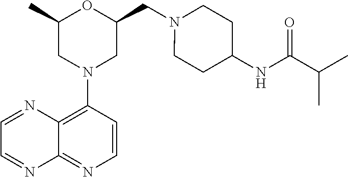 [SMILES: CC(C)C(=O)NC1CCN(C[C@H]2CN(c3ccnc4nccnc34)C[C@@H](C)O2)CC1] | 61 |
| 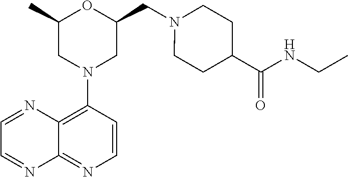 [SMILES: CCNC(=O)C1CCN(C[C@H]2CN(c3ccnc4nccnc34)C[C@@H](C)O2)CC1] | 62 |
| 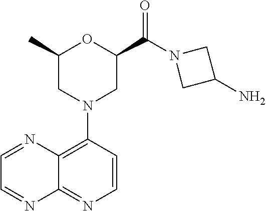 [SMILES: C[C@@H]1CN(c2ccnc3nccnc23)C[C@H](C(=O)N2CC(N)C2)O1] | 63 |
| 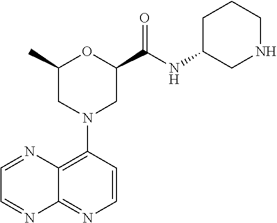 [SMILES: C[C@@H]1CN(c2ccnc3nccnc23)C[C@H](C(=O)N[C@@H]2CCCNC2)O1] | 64 |
| 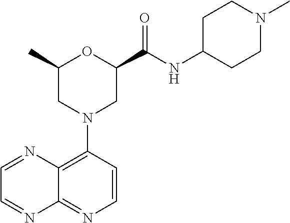 [SMILES: C[C@@H]1CN(c2ccnc3nccnc23)C[C@H](C(=O)NC2CCN(C)CC2)O1] | 65 |
| 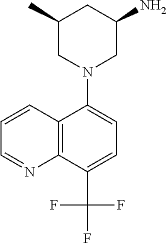 [SMILES: C[C@H]1C[C@@H](N)CN(c2ccc(C(F)(F)F)c3ncccc23)C1] | 66 |
| 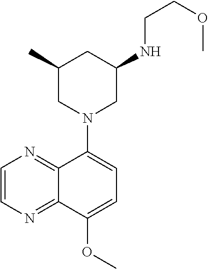 [SMILES: COCCN[C@@H]1C[C@H](C)CN(c2ccc(OC)c3nccnc23)C1] | 67 |
| 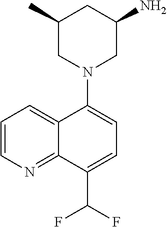 [SMILES: C[C@H]1C[C@@H](N)CN(c2ccc(C(F)F)c3ncccc23)C1] | 68 |
| 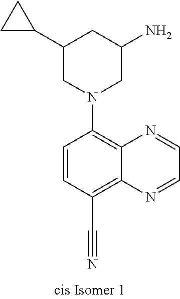 [SMILES: N#Cc1ccc(N2CC(N)CC(C3CC3)C2)c2nccnc12] | 69 |
| 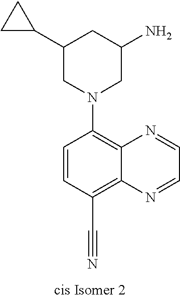 [SMILES: N#Cc1ccc(N2CC(N)CC(C3CC3)C2)c2nccnc12] | 70 |
| 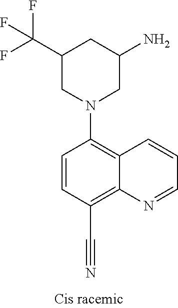 [SMILES: N#Cc1ccc(N2CC(N)CC(C(F)(F)F)C2)c2cccnc12] | 71 |
| 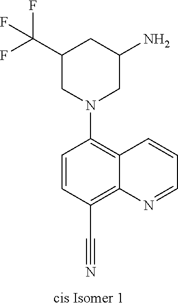 [SMILES: N#Cc1ccc(N2CC(N)CC(C(F)(F)F)C2)c2cccnc12] | 72 |
| 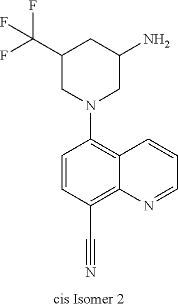 [SMILES: N#Cc1ccc(N2CC(N)CC(C(F)(F)F)C2)c2cccnc12] | 73 |
| 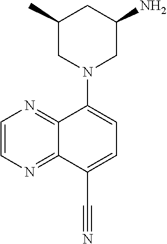 [SMILES: C[C@H]1C[C@@H](N)CN(c2ccc(C#N)c3nccnc23)C1] | 74 |
| 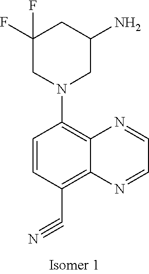 [SMILES: N#Cc1ccc(N2CC(N)CC(F)(F)C2)c2nccnc12] | 75 |
| 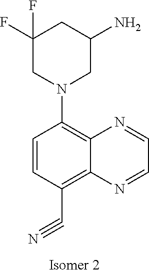 [SMILES: N#Cc1ccc(N2CC(N)CC(F)(F)C2)c2nccnc12] | 76 |
| 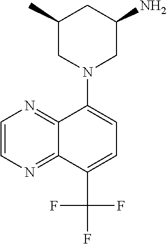 [SMILES: C[C@H]1C[C@@H](N)CN(c2ccc(C(F)(F)F)c3nccnc23)C1] | 77 |
| 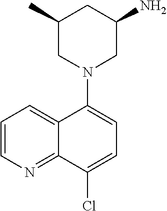 [SMILES: C[C@H]1C[C@@H](N)CN(c2ccc(Cl)c3ncccc23)C1] | 78 |
| 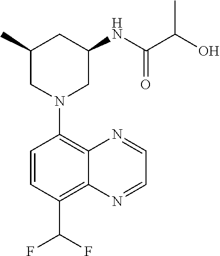 [SMILES: CC(O)C(=O)N[C@@H]1C[C@H](C)CN(c2ccc(C(F)F)c3nccnc23)C1] | 79 |
| 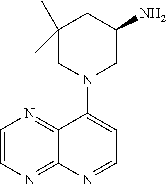 [SMILES: CC1(C)C[C@@H](N)CN(c2ccnc3nccnc23)C1] | 80 |
| 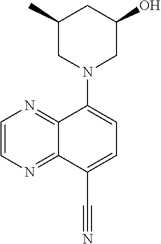 [SMILES: C[C@H]1C[C@@H](O)CN(c2ccc(C#N)c3nccnc23)C1] | 81 |
| 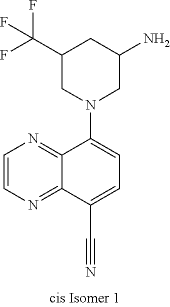 [SMILES: N#Cc1ccc(N2CC(N)CC(C(F)(F)F)C2)c2nccnc12] | 82 |
| 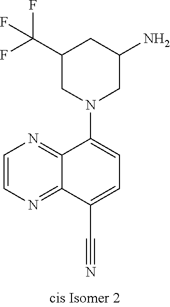 [SMILES: N#Cc1ccc(N2CC(N)CC(C(F)(F)F)C2)c2nccnc12] | 83 |
| 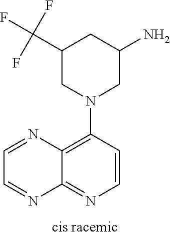 [SMILES: NC1CC(C(F)(F)F)CN(c2ccnc3nccnc23)C1] | 84 |
| 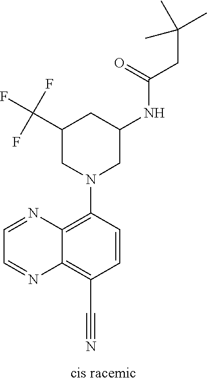 [SMILES: CC(C)(C)CC(=O)NC1CC(C(F)(F)F)CN(c2ccc(C#N)c3nccnc23)C1] | 85 |
| 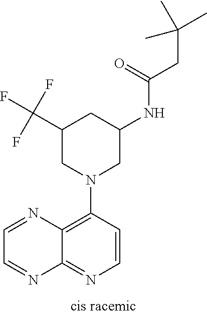 [SMILES: CC(C)(C)CC(=O)NC1CC(C(F)(F)F)CN(c2ccnc3nccnc23)C1] | 86 |
| 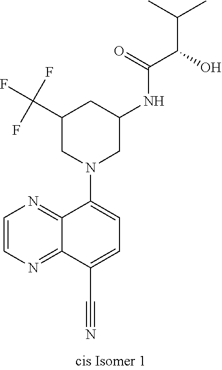 [SMILES: CC(C)[C@H](O)C(=O)NC1CC(C(F)(F)F)CN(c2ccc(C#N)c3nccnc23)C1] | 87 |
| 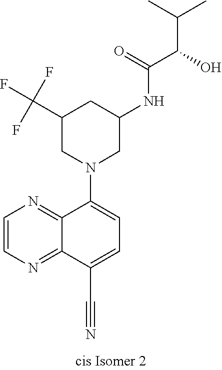 [SMILES: CC(C)[C@H](O)C(=O)NC1CC(C(F)(F)F)CN(c2ccc(C#N)c3nccnc23)C1] | 88 |
| 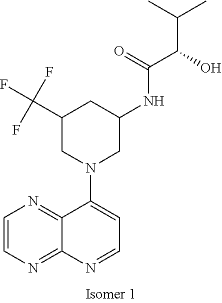 [SMILES: CC(C)[C@H](O)C(=O)NC1CC(C(F)(F)F)CN(c2ccnc3nccnc23)C1] | 89 |
| 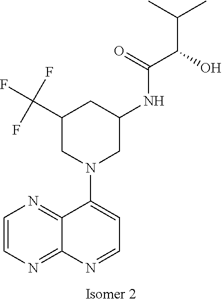 [SMILES: CC(C)[C@H](O)C(=O)NC1CC(C(F)(F)F)CN(c2ccnc3nccnc23)C1] | 90 |
| 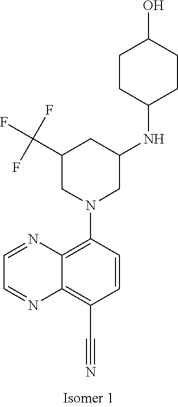 [SMILES: N#Cc1ccc(N2CC(NC3CCC(O)CC3)CC(C(F)(F)F)C2)c2nccnc12] | 91 |
| 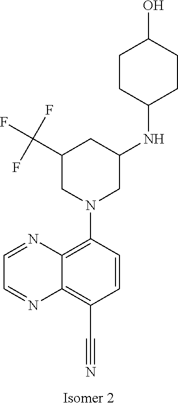 [SMILES: N#Cc1ccc(N2CC(NC3CCC(O)CC3)CC(C(F)(F)F)C2)c2nccnc12] | 92 |
| 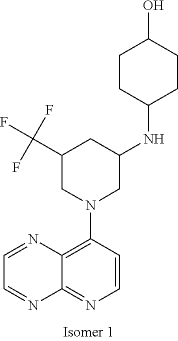 [SMILES: OC1CCC(NC2CC(C(F)(F)F)CN(c3ccnc4nccnc34)C2)CC1] | 93 |
| 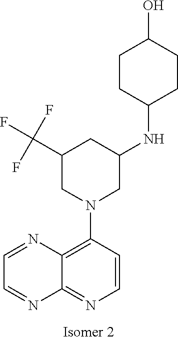 [SMILES: OC1CCC(NC2CC(C(F)(F)F)CN(c3ccnc4nccnc34)C2)CC1] | 94 |
| 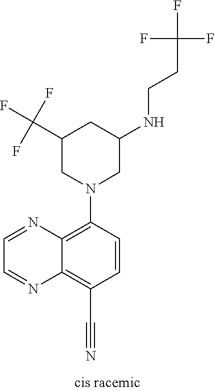 [SMILES: N#Cc1ccc(N2CC(NCCC(F)(F)F)CC(C(F)(F)F)C2)c2nccnc12] | 95 |
| 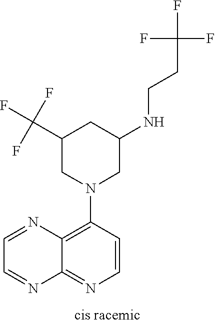 [SMILES: FC(F)(F)CCNC1CC(C(F)(F)F)CN(c2ccnc3nccnc23)C1] | 96 |
| 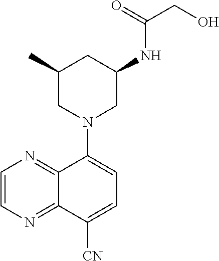 [SMILES: C[C@H]1C[C@@H](NC(=O)CO)CN(c2ccc(C#N)c3nccnc23)C1] | 97 |
| 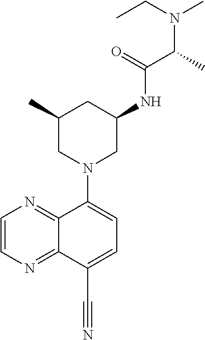 [SMILES: CCN(C)[C@H](C)C(=O)N[C@@H]1C[C@H](C)CN(c2ccc(C#N)c3nccnc23)C1] | 98 |
| 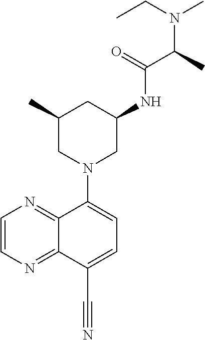 [SMILES: CCN(C)[C@@H](C)C(=O)N[C@@H]1C[C@H](C)CN(c2ccc(C#N)c3nccnc23)C1] | 99 |
| 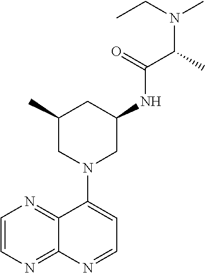 [SMILES: CCN(C)[C@H](C)C(=O)N[C@@H]1C[C@H](C)CN(c2ccnc3nccnc23)C1] | 100 |
| 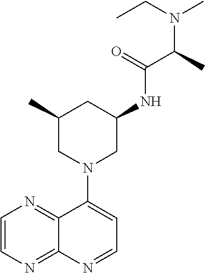 [SMILES: CCN(C)[C@@H](C)C(=O)N[C@@H]1C[C@H](C)CN(c2ccnc3nccnc23)C1] | 101 |
| 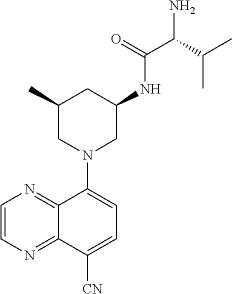 [SMILES: CC(C)[C@@H](N)C(=O)N[C@@H]1C[C@H](C)CN(c2ccc(C#N)c3nccnc23)C1] | 102 |
| 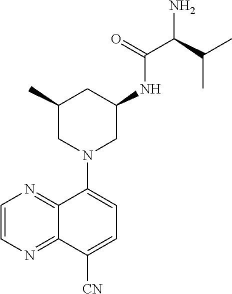 [SMILES: CC(C)[C@H](N)C(=O)N[C@@H]1C[C@H](C)CN(c2ccc(C#N)c3nccnc23)C1] | 103 |
| 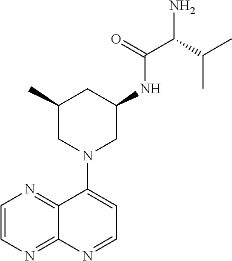 [SMILES: CC(C)[C@@H](N)C(=O)N[C@@H]1C[C@H](C)CN(c2ccnc3nccnc23)C1] | 104 |
| 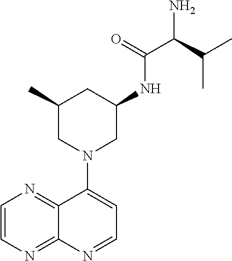 [SMILES: CC(C)[C@H](N)C(=O)N[C@@H]1C[C@H](C)CN(c2ccnc3nccnc23)C1] | 105 |
| 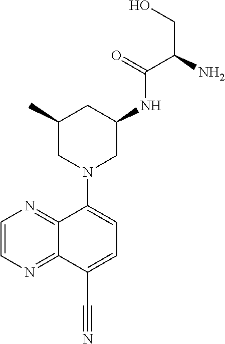 [SMILES: C[C@H]1C[C@@H](NC(=O)[C@H](N)CO)CN(c2ccc(C#N)c3nccnc23)C1] | 106 |
| 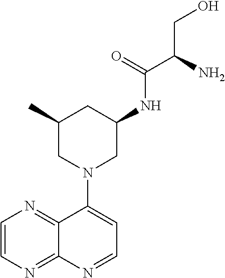 [SMILES: C[C@H]1C[C@@H](NC(=O)[C@H](N)CO)CN(c2ccnc3nccnc23)C1] | 107 |
| 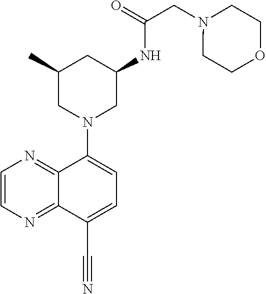 [SMILES: C[C@H]1C[C@@H](NC(=O)CN2CCOCC2)CN(c2ccc(C#N)c3nccnc23)C1] | 108 |
| 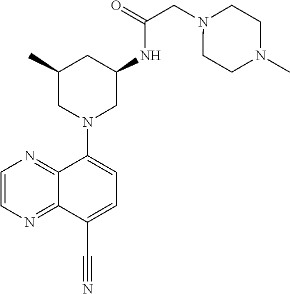 [SMILES: C[C@H]1C[C@@H](NC(=O)CN2CCN(C)CC2)CN(c2ccc(C#N)c3nccnc23)C1] | 109 |
| 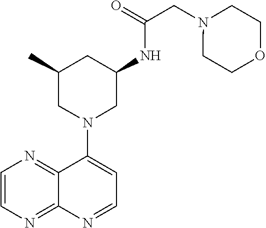 [SMILES: C[C@H]1C[C@@H](NC(=O)CN2CCOCC2)CN(c2ccnc3nccnc23)C1] | 110 |
| 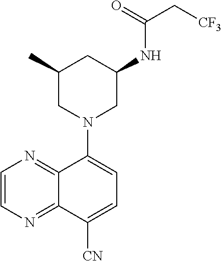 [SMILES: C[C@H]1C[C@@H](NC(=O)CC(F)(F)F)CN(c2ccc(C#N)c3nccnc23)C1] | 111 |
| 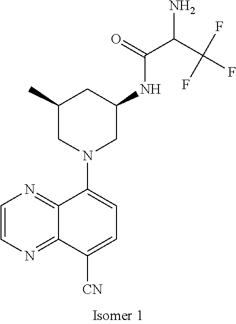 [SMILES: C[C@H]1C[C@@H](NC(=O)C(N)C(F)(F)F)CN(c2ccc(C#N)c3nccnc23)C1] | 112 |
| 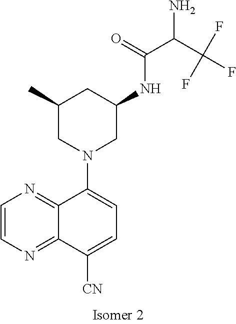 [SMILES: C[C@H]1C[C@@H](NC(=O)C(N)C(F)(F)F)CN(c2ccc(C#N)c3nccnc23)C1] | 113 |
| 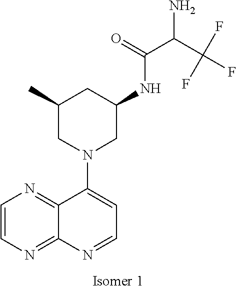 [SMILES: C[C@H]1C[C@@H](NC(=O)C(N)C(F)(F)F)CN(c2ccnc3nccnc23)C1] | 114 |
| 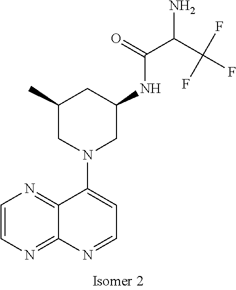 [SMILES: C[C@H]1C[C@@H](NC(=O)C(N)C(F)(F)F)CN(c2ccnc3nccnc23)C1] | 115 |
| 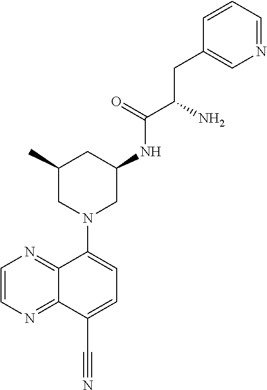 [SMILES: C[C@H]1C[C@@H](NC(=O)[C@@H](N)Cc2cccnc2)CN(c2ccc(C#N)c3nccnc23)C1] | 116 |
| 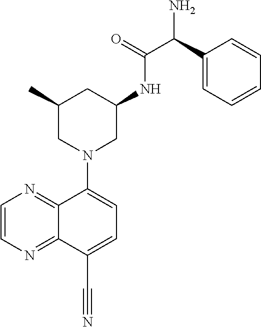 [SMILES: C[C@H]1C[C@@H](NC(=O)[C@@H](N)c2ccccc2)CN(c2ccc(C#N)c3nccnc23)C1] | 117 |
| 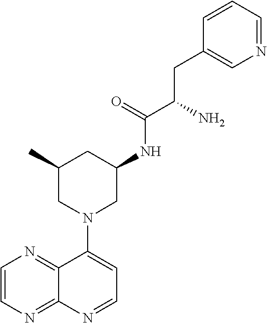 [SMILES: C[C@H]1C[C@@H](NC(=O)[C@@H](N)Cc2cccnc2)CN(c2ccnc3nccnc23)C1] | 118 |
| 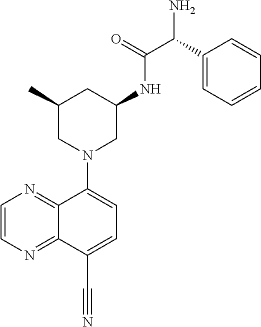 [SMILES: C[C@H]1C[C@@H](NC(=O)[C@H](N)c2ccccc2)CN(c2ccc(C#N)c3nccnc23)C1] | 119 |
| 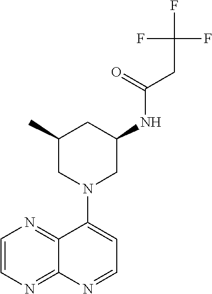 [SMILES: C[C@H]1C[C@@H](NC(=O)CC(F)(F)F)CN(c2ccnc3nccnc23)C1] | 120 |
| 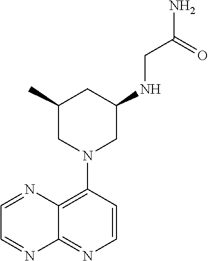 [SMILES: C[C@H]1C[C@@H](NCC(N)=O)CN(c2ccnc3nccnc23)C1] | 121 |
| 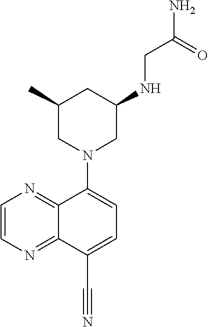 [SMILES: C[C@H]1C[C@@H](NCC(N)=O)CN(c2ccc(C#N)c3nccnc23)C1] | 122 |
| 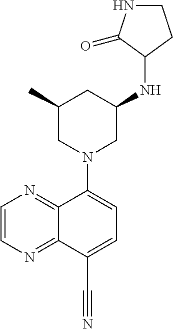 [SMILES: C[C@H]1C[C@@H](NC2CCNC2=O)CN(c2ccc(C#N)c3nccnc23)C1] | 123 |
| 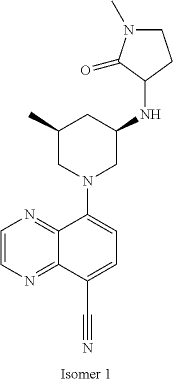 [SMILES: C[C@H]1C[C@@H](NC2CCN(C)C2=O)CN(c2ccc(C#N)c3nccnc23)C1] | 124 |
| 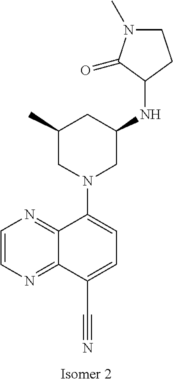 [SMILES: C[C@H]1C[C@@H](NC2CCN(C)C2=O)CN(c2ccc(C#N)c3nccnc23)C1] | 125 |
| 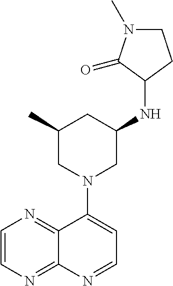 [SMILES: C[C@H]1C[C@@H](NC2CCN(C)C2=O)CN(c2ccnc3nccnc23)C1] | 126 |
| 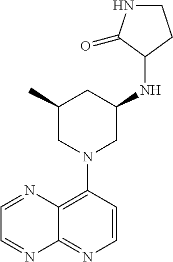 [SMILES: C[C@H]1C[C@@H](NC2CCNC2=O)CN(c2ccnc3nccnc23)C1] | 127 |
| 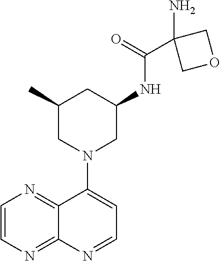 [SMILES: C[C@H]1C[C@@H](NC(=O)C2(N)COC2)CN(c2ccnc3nccnc23)C1] | 128 |
| 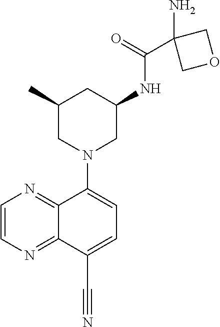 [SMILES: C[C@H]1C[C@@H](NC(=O)C2(N)COC2)CN(c2ccc(C#N)c3nccnc23)C1] | 129 |
| 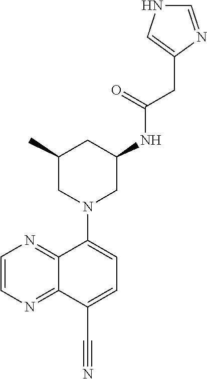 [SMILES: C[C@H]1C[C@@H](NC(=O)Cc2c[nH]cn2)CN(c2ccc(C#N)c3nccnc23)C1] | 130 |
| 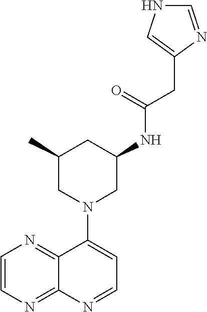 [SMILES: C[C@H]1C[C@@H](NC(=O)Cc2c[nH]cn2)CN(c2ccnc3nccnc23)C1] | 131 |
| 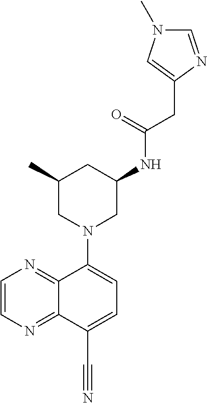 [SMILES: C[C@H]1C[C@@H](NC(=O)Cc2cn(C)cn2)CN(c2ccc(C#N)c3nccnc23)C1] | 132 |
| 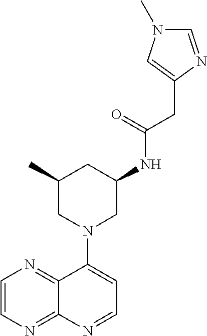 [SMILES: C[C@H]1C[C@@H](NC(=O)Cc2cn(C)cn2)CN(c2ccnc3nccnc23)C1] | 133 |
| 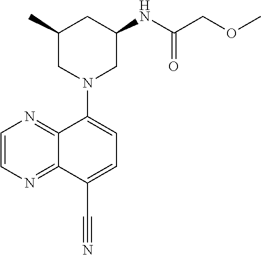 [SMILES: COCC(=O)N[C@@H]1C[C@H](C)CN(c2ccc(C#N)c3nccnc23)C1] | 134 |
| 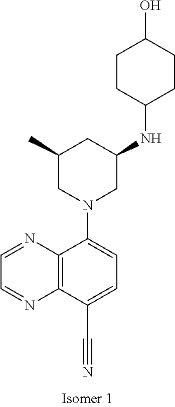 [SMILES: C[C@H]1C[C@@H](NC2CCC(O)CC2)CN(c2ccc(C#N)c3nccnc23)C1] | 135 |
| 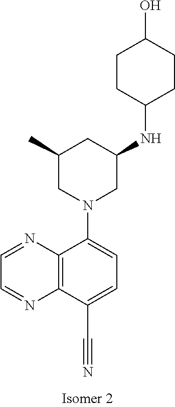 [SMILES: C[C@H]1C[C@@H](NC2CCC(O)CC2)CN(c2ccc(C#N)c3nccnc23)C1] | 136 |
| 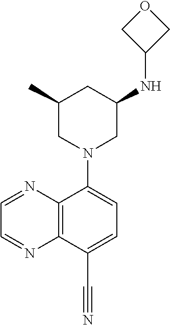 [SMILES: C[C@H]1C[C@@H](NC2COC2)CN(c2ccc(C#N)c3nccnc23)C1] | 137 |
| 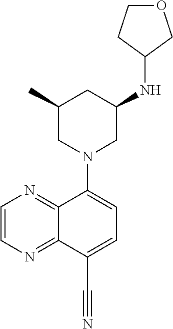 [SMILES: C[C@H]1C[C@@H](NC2CCOC2)CN(c2ccc(C#N)c3nccnc23)C1] | 138 |
| 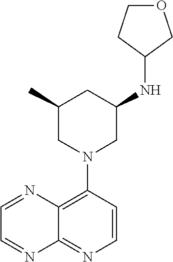 [SMILES: C[C@H]1C[C@@H](NC2CCOC2)CN(c2ccnc3nccnc23)C1] | 139 |
| 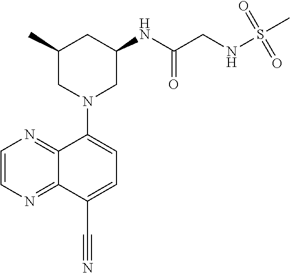 [SMILES: C[C@H]1C[C@@H](NC(=O)CNS(C)(=O)=O)CN(c2ccc(C#N)c3nccnc23)C1] | 140 |
| 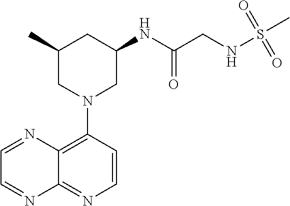 [SMILES: C[C@H]1C[C@@H](NC(=O)CNS(C)(=O)=O)CN(c2ccnc3nccnc23)C1] | 141 |
| 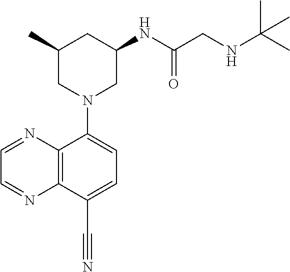 [SMILES: C[C@H]1C[C@@H](NC(=O)CNC(C)(C)C)CN(c2ccc(C#N)c3nccnc23)C1] | 142 |
| 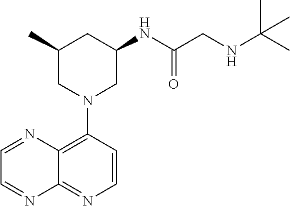 [SMILES: C[C@H]1C[C@@H](NC(=O)CNC(C)(C)C)CN(c2ccnc3nccnc23)C1] | 143 |
| 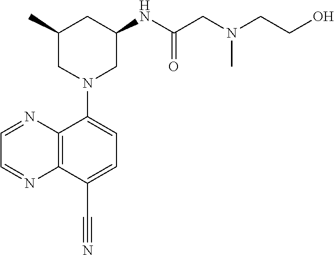 [SMILES: C[C@H]1C[C@@H](NC(=O)CN(C)CCO)CN(c2ccc(C#N)c3nccnc23)C1] | 144 |
| 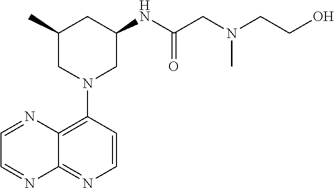 [SMILES: C[C@H]1C[C@@H](NC(=O)CN(C)CCO)CN(c2ccnc3nccnc23)C1] | 145 |
| 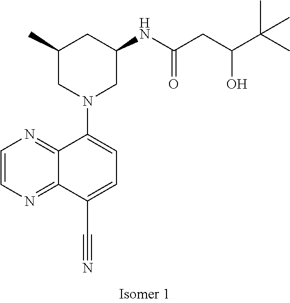 [SMILES: C[C@H]1C[C@@H](NC(=O)CC(O)C(C)(C)C)CN(c2ccc(C#N)c3nccnc23)C1] | 146 |
| 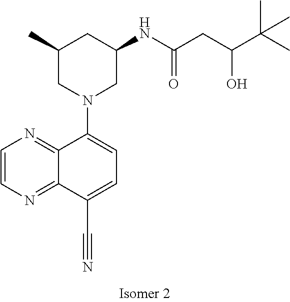 [SMILES: C[C@H]1C[C@@H](NC(=O)CC(O)C(C)(C)C)CN(c2ccc(C#N)c3nccnc23)C1] | 147 |
| 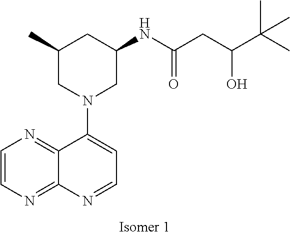 [SMILES: C[C@H]1C[C@@H](NC(=O)CC(O)C(C)(C)C)CN(c2ccnc3nccnc23)C1] | 148 |
| 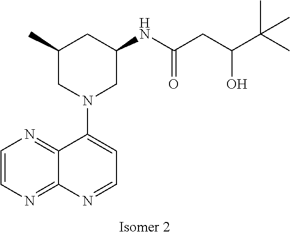 [SMILES: C[C@H]1C[C@@H](NC(=O)CC(O)C(C)(C)C)CN(c2ccnc3nccnc23)C1] | 149 |
| 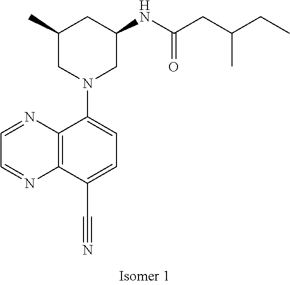 [SMILES: CCC(C)CC(=O)N[C@@H]1C[C@H](C)CN(c2ccc(C#N)c3nccnc23)C1] | 150 |
| 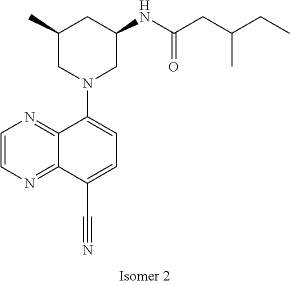 [SMILES: CCC(C)CC(=O)N[C@@H]1C[C@H](C)CN(c2ccc(C#N)c3nccnc23)C1] | 151 |
| 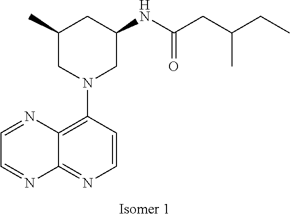 [SMILES: CCC(C)CC(=O)N[C@@H]1C[C@H](C)CN(c2ccnc3nccnc23)C1] | 152 |
| 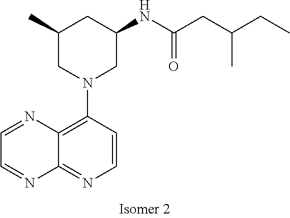 [SMILES: CCC(C)CC(=O)N[C@@H]1C[C@H](C)CN(c2ccnc3nccnc23)C1] | 153 |
| 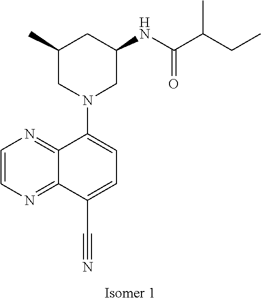 [SMILES: CCC(C)C(=O)N[C@@H]1C[C@H](C)CN(c2ccc(C#N)c3nccnc23)C1] | 154 |
| 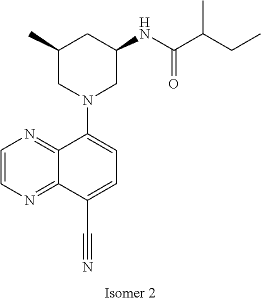 [SMILES: CCC(C)C(=O)N[C@@H]1C[C@H](C)CN(c2ccc(C#N)c3nccnc23)C1] | 155 |
| 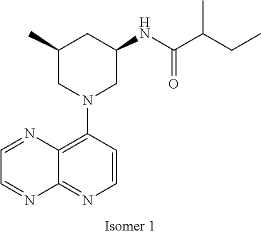 [SMILES: CCC(C)C(=O)N[C@@H]1C[C@H](C)CN(c2ccnc3nccnc23)C1] | 156 |
| 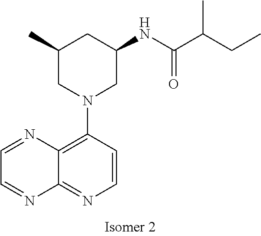 [SMILES: CCC(C)C(=O)N[C@@H]1C[C@H](C)CN(c2ccnc3nccnc23)C1] | 157 |
| 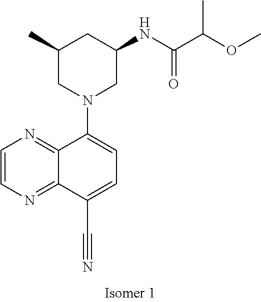 [SMILES: COC(C)C(=O)N[C@@H]1C[C@H](C)CN(c2ccc(C#N)c3nccnc23)C1] | 158 |
| 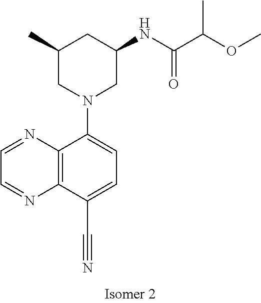 [SMILES: COC(C)C(=O)N[C@@H]1C[C@H](C)CN(c2ccc(C#N)c3nccnc23)C1] | 159 |
| 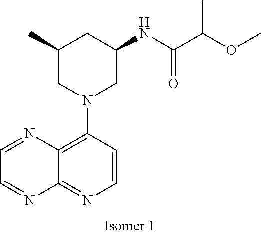 [SMILES: COC(C)C(=O)N[C@@H]1C[C@H](C)CN(c2ccnc3nccnc23)C1] | 160 |
| 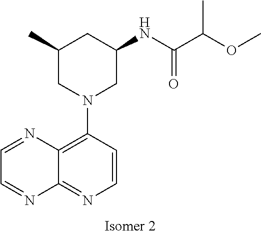 [SMILES: COC(C)C(=O)N[C@@H]1C[C@H](C)CN(c2ccnc3nccnc23)C1] | 161 |
| 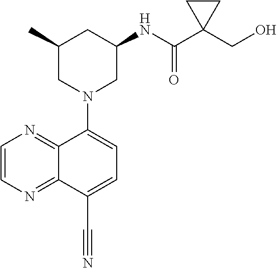 [SMILES: C[C@H]1C[C@@H](NC(=O)C2(CO)CC2)CN(c2ccc(C#N)c3nccnc23)C1] | 162 |
| 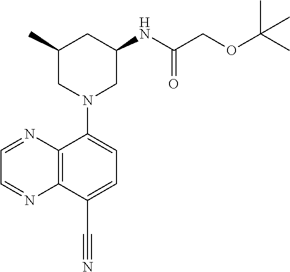 [SMILES: C[C@H]1C[C@@H](NC(=O)COC(C)(C)C)CN(c2ccc(C#N)c3nccnc23)C1] | 163 |
| 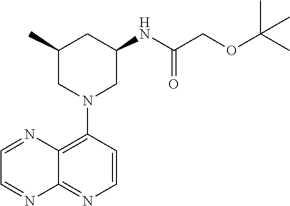 [SMILES: C[C@H]1C[C@@H](NC(=O)COC(C)(C)C)CN(c2ccnc3nccnc23)C1] | 164 |
| 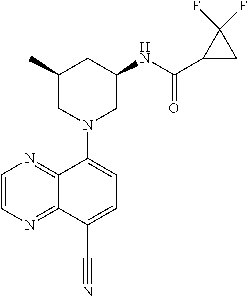 [SMILES: C[C@H]1C[C@@H](NC(=O)C2CC2(F)F)CN(c2ccc(C#N)c3nccnc23)C1] | 165 |
| 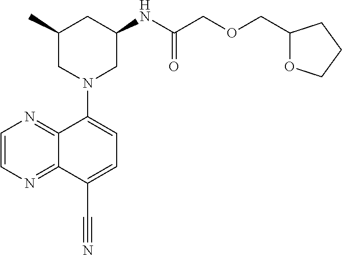 [SMILES: C[C@H]1C[C@@H](NC(=O)COCC2CCCO2)CN(c2ccc(C#N)c3nccnc23)C1] | 166 |
| 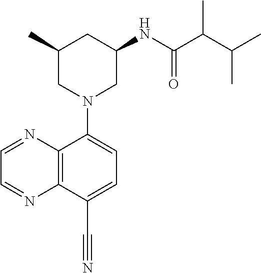 [SMILES: CC(C)C(C)C(=O)N[C@@H]1C[C@H](C)CN(c2ccc(C#N)c3nccnc23)C1] | 167 |
| 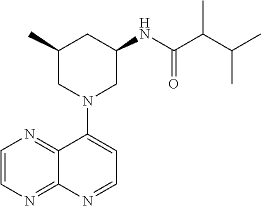 [SMILES: CC(C)C(C)C(=O)N[C@@H]1C[C@H](C)CN(c2ccnc3nccnc23)C1] | 168 |
| 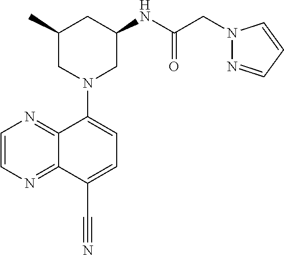 [SMILES: C[C@H]1C[C@@H](NC(=O)Cn2cccn2)CN(c2ccc(C#N)c3nccnc23)C1] | 169 |
| 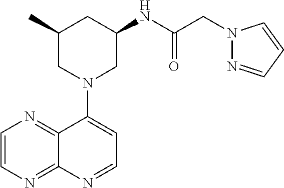 [SMILES: C[C@H]1C[C@@H](NC(=O)Cn2cccn2)CN(c2ccnc3nccnc23)C1] | 170 |
| 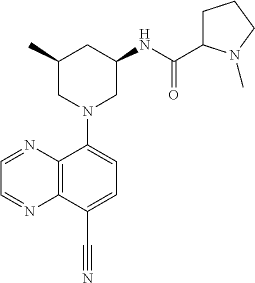 [SMILES: C[C@H]1C[C@@H](NC(=O)C2CCCN2C)CN(c2ccc(C#N)c3nccnc23)C1] | 171 |
| 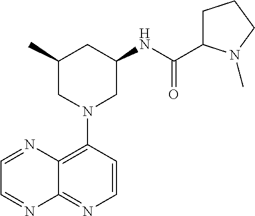 [SMILES: C[C@H]1C[C@@H](NC(=O)C2CCCN2C)CN(c2ccnc3nccnc23)C1] | 172 |
| 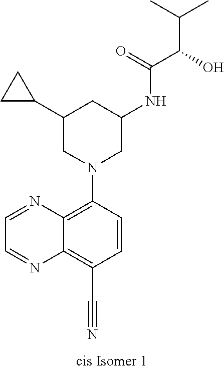 [SMILES: CC(C)[C@H](O)C(=O)NC1CC(C2CC2)CN(c2ccc(C#N)c3nccnc23)C1] | 173 |
| 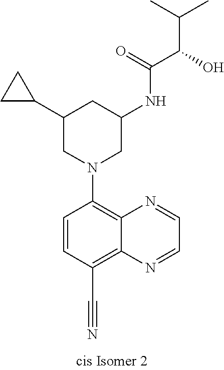 [SMILES: CC(C)[C@H](O)C(=O)NC1CC(C2CC2)CN(c2ccc(C#N)c3nccnc23)C1] | 174 |
| 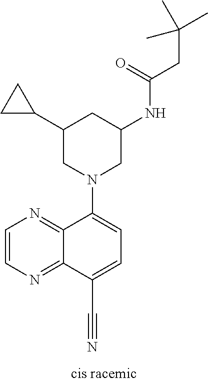 [SMILES: CC(C)(C)CC(=O)NC1CC(C2CC2)CN(c2ccc(C#N)c3nccnc23)C1] | 175 |
| 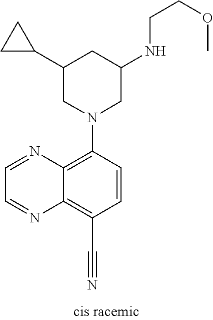 [SMILES: COCCNC1CC(C2CC2)CN(c2ccc(C#N)c3nccnc23)C1] | 176 |
| 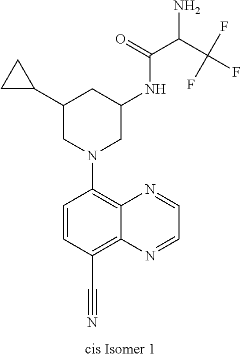 [SMILES: N#Cc1ccc(N2CC(NC(=O)C(N)C(F)(F)F)CC(C3CC3)C2)c2nccnc12] | 177 |
| 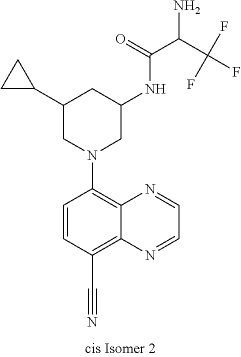 [SMILES: N#Cc1ccc(N2CC(NC(=O)C(N)C(F)(F)F)CC(C3CC3)C2)c2nccnc12] | 178 |
| 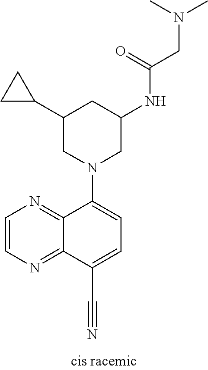 [SMILES: CN(C)CC(=O)NC1CC(C2CC2)CN(c2ccc(C#N)c3nccnc23)C1] | 179 |
| 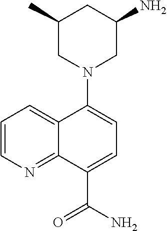 [SMILES: C[C@H]1C[C@@H](N)CN(c2ccc(C(N)=O)c3ncccc23)C1] | 180 |
| 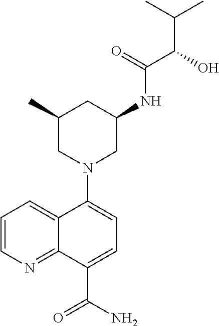 [SMILES: CC(C)[C@H](O)C(=O)N[C@@H]1C[C@H](C)CN(c2ccc(C(N)=O)c3ncccc23)C1] | 181 |
| 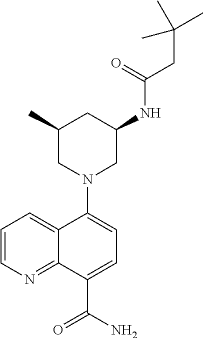 [SMILES: C[C@H]1C[C@@H](NC(=O)CC(C)(C)C)CN(c2ccc(C(N)=O)c3ncccc23)C1] | 182 |
| 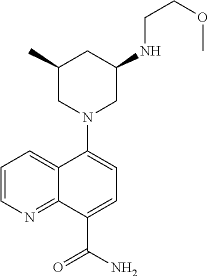 [SMILES: COCCN[C@@H]1C[C@H](C)CN(c2ccc(C(N)=O)c3ncccc23)C1] | 183 |
| 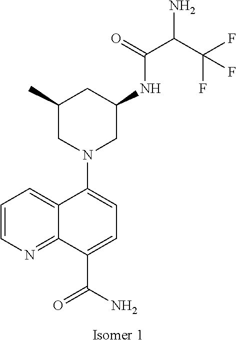 [SMILES: C[C@H]1C[C@@H](NC(=O)C(N)C(F)(F)F)CN(c2ccc(C(N)=O)c3ncccc23)C1] | 184 |
| 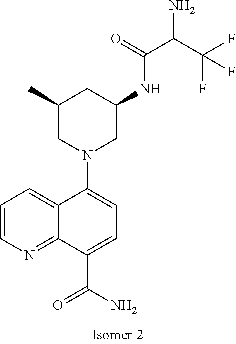 [SMILES: C[C@H]1C[C@@H](NC(=O)C(N)C(F)(F)F)CN(c2ccc(C(N)=O)c3ncccc23)C1] | 185 |
| 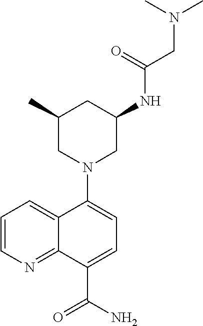 [SMILES: C[C@H]1C[C@@H](NC(=O)CN(C)C)CN(c2ccc(C(N)=O)c3ncccc23)C1] | 186 |
| 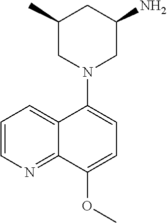 [SMILES: COc1ccc(N2C[C@@H](C)C[C@@H](N)C2)c2cccnc12] | 187 |
| 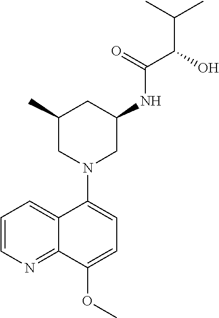 [SMILES: COc1ccc(N2C[C@@H](C)C[C@@H](NC(=O)[C@@H](O)C(C)C)C2)c2cccnc12] | 188 |
| 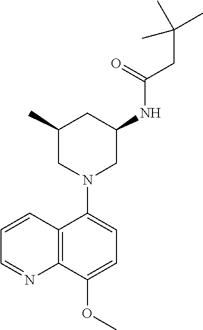 [SMILES: COc1ccc(N2C[C@@H](C)C[C@@H](NC(=O)CC(C)(C)C)C2)c2cccnc12] | 189 |
| 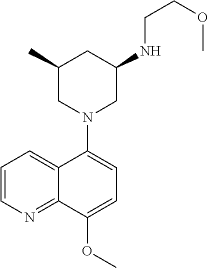 [SMILES: COCCN[C@@H]1C[C@H](C)CN(c2ccc(OC)c3ncccc23)C1] | 190 |
| 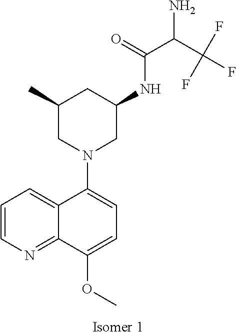 [SMILES: COc1ccc(N2C[C@@H](C)C[C@@H](NC(=O)C(N)C(F)(F)F)C2)c2cccnc12] | 191 |
| 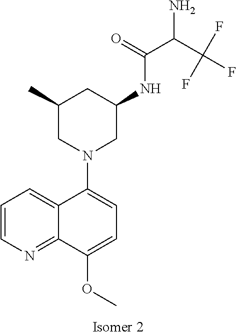 [SMILES: COc1ccc(N2C[C@@H](C)C[C@@H](NC(=O)C(N)C(F)(F)F)C2)c2cccnc12] | 192 |
| 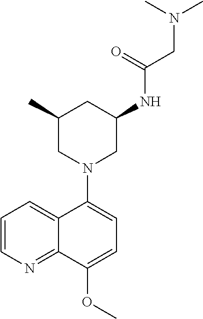 [SMILES: COc1ccc(N2C[C@@H](C)C[C@@H](NC(=O)CN(C)C)C2)c2cccnc12] | 193 |
| 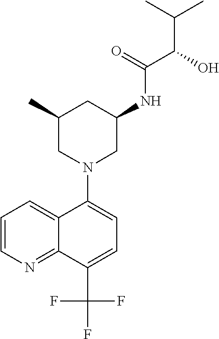 [SMILES: CC(C)[C@H](O)C(=O)N[C@@H]1C[C@H](C)CN(c2ccc(C(F)(F)F)c3ncccc23)C1] | 194 |
| 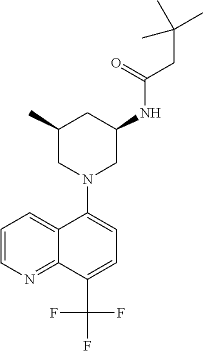 [SMILES: C[C@H]1C[C@@H](NC(=O)CC(C)(C)C)CN(c2ccc(C(F)(F)F)c3ncccc23)C1] | 195 |
| 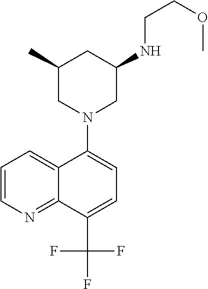 [SMILES: COCCN[C@@H]1C[C@H](C)CN(c2ccc(C(F)(F)F)c3ncccc23)C1] | 196 |
| 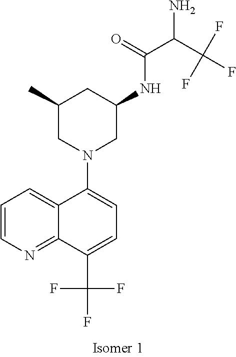 [SMILES: C[C@H]1C[C@@H](NC(=O)C(N)C(F)(F)F)CN(c2ccc(C(F)(F)F)c3ncccc23)C1] | 197 |
| 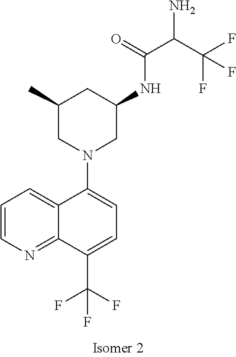 [SMILES: C[C@H]1C[C@@H](NC(=O)C(N)C(F)(F)F)CN(c2ccc(C(F)(F)F)c3ncccc23)C1] | 198 |
| 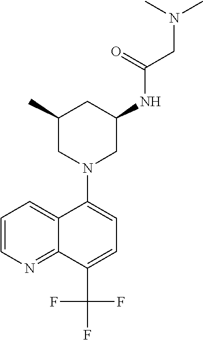 [SMILES: C[C@H]1C[C@@H](NC(=O)CN(C)C)CN(c2ccc(C(F)(F)F)c3ncccc23)C1] | 199 |
| 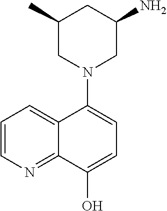 [SMILES: C[C@H]1C[C@@H](N)CN(c2ccc(O)c3ncccc23)C1] | 200 |
| 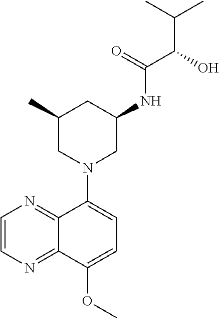 [SMILES: COc1ccc(N2C[C@@H](C)C[C@@H](NC(=O)[C@@H](O)C(C)C)C2)c2nccnc12] | 201 |
| 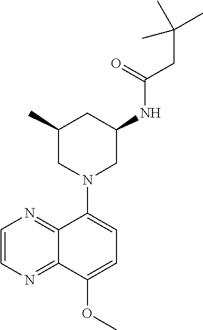 [SMILES: COc1ccc(N2C[C@@H](C)C[C@@H](NC(=O)CC(C)(C)C)C2)c2nccnc12] | 202 |
| 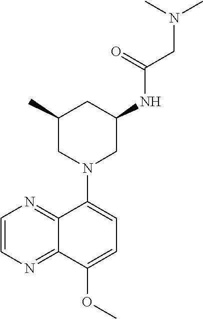 [SMILES: COc1ccc(N2C[C@@H](C)C[C@@H](NC(=O)CN(C)C)C2)c2nccnc12] | 203 |
| 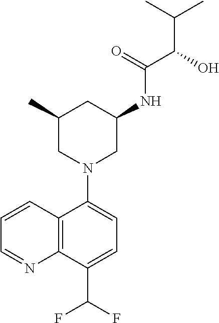 [SMILES: CC(C)[C@H](O)C(=O)N[C@@H]1C[C@H](C)CN(c2ccc(C(F)F)c3ncccc23)C1] | 204 |
| 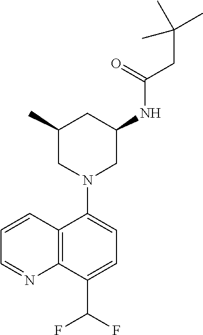 [SMILES: C[C@H]1C[C@@H](NC(=O)CC(C)(C)C)CN(c2ccc(C(F)F)c3ncccc23)C1] | 205 |
| 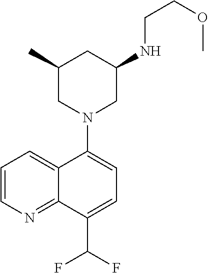 [SMILES: COCCN[C@@H]1C[C@H](C)CN(c2ccc(C(F)F)c3ncccc23)C1] | 206 |
| 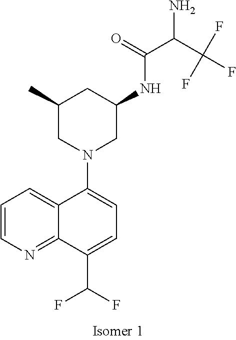 [SMILES: C[C@H]1C[C@@H](NC(=O)C(N)C(F)(F)F)CN(c2ccc(C(F)F)c3ncccc23)C1] | 207 |
| 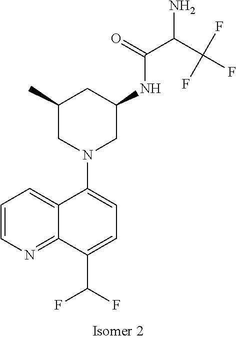 [SMILES: C[C@H]1C[C@@H](NC(=O)C(N)C(F)(F)F)CN(c2ccc(C(F)F)c3ncccc23)C1] | 208 |
| 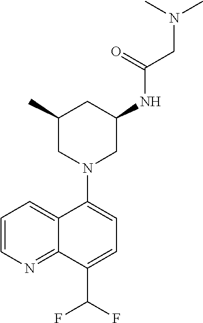 [SMILES: C[C@H]1C[C@@H](NC(=O)CN(C)C)CN(c2ccc(C(F)F)c3ncccc23)C1] | 209 |
| 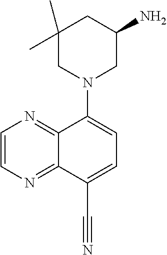 [SMILES: CC1(C)C[C@@H](N)CN(c2ccc(C#N)c3nccnc23)C1] | 210 |
| 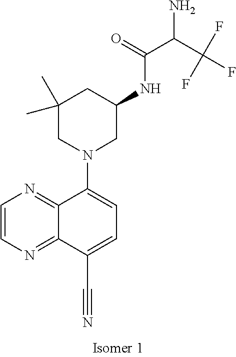 [SMILES: CC1(C)C[C@@H](NC(=O)C(N)C(F)(F)F)CN(c2ccc(C#N)c3nccnc23)C1] | 211 |
| 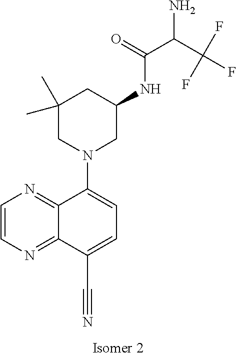 [SMILES: CC1(C)C[C@@H](NC(=O)C(N)C(F)(F)F)CN(c2ccc(C#N)c3nccnc23)C1] | 212 |
| 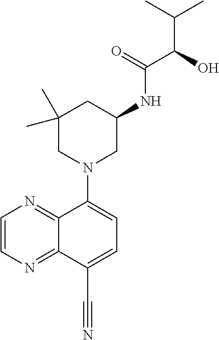 [SMILES: CC(C)[C@@H](O)C(=O)N[C@H]1CN(c2ccc(C#N)c3nccnc23)CC(C)(C)C1] | 213 |
| 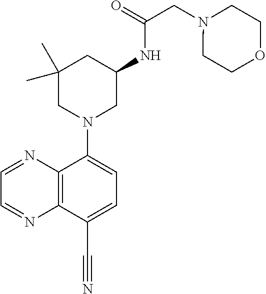 [SMILES: CC1(C)C[C@@H](NC(=O)CN2CCOCC2)CN(c2ccc(C#N)c3nccnc23)C1] | 214 |
| 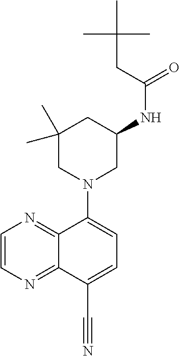 [SMILES: CC(C)(C)CC(=O)N[C@H]1CN(c2ccc(C#N)c3nccnc23)CC(C)(C)C1] | 215 |
| 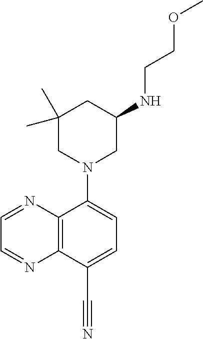 [SMILES: COCCN[C@H]1CN(c2ccc(C#N)c3nccnc23)CC(C)(C)C1] | 216 |
| 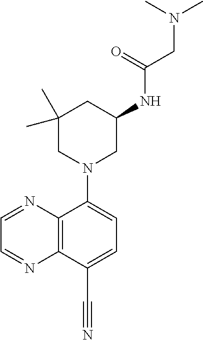 [SMILES: CN(C)CC(=O)N[C@H]1CN(c2ccc(C#N)c3nccnc23)CC(C)(C)C1] | 217 |
| 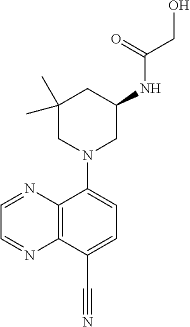 [SMILES: CC1(C)C[C@@H](NC(=O)CO)CN(c2ccc(C#N)c3nccnc23)C1] | 218 |
| 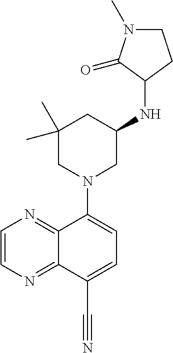 [SMILES: CN1CCC(N[C@H]2CN(c3ccc(C#N)c4nccnc34)CC(C)(C)C2)C1=O] | 219 |
| 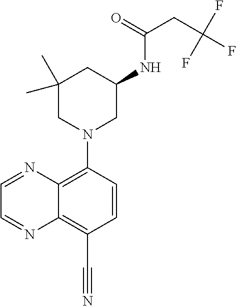 [SMILES: CC1(C)C[C@@H](NC(=O)CC(F)(F)F)CN(c2ccc(C#N)c3nccnc23)C1] | 220 |
| 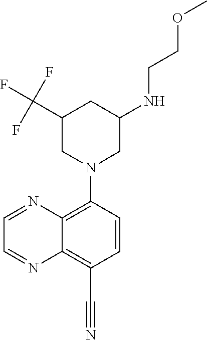 [SMILES: COCCNC1CC(C(F)(F)F)CN(c2ccc(C#N)c3nccnc23)C1] | 221 |
| 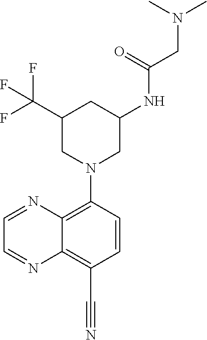 [SMILES: CN(C)CC(=O)NC1CC(C(F)(F)F)CN(c2ccc(C#N)c3nccnc23)C1] | 222 |
| 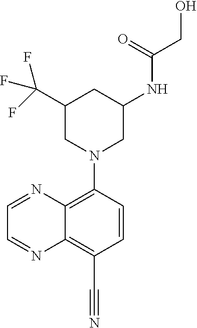 [SMILES: N#Cc1ccc(N2CC(NC(=O)CO)CC(C(F)(F)F)C2)c2nccnc12] | 223 |
| 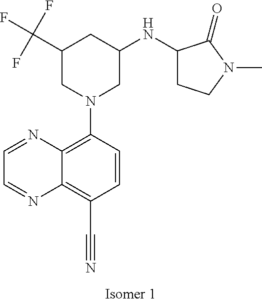 [SMILES: CN1CCC(NC2CC(C(F)(F)F)CN(c3ccc(C#N)c4nccnc34)C2)C1=O] | 224 |
| 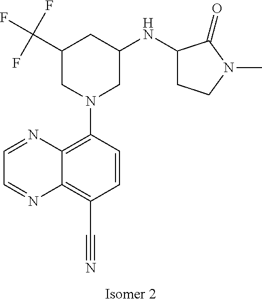 [SMILES: CN1CCC(NC2CC(C(F)(F)F)CN(c3ccc(C#N)c4nccnc34)C2)C1=O] | 225 |
| 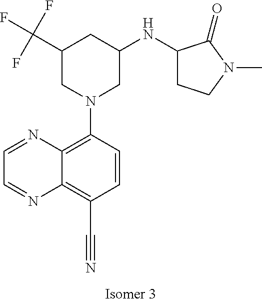 [SMILES: CN1CCC(NC2CC(C(F)(F)F)CN(c3ccc(C#N)c4nccnc34)C2)C1=O] | 226 |
| 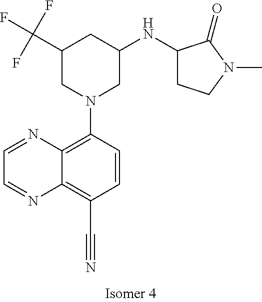 [SMILES: CN1CCC(NC2CC(C(F)(F)F)CN(c3ccc(C#N)c4nccnc34)C2)C1=O] | 227 |
| 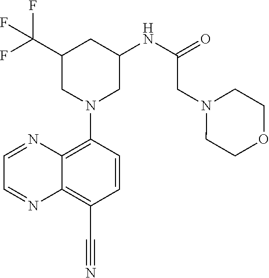 [SMILES: N#Cc1ccc(N2CC(NC(=O)CN3CCOCC3)CC(C(F)(F)F)C2)c2nccnc12] | 228 |
| 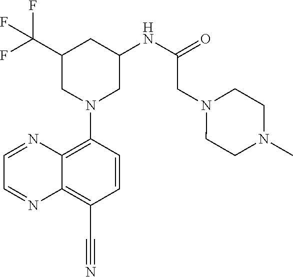 [SMILES: CN1CCN(CC(=O)NC2CC(C(F)(F)F)CN(c3ccc(C#N)c4nccnc34)C2)CC1] | 229 |
| 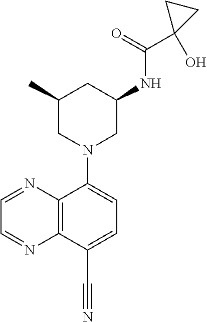 [SMILES: C[C@H]1C[C@@H](NC(=O)C2(O)CC2)CN(c2ccc(C#N)c3nccnc23)C1] | 230 |
| 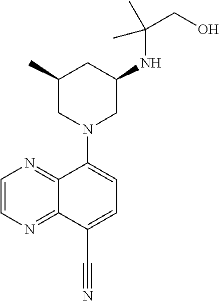 [SMILES: C[C@H]1C[C@@H](NC(C)(C)CO)CN(c2ccc(C#N)c3nccnc23)C1] | 231 |
| 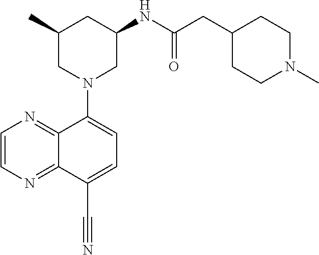 [SMILES: C[C@H]1C[C@@H](NC(=O)CC2CCN(C)CC2)CN(c2ccc(C#N)c3nccnc23)C1] | 232 |
| 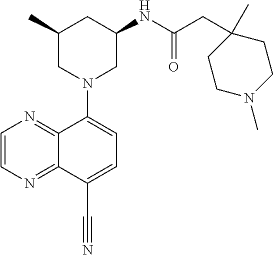 [SMILES: C[C@H]1C[C@@H](NC(=O)CC2(C)CCN(C)CC2)CN(c2ccc(C#N)c3nccnc23)C1] | 233 |
| 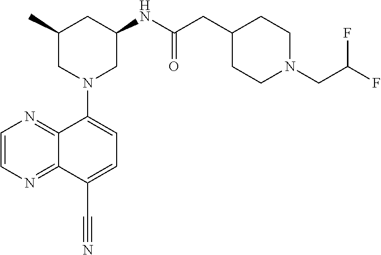 [SMILES: C[C@H]1C[C@@H](NC(=O)CC2CCN(CC(F)F)CC2)CN(c2ccc(C#N)c3nccnc23)C1] | 234 |
| 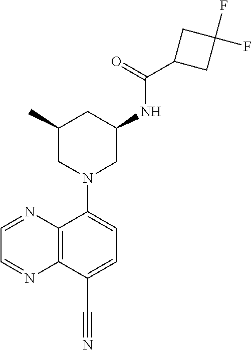 [SMILES: C[C@H]1C[C@@H](NC(=O)C2CC(F)(F)C2)CN(c2ccc(C#N)c3nccnc23)C1] | 235 |
| 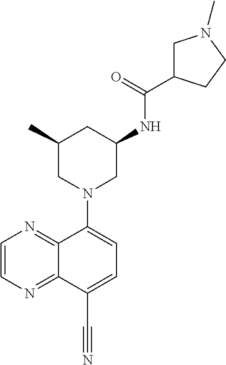 [SMILES: C[C@H]1C[C@@H](NC(=O)C2CCN(C)C2)CN(c2ccc(C#N)c3nccnc23)C1] | 236 |
| 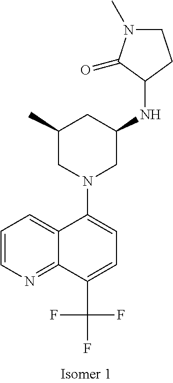 [SMILES: C[C@H]1C[C@@H](NC2CCN(C)C2=O)CN(c2ccc(C(F)(F)F)c3ncccc23)C1] | 237 |
| 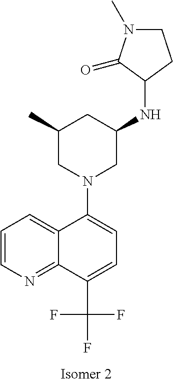 [SMILES: C[C@H]1C[C@@H](NC2CCN(C)C2=O)CN(c2ccc(C(F)(F)F)c3ncccc23)C1] | 238 |
| 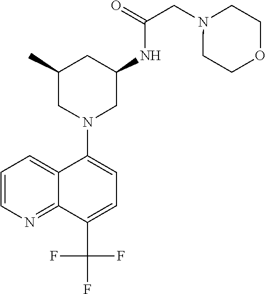 [SMILES: C[C@H]1C[C@@H](NC(=O)CN2CCOCC2)CN(c2ccc(C(F)(F)F)c3ncccc23)C1] | 239 |
| 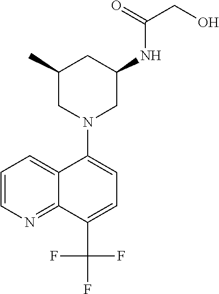 [SMILES: C[C@H]1C[C@@H](NC(=O)CO)CN(c2ccc(C(F)(F)F)c3ncccc23)C1] | 240 |
| 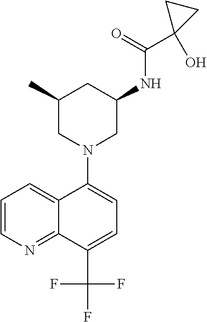 [SMILES: C[C@H]1C[C@@H](NC(=O)C2(O)CC2)CN(c2ccc(C(F)(F)F)c3ncccc23)C1] | 241 |
| 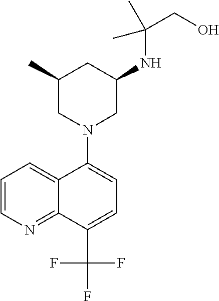 [SMILES: C[C@H]1C[C@@H](NC(C)(C)CO)CN(c2ccc(C(F)(F)F)c3ncccc23)C1] | 242 |
| 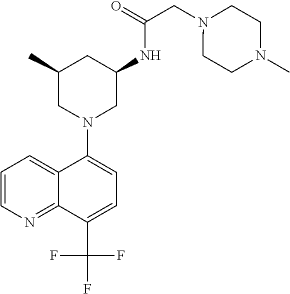 [SMILES: C[C@H]1C[C@@H](NC(=O)CN2CCN(C)CC2)CN(c2ccc(C(F)(F)F)c3ncccc23)C1] | 243 |
| 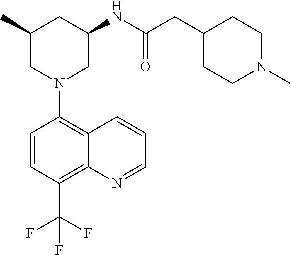 | 244 |
| 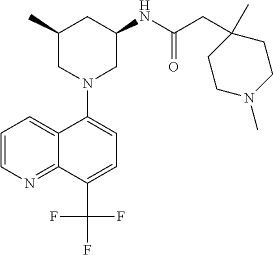 [SMILES: C[C@H]1C[C@@H](NC(=O)CC2(C)CCN(C)CC2)CN(c2ccc(C(F)(F)F)c3ncccc23)C1] | 245 |
| 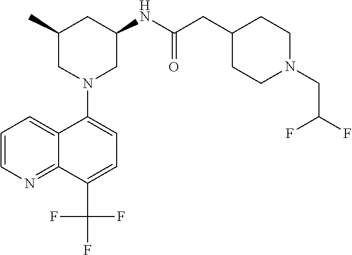 [SMILES: C[C@H]1C[C@@H](NC(=O)CC2CCN(CC(F)F)CC2)CN(c2ccc(C(F)(F)F)c3ncccc23)C1] | 246 |
| 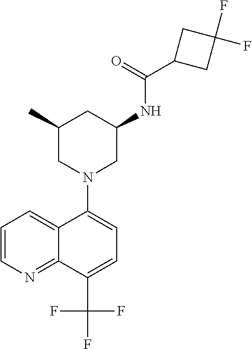 [SMILES: C[C@H]1C[C@@H](NC(=O)C2CC(F)(F)C2)CN(c2ccc(C(F)(F)F)c3ncccc23)C1] | 247 |
| 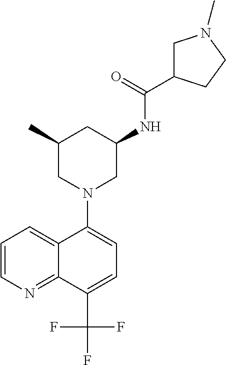 [SMILES: C[C@H]1C[C@@H](NC(=O)C2CCN(C)C2)CN(c2ccc(C(F)(F)F)c3ncccc23)C1] | 248 |
| 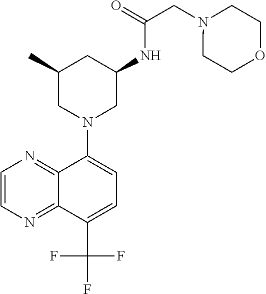 [SMILES: C[C@H]1C[C@@H](NC(=O)CN2CCOCC2)CN(c2ccc(C(F)(F)F)c3nccnc23)C1] | 249 |
| 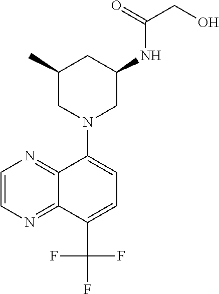 [SMILES: C[C@H]1C[C@@H](NC(=O)CO)CN(c2ccc(C(F)(F)F)c3nccnc23)C1] | 250 |
| 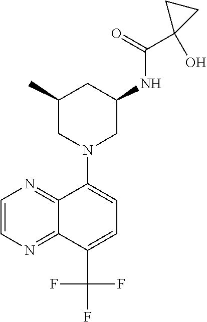 [SMILES: C[C@H]1C[C@@H](NC(=O)C2(O)CC2)CN(c2ccc(C(F)(F)F)c3nccnc23)C1] | 251 |
| 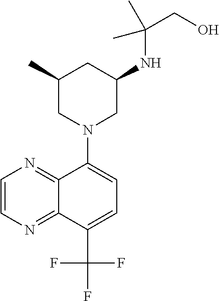 [SMILES: C[C@H]1C[C@@H](NC(C)(C)CO)CN(c2ccc(C(F)(F)F)c3nccnc23)C1] | 252 |
| 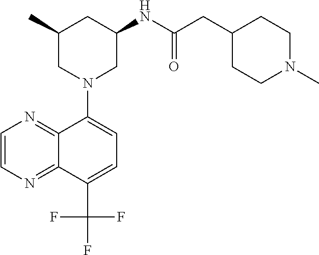 [SMILES: C[C@H]1C[C@@H](NC(=O)CC2CCN(C)CC2)CN(c2ccc(C(F)(F)F)c3nccnc23)C1] | 253 |
| 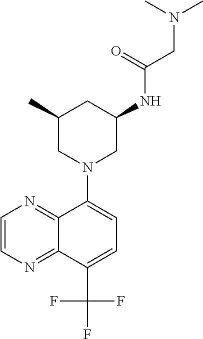 [SMILES: C[C@H]1C[C@@H](NC(=O)CN(C)C)CN(c2ccc(C(F)(F)F)c3nccnc23)C1] | 254 |
| 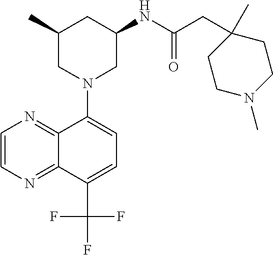 [SMILES: C[C@H]1C[C@@H](NC(=O)CC2(C)CCN(C)CC2)CN(c2ccc(C(F)(F)F)c3nccnc23)C1] | 255 |
| 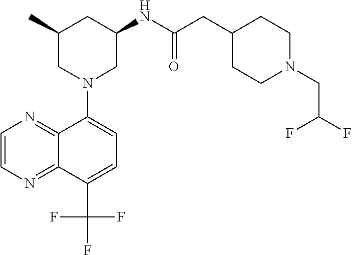 [SMILES: C[C@H]1C[C@@H](NC(=O)CC2CCN(CC(F)F)CC2)CN(c2ccc(C(F)(F)F)c3nccnc23)C1] | 256 |
| 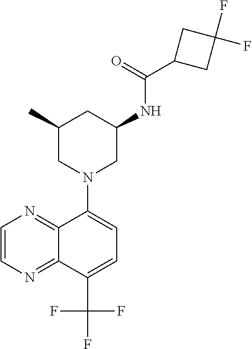 [SMILES: C[C@H]1C[C@@H](NC(=O)C2CC(F)(F)C2)CN(c2ccc(C(F)(F)F)c3nccnc23)C1] | 257 |
| 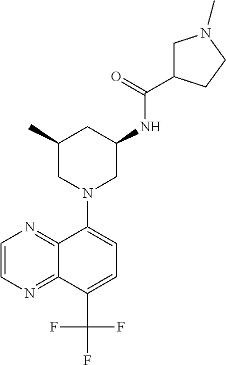 [SMILES: C[C@H]1C[C@@H](NC(=O)C2CCN(C)C2)CN(c2ccc(C(F)(F)F)c3nccnc23)C1] | 258 |
| 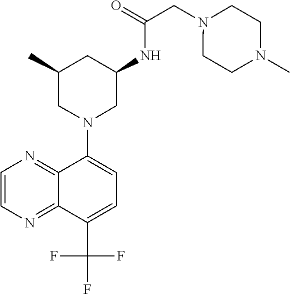 [SMILES: C[C@H]1C[C@@H](NC(=O)CN2CCN(C)CC2)CN(c2ccc(C(F)(F)F)c3nccnc23)C1] | 259 |
| 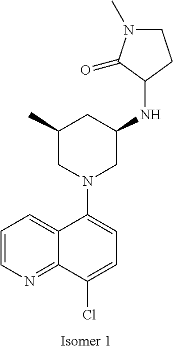 [SMILES: C[C@H]1C[C@@H](NC2CCN(C)C2=O)CN(c2ccc(Cl)c3ncccc23)C1] | 260 |
| 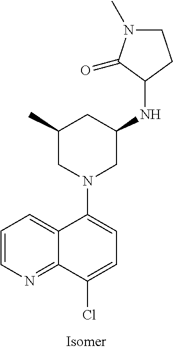 [SMILES: C[C@H]1C[C@@H](NC2CCN(C)C2=O)CN(c2ccc(Cl)c3ncccc23)C1] | 261 |
| 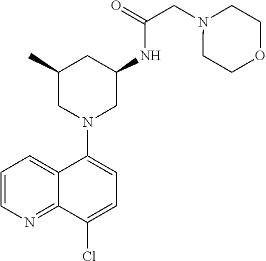 [SMILES: C[C@H]1C[C@@H](NC(=O)CN2CCOCC2)CN(c2ccc(Cl)c3ncccc23)C1] | 262 |
| 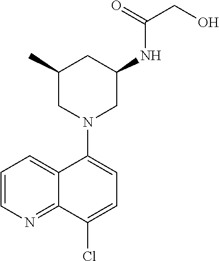 [SMILES: C[C@H]1C[C@@H](NC(=O)CO)CN(c2ccc(Cl)c3ncccc23)C1] | 263 |
| 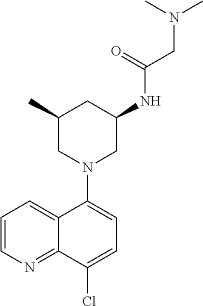 [SMILES: C[C@H]1C[C@@H](NC(=O)CN(C)C)CN(c2ccc(Cl)c3ncccc23)C1] | 264 |
| 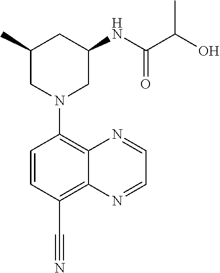 [SMILES: CC(O)C(=O)N[C@@H]1C[C@H](C)CN(c2ccc(C#N)c3nccnc23)C1] | 265 |
| 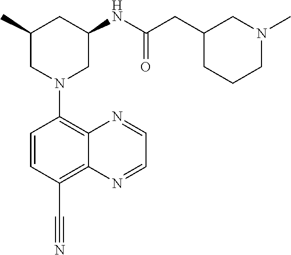 | 266 |
| 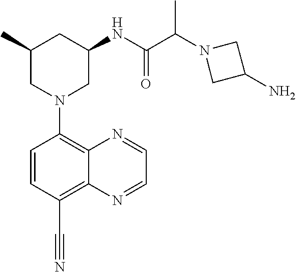 [SMILES: CC(C(=O)N[C@@H]1C[C@H](C)CN(c2ccc(C#N)c3nccnc23)C1)N1CC(N)C1] | 267 |
| 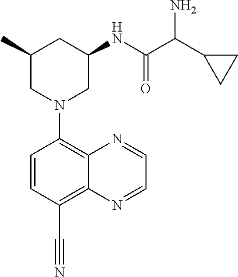 [SMILES: C[C@H]1C[C@@H](NC(=O)C(N)C2CC2)CN(c2ccc(C#N)c3nccnc23)C1] | 268 |
| 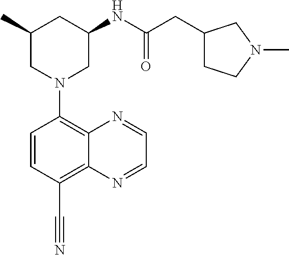 | 269 |
| 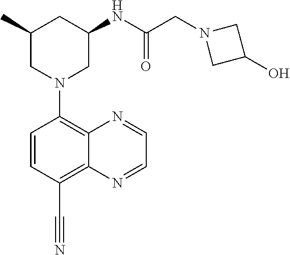 [SMILES: C[C@H]1C[C@@H](NC(=O)CN2CC(O)C2)CN(c2ccc(C#N)c3nccnc23)C1] | 270 |
| 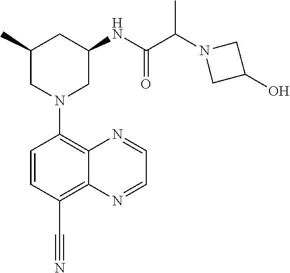 [SMILES: CC(C(=O)N[C@@H]1C[C@H](C)CN(c2ccc(C#N)c3nccnc23)C1)N1CC(O)C1] | 271 |
| 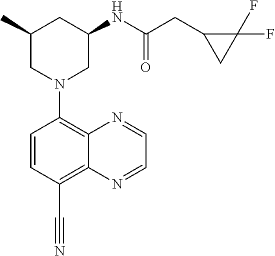 [SMILES: C[C@H]1C[C@@H](NC(=O)CC2CC2(F)F)CN(c2ccc(C#N)c3nccnc23)C1] | 272 |
| 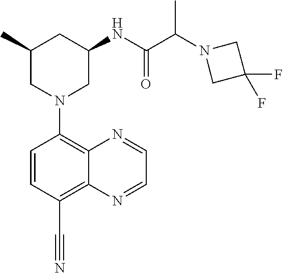 [SMILES: CC(C(=O)N[C@@H]1C[C@H](C)CN(c2ccc(C#N)c3nccnc23)C1)N1CC(F)(F)C1] | 273 |
| 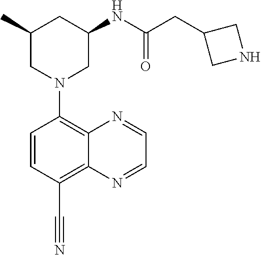 [SMILES: C[C@H]1C[C@@H](NC(=O)CC2CNC2)CN(c2ccc(C#N)c3nccnc23)C1] | 274 |
| 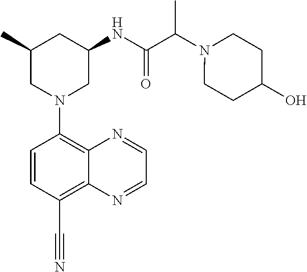 [SMILES: CC(C(=O)N[C@@H]1C[C@H](C)CN(c2ccc(C#N)c3nccnc23)C1)N1CCC(O)CC1] | 775 |
| 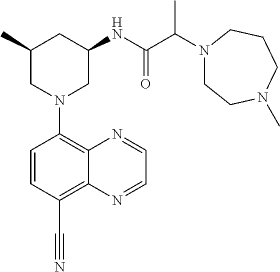 | 276 |
| 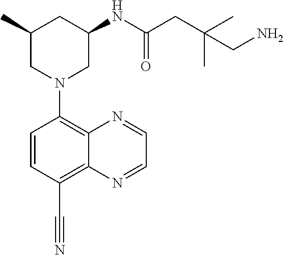 [SMILES: C[C@H]1C[C@@H](NC(=O)CC(C)(C)CN)CN(c2ccc(C#N)c3nccnc23)C1] | 277 |
| 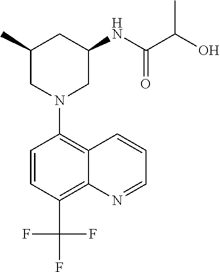 [SMILES: CC(O)C(=O)N[C@@H]1C[C@H](C)CN(c2ccc(C(F)(F)F)c3ncccc23)C1] | 278 |
| 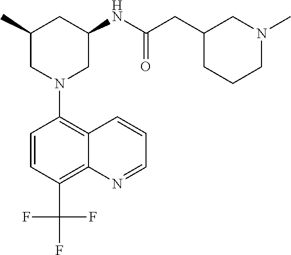 | 279 |
| 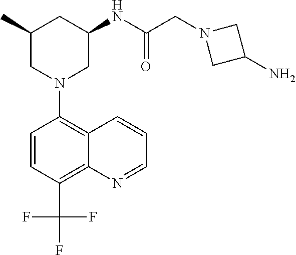 [SMILES: C[C@H]1C[C@@H](NC(=O)CN2CC(N)C2)CN(c2ccc(C(F)(F)F)c3ncccc23)C1] | 280 |
| 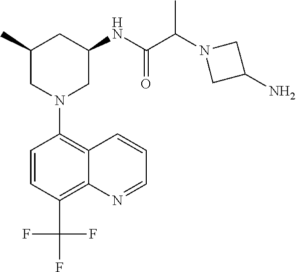 [SMILES: CC(C(=O)N[C@@H]1C[C@H](C)CN(c2ccc(C(F)(F)F)c3ncccc23)C1)N1CC(N)C1] | 281 |
| 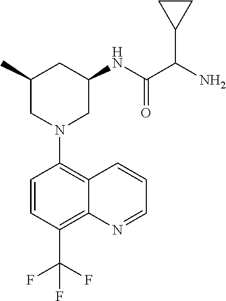 [SMILES: C[C@H]1C[C@@H](NC(=O)C(N)C2CC2)CN(c2ccc(C(F)(F)F)c3ncccc23)C1] | 282 |
| 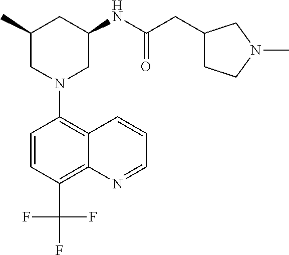 | 283 |
| 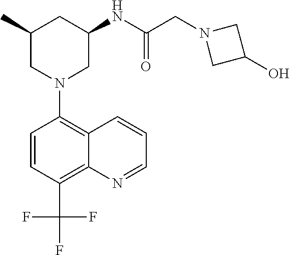 [SMILES: C[C@H]1C[C@@H](NC(=O)CN2CC(O)C2)CN(c2ccc(C(F)(F)F)c3ncccc23)C1] | 284 |
| 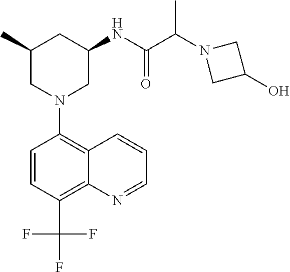 [SMILES: CC(C(=O)N[C@@H]1C[C@H](C)CN(c2ccc(C(F)(F)F)c3ncccc23)C1)N1CC(O)C1] | 285 |
| 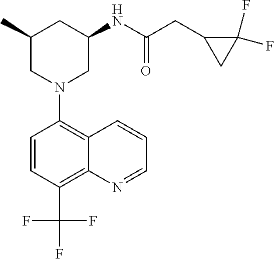 [SMILES: C[C@H]1C[C@@H](NC(=O)CC2CC2(F)F)CN(c2ccc(C(F)(F)F)c3ncccc23)C1] | 286 |
| 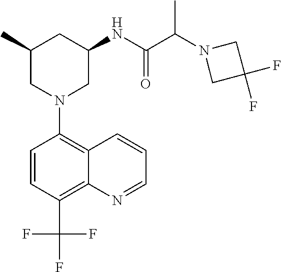 [SMILES: CC(C(=O)N[C@@H]1C[C@H](C)CN(c2ccc(C(F)(F)F)c3ncccc23)C1)N1CC(F)(F)C1] | 287 |
| 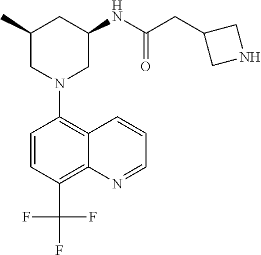 [SMILES: C[C@H]1C[C@@H](NC(=O)CC2CNC2)CN(c2ccc(C(F)(F)F)c3ncccc23)C1] | 288 |
| 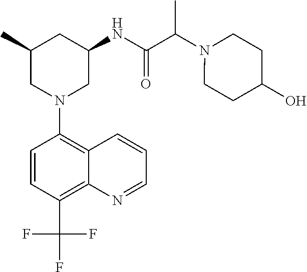 [SMILES: CC(C(=O)N[C@@H]1C[C@H](C)CN(c2ccc(C(F)(F)F)c3ncccc23)C1)N1CCC(O)CC1] | 289 |
| 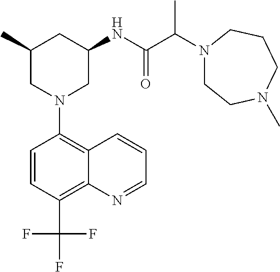 | 290 |
| 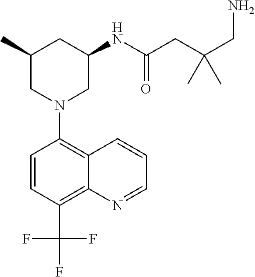 [SMILES: C[C@H]1C[C@@H](NC(=O)CC(C)(C)CN)CN(c2ccc(C(F)(F)F)c3ncccc23)C1] | 291 |
| 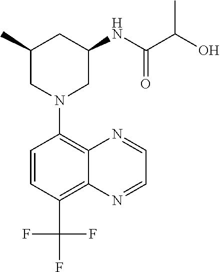 [SMILES: CC(O)C(=O)N[C@@H]1C[C@H](C)CN(c2ccc(C(F)(F)F)c3nccnc23)C1] | 292 |
| 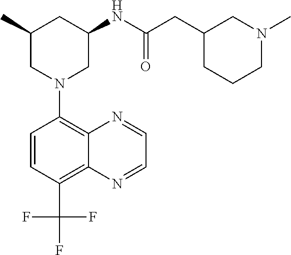 | 293 |
| 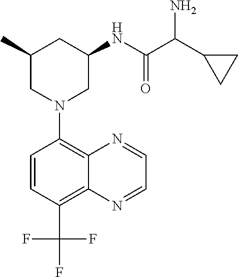 [SMILES: C[C@H]1C[C@@H](NC(=O)C(N)C2CC2)CN(c2ccc(C(F)(F)F)c3nccnc23)C1] | 294 |
| 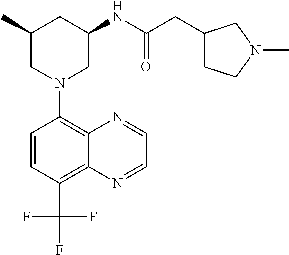 | 295 |
| 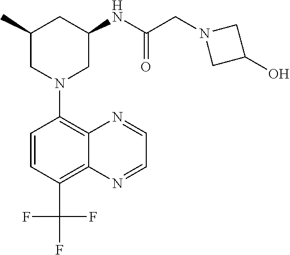 [SMILES: C[C@H]1C[C@@H](NC(=O)CN2CC(O)C2)CN(c2ccc(C(F)(F)F)c3nccnc23)C1] | 296 |
| 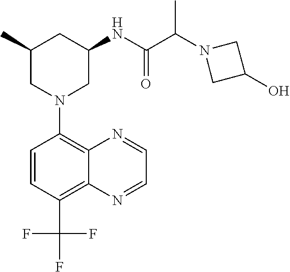 [SMILES: CC(C(=O)N[C@@H]1C[C@H](C)CN(c2ccc(C(F)(F)F)c3nccnc23)C1)N1CC(O)C1] | 297 |
| 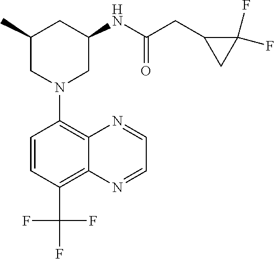 [SMILES: C[C@H]1C[C@@H](NC(=O)CC2CC2(F)F)CN(c2ccc(C(F)(F)F)c3nccnc23)C1] | 298 |
| 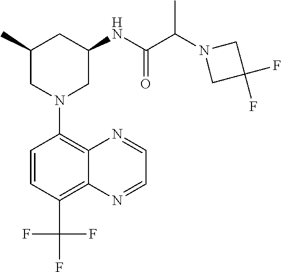 [SMILES: CC(C(=O)N[C@@H]1C[C@H](C)CN(c2ccc(C(F)(F)F)c3nccnc23)C1)N1CC(F)(F)C1] | 299 |
| 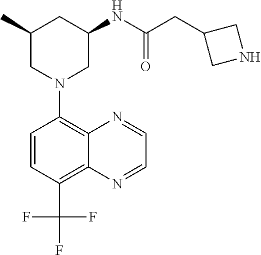 [SMILES: C[C@H]1C[C@@H](NC(=O)CC2CNC2)CN(c2ccc(C(F)(F)F)c3nccnc23)C1] | 300 |
| 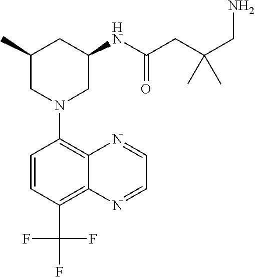 [SMILES: C[C@H]1C[C@@H](NC(=O)CC(C)(C)CN)CN(c2ccc(C(F)(F)F)c3nccnc23)C1] | 301 |
| 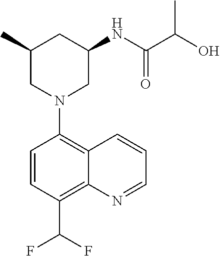 [SMILES: CC(O)C(=O)N[C@@H]1C[C@H](C)CN(c2ccc(C(F)F)c3ncccc23)C1] | 302 |
| 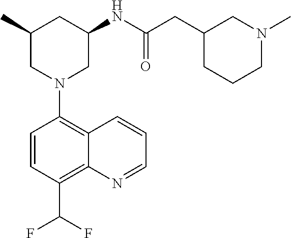 | 303 |
| 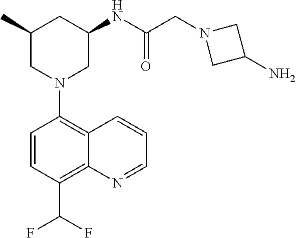 [SMILES: C[C@H]1C[C@@H](NC(=O)CN2CC(N)C2)CN(c2ccc(C(F)F)c3ncccc23)C1] | 304 |
| 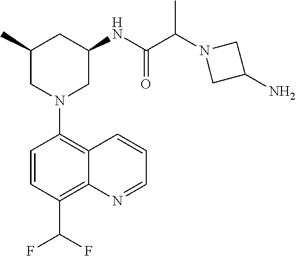 [SMILES: CC(C(=O)N[C@@H]1C[C@H](C)CN(c2ccc(C(F)F)c3ncccc23)C1)N1CC(N)C1] | 305 |
| 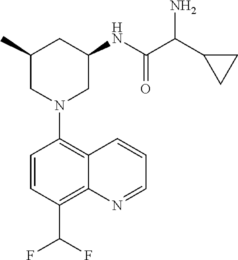 [SMILES: C[C@H]1C[C@@H](NC(=O)C(N)C2CC2)CN(c2ccc(C(F)F)c3ncccc23)C1] | 306 |
| 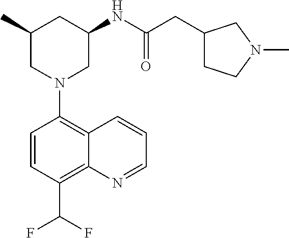 | 307 |
| 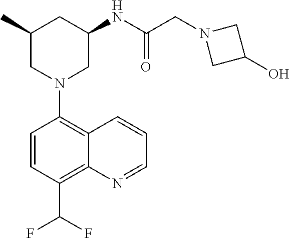 [SMILES: C[C@H]1C[C@@H](NC(=O)CN2CC(O)C2)CN(c2ccc(C(F)F)c3ncccc23)C1] | 308 |
| 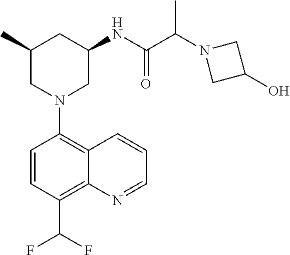 [SMILES: CC(C(=O)N[C@@H]1C[C@H](C)CN(c2ccc(C(F)F)c3ncccc23)C1)N1CC(O)C1] | 309 |
| 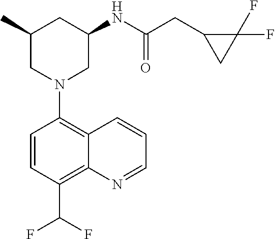 [SMILES: C[C@H]1C[C@@H](NC(=O)CC2CC2(F)F)CN(c2ccc(C(F)F)c3ncccc23)C1] | 310 |
| 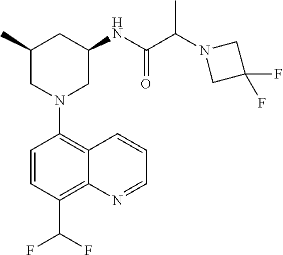 [SMILES: CC(C(=O)N[C@@H]1C[C@H](C)CN(c2ccc(C(F)F)c3ncccc23)C1)N1CC(F)(F)C1] | 311 |
| 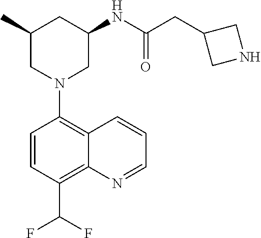 [SMILES: C[C@H]1C[C@@H](NC(=O)CC2CNC2)CN(c2ccc(C(F)F)c3ncccc23)C1] | 312 |
| 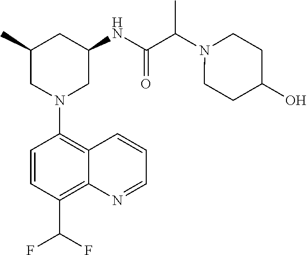 [SMILES: CC(C(=O)N[C@@H]1C[C@H](C)CN(c2ccc(C(F)F)c3ncccc23)C1)N1CCC(O)CC1] | 313 |
| 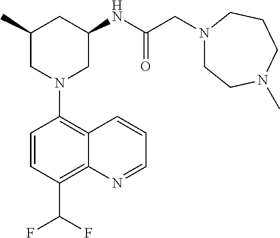 | 314 |
| 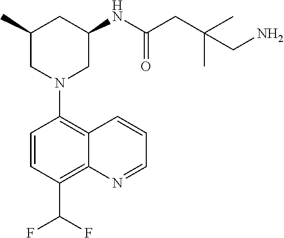 [SMILES: C[C@H]1C[C@@H](NC(=O)CC(C)(C)CN)CN(c2ccc(C(F)F)c3ncccc23)C1] | 315 |
| 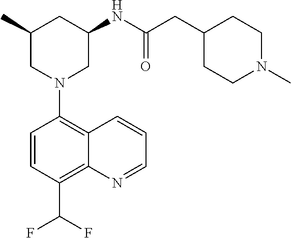 | 316 |
| 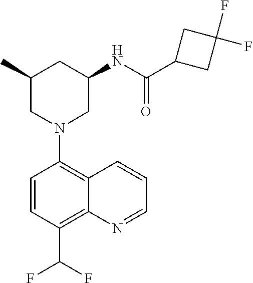 [SMILES: C[C@H]1C[C@@H](NC(=O)C2CC(F)(F)C2)CN(c2ccc(C(F)F)c3ncccc23)C1] | 317 |
| 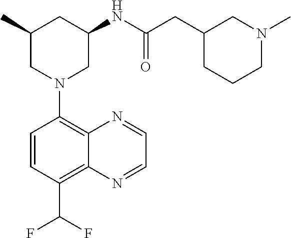 | 318 |
| 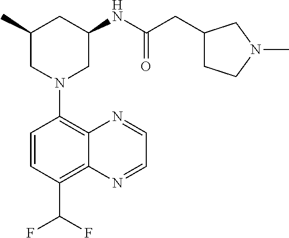 | 319 |
| 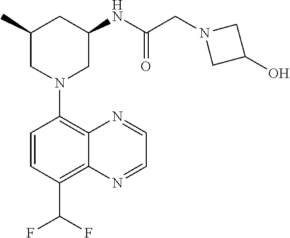 [SMILES: C[C@H]1C[C@@H](NC(=O)CN2CC(O)C2)CN(c2ccc(C(F)F)c3nccnc23)C1] | 320 |
| 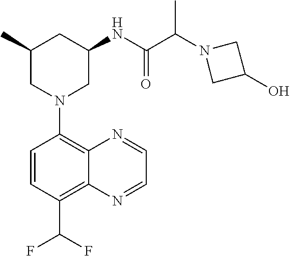 [SMILES: CC(C(=O)N[C@@H]1C[C@H](C)CN(c2ccc(C(F)F)c3nccnc23)C1)N1CC(O)C1] | 321 |
| 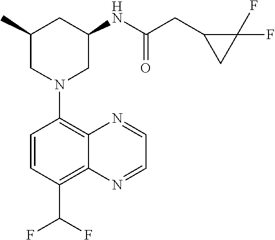 [SMILES: C[C@H]1C[C@@H](NC(=O)CC2CC2(F)F)CN(c2ccc(C(F)F)c3nccnc23)C1] | 322 |
| 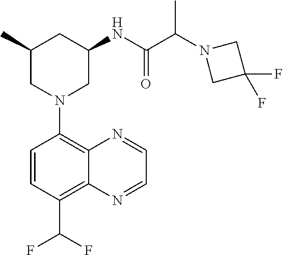 [SMILES: CC(C(=O)N[C@@H]1C[C@H](C)CN(c2ccc(C(F)F)c3nccnc23)C1)N1CC(F)(F)C1] | 323 |
| 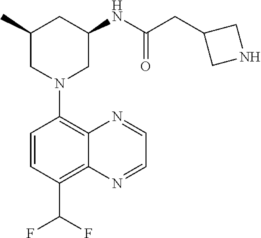 [SMILES: C[C@H]1C[C@@H](NC(=O)CC2CNC2)CN(c2ccc(C(F)F)c3nccnc23)C1] | 324 |
| 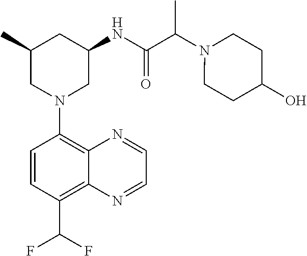 [SMILES: CC(C(=O)N[C@@H]1C[C@H](C)CN(c2ccc(C(F)F)c3nccnc23)C1)N1CCC(O)CC1] | 325 |
| 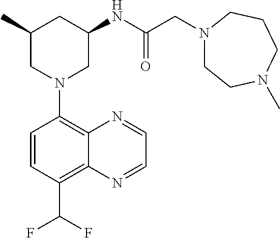 | 326 |
| 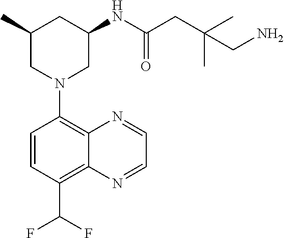 [SMILES: C[C@H]1C[C@@H](NC(=O)CC(C)(C)CN)CN(c2ccc(C(F)F)c3nccnc23)C1] | 327 |
| 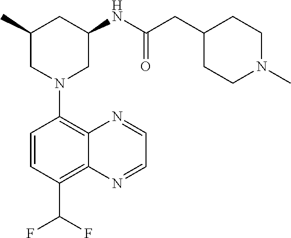 | 328 |
| 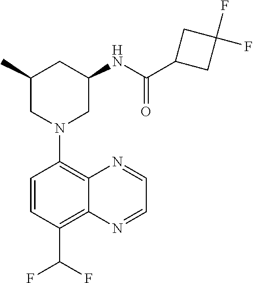 [SMILES: C[C@H]1C[C@@H](NC(=O)C2CC(F)(F)C2)CN(c2ccc(C(F)F)c3nccnc23)C1] | 329 |
| 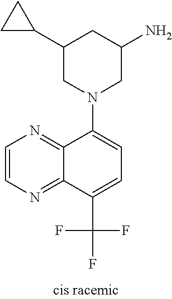 [SMILES: NC1CC(C2CC2)CN(c2ccc(C(F)(F)F)c3nccnc23)C1] | 330 |
| 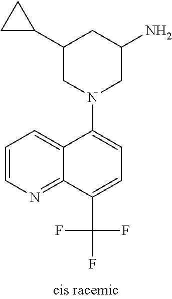 [SMILES: NC1CC(C2CC2)CN(c2ccc(C(F)(F)F)c3ncccc23)C1] | 331 |
| 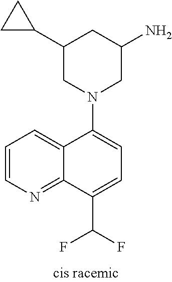 [SMILES: NC1CC(C2CC2)CN(c2ccc(C(F)F)c3ncccc23)C1] | 332 |
| 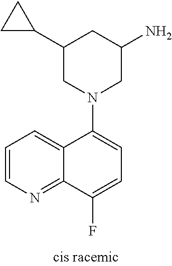 [SMILES: NC1CC(C2CC2)CN(c2ccc(F)c3ncccc23)C1] | 333 |
| 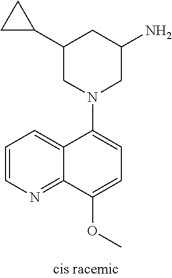 [SMILES: COc1ccc(N2CC(N)CC(C3CC3)C2)c2cccnc12] | 334 |
| 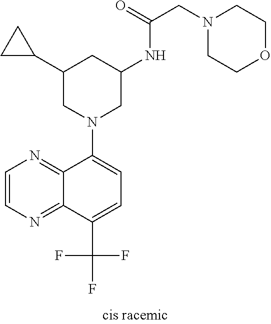 [SMILES: O=C(CN1CCOCC1)NC1CC(C2CC2)CN(c2ccc(C(F)(F)F)c3nccnc23)C1] | 335 |
| 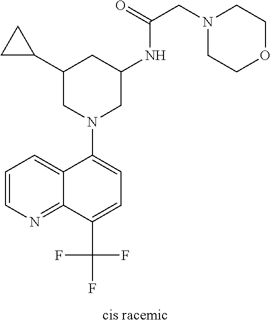 [SMILES: O=C(CN1CCOCC1)NC1CC(C2CC2)CN(c2ccc(C(F)(F)F)c3ncccc23)C1] | 336 |
| 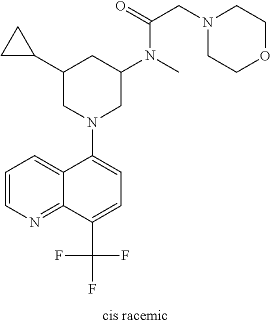 [SMILES: CN(C(=O)CN1CCOCC1)C1CC(C2CC2)CN(c2ccc(C(F)(F)F)c3ncccc23)C1] | 337 |
| 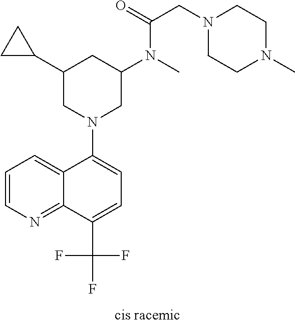 [SMILES: CN1CCN(CC(=O)N(C)C2CC(C3CC3)CN(c3ccc(C(F)(F)F)c4ncccc34)C2)CC1] | 338 |
| 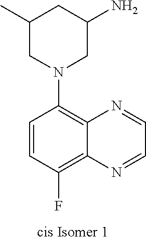 [SMILES: CC1CC(N)CN(c2ccc(F)c3nccnc23)C1] | 339 |
| 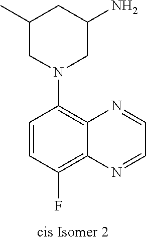 [SMILES: CC1CC(N)CN(c2ccc(F)c3nccnc23)C1] | 340 |
| 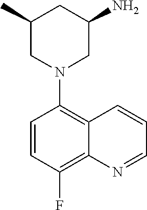 [SMILES: C[C@H]1C[C@@H](N)CN(c2ccc(F)c3ncccc23)C1] | 341 |
| 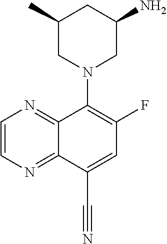 [SMILES: C[C@H]1C[C@@H](N)CN(c2c(F)cc(C#N)c3nccnc23)C1] | 342 |
| 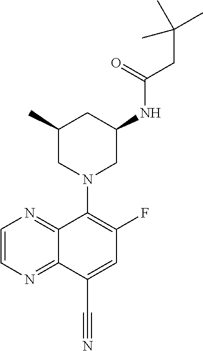 [SMILES: C[C@H]1C[C@@H](NC(=O)CC(C)(C)C)CN(c2c(F)cc(C#N)c3nccnc23)C1] | 343 |
| 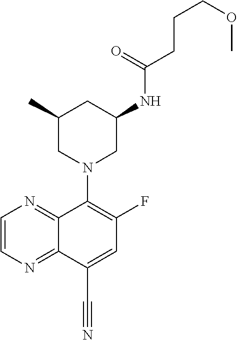 [SMILES: COCCCC(=O)N[C@@H]1C[C@H](C)CN(c2c(F)cc(C#N)c3nccnc23)C1] | 344 |
| 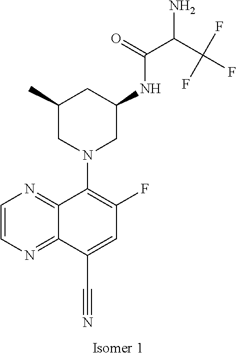 [SMILES: C[C@H]1C[C@@H](NC(=O)C(N)C(F)(F)F)CN(c2c(F)cc(C#N)c3nccnc23)C1] | 345 |
| 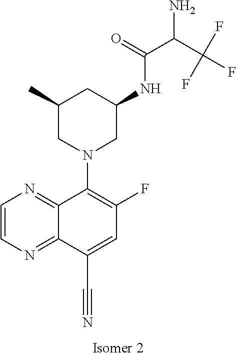 [SMILES: C[C@H]1C[C@@H](NC(=O)C(N)C(F)(F)F)CN(c2c(F)cc(C#N)c3nccnc23)C1] | 346 |
| 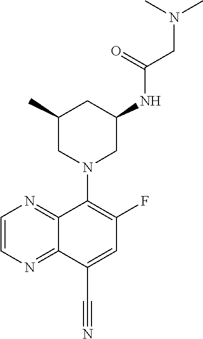 [SMILES: C[C@H]1C[C@@H](NC(=O)CN(C)C)CN(c2c(F)cc(C#N)c3nccnc23)C1] | 347 |
| 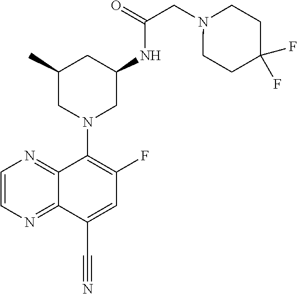 [SMILES: C[C@H]1C[C@@H](NC(=O)CN2CCC(F)(F)CC2)CN(c2c(F)cc(C#N)c3nccnc23)C1] | 348 |
| 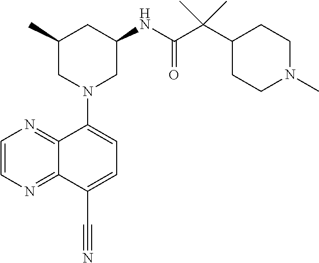 [SMILES: C[C@H]1C[C@@H](NC(=O)C(C)(C)C2CCN(C)CC2)CN(c2ccc(C#N)c3nccnc23)C1] | 349 |
| 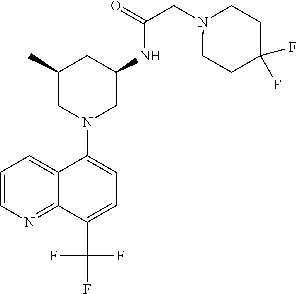 [SMILES: C[C@H]1C[C@@H](NC(=O)CN2CCC(F)(F)CC2)CN(c2ccc(C(F)(F)F)c3ncccc23)C1] | 350 |
| 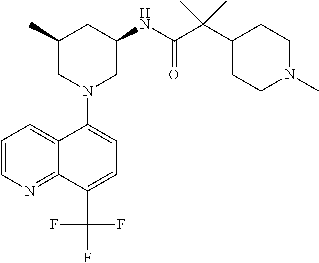 [SMILES: C[C@H]1C[C@@H](NC(=O)C(C)(C)C2CCN(C)CC2)CN(c2ccc(C(F)(F)F)c3ncccc23)C1] | 351 |
| 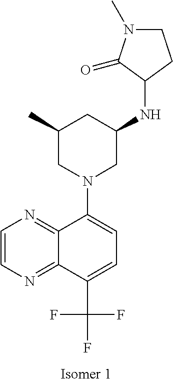 [SMILES: C[C@H]1C[C@@H](NC2CCN(C)C2=O)CN(c2ccc(C(F)(F)F)c3nccnc23)C1] | 352 |
| 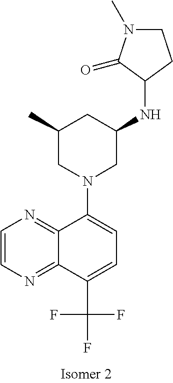 [SMILES: C[C@H]1C[C@@H](NC2CCN(C)C2=O)CN(c2ccc(C(F)(F)F)c3nccnc23)C1] | 353 |
| 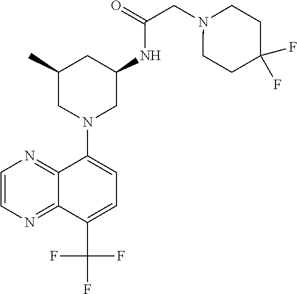 [SMILES: C[C@H]1C[C@@H](NC(=O)CN2CCC(F)(F)CC2)CN(c2ccc(C(F)(F)F)c3nccnc23)C1] | 354 |
| 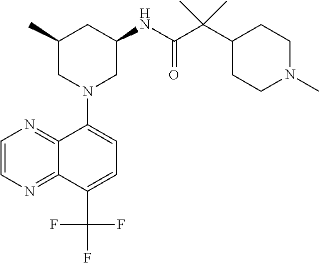 [SMILES: C[C@H]1C[C@@H](NC(=O)C(C)(C)C2CCN(C)CC2)CN(c2ccc(C(F)(F)F)c3nccnc23)C1] | 355 |
| 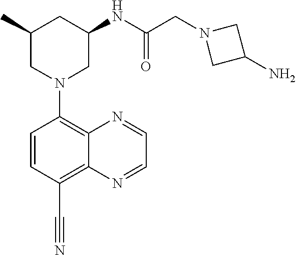 [SMILES: C[C@H]1C[C@@H](NC(=O)CN2CC(N)C2)CN(c2ccc(C#N)c3nccnc23)C1] | 356 |
| 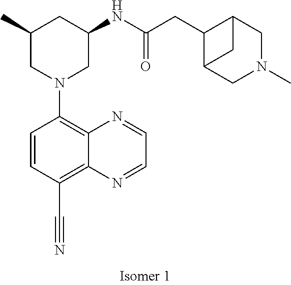 | 357 |
| 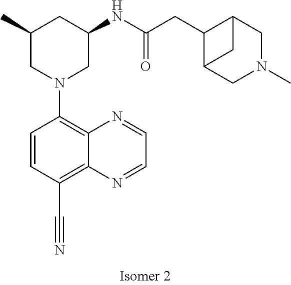 | 358 |
| 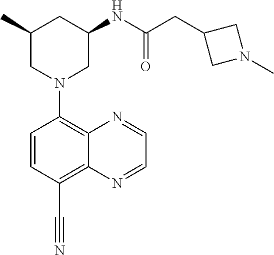 | 359 |
| 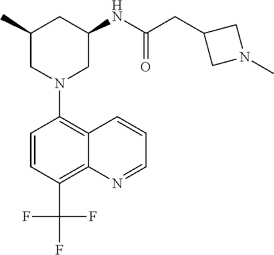 | 360 |
| 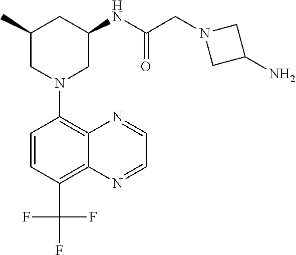 [SMILES: C[C@H]1C[C@@H](NC(=O)CN2CC(N)C2)CN(c2ccc(C(F)(F)F)c3nccnc23)C1] | 361 |
| 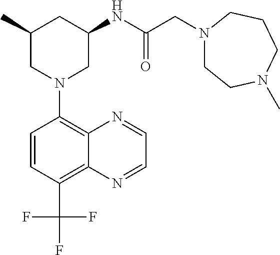 | 362 |
| 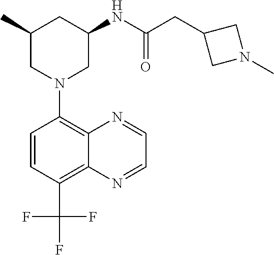 | 363 |
| 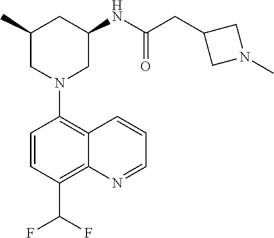 | 364 |
| 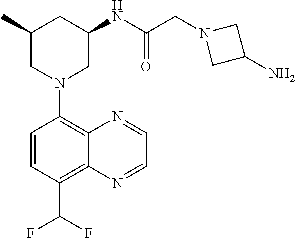 [SMILES: C[C@H]1C[C@@H](NC(=O)CN2CC(N)C2)CN(c2ccc(C(F)F)c3nccnc23)C1] | 365 |
| 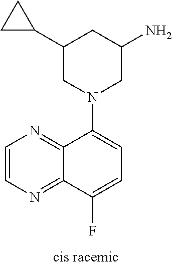 [SMILES: NC1CC(C2CC2)CN(c2ccc(F)c3nccnc23)C1] | 366 |
| 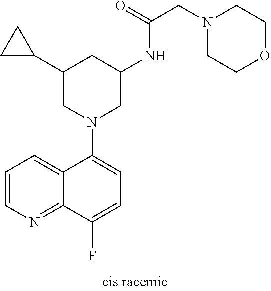 [SMILES: O=C(CN1CCOCC1)NC1CC(C2CC2)CN(c2ccc(F)c3ncccc23)C1] | 367 |
| 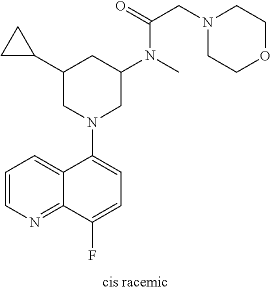 [SMILES: CN(C(=O)CN1CCOCC1)C1CC(C2CC2)CN(c2ccc(F)c3ncccc23)C1] | 368 |
| 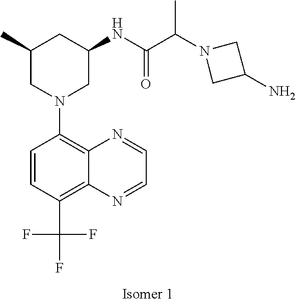 [SMILES: CC(C(=O)N[C@@H]1C[C@H](C)CN(c2ccc(C(F)(F)F)c3nccnc23)C1)N1CC(N)C1] | 369 |
| 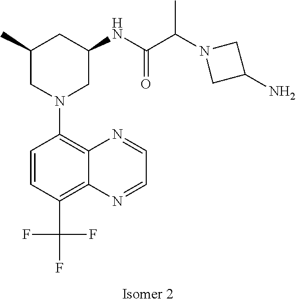 [SMILES: CC(C(=O)N[C@@H]1C[C@H](C)CN(c2ccc(C(F)(F)F)c3nccnc23)C1)N1CC(N)C1] | 370 |
| 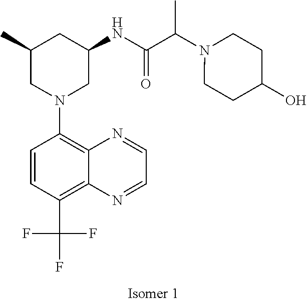 [SMILES: CC(C(=O)N[C@@H]1C[C@H](C)CN(c2ccc(C(F)(F)F)c3nccnc23)C1)N1CCC(O)CC1] | 371 |
| 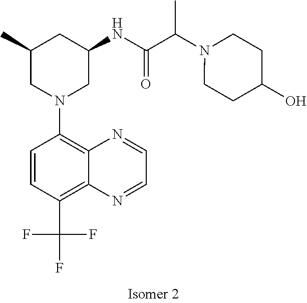 [SMILES: CC(C(=O)N[C@@H]1C[C@H](C)CN(c2ccc(C(F)(F)F)c3nccnc23)C1)N1CCC(O)CC1] | 377 |
| 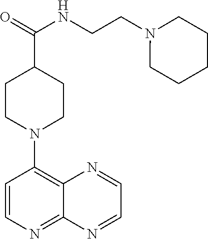 [SMILES: O=C(NCCN1CCCCC1)C1CCN(c2ccnc3nccnc23)CC1] | 373 |
| 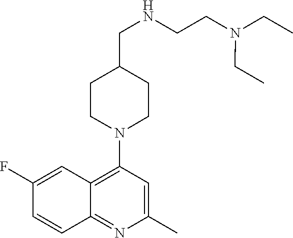 [SMILES: CCN(CC)CCNCC1CCN(c2cc(C)nc3ccc(F)cc23)CC1] | 374 |
| 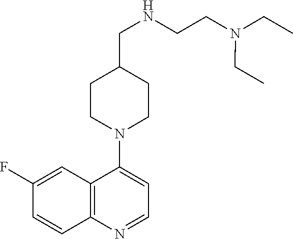 [SMILES: CCN(CC)CCNCC1CCN(c2ccnc3ccc(F)cc23)CC1] | 375 |
| 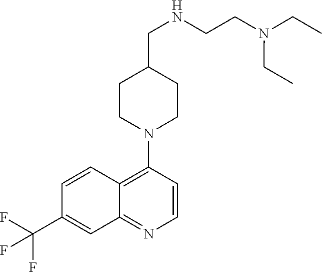 [SMILES: CCN(CC)CCNCC1CCN(c2ccnc3cc(C(F)(F)F)ccc23)CC1] | 376 |
| 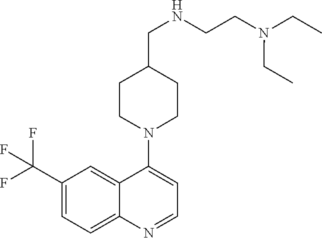 [SMILES: CCN(CC)CCNCC1CCN(c2ccnc3ccc(C(F)(F)F)cc23)CC1] | 377 |
| 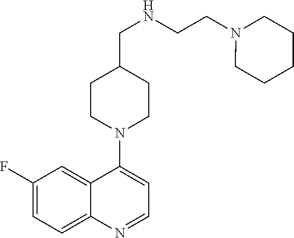 [SMILES: Fc1ccc2nccc(N3CCC(CNCCN4CCCCC4)CC3)c2c1] | 378 |
| 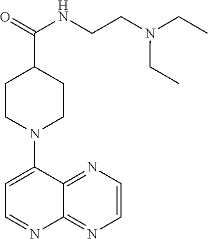 [SMILES: CCN(CC)CCNC(=O)C1CCN(c2ccnc3nccnc23)CC1] | 379 |
| 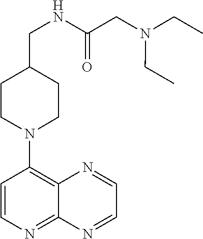 [SMILES: CCN(CC)CC(=O)NCC1CCN(c2ccnc3nccnc23)CC1] | 380 |
| 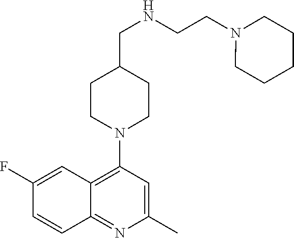 [SMILES: Cc1cc(N2CCC(CNCCN3CCCCC3)CC2)c2cc(F)ccc2n1] | 381 |
| 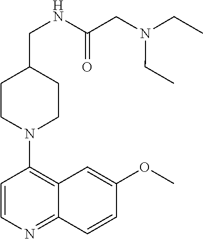 [SMILES: CCN(CC)CC(=O)NCC1CCN(c2ccnc3ccc(OC)cc23)CC1] | 382 |
| 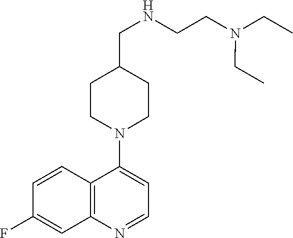 [SMILES: CCN(CC)CCNCC1CCN(c2ccnc3cc(F)ccc23)CC1] | 383 |
| 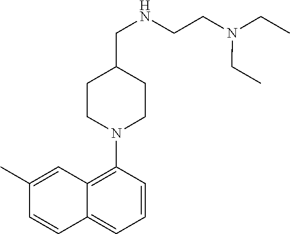 [SMILES: CCN(CC)CCNCC1CCN(c2cccc3ccc(C)cc23)CC1] | 384 |
| 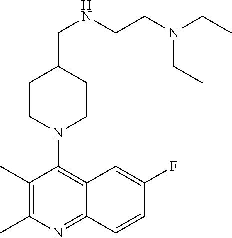 [SMILES: CCN(CC)CCNCC1CCN(c2c(C)c(C)nc3ccc(F)cc23)CC1] | 385 |
| 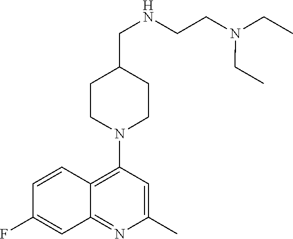 [SMILES: CCN(CC)CCNCC1CCN(c2cc(C)nc3cc(F)ccc23)CC1] | 386 |
| 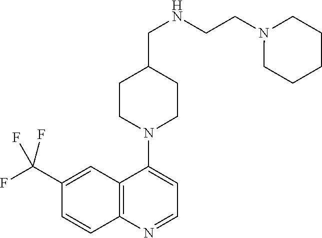 [SMILES: FC(F)(F)c1ccc2nccc(N3CCC(CNCCN4CCCCC4)CC3)c2c1] | 387 |
| 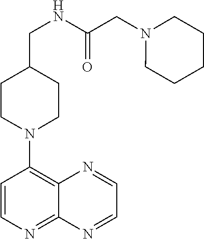 [SMILES: O=C(CN1CCCCC1)NCC1CCN(c2ccnc3nccnc23)CC1] | 388 |
| 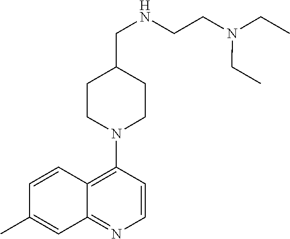 [SMILES: CCN(CC)CCNCC1CCN(c2ccnc3cc(C)ccc23)CC1] | 389 |
| 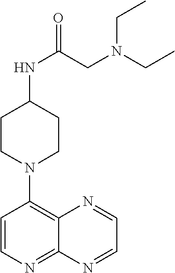 [SMILES: CCN(CC)CC(=O)NC1CCN(c2ccnc3nccnc23)CC1] | 390 |
| 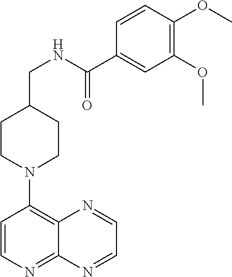 [SMILES: COc1ccc(C(=O)NCC2CCN(c3ccnc4nccnc34)CC2)cc1OC] | 391 |
| 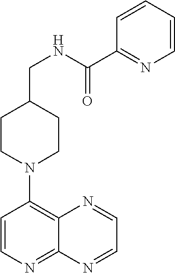 [SMILES: O=C(NCC1CCN(c2ccnc3nccnc23)CC1)c1ccccn1] | 392 |
| 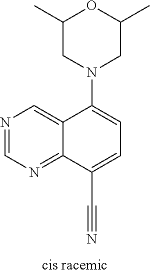 [SMILES: CC1CN(c2ccc(C#N)c3ncncc23)CC(C)O1] | 393 |
| 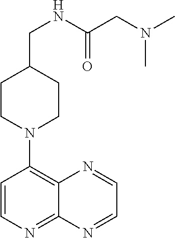 [SMILES: CN(C)CC(=O)NCC1CCN(c2ccnc3nccnc23)CC1] | 394 |
| 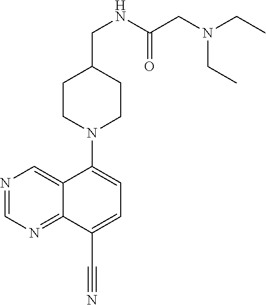 [SMILES: CCN(CC)CC(=O)NCC1CCN(c2ccc(C#N)c3ncncc23)CC1] | 395 |
| 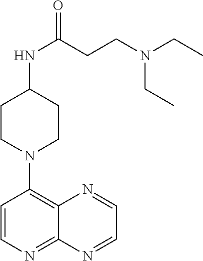 [SMILES: CCN(CC)CCC(=O)NC1CCN(c2ccnc3nccnc23)CC1] | 396 |
| 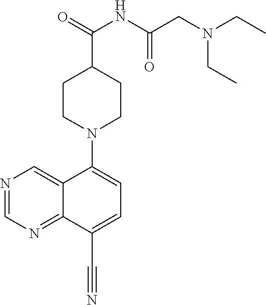 [SMILES: CCN(CC)CC(=O)NC(=O)C1CCN(c2ccc(C#N)c3ncncc23)CC1] | 397 |
| 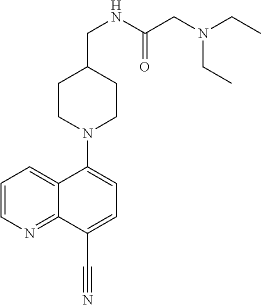 [SMILES: CCN(CC)CC(=O)NCC1CCN(c2ccc(C#N)c3ncccc23)CC1] | 398 |
| 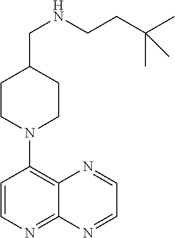 [SMILES: CC(C)(C)CCNCC1CCN(c2ccnc3nccnc23)CC1] | 399 |
| 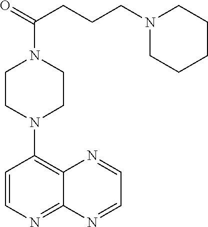 [SMILES: O=C(CCCN1CCCCC1)N1CCN(c2ccnc3nccnc23)CC1] | 400 |
| 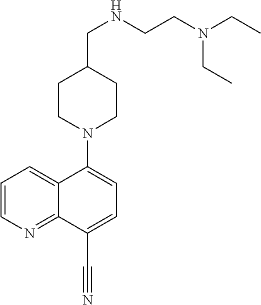 [SMILES: CCN(CC)CCNCC1CCN(c2ccc(C#N)c3ncccc23)CC1] | 401 |
| 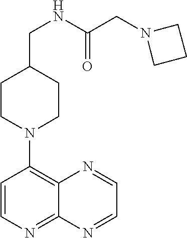 [SMILES: O=C(CN1CCC1)NCC1CCN(c2ccnc3nccnc23)CC1] | 402 |
| 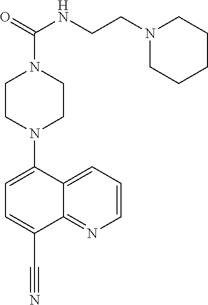 [SMILES: N#Cc1ccc(N2CCN(C(=O)NCCN3CCCCC3)CC2)c2cccnc12] | 403 |
| 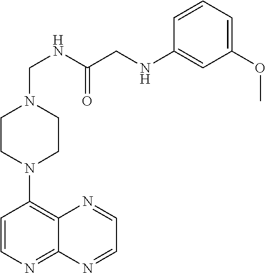 [SMILES: COc1cccc(NCC(=O)NCN2CCN(c3ccnc4nccnc34)CC2)c1] | 404 |
| 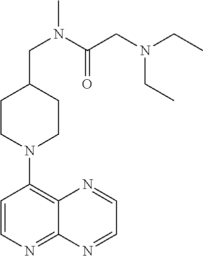 [SMILES: CCN(CC)CC(=O)N(C)CC1CCN(c2ccnc3nccnc23)CC1] | 405 |
| 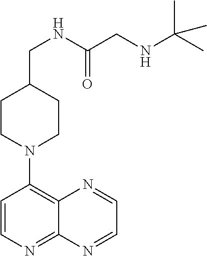 [SMILES: CC(C)(C)NCC(=O)NCC1CCN(c2ccnc3nccnc23)CC1] | 406 |
| 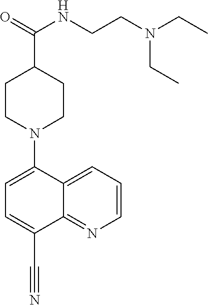 [SMILES: CCN(CC)CCNC(=O)C1CCN(c2ccc(C#N)c3ncccc23)CC1] | 407 |
| 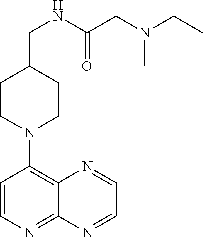 [SMILES: CCN(C)CC(=O)NCC1CCN(c2ccnc3nccnc23)CC1] | 408 |
| 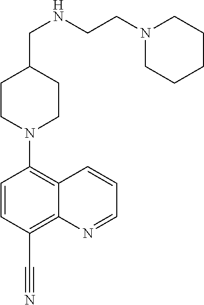 [SMILES: N#Cc1ccc(N2CCC(CNCCN3CCCCC3)CC2)c2cccnc12] | 409 |
| 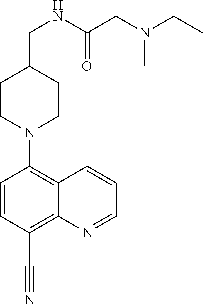 [SMILES: CCN(C)CC(=O)NCC1CCN(c2ccc(C#N)c3ncccc23)CC1] | 410 |
| 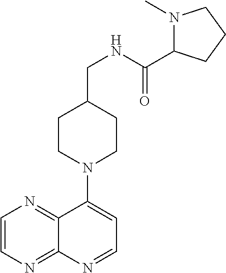 [SMILES: CN1CCCC1C(=O)NCC1CCN(c2ccnc3nccnc23)CC1] | 411 |
| 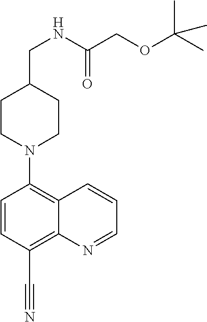 [SMILES: CC(C)(C)OCC(=O)NCC1CCN(c2ccc(C#N)c3ncccc23)CC1] | 412 |
| 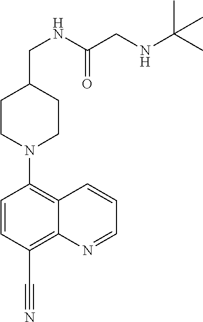 [SMILES: CC(C)(C)NCC(=O)NCC1CCN(c2ccc(C#N)c3ncccc23)CC1] | 413 |
| 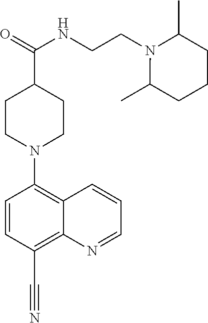 [SMILES: CC1CCCC(C)N1CCNC(=O)C1CCN(c2ccc(C#N)c3ncccc23)CC1] | 414 |
| 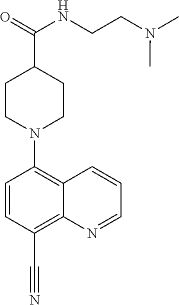 [SMILES: CN(C)CCNC(=O)C1CCN(c2ccc(C#N)c3ncccc23)CC1] | 415 |
| 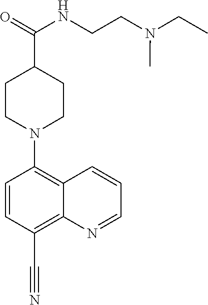 [SMILES: CCN(C)CCNC(=O)C1CCN(c2ccc(C#N)c3ncccc23)CC1] | 416 |
| 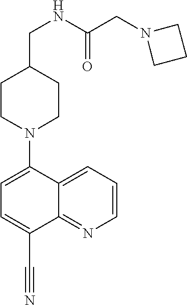 [SMILES: N#Cc1ccc(N2CCC(CNC(=O)CN3CCC3)CC2)c2cccnc12] | 417 |
| 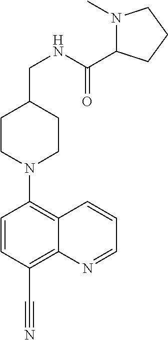 [SMILES: CN1CCCC1C(=O)NCC1CCN(c2ccc(C#N)c3ncccc23)CC1] | 418 |
| 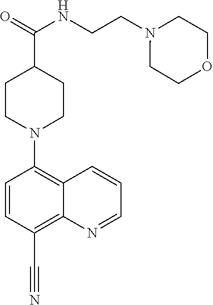 [SMILES: N#Cc1ccc(N2CCC(C(=O)NCCN3CCOCC3)CC2)c2cccnc12] | 419 |
| 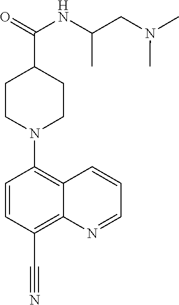 [SMILES: CC(CN(C)C)NC(=O)C1CCN(c2ccc(C#N)c3ncccc23)CC1] | 420 |
| 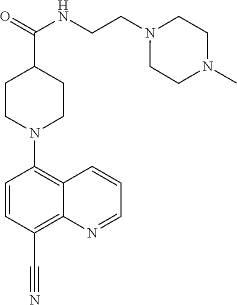 [SMILES: CN1CCN(CCNC(=O)C2CCN(c3ccc(C#N)c4ncccc34)CC2)CC1] | 421 |
| 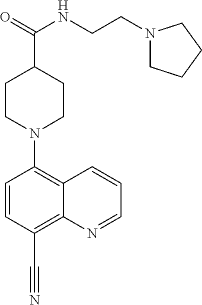 [SMILES: N#Cc1ccc(N2CCC(C(=O)NCCN3CCCC3)CC2)c2cccnc12] | 422 |
| 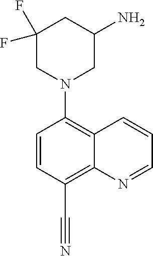 [SMILES: N#Cc1ccc(N2CC(N)CC(F)(F)C2)c2cccnc12] | 423 |
| 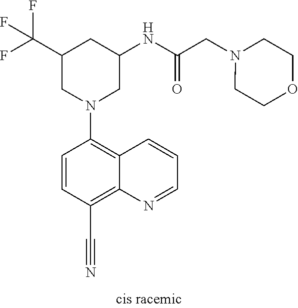 [SMILES: N#Cc1ccc(N2CC(NC(=O)CN3CCOCC3)CC(C(F)(F)F)C2)c2cccnc12] | 424 |
| 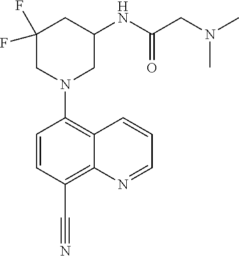 [SMILES: CN(C)CC(=O)NC1CN(c2ccc(C#N)c3ncccc23)CC(F)(F)C1] | 425 |
| 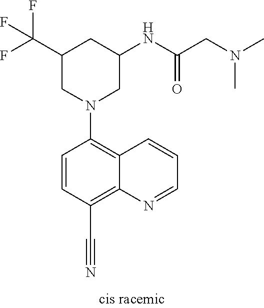 [SMILES: CN(C)CC(=O)NC1CC(C(F)(F)F)CN(c2ccc(C#N)c3ncccc23)C1] | 426 |
| 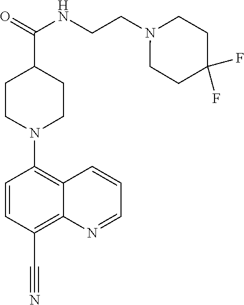 [SMILES: N#Cc1ccc(N2CCC(C(=O)NCCN3CCC(F)(F)CC3)CC2)c2cccnc12] | 427 |
| 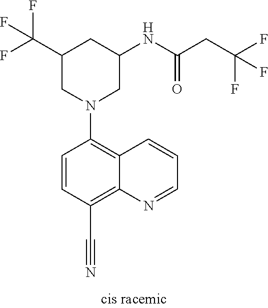 [SMILES: N#Cc1ccc(N2CC(NC(=O)CC(F)(F)F)CC(C(F)(F)F)C2)c2cccnc12] | 428 |
| 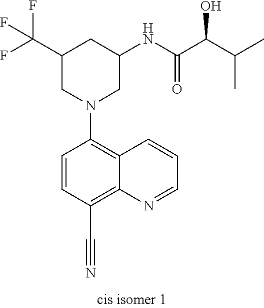 [SMILES: CC(C)[C@H](O)C(=O)NC1CC(C(F)(F)F)CN(c2ccc(C#N)c3ncccc23)C1] | 429 |
| 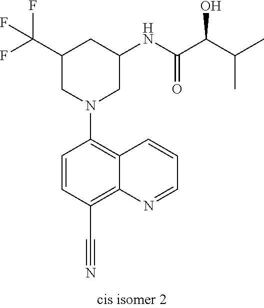 [SMILES: CC(C)[C@H](O)C(=O)NC1CC(C(F)(F)F)CN(c2ccc(C#N)c3ncccc23)C1] | 430 |
| 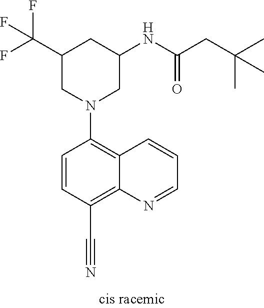 [SMILES: CC(C)(C)CC(=O)NC1CC(C(F)(F)F)CN(c2ccc(C#N)c3ncccc23)C1] | 431 |
| 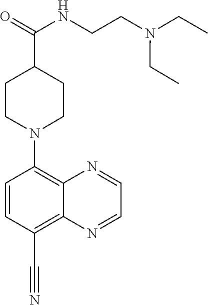 [SMILES: CCN(CC)CCNC(=O)C1CCN(c2ccc(C#N)c3nccnc23)CC1] | 432 |
| 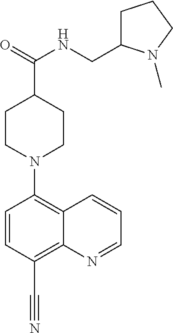 [SMILES: CN1CCCC1CNC(=O)C1CCN(c2ccc(C#N)c3ncccc23)CC1] | 433 |
| 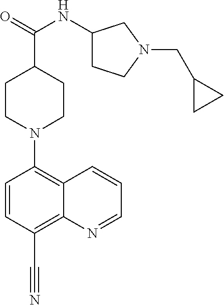 [SMILES: N#Cc1ccc(N2CCC(C(=O)NC3CCN(CC4CC4)C3)CC2)c2cccnc12] | 434 |
| 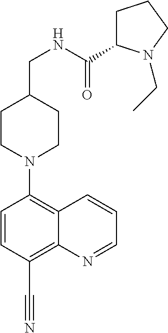 [SMILES: CCN1CCC[C@H]1C(=O)NCC1CCN(c2ccc(C#N)c3ncccc23)CC1] | 435 |
| 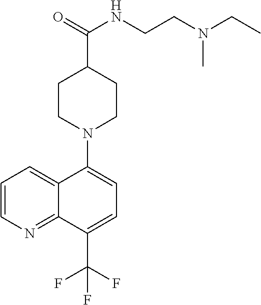 [SMILES: CCN(C)CCNC(=O)C1CCN(c2ccc(C(F)(F)F)c3ncccc23)CC1] | 436 |
| 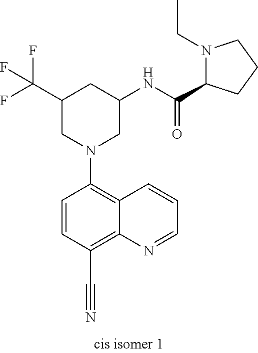 [SMILES: CCN1CCC[C@H]1C(=O)NC1CC(C(F)(F)F)CN(c2ccc(C#N)c3ncccc23)C1] | 437 |
| 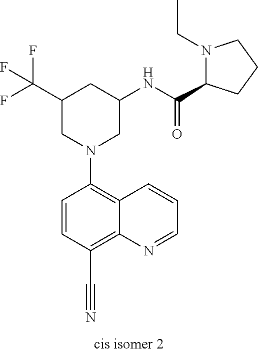 [SMILES: CCN1CCC[C@H]1C(=O)NC1CC(C(F)(F)F)CN(c2ccc(C#N)c3ncccc23)C1] | 438 |
| 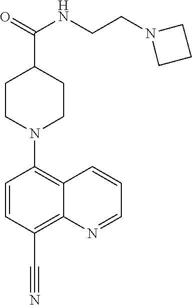 [SMILES: N#Cc1ccc(N2CCC(C(=O)NCCN3CCC3)CC2)c2cccnc12] | 439 |
| 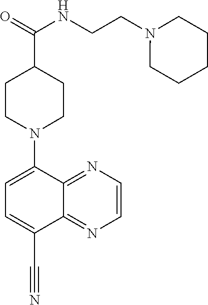 [SMILES: N#Cc1ccc(N2CCC(C(=O)NCCN3CCCCC3)CC2)c2nccnc12] | 440 |
| 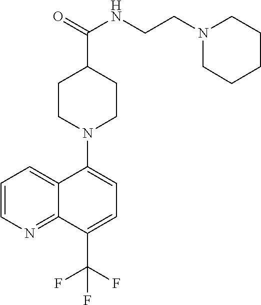 [SMILES: O=C(NCCN1CCCCC1)C1CCN(c2ccc(C(F)(F)F)c3ncccc23)CC1] | 441 |
| 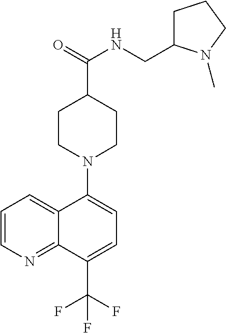 [SMILES: CN1CCCC1CNC(=O)C1CCN(c2ccc(C(F)(F)F)c3ncccc23)CC1] | 442 |
| 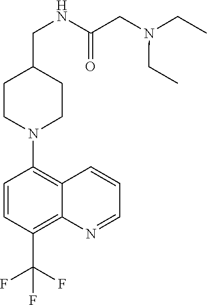 [SMILES: CCN(CC)CC(=O)NCC1CCN(c2ccc(C(F)(F)F)c3ncccc23)CC1] | 443 |
| 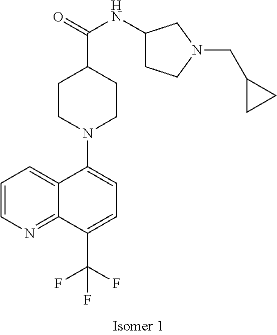 [SMILES: O=C(NC1CCN(CC2CC2)C1)C1CCN(c2ccc(C(F)(F)F)c3ncccc23)CC1] | 444 |
| 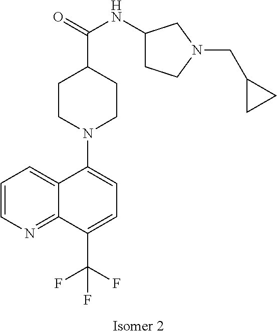 [SMILES: O=C(NC1CCN(CC2CC2)C1)C1CCN(c2ccc(C(F)(F)F)c3ncccc23)CC1] | 445 |
| 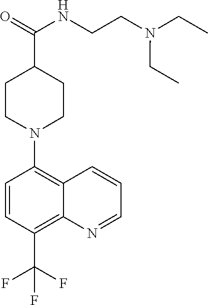 [SMILES: CCN(CC)CCNC(=O)C1CCN(c2ccc(C(F)(F)F)c3ncccc23)CC1] | 446 |
| 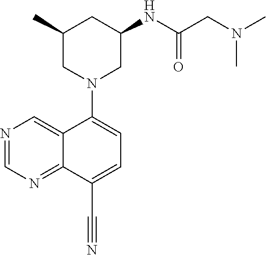 [SMILES: C[C@H]1C[C@@H](NC(=O)CN(C)C)CN(c2ccc(C#N)c3ncncc23)C1] | 447 |
| 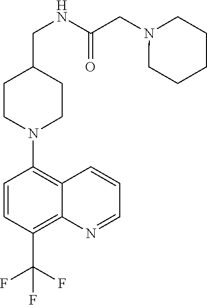 [SMILES: O=C(CN1CCCCC1)NCC1CCN(c2ccc(C(F)(F)F)c3ncccc23)CC1] | 448 |
| 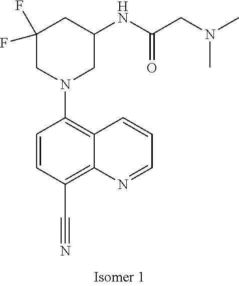 [SMILES: CN(C)CC(=O)NC1CN(c2ccc(C#N)c3ncccc23)CC(F)(F)C1] | 449 |
| 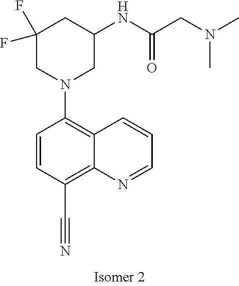 [SMILES: CN(C)CC(=O)NC1CN(c2ccc(C#N)c3ncccc23)CC(F)(F)C1] | 450 |
| 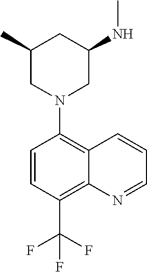 [SMILES: CN[C@@H]1C[C@H](C)CN(c2ccc(C(F)(F)F)c3ncccc23)C1] | 451 |
| 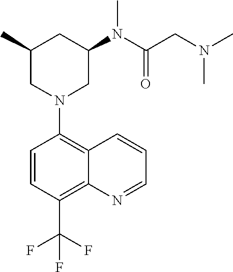 | 452 |
| 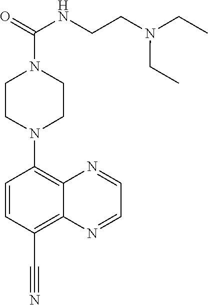 [SMILES: CCN(CC)CCNC(=O)N1CCN(c2ccc(C#N)c3nccnc23)CC1] | 453 |
| 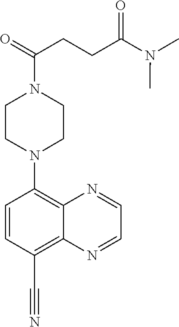 [SMILES: CN(C)C(=O)CCC(=O)N1CCN(c2ccc(C#N)c3nccnc23)CC1] | 454 |
| 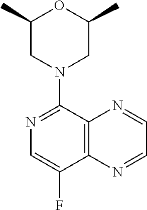 [SMILES: C[C@@H]1CN(c2ncc(F)c3nccnc23)C[C@H](C)O1] | 455 |
| 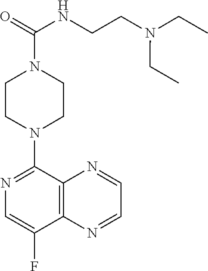 [SMILES: CCN(CC)CCNC(=O)N1CCN(c2ncc(F)c3nccnc23)CC1] | 456 |
| 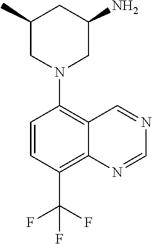 [SMILES: C[C@H]1C[C@@H](N)CN(c2ccc(C(F)(F)F)c3ncncc23)C1] | 457 |
| 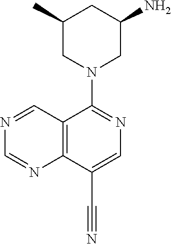 [SMILES: C[C@H]1C[C@@H](N)CN(c2ncc(C#N)c3ncncc23)C1] | 458 |
| 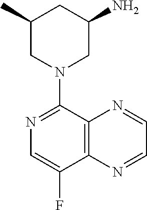 [SMILES: C[C@H]1C[C@@H](N)CN(c2ncc(F)c3nccnc23)C1] | 459 |
| 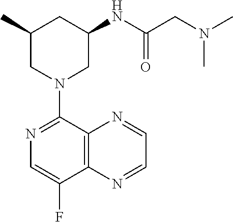 | 460 |
| 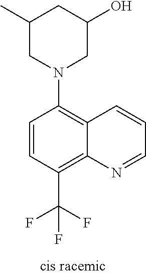 [SMILES: CC1CC(O)CN(c2ccc(C(F)(F)F)c3ncccc23)C1] | 461 |
| 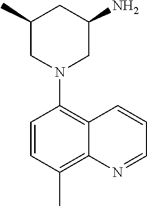 [SMILES: Cc1ccc(N2C[C@@H](C)C[C@@H](N)C2)c2cccnc12] | 462 |
| 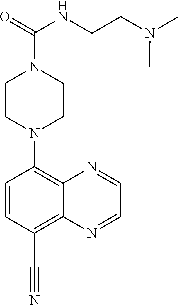 [SMILES: CN(C)CCNC(=O)N1CCN(c2ccc(C#N)c3nccnc23)CC1] | 463 |
| 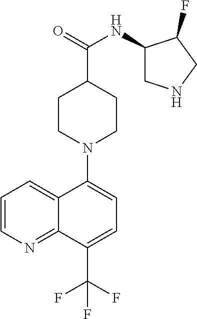 [SMILES: O=C(N[C@@H]1CNC[C@@H]1F)C1CCN(c2ccc(C(F)(F)F)c3ncccc23)CC1] | 464 |
| 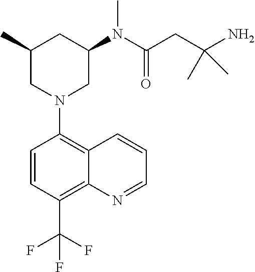 [SMILES: C[C@H]1C[C@@H](N(C)C(=O)CC(C)(C)N)CN(c2ccc(C(F)(F)F)c3ncccc23)C1] | 465 |
| 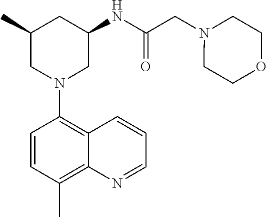 [SMILES: Cc1ccc(N2C[C@@H](C)C[C@@H](NC(=O)CN3CCOCC3)C2)c2cccnc12] | 466 |
| 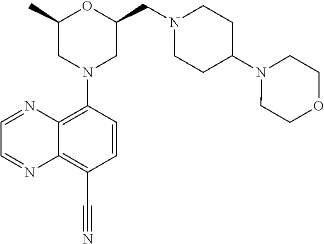 [SMILES: C[C@@H]1CN(c2ccc(C#N)c3nccnc23)C[C@H](CN2CCC(N3CCOCC3)CC2)O1] | 467 |
| 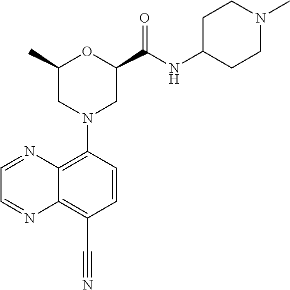 [SMILES: C[C@@H]1CN(c2ccc(C#N)c3nccnc23)C[C@H](C(=O)NC2CCN(C)CC2)O1] | 468 |
| 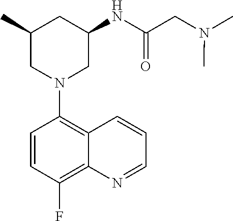 | 469 |
| 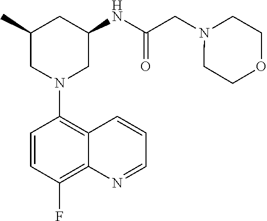 [SMILES: C[C@H]1C[C@@H](NC(=O)CN2CCOCC2)CN(c2ccc(F)c3ncccc23)C1] | 470 |
| 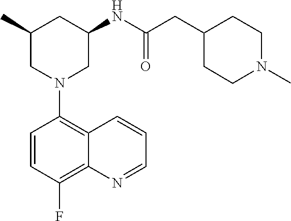 | 471 |
| 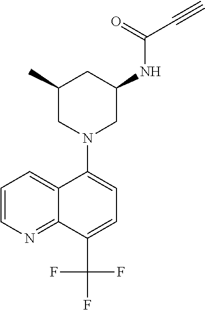 [SMILES: C#CC(=O)N[C@@H]1C[C@H](C)CN(c2ccc(C(F)(F)F)c3ncccc23)C1] | 472 |
| 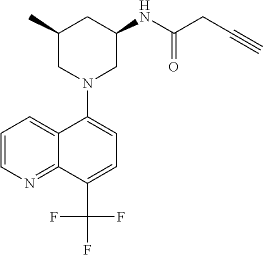 [SMILES: C#CCC(=O)N[C@@H]1C[C@H](C)CN(c2ccc(C(F)(F)F)c3ncccc23)C1] | 473 |
| 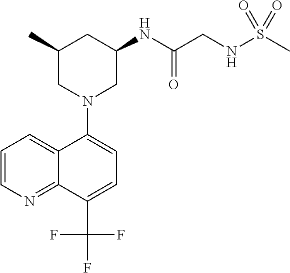 [SMILES: C[C@H]1C[C@@H](NC(=O)CNS(C)(=O)=O)CN(c2ccc(C(F)(F)F)c3ncccc23)C1] | 474 |
| 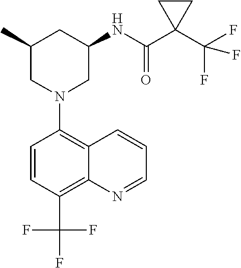 [SMILES: C[C@H]1C[C@@H](NC(=O)C2(C(F)(F)F)CC2)CN(c2ccc(C(F)(F)F)c3ncccc23)C1] | 475 |
| 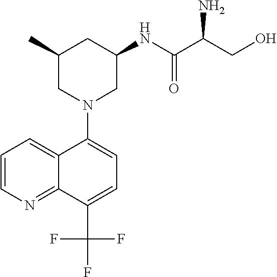 [SMILES: C[C@H]1C[C@@H](NC(=O)[C@@H](N)CO)CN(c2ccc(C(F)(F)F)c3ncccc23)C1] | 476 |
| 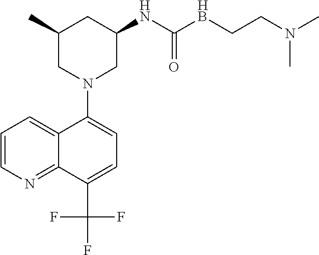 | 477 |
| 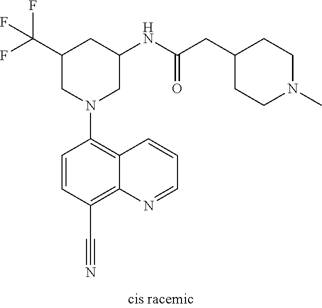 [SMILES: CN1CCC(CC(=O)NC2CC(C(F)(F)F)CN(c3ccc(C#N)c4ncccc34)C2)CC1] | 478 |
| 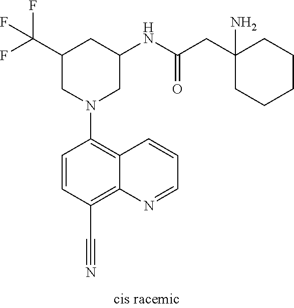 [SMILES: N#Cc1ccc(N2CC(NC(=O)CC3(N)CCCCC3)CC(C(F)(F)F)C2)c2cccnc12] | 479 |
| 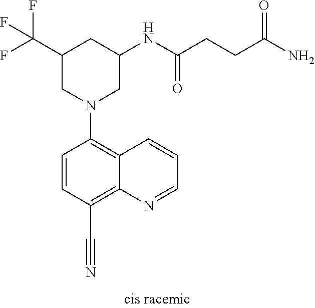 [SMILES: N#Cc1ccc(N2CC(NC(=O)CCC(N)=O)CC(C(F)(F)F)C2)c2cccnc12] | 480 |
| 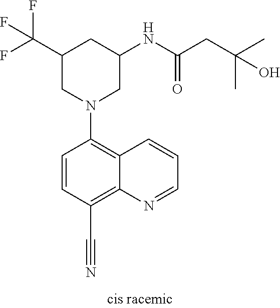 [SMILES: CC(C)(O)CC(=O)NC1CC(C(F)(F)F)CN(c2ccc(C#N)c3ncccc23)C1] | 481 |
| 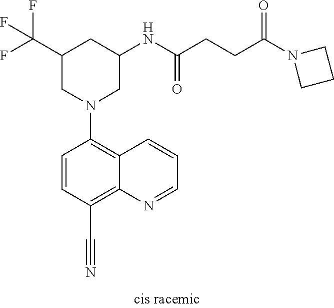 [SMILES: N#Cc1ccc(N2CC(NC(=O)CCC(=O)N3CCC3)CC(C(F)(F)F)C2)c2cccnc12] | 482 |
| 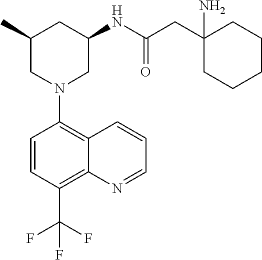 [SMILES: C[C@H]1C[C@@H](NC(=O)CC2(N)CCCCC2)CN(c2ccc(C(F)(F)F)c3ncccc23)C1] | 483 |
| 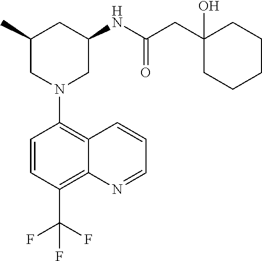 [SMILES: C[C@H]1C[C@@H](NC(=O)CC2(O)CCCCC2)CN(c2ccc(C(F)(F)F)c3ncccc23)C1] | 484 |
| 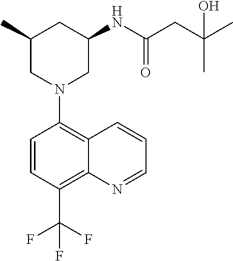 [SMILES: C[C@H]1C[C@@H](NC(=O)CC(C)(C)O)CN(c2ccc(C(F)(F)F)c3ncccc23)C1] | 485 |
| 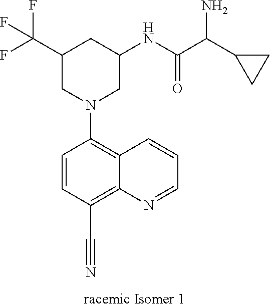 [SMILES: N#Cc1ccc(N2CC(NC(=O)C(N)C3CC3)CC(C(F)(F)F)C2)c2cccnc12] | 486 |
| 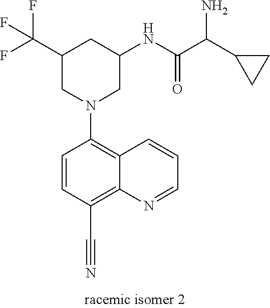 [SMILES: N#Cc1ccc(N2CC(NC(=O)C(N)C3CC3)CC(C(F)(F)F)C2)c2cccnc12] | 487 |
| 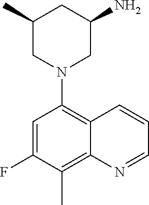 [SMILES: Cc1c(F)cc(N2C[C@@H](C)C[C@@H](N)C2)c2cccnc12] | 488 |
| 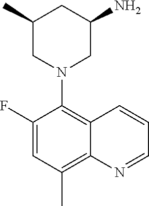 [SMILES: Cc1cc(F)c(N2C[C@@H](C)C[C@@H](N)C2)c2cccnc12] | 489 |
| 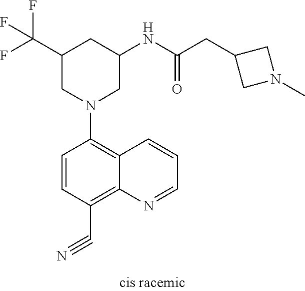 [SMILES: CN1CC(CC(=O)NC2CC(C(F)(F)F)CN(c3ccc(C#N)c4ncccc34)C2)C1] | 490 |
| 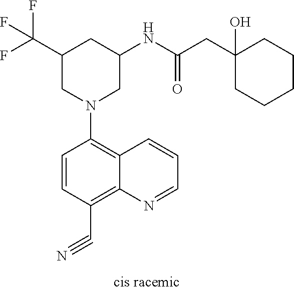 [SMILES: N#Cc1ccc(N2CC(NC(=O)CC3(O)CCCCC3)CC(C(F)(F)F)C2)c2cccnc12] | 491 |
| 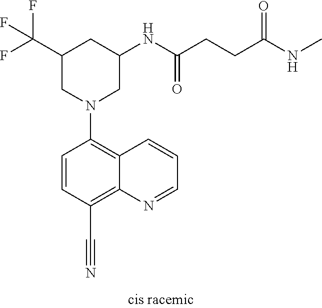 [SMILES: CNC(=O)CCC(=O)NC1CC(C(F)(F)F)CN(c2ccc(C#N)c3ncccc23)C1] | 492 |
| 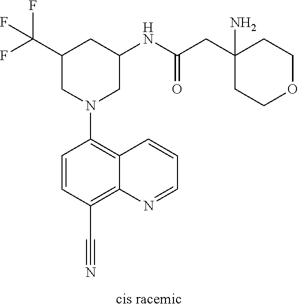 [SMILES: N#Cc1ccc(N2CC(NC(=O)CC3(N)CCOCC3)CC(C(F)(F)F)C2)c2cccnc12] | 493 |
| 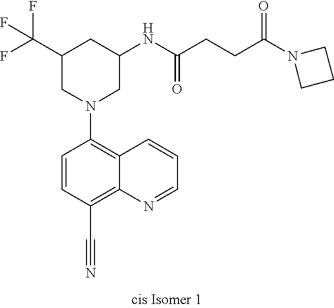 [SMILES: N#Cc1ccc(N2CC(NC(=O)CCC(=O)N3CCC3)CC(C(F)(F)F)C2)c2cccnc12] | 494 |
| 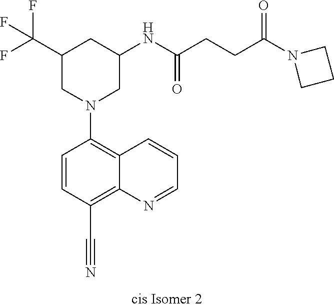 [SMILES: N#Cc1ccc(N2CC(NC(=O)CCC(=O)N3CCC3)CC(C(F)(F)F)C2)c2cccnc12] | 495 |
| 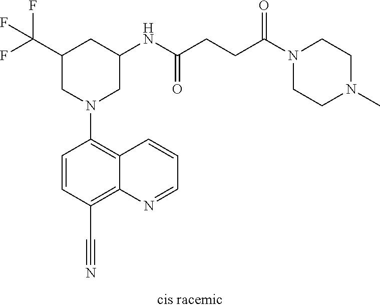 [SMILES: CN1CCN(C(=O)CCC(=O)NC2CC(C(F)(F)F)CN(c3ccc(C#N)c4ncccc34)C2)CC1] | 496 |
| 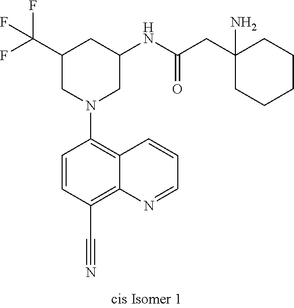 [SMILES: N#Cc1ccc(N2CC(NC(=O)CC3(N)CCCCC3)CC(C(F)(F)F)C2)c2cccnc12] | 497 |
| 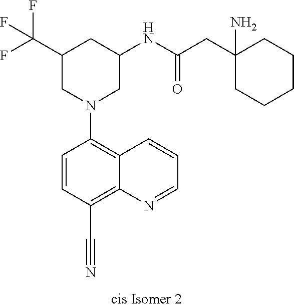 [SMILES: N#Cc1ccc(N2CC(NC(=O)CC3(N)CCCCC3)CC(C(F)(F)F)C2)c2cccnc12] | 498 |
| 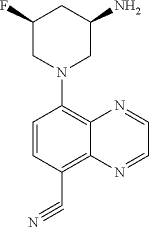 [SMILES: N#Cc1ccc(N2C[C@H](N)C[C@H](F)C2)c2nccnc12] | 499 |
| 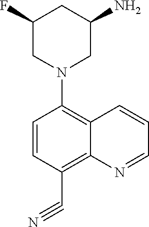 [SMILES: N#Cc1ccc(N2C[C@H](N)C[C@H](F)C2)c2cccnc12] | 500 |
| 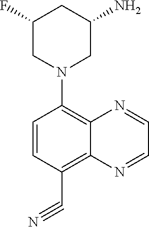 [SMILES: N#Cc1ccc(N2C[C@@H](N)C[C@@H](F)C2)c2nccnc12] | 501 |
| 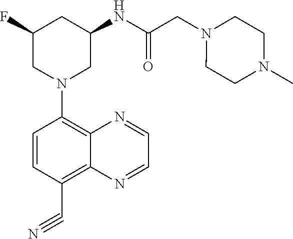 [SMILES: CN1CCN(CC(=O)N[C@@H]2C[C@H](F)CN(c3ccc(C#N)c4nccnc34)C2)CC1] | 502 |
| 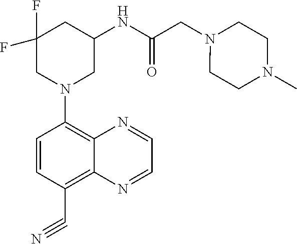 [SMILES: CN1CCN(CC(=O)NC2CN(c3ccc(C#N)c4nccnc34)CC(F)(F)C2)CC1] | 503 |
| 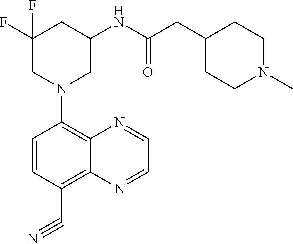 [SMILES: CN1CCC(CC(=O)NC2CN(c3ccc(C#N)c4nccnc34)CC(F)(F)C2)CC1] | 504 |
| 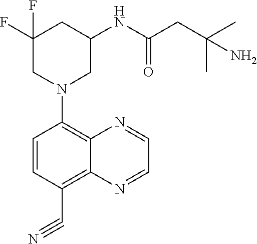 [SMILES: CC(C)(N)CC(=O)NC1CN(c2ccc(C#N)c3nccnc23)CC(F)(F)C1] | 505 |
| 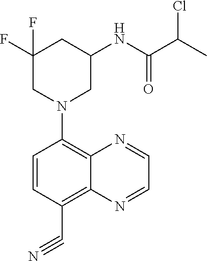 [SMILES: CC(Cl)C(=O)NC1CN(c2ccc(C#N)c3nccnc23)CC(F)(F)C1] | 506 |
| 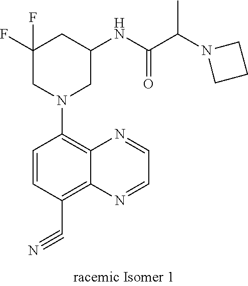 [SMILES: CC(C(=O)NC1CN(c2ccc(C#N)c3nccnc23)CC(F)(F)C1)N1CCC1] | 507 |
| 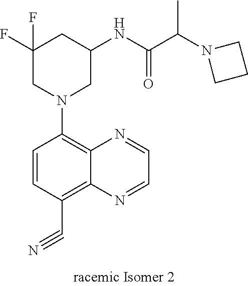 [SMILES: CC(C(=O)NC1CN(c2ccc(C#N)c3nccnc23)CC(F)(F)C1)N1CCC1] | 508 |
| 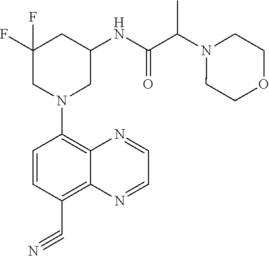 [SMILES: CC(C(=O)NC1CN(c2ccc(C#N)c3nccnc23)CC(F)(F)C1)N1CCOCC1] | 509 |
| 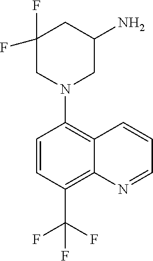 [SMILES: NC1CN(c2ccc(C(F)(F)F)c3ncccc23)CC(F)(F)C1] | 510 |
| 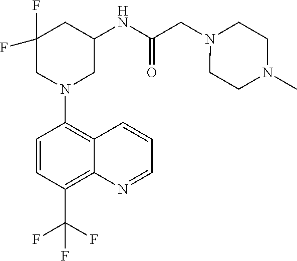 [SMILES: CN1CCN(CC(=O)NC2CN(c3ccc(C(F)(F)F)c4ncccc34)CC(F)(F)C2)CC1] | 511 |
| 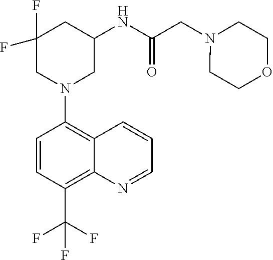 [SMILES: O=C(CN1CCOCC1)NC1CN(c2ccc(C(F)(F)F)c3ncccc23)CC(F)(F)C1] | 512 |
| 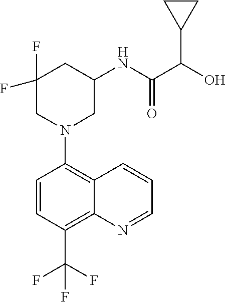 [SMILES: O=C(NC1CN(c2ccc(C(F)(F)F)c3ncccc23)CC(F)(F)C1)C(O)C1CC1] | 513 |
| 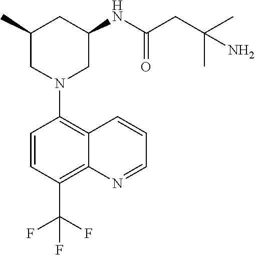 [SMILES: C[C@H]1C[C@@H](NC(=O)CC(C)(C)N)CN(c2ccc(C(F)(F)F)c3ncccc23)C1] | 514 |
| 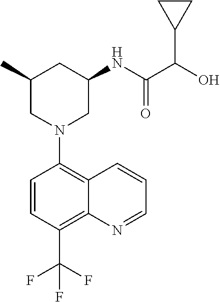 [SMILES: C[C@H]1C[C@@H](NC(=O)C(O)C2CC2)CN(c2ccc(C(F)(F)F)c3ncccc23)C1] | 515 |
| 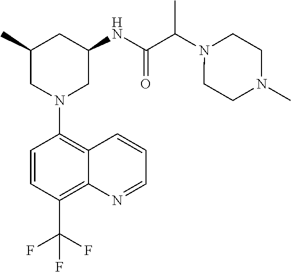 | 516 |
| 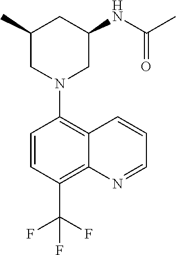 [SMILES: CC(=O)N[C@@H]1C[C@H](C)CN(c2ccc(C(F)(F)F)c3ncccc23)C1] | 517 |
| 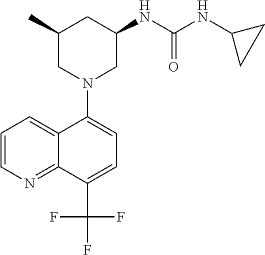 [SMILES: C[C@H]1C[C@@H](NC(=O)NC2CC2)CN(c2ccc(C(F)(F)F)c3ncccc23)C1] | 518 |
| 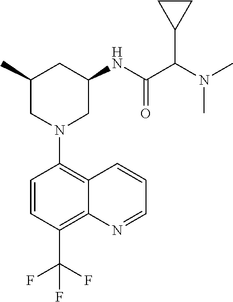 | 519 |
| 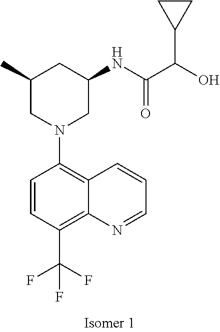 [SMILES: C[C@H]1C[C@@H](NC(=O)C(O)C2CC2)CN(c2ccc(C(F)(F)F)c3ncccc23)C1] | 520 |
| 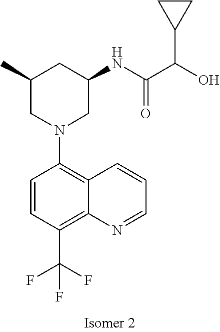 [SMILES: C[C@H]1C[C@@H](NC(=O)C(O)C2CC2)CN(c2ccc(C(F)(F)F)c3ncccc23)C1] | 521 |
| 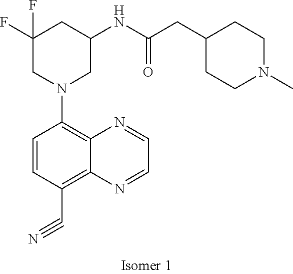 [SMILES: CN1CCC(CC(=O)NC2CN(c3ccc(C#N)c4nccnc34)CC(F)(F)C2)CC1] | 522 |
| 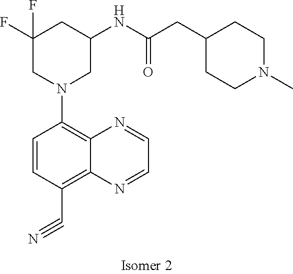 [SMILES: CN1CCC(CC(=O)NC2CN(c3ccc(C#N)c4nccnc34)CC(F)(F)C2)CC1] | 523 |
| 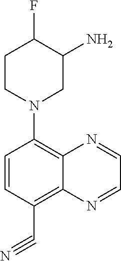 [SMILES: N#Cc1ccc(N2CCC(F)C(N)C2)c2nccnc12] | 524 |
| 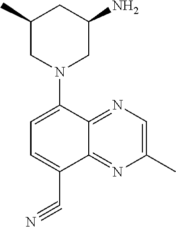 | 525 |
| 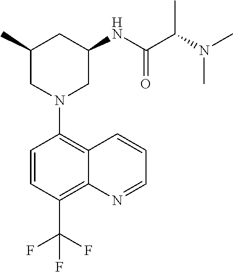 | 526 |
| 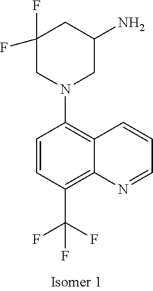 [SMILES: NC1CN(c2ccc(C(F)(F)F)c3ncccc23)CC(F)(F)C1] | 527 |
| 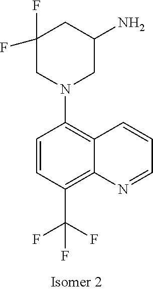 [SMILES: NC1CN(c2ccc(C(F)(F)F)c3ncccc23)CC(F)(F)C1] | 528 |
| 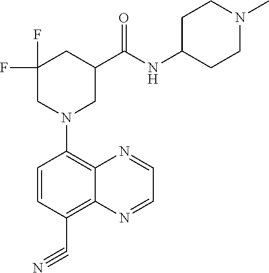 [SMILES: CN1CCC(NC(=O)C2CN(c3ccc(C#N)c4nccnc34)CC(F)(F)C2)CC1] | 529 |
| 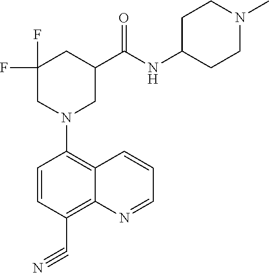 [SMILES: CN1CCC(NC(=O)C2CN(c3ccc(C#N)c4ncccc34)CC(F)(F)C2)CC1] | 530 |
| 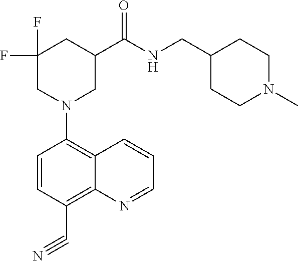 [SMILES: CN1CCC(CNC(=O)C2CN(c3ccc(C#N)c4ncccc34)CC(F)(F)C2)CC1] | 531 |
| 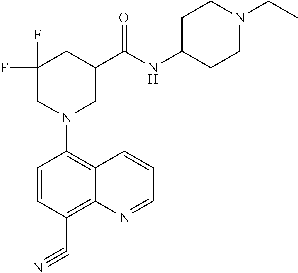 [SMILES: CCN1CCC(NC(=O)C2CN(c3ccc(C#N)c4ncccc34)CC(F)(F)C2)CC1] | 532 |
| 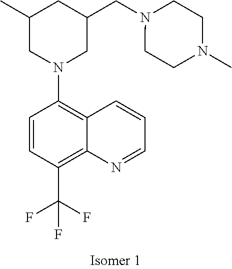 | 533 |
| 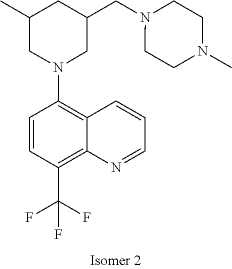 | 534 |
| 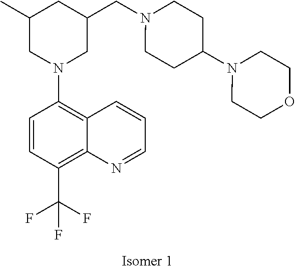 [SMILES: CC1CC(CN2CCC(N3CCOCC3)CC2)CN(c2ccc(C(F)(F)F)c3ncccc23)C1] | 535 |
| 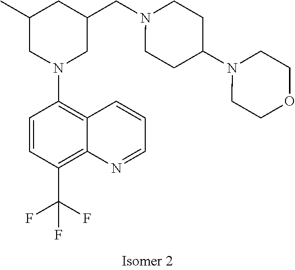 [SMILES: CC1CC(CN2CCC(N3CCOCC3)CC2)CN(c2ccc(C(F)(F)F)c3ncccc23)C1] | 536 |
| 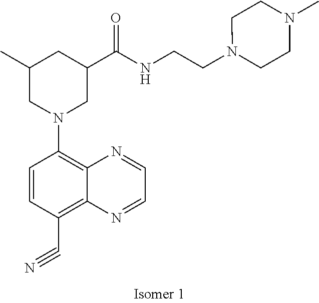 | 537 |
| 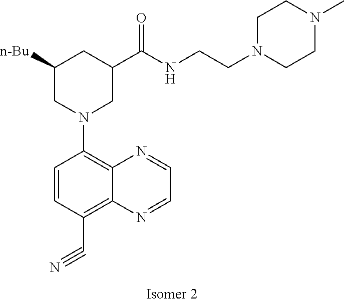 | 538 |
| 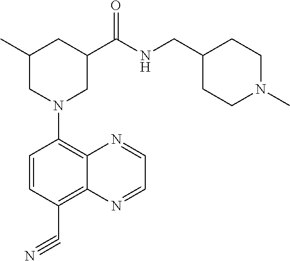 | 539 |
| 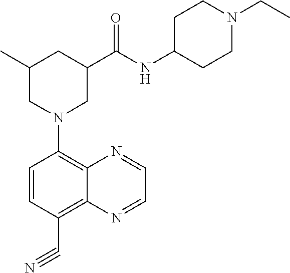 [SMILES: CCN1CCC(NC(=O)C2CC(C)CN(c3ccc(C#N)c4nccnc34)C2)CC1] | 540 |

6. The method according to claim 1, wherein the compound is selected from the group consisting of a compound shown in the following Table:

| | |
|---|---|
| 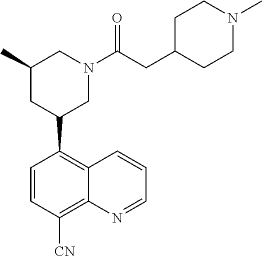 | Compound 1 |
| 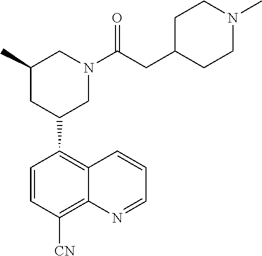 | Compound 2 |
| 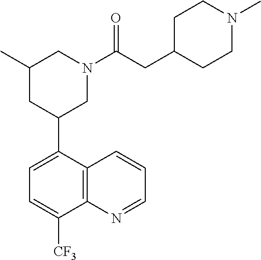 | Compound 3 |
| 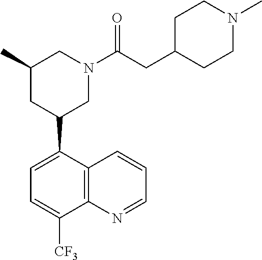 | Compound 4 |
| 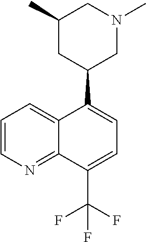 [SMILES: C[C@@H]1C[C@H](c2ccc(C(F)(F)F)c3ncccc23)CN(C)C1] | Compound 5 |
| 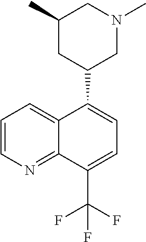 | Compound 6 |
| 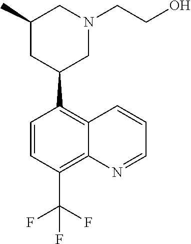 [SMILES: C[C@@H]1C[C@H](c2ccc(C(F)(F)F)c3ncccc23)CN(CCO)C1] | Compound 7 |
| 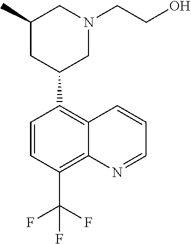 [SMILES: C[C@@H]1C[C@@H](c2ccc(C(F)(F)F)c3ncccc23)CN(CCO)C1] | Compound 8 |
| 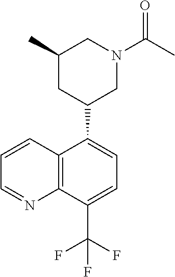 | Compound 9 |
| 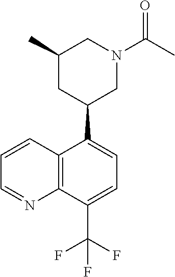 [SMILES: CC(=O)N1C[C@H](C)C[C@H](c2ccc(C(F)(F)F)c3ncccc23)C1] | Compound 10 |
| 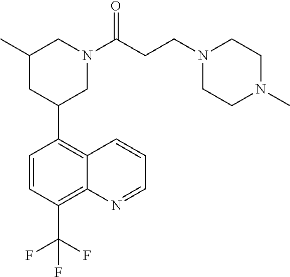 | Compound 11 |
| 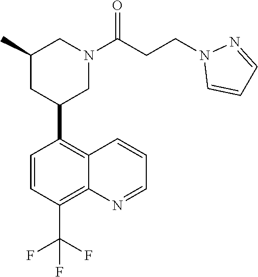 | Compound 12 |
| 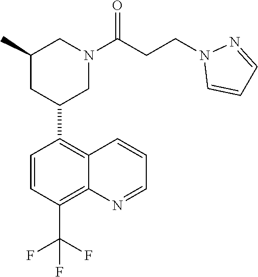 | Compound 13 |
| 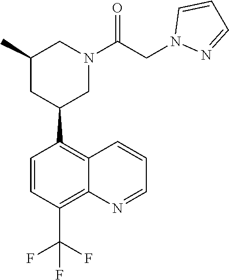 [SMILES: C[C@@H]1C[C@H](c2ccc(C(F)(F)F)c3ncccc23)CN(C(=O)Cn2cccn2)C1] | Compound 14 |
| 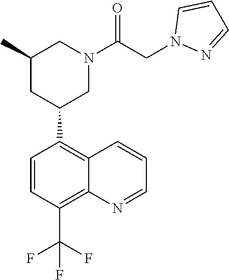 [SMILES: C[C@@H]1C[C@@H](c2ccc(C(F)(F)F)c3ncccc23)CN(C(=O)Cn2cccn2)C1] | Compound 15 |
| 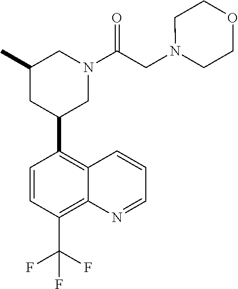 | Compound 16 |
| 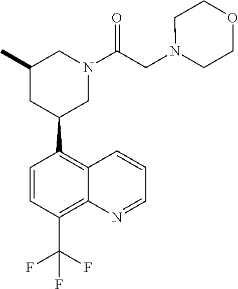 [SMILES: C[C@@H]1C[C@H](c2ccc(C(F)(F)F)c3ncccc23)CN(C(=O)CN2CCOCC2)C1] | Compound 17 |
| 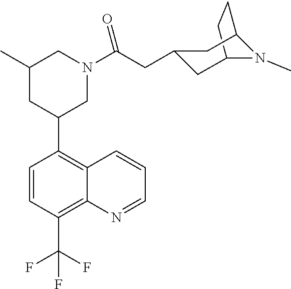 | Compound 18 |
| 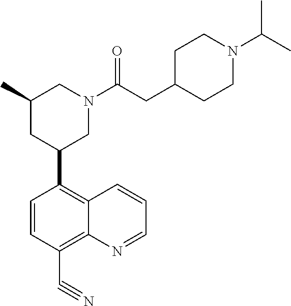 | Compound 19 |
| 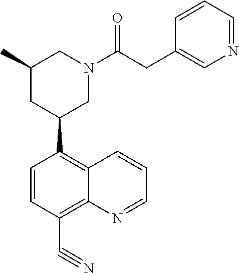 [SMILES: C[C@@H]1C[C@H](c2ccc(C#N)c3ncccc23)CN(C(=O)Cc2cccnc2)C1] | Compound 20 |
| 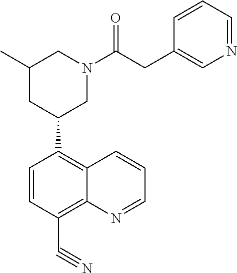 [SMILES: CC1C[C@@H](c2ccc(C#N)c3ncccc23)CN(C(=O)Cc2cccnc2)C1] | Compound 21 |
| 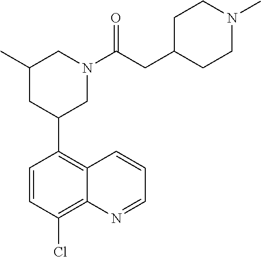 | Compound 22 |
| 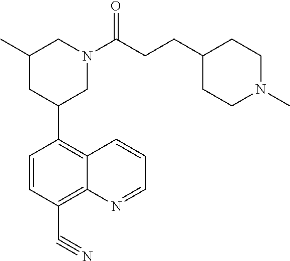 | Compound 23 |
| 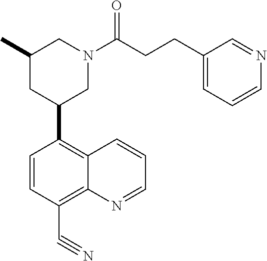 | Compound 24 |
| 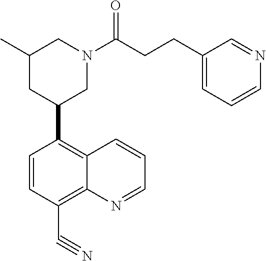 | Compound 25 |
| 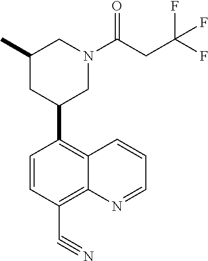 | Compound 26 |
| 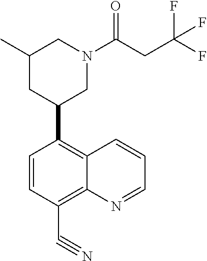 | Compound 27 |
| 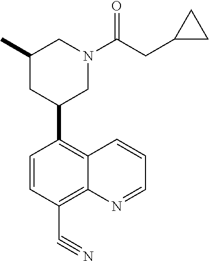 | Compound 28 |
| 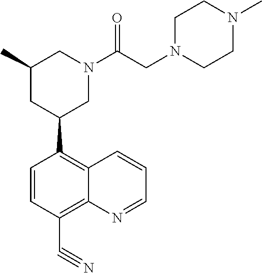 | Compound 29 |
| 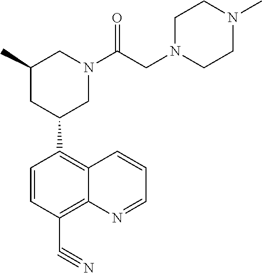 | Compound 30 |
| 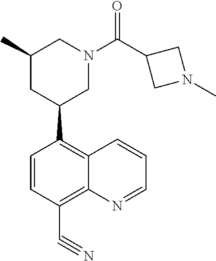 | Compound 31 |
| 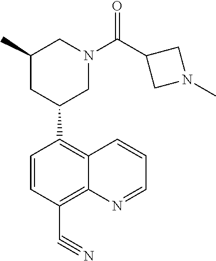 | Compound 32 |
| 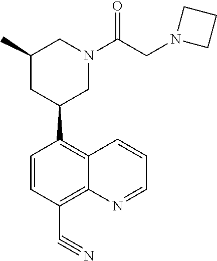 [SMILES: C[C@@H]1C[C@H](c2ccc(C#N)c3ncccc23)CN(C(=O)CN2CCC2)C1] | Compound 33 |
| 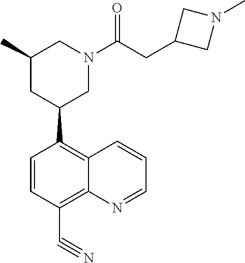 | Compound 34 |
| 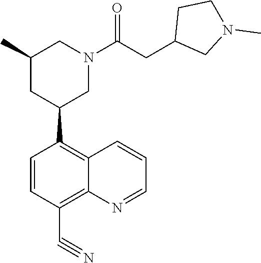 | Compound 35 |
| 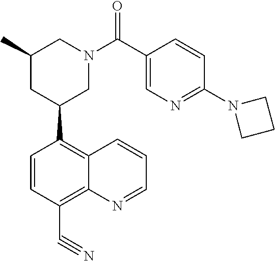 [SMILES: C[C@@H]1C[C@H](c2ccc(C#N)c3ncccc23)CN(C(=O)c2ccc(N3CCC3)nc2)C1] | Compound 36 |
| 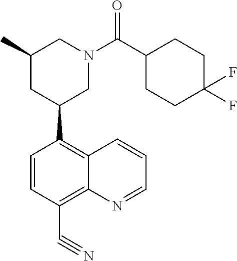 [SMILES: C[C@@H]1C[C@H](c2ccc(C#N)c3ncccc23)CN(C(=O)C2CCC(F)(F)CC2)C1] | Compound 37 |
| 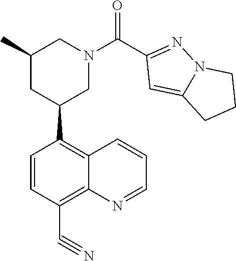 [SMILES: C[C@@H]1C[C@H](c2ccc(C#N)c3ncccc23)CN(C(=O)c2cc3n(n2)CCC3)C1] | Compound 38 |
| 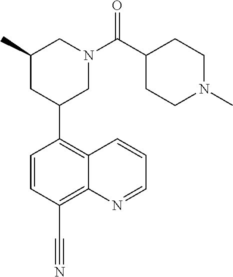 | Compound 39 |
| 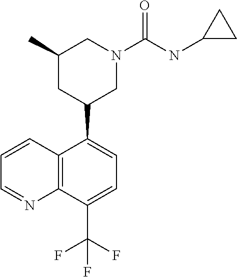 | Compound 40 |
| 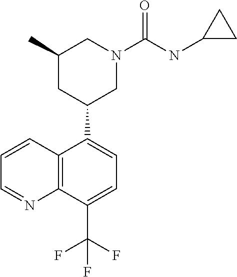 [SMILES: C[C@@H]1C[C@@H](c2ccc(C(F)(F)F)c3ncccc23)CN(C(=O)NC2CC2)C1] | Compound 41 |
| 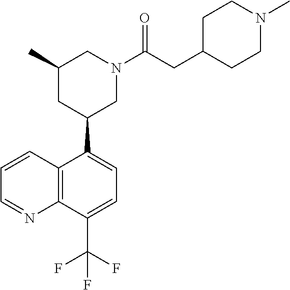 [SMILES: C[C@@H]1C[C@H](c2ccc(C(F)(F)F)c3ncccc23)CN(C(=O)CC2CCN(C)CC2)C1] | Compound 42 |
| 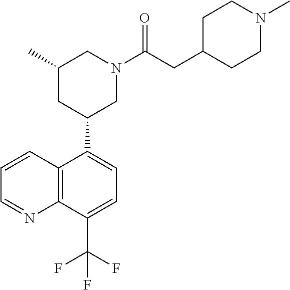 [SMILES: C[C@H]1C[C@@H](c2ccc(C(F)(F)F)c3ncccc23)CN(C(=O)CC2CCN(C)CC2)C1] | Compound 43 |
| 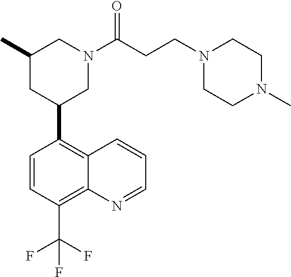 | Compound 44 |
| 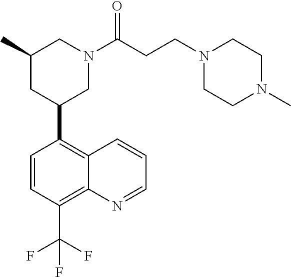 | Compound 45 |
| 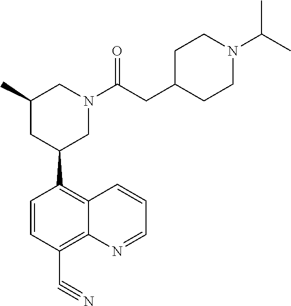 [SMILES: CC(C)N1CCC(CC(=O)N2C[C@H](C)C[C@H](c3ccc(C#N)c4ncccc34)C2)CC1] | Compound 46 |
| 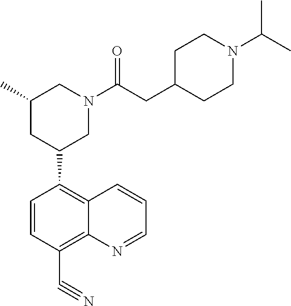 | Compound 47 |
| 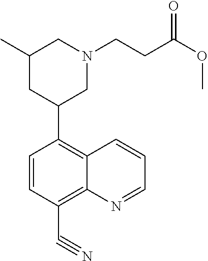 | Compound 48 |
| 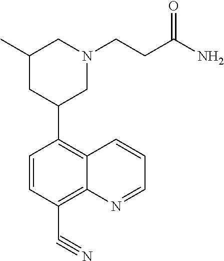 [SMILES: CC1CC(c2ccc(C#N)c3ncccc23)CN(CCC(N)=O)C1] | Compound 49 |
| 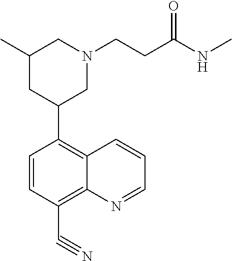 | Compound 50 |
| 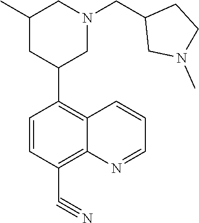 [SMILES: CC1CC(c2ccc(C#N)c3ncccc23)CN(CC2CCN(C)C2)C1] | Compound 51 |
| 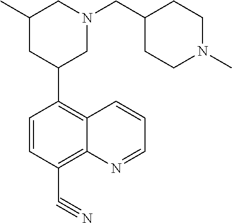 | Compound 52 |
| 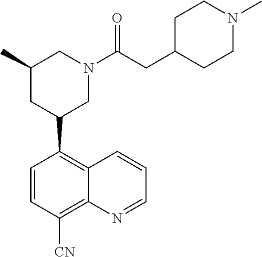 | Compound 53 |
| 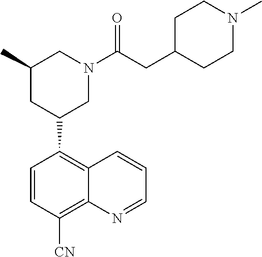 | Compound 54 |
| 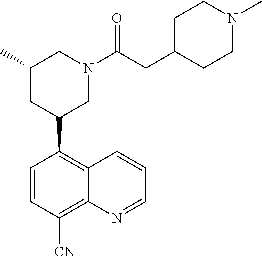 | Compound 55 |
| 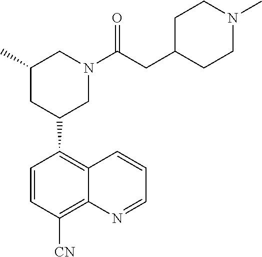 | Compound 56 |
| 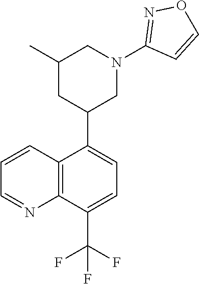 [SMILES: CC1CC(c2ccc(C(F)(F)F)c3ncccc23)CN(c2ccon2)C1] | Compound 57 |
| 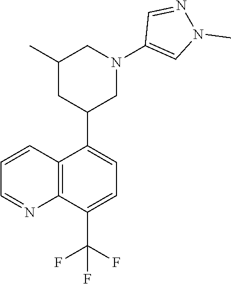 [SMILES: CC1CC(c2ccc(C(F)(F)F)c3ncccc23)CN(c2cnn(C)c2)C1] | Compound 58 |
| 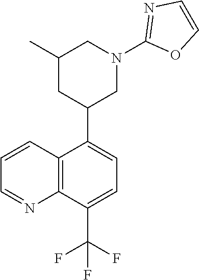 [SMILES: CC1CC(c2ccc(C(F)(F)F)c3ncccc23)CN(c2ncco2)C1] | Compound 59 |
| 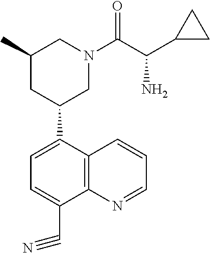 [SMILES: C[C@@H]1C[C@@H](c2ccc(C#N)c3ncccc23)CN(C(=O)[C@@H](N)C2CC2)C1] | Compound 60 |
| 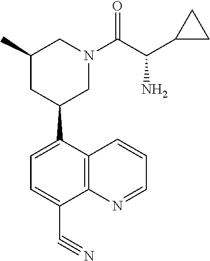 [SMILES: C[C@@H]1C[C@H](c2ccc(C#N)c3ncccc23)CN(C(=O)[C@@H](N)C2CC2)C1] | Compound 61 |
| 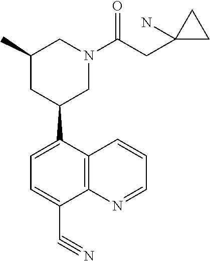 [SMILES: C[C@@H]1C[C@H](c2ccc(C#N)c3ncccc23)CN(C(=O)CC2(N)CC2)C1] | Compound 62 |
| 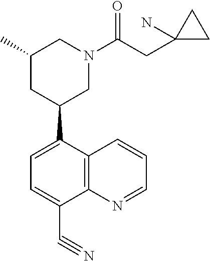 [SMILES: C[C@H]1C[C@H](c2ccc(C#N)c3ncccc23)CN(C(=O)CC2(N)CC2)C1] | Compound 63 |
| 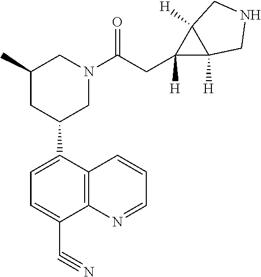 [SMILES: C[C@@H]1C[C@@H](c2ccc(C#N)c3ncccc23)CN(C(=O)C[C@H]2[C@@H]3CNC[C@@H]32)C1] | Compound 64 |
| 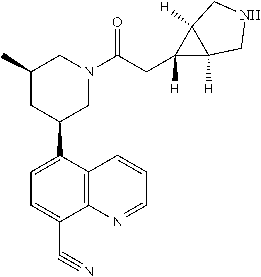 [SMILES: C[C@@H]1C[C@H](c2ccc(C#N)c3ncccc23)CN(C(=O)C[C@H]2[C@@H]3CNC[C@@H]32)C1] | Compound 65 |
| 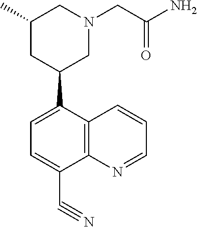 [SMILES: C[C@H]1C[C@H](c2ccc(C#N)c3ncccc23)CN(CC(N)=O)C1] | Compound 66 |
| 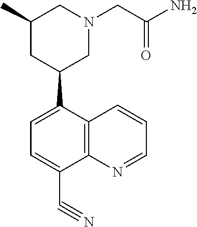 [SMILES: C[C@@H]1C[C@H](c2ccc(C#N)c3ncccc23)CN(CC(N)=O)C1] | Compound 67 |
| 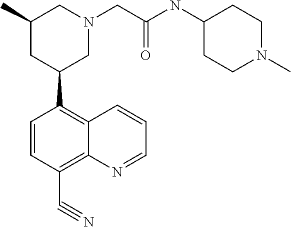 | Compound 68 |
| 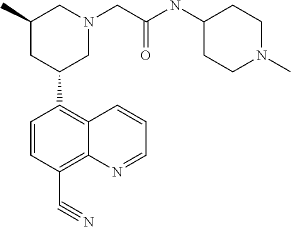 | Compound 69 |
| 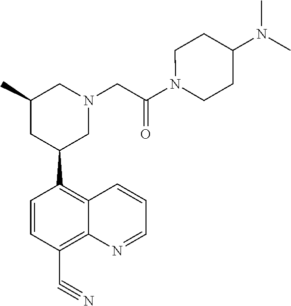 | Compound 70 |
| 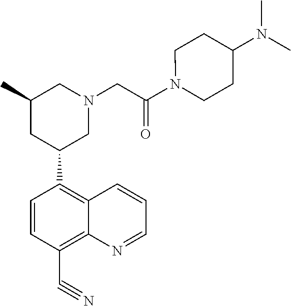 | Compound 71 |
| 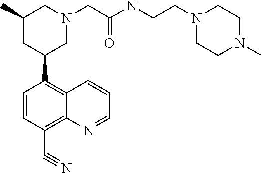 | Compound 72 |
| 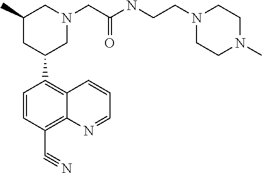 | Compound 73 |
| 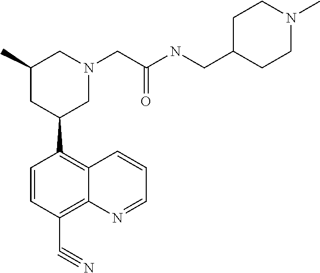 | Compound 74 |
| 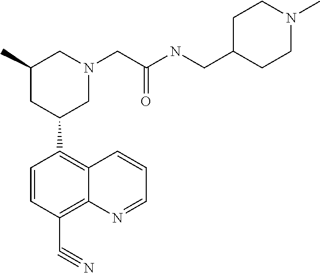 | Compound 75 |
| 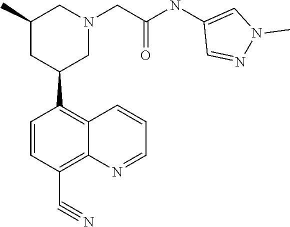 | Compound 76 |
| 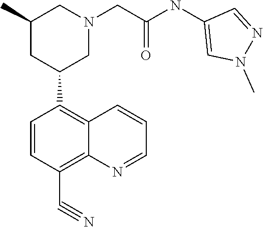 [SMILES: C[C@@H]1C[C@@H](c2ccc(C#N)c3ncccc23)CN(CC(=O)Nc2cnn(C)c2)C1] | Compound 77 |
| 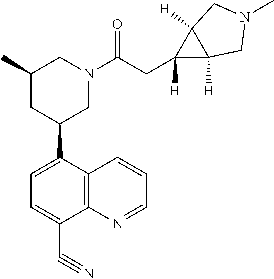 | Compound 78 |
| 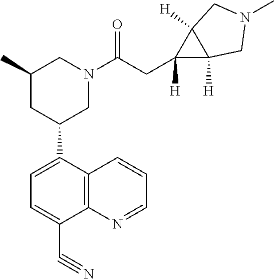 | Compound 79 |
| 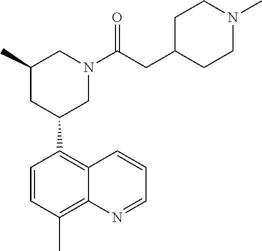 | Compound 80 |
| 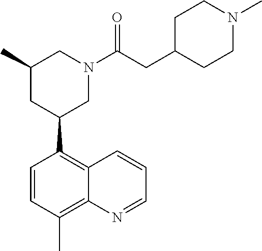 | Compound 81 |
| 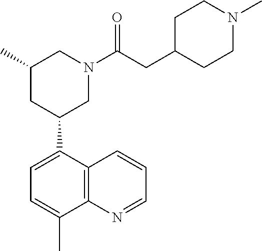 | Compound 82 |
| 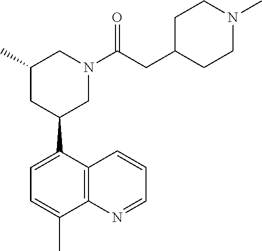 | Compound 83 |
| 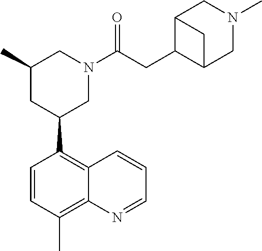 | Compound 84 |
| 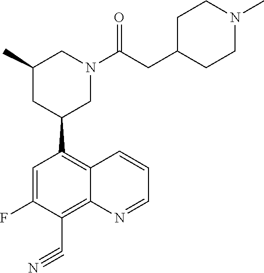 | Compound 85 |
| 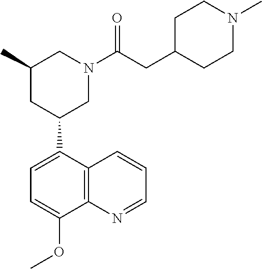 | Compound 86 |
| 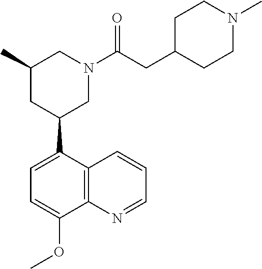 | Compound 87 |
| 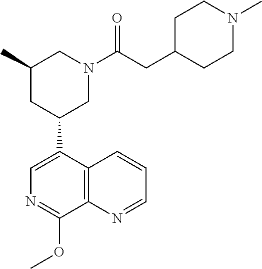 | Compound 88 |
| 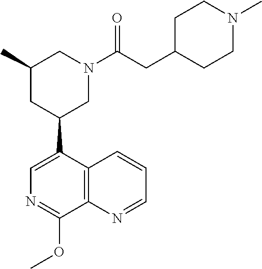 | Compound 89 |
| 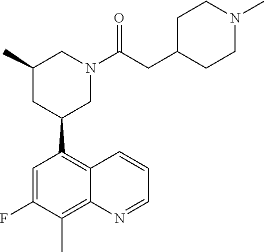 | Compound 90 |
| 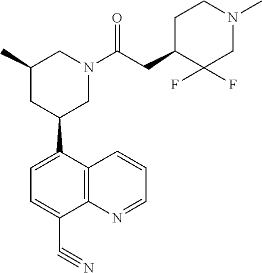 | Compound 91 |
| 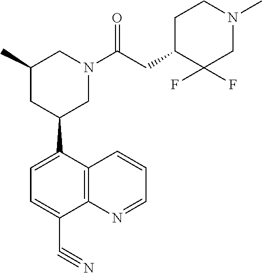 | Compound 92 |
| 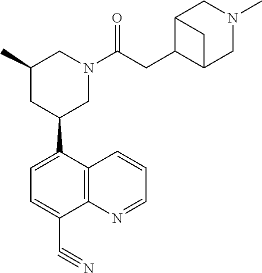 | Compound 93 |
| 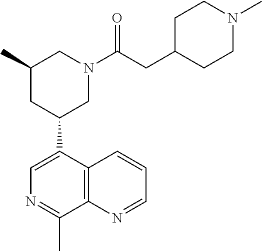 | Compound 94 |
| 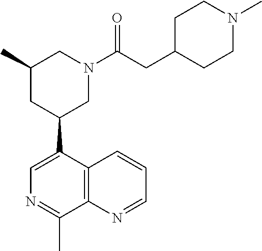 | Compound 95 |
| 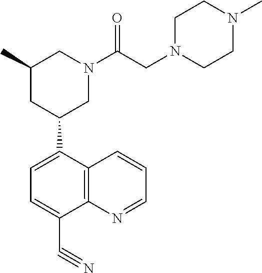 | Compound 96 |
| 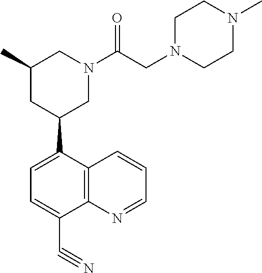 | Compound 97 |
| 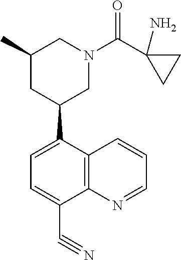 [SMILES: C[C@@H]1C[C@H](c2ccc(C#N)c3ncccc23)CN(C(=O)C2(N)CC2)C1] | Compound 98 |
| 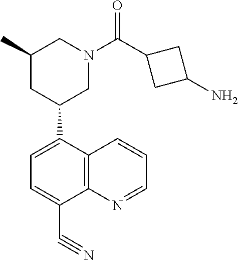 [SMILES: C[C@@H]1C[C@@H](c2ccc(C#N)c3ncccc23)CN(C(=O)C2CC(N)C2)C1] | Compound 99 |
| 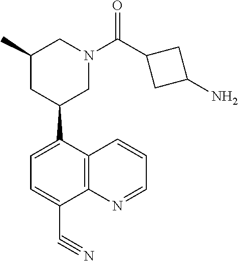 [SMILES: C[C@@H]1C[C@H](c2ccc(C#N)c3ncccc23)CN(C(=O)C2CC(N)C2)C1] | Compound 100 |
| 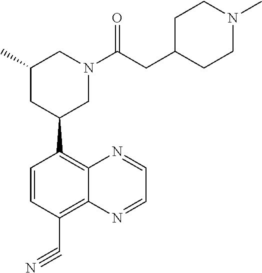 | Compound 101 |
| 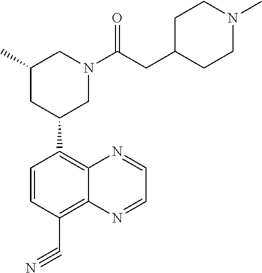 | Compound 102 |
| 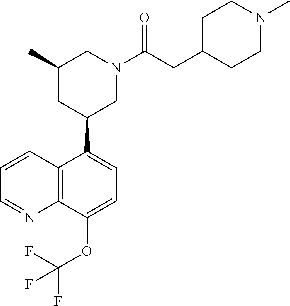 [SMILES: C[C@@H]1C[C@H](c2ccc(OC(F)(F)F)c3ncccc23)CN(C(=O)CC2CCN(C)CC2)C1] | Compound 103 |
| 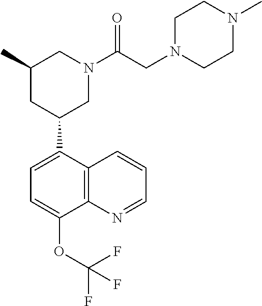 | Compound 104 |
| 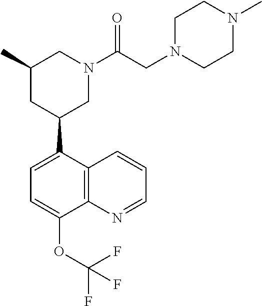 | Compound 105 |
| 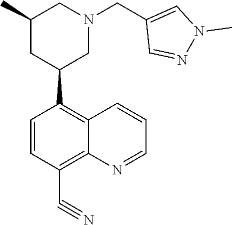 | Compound 106 |
| 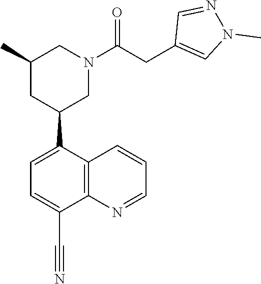 | Compound 107 |
| 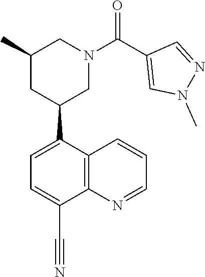 [SMILES: C[C@@H]1C[C@H](c2ccc(C#N)c3ncccc23)CN(C(=O)c2cnn(C)c2)C1] | Compound 108 |
| 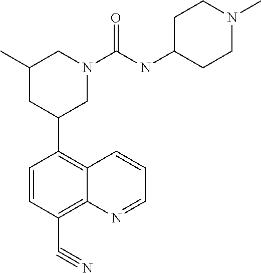 | Compound 109 |
| 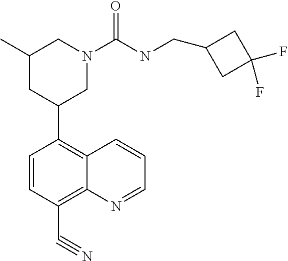 [SMILES: CC1CC(c2ccc(C#N)c3ncccc23)CN(C(=O)NCC2CC(F)(F)C2)C1] | Compound 110 |
| 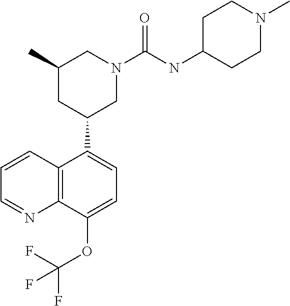 [SMILES: C[C@@H]1C[C@@H](c2ccc(OC(F)(F)F)c3ncccc23)CN(C(=O)NC2CCN(C)CC2)C1] | Compound 111 |
| 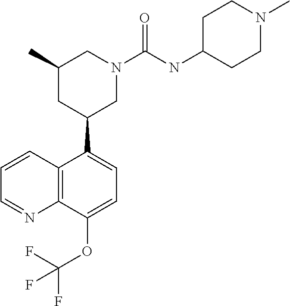 [SMILES: C[C@@H]1C[C@H](c2ccc(OC(F)(F)F)c3ncccc23)CN(C(=O)NC2CCN(C)CC2)C1] | Compound 112 |
| 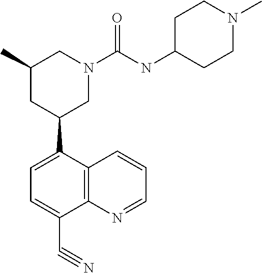 | Compound 113 |
| 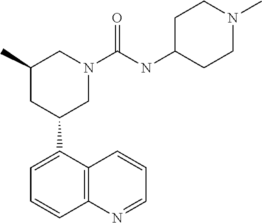 | Compound 114 |
| 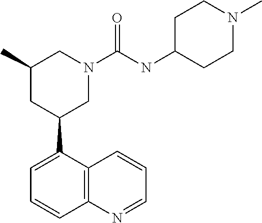 | Compound 115 |
| 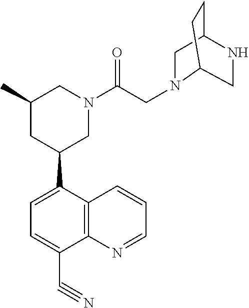 [SMILES: C[C@@H]1C[C@H](c2ccc(C#N)c3ncccc23)CN(C(=O)CN2CC3CCC2CN3)C1] | Compound 116 |
| 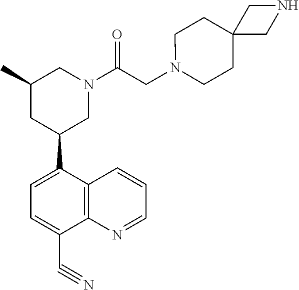 [SMILES: C[C@@H]1C[C@H](c2ccc(C#N)c3ncccc23)CN(C(=O)CN2CCC3(CC2)CNC3)C1] | Compound 117 |
| 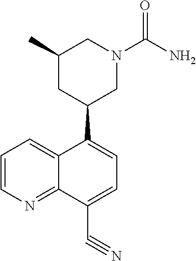 [SMILES: C[C@@H]1C[C@H](c2ccc(C#N)c3ncccc23)CN(C(N)=O)C1] | Compound 118 |
| 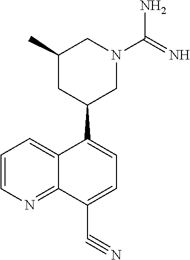 [SMILES: C[C@@H]1C[C@H](c2ccc(C#N)c3ncccc23)CN(C(=N)N)C1] | Compound 119 |
| 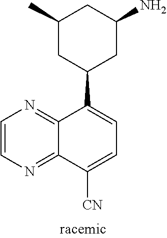 [SMILES: C[C@H]1C[C@@H](N)C[C@@H](c2ccc(C#N)c3nccnc23)C1] | Compound 120 |
| 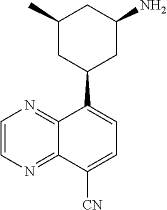 [SMILES: C[C@H]1C[C@@H](N)C[C@@H](c2ccc(C#N)c3nccnc23)C1] | Compound 121 |
| 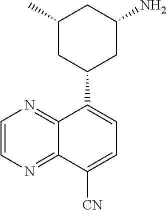 [SMILES: C[C@@H]1C[C@H](N)C[C@H](c2ccc(C#N)c3nccnc23)C1] | Compound 122 |
| 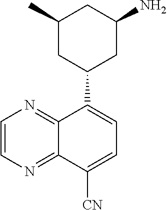 [SMILES: C[C@H]1C[C@@H](N)C[C@H](c2ccc(C#N)c3nccnc23)C1] | Compound 123 |
| 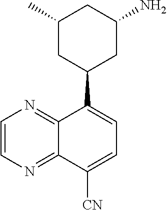 [SMILES: C[C@@H]1C[C@H](N)C[C@@H](c2ccc(C#N)c3nccnc23)C1] | Compound 124 |
| 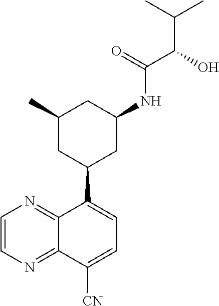 [SMILES: CC(C)[C@H](O)C(=O)N[C@@H]1C[C@H](C)C[C@H](c2ccc(C#N)c3nccnc23)C1] | compound 125 |
| 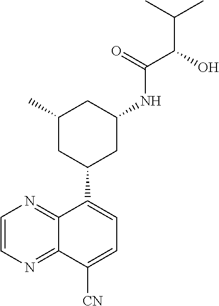 [SMILES: CC(C)[C@H](O)C(=O)N[C@H]1C[C@@H](C)C[C@@H](c2ccc(C#N)c3nccnc23)C1] | compound 126 |
| 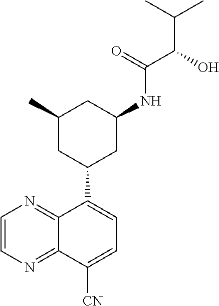 [SMILES: CC(C)[C@H](O)C(=O)N[C@@H]1C[C@H](C)C[C@@H](c2ccc(C#N)c3nccnc23)C1] | Compound 127 |
| 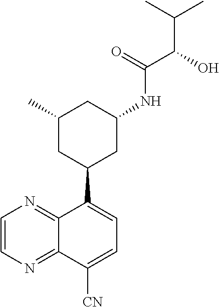 [SMILES: CC(C)[C@H](O)C(=O)N[C@H]1C[C@@H](C)C[C@H](c2ccc(C#N)c3nccnc23)C1] | Compound 128 |
| 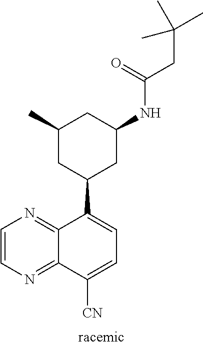 [SMILES: C[C@H]1C[C@@H](NC(=O)CC(C)(C)C)C[C@@H](c2ccc(C#N)c3nccnc23)C1] | Compound 129 |
| 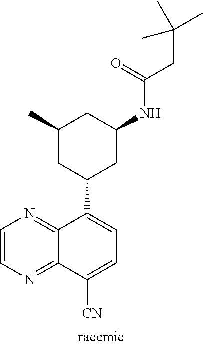 [SMILES: C[C@H]1C[C@@H](NC(=O)CC(C)(C)C)C[C@H](c2ccc(C#N)c3nccnc23)C1] | Compound 130 |
| 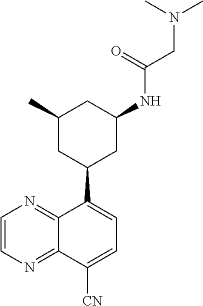 [SMILES: C[C@H]1C[C@@H](NC(=O)CN(C)C)C[C@@H](c2ccc(C#N)c3nccnc23)C1] | Compound 131 |
| 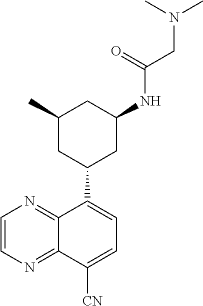 [SMILES: C[C@H]1C[C@@H](NC(=O)CN(C)C)C[C@H](c2ccc(C#N)c3nccnc23)C1] | Compound 132 |
| 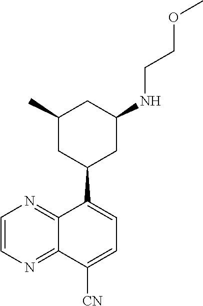 [SMILES: COCCN[C@@H]1C[C@H](C)C[C@H](c2ccc(C#N)c3nccnc23)C1] | Compound 133 |
| 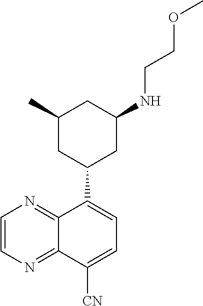 [SMILES: COCCN[C@@H]1C[C@H](C)C[C@@H](c2ccc(C#N)c3nccnc23)C1] | Compound 134 |
| 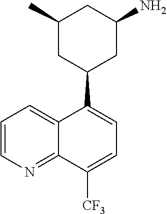 [SMILES: C[C@H]1C[C@@H](N)C[C@@H](c2ccc(C(F)(F)F)c3ncccc23)C1] | Compound 135 |
| 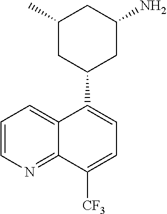 [SMILES: C[C@@H]1C[C@H](N)C[C@H](c2ccc(C(F)(F)F)c3ncccc23)C1] | Compound 136 |
| 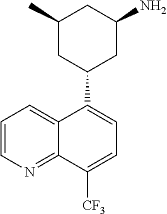 [SMILES: C[C@H]1C[C@@H](N)C[C@H](c2ccc(C(F)(F)F)c3ncccc23)C1] | Compound 137 |
| 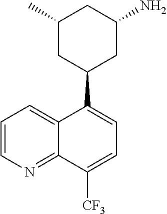 [SMILES: C[C@@H]1C[C@H](N)C[C@@H](c2ccc(C(F)(F)F)c3ncccc23)C1] | Compound 138 |
| 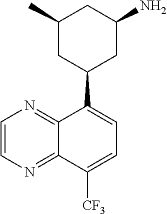 [SMILES: C[C@H]1C[C@@H](N)C[C@@H](c2ccc(C(F)(F)F)c3nccnc23)C1] | Compound 139 |
| 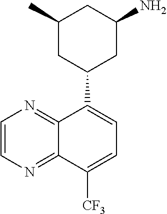 [SMILES: C[C@H]1C[C@@H](N)C[C@H](c2ccc(C(F)(F)F)c3nccnc23)C1] | Compound 140 |
| 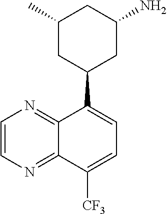 [SMILES: C[C@@H]1C[C@H](N)C[C@@H](c2ccc(C(F)(F)F)c3nccnc23)C1] | Compound 141 |
| 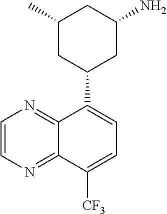 [SMILES: C[C@@H]1C[C@H](N)C[C@H](c2ccc(C(F)(F)F)c3nccnc23)C1] | Compound 142 |
| 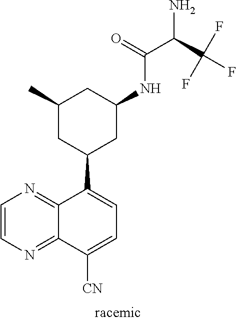 [SMILES: C[C@H]1C[C@@H](NC(=O)[C@@H](N)C(F)(F)F)C[C@@H](c2ccc(C#N)c3nccnc23)C1] | Compound 143 |
| 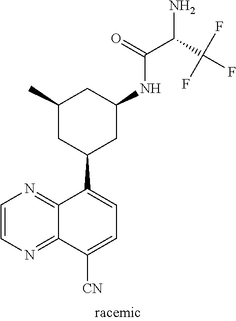 [SMILES: C[C@H]1C[C@@H](NC(=O)[C@H](N)C(F)(F)F)C[C@@H](c2ccc(C#N)c3nccnc23)C1] | Compound 144 |
| 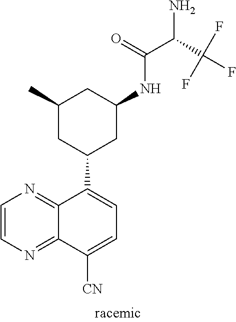 [SMILES: C[C@H]1C[C@@H](NC(=O)[C@H](N)C(F)(F)F)C[C@H](c2ccc(C#N)c3nccnc23)C1] | Compound 145 |
| 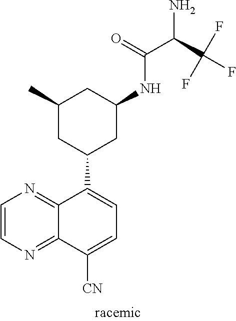 [SMILES: C[C@H]1C[C@@H](NC(=O)[C@@H](N)C(F)(F)F)C[C@H](c2ccc(C#N)c3nccnc23)C1] | Compound 146 |
| 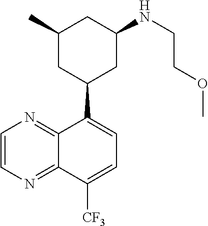 [SMILES: COCCN[C@@H]1C[C@H](C)C[C@H](c2ccc(C(F)(F)F)c3nccnc23)C1] | Compound 147 |
| 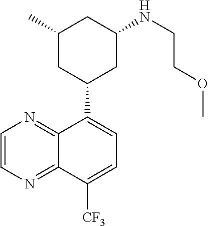 [SMILES: COCCN[C@H]1C[C@@H](C)C[C@@H](c2ccc(C(F)(F)F)c3nccnc23)C1] | Compound 148 |
| 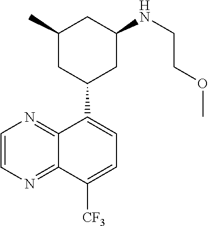 [SMILES: COCCN[C@@H]1C[C@H](C)C[C@@H](c2ccc(C(F)(F)F)c3nccnc23)C1] | Compound 149 |
| 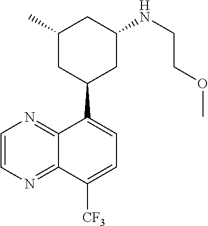 [SMILES: COCCN[C@H]1C[C@@H](C)C[C@H](c2ccc(C(F)(F)F)c3nccnc23)C1] | Compound 150 |
| 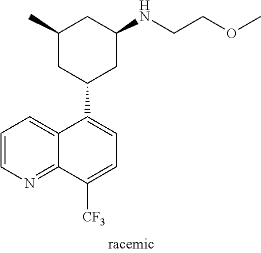 [SMILES: COCCN[C@@H]1C[C@H](C)C[C@@H](c2ccc(C(F)(F)F)c3ncccc23)C1] | Compound 151 |
| 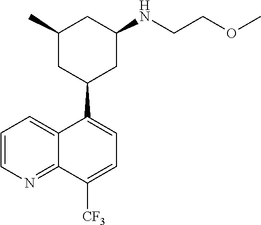 [SMILES: COCCN[C@@H]1C[C@H](C)C[C@H](c2ccc(C(F)(F)F)c3ncccc23)C1] | Compound 152 |
| 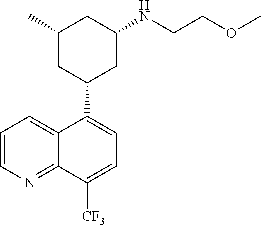 [SMILES: COCCN[C@H]1C[C@@H](C)C[C@@H](c2ccc(C(F)(F)F)c3ncccc23)C1] | Compound 153 |
| 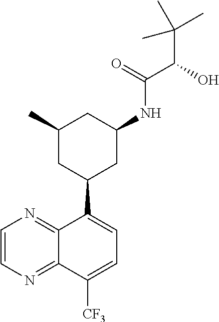 [SMILES: C[C@H]1C[C@@H](NC(=O)[C@@H](O)C(C)(C)C)C[C@@H](c2ccc(C(F)(F)F)c3nccnc23)C1] | Compound 154 |
| 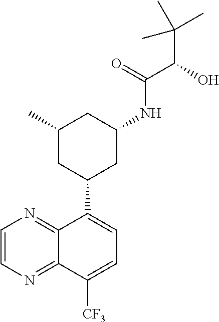 [SMILES: C[C@@H]1C[C@H](NC(=O)[C@@H](O)C(C)(C)C)C[C@H](c2ccc(C(F)(F)F)c3nccnc23)C1] | Compound 155 |
| 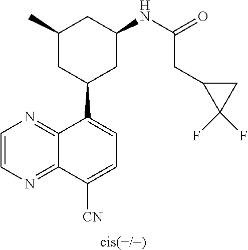 [SMILES: C[C@H]1C[C@@H](NC(=O)CC2CC2(F)F)C[C@@H](c2ccc(C#N)c3nccnc23)C1] | Compound 156 |
| 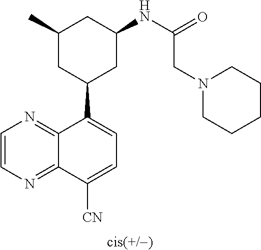 [SMILES: C[C@H]1C[C@@H](NC(=O)CN2CCCCC2)C[C@@H](c2ccc(C#N)c3nccnc23)C1] | Compound 157 |
| 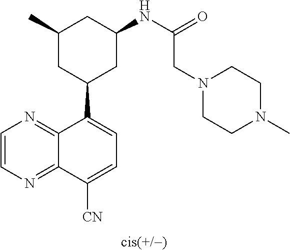 [SMILES: C[C@H]1C[C@@H](NC(=O)CN2CCN(C)CC2)C[C@@H](c2ccc(C#N)c3nccnc23)C1] | Compound 158 |
| 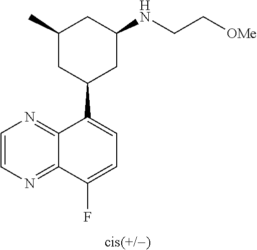 [SMILES: COCCN[C@@H]1C[C@H](C)C[C@H](c2ccc(F)c3nccnc23)C1] | Compound 159 |
| 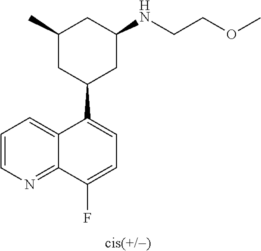 [SMILES: COCCN[C@@H]1C[C@H](C)C[C@H](c2ccc(F)c3ncccc23)C1] | Compound 160 |
